(19) United States
(12) Patent Application Publication
(10) Pub. No.: US 2016/0220569 A1
(43) Pub. Date: Aug. 4, 2016

(54) CDK4/6 Inhibitor Dosage Formulations For The Protection Of Hematopoietic Stem And Progenitor Cells During Chemotherapy

(71) Applicant: G1 Therapeutics, Inc., Research Triangle Park, NC (US)

(72) Inventors: Jay Copeland Strum, Hillsborough, NC (US); John Emerson Bisi, Apex, NC (US); Patrick Joseph Roberts, Durham, NC (US); Jessica Sorrentino, Durham, NC (US); Hannah Storrie-White, Chapel Hill, NC (US)

(73) Assignee: G1 Therapeutics, Inc., Research Triangle Park, NC (US)

(21) Appl. No.: 15/015,070

(22) Filed: Feb. 3, 2016

Related U.S. Application Data
(60) Provisional application No. 62/165,542, filed on May 22, 2015.
(60) Provisional application No. 62/111,573, filed on Feb. 3, 2015.

(51) Int. Cl. A61K 31/519; A61K 9/00; A61K 31/4745; A61K 31/7048; A61K 31/555

(52) U.S. Cl. CPC A61K 31/519; A61K 31/7048; A61K 31/555; A61K 31/4745; A61K 9/0019

(57) ABSTRACT

This invention is in the area of dosage formulations and methods of administering a CDK4/6 inhibitor for the transient protection of healthy cells, and in particular hematopoietic stem and progenitor cells (HSPC), from damage associated with DNA damaging chemotherapeutic agents in subjects undergoing DNA damaging chemotherapeutic therapies for the treatment of proliferative disorders. In one aspect, improved protection of healthy cells is disclosed using a dosage that provides desirable pharmacokinetic and pharmacodynamic characteristics, including AUC, Tmax, Cmax, dosage-corrected AUC, and dosage-corrected Cmax. In another aspect, a method of treating a subject undergoing chemotherapy for the treatment of a CDK 4/6-replication independent cellular proliferation disorder by administering Compound 1 is provided.

### STATEMENT OF RELATED APPLICATIONS

This application claims the benefit of provisional U.S. Application No. 62/111,573 filed Feb. 3, 2015 and provisional U.S. Application No. 62/165,542 filed May 22, 2015, both of which are incorporated by reference herein.

### FIELD OF THE INVENTION

This invention is in the area of dosage formulations and methods of administering a CDK4/6 inhibitor for the transient protection of healthy cells, and in particular hematopoietic stem and progenitor cells (HSPC), from damage associated with DNA damaging chemotherapeutic agents in subjects undergoing DNA damaging chemotherapeutic therapies for the treatment of proliferative disorders. In one aspect, improved protection of healthy cells is disclosed using a dosage that provides desirable pharmacokinetic and pharmacodynamic characteristics, including AUC, Tmax, Cmax, dosage-corrected AUC, and dosage-corrected Cmax.

### BACKGROUND

Myelosuppression continues to represent the major dose-limiting toxicity of cancer chemotherapy, resulting in considerable morbidity along with the potential need to require a reduction in chemotherapy dose intensity, which may compromise disease control and survival. Considerable evidence from prospective and retrospective randomized clinical trials clearly shows that chemotherapy-induced myelosuppression compromises long-term disease control and survival (Lyman, G. H., Chemotherapy dose intensity and quality cancer care (Oncology (Williston Park), 2006. 20(14 Suppl 9): p. 16-25)). Furthermore, treatment regimens for, example, lung, breast, and colorectal cancer recommended in the National Comprehensive Cancer Network guidelines are increasingly associated with significant myelosuppression yet are increasingly recommended for treating early-stage disease as well as advanced-stage or metastatic disease (Smith, R. E., Trends in recommendations for myelosuppressive chemotherapy for the treatment of solid tumors. J Natl Compr Canc Netw, 2006. 4(7): p. 649-58). This trend toward more intensive treatment of patients with toxic chemotherapies creates demand for improved measures to minimize the risk of myelosuppression and complications while optimizing the relative dose-intensity of the chemotherapy.

In addition to bone marrow suppression, chemotherapeutic agents can adversely affect other healthy cells such as renal epithelial cells, resulting potentially in the development of acute kidney injury due to the death of the tubular epithelia. Acute kidney injury can lead to chronic kidney disease, multi-organ failure, sepsis, and death.

One mechanism to minimize myelosuppression, nephrotoxicity, and other chemotherapeutic cytotoxicities is to reduce the planned dose intensity of chemotherapies. Dose reductions or cycle delays, for example treatment holidays, however, diminish the effectiveness and ultimately compromise long-term disease control and survival. These complications often lead to poor treatment outcomes for patients with cancer. In fact, a retrospective study by Socinski et al in the Journal of Clinical Oncology in 2009 showed that out of 908 patients with newly diagnosed small cell lung cancer 455 received etoposide/carboplatin treatment, but only 199 patients had partial responses to treatment and only 1 had a complete response. vonPawel et al also published a retrospective study in the Journal of Clinical Oncology (2014) and showed that treatment outcomes for 2nd-line small cell lung cancer patients was even worse, with only 35 of the 213 patients on topotecan 1.5 mg/m² QDx5 dosing having a partial response to treatment and only 1 having a complete response.

Small molecules have been used to reduce some of the side effects of certain chemotherapeutic compounds. For example, leukovorin has been used to mitigate the effects of methotrexate on bone marrow cells and on gastrointestinal mucosa cells. Amifostine has been used to reduce the incidence of neutropenia-related fever and mucositis in patients receiving alkylating or platinum-containing chemotherapeutics. Also, dexrazoxane has been used to provide cardioprotection from anthracycline anti-cancer compounds. Unfortunately, there is concern that many chemoprotectants, such as dexrazoxane and amifostine, can decrease the efficacy of chemotherapy given concomitantly.

Additional chemoprotectant therapies, particularly with chemotherapy associated anemia and neutropenia, include the use of growth factors. Hematopoietic growth factors are available on the market as recombinant proteins. These proteins include granulocyte colony stimulating factor (G-CSF) and granulocyte-macrophage colony stimulating factor (GM-CSF) and their derivatives for the treatment of neutropenia, and erythropoietin (EPO) and its derivatives for the treatment of anemia. However, these recombinant proteins are expensive. Moreover, EPO has significant toxicity in cancer patients, leading to increased thrombosis, relapse, and death in several large randomized trials. G-CSF and GM-CSF may increase the late (>2 years post-therapy) risk of secondary bone marrow disorders such as leukemia and myelodysplasia. Consequently, their use is restricted and not readily available to all patients in need. Further, while growth factors can hasten recovery of some blood cell lineages, no therapy exists to treat suppression of platelets, macrophages, T-cells or B-cells.

Roberts et al in 2012 reported that Pfizer compound PD-0332991 (palbociclib) induced a transient cell cycle arrest in CDK4/6 dependent subsets of healthy cells such as HSPCs (see Roberts et al. Multiple Roles of Cyclin-Dependent Kinase 4/6 Inhibitors in Cancer Therapy. JNCI 2012; 104(6):476-487). This compound has been approved as an anti-neoplastic agent against estrogen-positive, HER2-negative breast cancer.

Hematopoietic stem cells give rise to progenitor cells which in turn give rise to all the differentiated components of blood as shown in FIG. 1 (e.g., lymphocytes, erythrocytes, platelets, granulocytes, monocytes). HSPCs require the activity of CDK4/6 for proliferation (see Roberts et al. Multiple Roles of Cyclin-Dependent Kinase 4/6 Inhibitors in Cancer Therapy. JNCI 2012; 104(6):476-487). In healthy kidneys, the renal epithelium infrequently enters the cell cycle (about 1% of epithelial cells). After a renal insult, however, a robust increase in epithelial proliferation occurs (see Humphreys, B. D. et al. Intrinsic epithelial cells repair the kidney after injury. Cell Stem Cell 2, 284-91 (2008)). Importantly, following renal injury, surviving renal epithelial cells replicate to repair damage to the kidney tubular epithelium (see Humphreys, B. D. et al. Repair of injured proximal tubule does not involve specialized progenitors. Proc Natl Acad Sci USA 108, 9226-31 (2011)). See also WO 2010132725 filed by Sharpless et al.

A number of CDK 4/6 inhibitors have been identified, including specific pyrido[2,3-d]pyrimidines, 2-anilinopyrimidines, diaryl ureas, benzoyl-2,4-diaminothiazoles, indolo[6,7-a]pyrrolo[3,4-c]carbazoles, and oxindoles (see P. S. Sharma, R. Sharma, R. Tyagi, Curr. Cancer Drug Targets 8 (2008) 53-75). WO 03/062236 identifies a series of 2-(pyridin-2-ylamino-pyrido[2,3]pyrimidin-7-ones for the treatment of Rb positive cancers that show selectivity for CDK4/6, including 6-acetyl-8-cyclopentyl-5-methyl-2-(5-piperazin-1-yl-pyridin-2-ylammino)-8H-pyrido-[2,3-d]-pyrimidin-7-one (palbociclib). The clinical trial studies have reported rates of Grade 3/4 neutropenia and leukopenia with the use of palbociclib, resulting in 71% of patients requiring a dose interruption and 35% requiring a dose reduction; and adverse events leading to 10% of the discontinuations (see Finn, Abstract S1-6, SABCS 2012). These side effects may be caused by the undesirable pharmacokinetics of palbociclib, which has a relatively long T1/2 of roughly 26.7 hours, resulting in an accumulative concentration build-up of the CDK4/6 inhibitor and a persistent quiescence of HPSC replication.

Other CDk inhibitors have also shown clinical insufficiencies. For example, administration of ribociclib is associated with QT prolongation and requires strict I/E criteria in trials and ECG monitoring (Infante J R, et al Mol Cancer Ther. 2013; 12(11 suppl):Abstract A276). Abemaciclib, for example, has been associated with gastrointestinal toxicity due to insufficient selectivity with greater than 50% diarrhea in clinical trials (see. Clinical Activity of Abemaciclib (LY2835219), a Cell Cycle Inhibitor Selective for CDK4 and CDK6, in Patients with Relapsed or Refractory Mantle Cell Lymphoma, Abstract 3067, ASCO 2014).

VanderWel et al. describe an iodine-containing pyrido[2,3-d]pyrimidine-7-one (CKIA) as a potent and selective CDK4 inhibitor (see VanderWel et al., J. Med. Chem. 48 (2005) 2371-2387). WO 99/15500 filed by Glaxo Group Ltd discloses protein kinase and serine/threonine kinase inhibitors.

WO 2010/020675 filed by Novartis AG describes pyrrolopyrimidine compounds as CDK inhibitors. WO 2011/101409 also filed by Novartis describes pyrrolopyrimidines with CDK 4/6 inhibitory activity.

WO 2005/052147 filed by Novartis and WO 2006/074985 filed by Janssen Pharma disclose additional CDK4 inhibitors.

US 2007/0179118 filed by Barvian et al. teaches the use of CDK4 inhibitors to treat inflammation.

WO 2012/061156 filed by Tavares and assigned to G1 Therapeutics describes CDK inhibitors. In addition, U.S. Pat. Nos. 8,829,012, 8,822,683, 8,598,186, 8,691,830, 8,598,197, and 9,102,682, all assigned to G1 Therapeutics, describes CDK Inhibitors. U.S. Pat. No. 9,260,442 filed by Tavares and assigned to G1 Therapeutics describes Lactam Kinase inhibitors.

U.S. Patent Publication 2011/0224227 to Sharpless et al. describes the use of certain CDK4/6 inhibitors, such as PD0332991 and 2BrIC (see Zhu, et al., J. Med. Chem., 46 (11) 2027-2030 (2003); PCT/US2009/059281) to reduce or prevent the effects of cytotoxic compounds on HSPCs in a subject undergoing chemotherapeutic treatments. See also U.S. Patent Publication 2012/0100100. Stone, et al., Cancer Research 56, 3199-3202 (Jul. 1, 1996) describes reversible, p16-mediated cell cycle arrest as protection from chemotherapy.

U.S. Patent Publication 2014/0275066, assigned to G1 Therapeutics, describes the use of CDK4/6 inhibitors such as 2′-((5-(4-methylpiperazin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one for the protection of healthy hematopoietic stem and progenitor cells in a subject receiving a DNA-damaging chemotherapeutic agent for the treatment of CDK 4/6-replication independent cellular proliferation disorders.

PCT Application No; PCT/US15/49756 filed by G1 Therapeutics describes the use of CDK4/6 inhibitors and certain topoisomerase inhibitors in combination for the treatment of certain CDK 4/6-replication independent cellular proliferation disorders.

Accordingly, it is an object of the present invention to provide dosages and methods to treat patients which provide desirable pharmacokinetic characteristics for subjects undergoing chemotherapy allowing for the short, transient protection of hematopoietic stem and progenitor cells, daily dosing of the inhibitor without accumulation of the drug in blood plasma, the rapid reentry of these cells into the cell cycle following the dissolution of the chemotherapeutic effect, reduced side effects and/or off-target effects of chemoprotectants, and the reduction for the need of the chemotherapy cessation for treatment holidays.

### SUMMARY OF THE INVENTION

The invention provides particular dosing and blood profile ranges of the CDK4/6 inhibitor compound 2′-((5-(4-methylpiperazin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one (Compound 1), and methods using said dosages, for treating a subject undergoing DNA-damaging chemotherapeutic therapy for the treatment of a CDK 4/6-replication independent cellular proliferation disorder, for example, small cell lung cancer, triple negative breast cancer, bladder cancer, and HPV+ head and neck cancer. The dosages and methods described herein provide desirable pharmacokinetic (PK) and pharmacodynamic (PD) characteristics which are designed to minimize the effect of chemotherapeutic agent toxicity on CDK4/6 replication dependent healthy cells, such as hematopoietic stem cells and hematopoietic progenitor cells (together referred to as HSPCs) and/or renal epithelial cells, in subjects, typically humans, that will be, are being, or have been exposed to a chemotherapeutic agent (typically a DNA-damaging agent) during the treatment of a CDK 4/6-replication independent cellular proliferation disorder. Such a strategy allows for the preservation of hematopoietic lineages and immune cell system functions during chemotherapeutic treatment, the ability to maintain chemotherapeutic dose and enhance anti-tumor activity, reduce incidence of febrile neutropenia, anemia, and low platelet counts, and reduce long-term adverse bone marrow complications associated with chemotherapy, for example, the reduction of bone marrow exhaustion following chemotherapeutic treatment, the preservation of bone marrow and the immune system. Without wishing to be bound to any one theory, it is believed that, in addition to protecting HSPCs, the use of Compound 1 to protect immune cell function may result in a robust anti-cancer immune response following chemotherapy.

As shown herein, the administration of Compound 1 in the dosing profiles and schedules described herein in combination with standard of care chemotherapeutic agents to treat the CDK4/6-replication independent proliferation disorder small cell lung cancer has shown an improved treatment outcome in both first-line and second-line small cell lung patients compared to the standard of care alone, including in historically refractory patient populations.

It has been discovered that dosing human subjects with Compound 1 to achieve the PK and PD profiles described herein provides a sufficiently long arrest of HSPCs in the G0/G1 phase of the cell cycle to provide protection from chemotherapy-induced DNA damage followed by re-initiation of hematopoiesis following chemotherapy exposure. Specifically, the invention includes administering Compound 1, which chemical formula is provided in FIG. 1, or a pharmaceutically acceptable composition, salt, isotopic analog, or prodrug thereof, to a subject, preferably a human, undergoing chemotherapy for the treatment of a CDK 4/6-replication independent cellular proliferation disorder, wherein the dosage administered to a subject provides a blood plasma level profile allowing a transient G1-arrest of CDK 4/6 replication dependent healthy cells, for example HSPCs and/or renal epithelial cells, in a subject during or following the subject's exposure to a chemotherapeutic agent, such as a DNA-damaging chemotherapeutic agent, while reducing the negative effects associated with HSPC proliferation cessation.

Importantly, it has also discovered that the dosing of Compound 1 as described herein allows for multi-day administration, for example consecutive dosing across 2, 3, 4, or 5 days, or more without significant increases or elevations in the PK and/or PD blood profile of Compound 1, for example no more than about a 10% increase in one or more PK and/or PD parameters, in subjects receiving such multi-day doses. Such dosing allows for the use of Compound 1 in a multi-day chemotherapeutic treatment regime without significant accumulation of the compound within a subject's plasma, reducing the risk of the development of myelosuppression from HSPC arrest during treatment and the allowance of a rapid reentry of HSPCs into the cell cycle.

It has also been discovered that, by administering a dose of Compound 1 as described herein, the amount of a subsequently administered chemotherapeutic agent, for example topotecan, needed to be therapeutically effective is lowered. For example, in one embodiment, the therapeutically effective dose of the chemotherapeutic agent is about 10-50% lower when administered following administration of Compound 1. Accordingly, an ideal or standard AUC for a chemotherapeutic agent administered following administration of Compound 1 is achieved at a lower dose than when the chemotherapeutic agent is administered alone. Such lowering of a therapeutic level may provide for the reduction of toxicity or off-target effects caused by the metabolism of the chemotherapeutic agent, while maintaining the anti-cancer effectiveness of the chemotherapeutic agent.

In one aspect of the present invention, provided herein is a dosing regimen comprising the administration of Compound 1 that provides a specific PK and/or PD blood profile followed by the administration of a chemotherapeutic agent for the treatment of the CDK 4/6-replication independent cellular proliferation disorder, wherein the CDK 4/6 replication independent cellular proliferation disorder is an Rb-negative cancer, for example small cell lung cancer, triple negative breast cancer, bladder cancer, or HPV+ head and neck cancer. In one embodiment, the CDK4/6-replication independent cellular proliferation disorder is small cell lung cancer.

In one embodiment, the CDK 4/6 replication independent cellular proliferation disorder is small cell lung cancer and the DNA-damaging chemotherapeutic agent is selected from the group consisting of carboplatin, cisplatin, etoposide, and topotecan, or a combination thereof. In one embodiment, the CDK 4/6 replication independent cellular proliferation disorder is small cell lung cancer and the DNA-damaging chemotherapeutic agent is etoposide. In one embodiment, the CDK 4/6 replication independent cellular proliferation disorder is small cell lung cancer and the DNA-damaging chemotherapeutic agent is carboplatin. In one embodiment, the CDK 4/6 replication independent cellular proliferation disorder is small cell lung cancer and the DNA-damaging chemotherapeutic agent is a combination therapeutic regime comprising carboplatin and etoposide. In one embodiment, the CDK 4/6 replication independent cellular proliferation disorder is small cell lung cancer and the DNA-damaging chemotherapeutic agent is cisplatin. In one embodiment, the CDK 4/6 replication independent cellular proliferation disorder is small cell lung cancer and the DNA-damaging chemotherapeutic agent is topotecan.

In one aspect, Compound 1 is intravenously administered to a subject prior to administration of a chemotherapeutic agent so that a single dose of Compound 1 provides a specific PK and/or PD blood profile as described herein. In one embodiment, the dose administered to the subject is between about 180 and about 215 mg/m². In one embodiment, the dose is between about 180 and about 280 mg/m². For example, the dose is about 180, 185, 190, 195, 200, 205, 210, 215, 220, 225, 230, 235, 240, 245, 250, 255, 260, 265, 270, 275, or 280 mg/m². In one embodiment, the dose is about 192 mg/m². In one embodiment, the dose is about 200 mg/m². In one embodiment, the dose is about 240 mg/m². In one embodiment, the dose administered provides for a mean AUC₍ₗₐₛₜ₎ measured at 24.5 hours or a mean Cmax as described below.

In one aspect, Compound 1 is intravenously administered to a subject prior to administration of a chemotherapeutic agent so that a single dose of Compound 1 provides a blood plasma level profile dosage-corrected mean Cmax ((ng/ml)/(mg/m2)) of between about 4 (ng/ml)/(mg/m2) and 12 (ng/ml)/(mg/m²). In one embodiment, the dosage-corrected mean Cmax ((ng/ml)/(mg/m²)) is about 4, 5, 6, 7, 8, 9, 10, 11, or 12 (ng/ml)/(mg/m²). In one embodiment, the dosage-corrected mean Cmax ((ng/ml)/(mg/m²)) is about 9.5 (ng/ml)/(mg/m²)±1.5 (ng/ml)/(mg/m²). In an alternative embodiment the dosage-corrected mean Cmax is about 9.5 (ng/ml)/(mg/m²)±1.9 (ng/ml)/(mg/m²) or 9.5 (ng/ml)/(mg/m²)±about 20%. In one embodiment, the dosage-corrected mean Cmax is about 6.0±20%. The dosage corrected mean Cmax is mean Cmax divided by the number of milligrams/m² of Compound 1 in the formulation.

In one aspect, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a dosage-corrected mean Cmax (ng/ml)/(mg/m²) of between about 4 (ng/ml)/(mg/m²) and 14 (ng/ml)/(mg/m²). In one embodiment, the dosage-corrected mean Cmax ((ng/ml)/(mg/m²)) is about 4, 5, 6, 7, 8, 9, 10, 11, 12, 13, or 14 (ng/ml)/(mg/m²). In one embodiment, the dosage-corrected mean Cmax ((ng/ml)/(mg/m2)) is about 9.5 (ng/ml)/(mg/m²) 1.5 (ng/ml)/(mg/m²). In an alternative embodiment the dosage-corrected mean Cmax is about 9.5 (ng/ml)/(mg/m²)±1.9 (ng/ml)/(mg/m²) or 9.5 (ng/ml)/(mg/m²)±about 20%. In one embodiment, the mean dose-corrected Cmax ((ng/ml)/(mg/m²)) is about 10.45 (ng/ml)/(mg/m²)±about 20%. In one embodiment, the dosage-corrected mean Cmax is about 6.0 ((ng/ml)/(mg/m²))±20%. In one embodiment, the dosage-corrected mean Cmax is about 6.5 ((ng/ml)/(mg/m²))±20%. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one aspect, Compound 1 is intravenously administered to a subject prior to administration of a chemotherapeutic agent so that a single dose of Compound 1 provides a blood plasma level profile mean Cmax (ng/ml) of between about 1000 ng/ml and 3500 ng/ml. In one embodiment, the mean Cmax (ng/ml) is between about 1400 ng/ml and about 3100 ng/ml. In one embodiment, the mean Cmax (ng/ml) of about 1400, 1450, 1500, 1550, 1600, 1650, 1700, 1750, 1800, 1850, 1900, 1950, 2000, 2050, 2100, 2150, 2200, 2250, 2300, 2400, 2450, 2500, 2600, 2650, 2700, 2750, 2800, 2850, 2900, 2950, 3000, 3050, or 3100 (ng/ml). In one embodiment, the mean Cmax (ng/ml) is about 2030 ng/ml±555 ng/ml. In an alternative embodiment the mean Cmax is about 2030 (ng/ml)±406 (ng/ml) or about 2030 (ng/ml)±about 20%. In one embodiment, a single dose provides a blood plasma profile with a mean Cmax of about 355 ng/ml to about 3100 ng/ml. In one embodiment, a single dose provides a blood plasma profile with a mean Cmax of at least about 1020 ng/ml. In one embodiment, the maximum mean concentration occurs at the end of the infusion period of Compound 1.

In one aspect, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound lwith a mean Cmax (ng/ml) of between about 1000 ng/ml and 3500 ng/ml. In one embodiment, the mean Cmax (ng/ml) is between about 1400 ng/ml and about 3100 ng/ml. In one embodiment, the mean Cmax (ng/ml) of about 1400, 1450, 1500, 1550, 1600, 1650, 1700, 1750, 1800, 1850, 1900, 1950, 2000, 2050, 2100, 2150, 2200, 2250, 2300, 2400, 2450, 2500, 2600, 2650, 2700, 2750, 2800, 2850, 2900, 2950, 3000, 3050, or 3100 (ng/ml). In one embodiment, the mean Cmax (ng/ml) is about 2030 ng/ml±555 ng/ml. In an alternative embodiment the mean Cmax is about 2030 (ng/ml)±406 (ng/ml) or about 2030 (ng/ml)±about 20%. In an alternative embodiment the mean Cmax is about 2230 (ng/ml)±about 20%. In one embodiment the mean Cmax is at least about 1020 ng/ml. In one embodiment, the maximum mean concentration occurs at the end of the infusion period of Compound 1. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one aspect, Compound 1 is intravenously administered to a subject prior to administration of a chemotherapeutic agent so that a single dose of Compound 1 provides a blood plasma level profile mean AUCₜ (ng*hr/ml) measured over about 24.5 hours after administration of between about 2000 h*ng/ml to about 4500 h*ng/ml. In one embodiment, the mean AUCₜ (ng*hr/ml) measured over about 24.5 hours after administration of between about 2300 h*ng/ml to about 4000 h*ng/ml. In one embodiment, the mean AUCₜ (ng*hr/ml) measured over about 24.5 hours after administration is about 2830 (ng*hr/ml)±474 (ng*hr/ml). In an alternative embodiment, the mean AUCₜ (ng*hr/ml) measured over about 24.5 hours after administration is about 2830 (ng*hr/ml)≅566 (ng*hr/ml) or about 2830 (ng*hr/ml)±about 20%. In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a mean AUCₜ (ng*hr/ml) measured over about 24.5 hours after administration of at least about 2040 ng*hr/ml. In one embodiment, the mean AUCₜ (ng*hr/ml) measured over about 72.5 hours after administration is between about 2300 h*ng/ml to about 4100 h*ng/ml. In one embodiment, the mean AUCₜ (ng*hr/ml) measured over about 72.5 hours after administration is about 3110 (ng*hr/ml)±515 (ng*hr/ml). In an alternative embodiment, the mean AUCₜ (ng*hr/ml) measured over about 72.5 hours after administration is about 3110 (ng*hr/ml)±622 (ng*hr/ml) or about 3110 (ng*hr/ml)±about 20%.

In one aspect, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a mean AUCₜ (ng*hr/ml) measured over about 24.5 hours after administration of between about 2300 h*ng/ml to about 4000 h*ng/ml. In one embodiment, the mean AUCₜ (ng*hr/ml) measured over about 24.5 hours after administration is about 2830 (ng*hr/ml)±550 (ng*hr/ml). In an alternative embodiment, the mean AUCₜ (ng*hr/ml) measured over about 24.5 hours after administration is about 2830 (ng*hr/ml)±560 (ng*hr/ml) or about 2830 (ng*hr/ml)±about 20%. In one embodiment, the mean AUCₜ (ng*hr/ml) measured over about 24.5 hours after administration is about 3020 (ng*hr/ml)±about 20%. In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a mean AUCₜ (ng*hr/ml) measured over about 24.5 hours after administration of at least about 2040 ng*hr/ml. In one embodiment, the mean AUCₜ (ng*hr/ml) measured over about 72.5 hours after administration is between about 2300 h*ng/ml to about 4100 h*ng/ml. In an alternative embodiment, the mean AUCₜ (ng*hr/ml) measured over about 72.5 hours after administration is about 3100 (ng*hr/ml)±620 (ng*hr/ml) or about 3100 (ng*hr/ml)±about 20%. In one embodiment, the mean AUCₜ (ng*hr/ml) measured over about 72.5 hours after administration is about 3410 (ng*hr/ml)±about 20%. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one aspect, Compound 1 is intravenously administered to a subject prior to administration of a chemotherapeutic agent so that a single dose of Compound 1 provides a blood plasma level profile dosage-corrected mean AUCₜ (h*ng/ml)/(mg/m²) of between about 6.0 (h*ng/ml)/(mg/m²) and 20 (h*ng/ml)/(mg/m²). In one embodiment, the dosage-corrected mean AUCₜ (h*ng/ml)/(mg/m²) is about 12.0 (h*ng/ml)/(mg/m²)±3.0 (h*ng/ml)/(mg/m²). In an alternative embodiment, the dosage-corrected mean AUCₜ (h*ng/ml)/(mg/m²) is about 12.0 (h*ng/ml)/(mg/m²)±2.4 (h*ng/ml)/(mg/m²) or about 12.0 (h*ng/ml)/(mg/m²)±about 20%. The dosage-corrected mean AUCₜ is mean AUCₜ divided by the number of milligrams/m² of Compound 1 in the formulation.

In one aspect, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a dosage-corrected mean AUCₜ (h*ng/ml)/(mg/m²) of between about 6 (h*ng/ml)/(mg/m²) and 20 (h*ng/ml)/(mg/m²). In one embodiment, the dosage-corrected mean AUCₜ (h*ng/ml)/(mg/m²) is about 15.0 (h*ng/ml)/(mg/m2)±3.0 (h*ng/ml)/(mg/m2) or about 15.0 (h*ng/ml)/(mg/m²)±about 20%. In one embodiment, the dosage-corrected mean AUCₜ (h*ng/ml)/(mg/m²) is at least about 8.35 (h*ng/ml)/(mg/m²). In one embodiment, the dosage-corrected mean AUCₜ (h*ng/ml)/(mg/m2) is about 16.5 (h*ng/ml)/(mg/m2)±about 20%. In one embodiment, the dosage-corrected mean AUCₜ (h*ng/ml)/(mg/m2) is at least about 10.0 (h*ng/ml)/(mg/m2). The dosage corrected AUCₜ is AUCₜ divided by the number of milligrams/m² of Compound 1 in the formulation. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one aspect, Compound 1 is intravenously administered to a subject prior to administration of a chemotherapeutic agent so that a single dose of Compound 1 provides a blood plasma level profile dosage-corrected mean AUC_{inf} (h*ng/ml)/(mg/m²) of between about 6 (h*ng/ml)/(mg/m²) and 20 (h*ng/ml)/(mg/m²). In one embodiment, the dosage-corrected mean AUC_{inf} (h*ng/ml)/(m g/m²) is about 15.5 (h*ng/ml)/(mg/m²)±3.5 (h*ng/ml)/(mg/m²) or about 15.5 (h*ng/ml)/(mg/m²)±about 20%. In one embodiment, the dosage-corrected mean AUC_{inf} (h*ng/ml)/(mg/m²) is about 17 (h*ng/ml)/(mg/m²)±about 20%. In one embodiment, the dosage-corrected mean AUC_{inf} (h*ng/ml)/(mg/m²) is at least about 10.5 (h*ng/ml)/(mg/m²). The dosage corrected AUC_{inf} is AUC_{inf} divided by the number of milligrams/m² of Compound 1 in the formulation.

In one aspect, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a dosage-corrected mean AUC_{inf} (h*ng/ml)/(mg/m²) of between about 6 (h*ng/ml)/(mg/m²) and 20 (h*ng/ml)/(mg/m²). In one embodiment, the dosage-corrected mean AUC_{inf} (h*ng/ml)/(mg/m²) is about 15.5 (h*ng/ml)/(mg/m²)±3.5 (h*ng/ml)/(mg/m²) or about 15.5 (h*ng/ml)/(mg/m²)±about 20%. In one embodiment, the dosage-corrected mean AUC_{inf} (h*ng/ml)/(m g/m²) is about 17 (h*ng/ml)/(mg/m²)±about 20%. The dosage-corrected mean AUC_{inf} is mean AUC_{inf} divided by the number of milligrams/m² of Compound 1 in the formulation. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one embodiment, Compound 1 is administered about 30 minutes prior to the administration of a chemotherapeutic agent, wherein Compound 1 is administered intravenously over about 30 minutes. In one embodiment, Compound 1 is administered to a patient having small cell lung cancer about 30 minutes prior to administration of carboplatin. In one embodiment, Compound 1 is administered to a patient having small cell lung cancer over about 30 minutes just prior to administration of etoposide. In one embodiment, Compound 1 is administered to a subject having small cell lung cancer over about 30 minutes just prior to administration of topotecan.

In one aspect, provided herein is a method of treating a subject undergoing chemotherapy for the treatment of small cell lung cancer by providing an intravenously administered formulation of Compound 1 on days 1, 2, and 3 about 30 minutes prior to the administration of etoposide and carboplatin on day 1, and etoposide on days 2 and 3, wherein the subject is provided etoposide and carboplatin on day 1, and etoposide on days 1, 2, and 3 during a 21-day therapeutic cycle, and wherein Compound 1 is administered in a dosage which provides any of the blood profile PK and/or PD parameters, or a combination of blood profile PK and/or PD parameters, as described herein. In one embodiment, compound 1 is administered to the subject over about 30 minutes prior to administration of etoposide and/or carboplatin. In one embodiment, Compound 1 is administered at a dosage of about 180 mg/m² to about 280 mg/m². In one embodiment, Compound 1 is administered at about 180, 185, 190, 195, 200, 205, 210, 215, 220, 225, 230, 235, 240, 245, 250, 255, 260, 265, 270, 275, or about 280 mg/m². In one embodiment, the etoposide is administered to the subject at a dosage of about 100 mg/m². In one embodiment, the carboplatin is administered to the subject at a dosage that achieves a target AUC of about 5 min*mg/m².

In one aspect, provided herein is a method of treating a subject undergoing chemotherapy for the treatment of small cell lung cancer by providing an intravenously administered formulation of Compound 1 on days 1, 2, 3, 4, and 5 about 30 minutes prior to the administration of topotecan on days 1, 2, 3, 4, and 5 during a 21-day therapeutic cycle, and wherein Compound 1 is administered in a dosage which provides any of the blood profile PK and/or PD parameters, or a combination of blood profile PK and/or PD parameters, as described herein. In one embodiment, Compound 1 is administered at a dosage of about 180 mg/m² to about 280 mg/m². In one embodiment, Compound 1 is administered at about 180, 185, 190, 195, 200, 205, 210, 215, 220, 225, 230, 235, 240, 245, 250, 255, 260, 265, 270, 275, or about 280 mg/m². In one embodiment, Compound 1 is administered at a dose of about 200 mg/m². In one embodiment, Compound 1 is administered at a dose of about 240 mg/m². In one embodiment, the topotecan is administered to the subject at a dosage of about 1.5 mg/m². In one embodiment, the topotecan is administered to the subject at a dosage of about 1.25 mg/m². In one embodiment, the topotecan is administered to the subject at a dosage of about 0.75 mg/m².

In one aspect, provided herein is a method of treating a subject undergoing chemotherapy for the treatment of small cell lung cancer by providing an intravenously administered formulation of Compound 1 on days 1, 2, 3 about 30 minutes prior to the administration of topotecan on days 1, 2, 3 during a 21-day therapeutic cycle, and wherein Compound 1 is administered in a dosage which provides any of the blood profile PK and/or PD parameters, or a combination of blood profile PK and/or PD parameters, as described herein. In one embodiment, Compound 1 is administered at a dosage of about 180 m g/m² to about 280 mg/m². In one embodiment, Compound 1 is administered at about 180, 185, 190, 195, 200, 205, 210, 215, 220, 225, 230, 235, 240, 245, 250, 255, 260, 265, 270, 275, or about 280 mg/m². In one embodiment, Compound 1 is administered at a dose of about 200 mg/m2. In one embodiment, Compound 1 is administered at a dose of about 240 mg/m². In one embodiment, the topotecan is administered to the subject at a dosage of about 1.25 mg/m².

In one aspect, provided herein is a method of reducing the therapeutically effective dose of a chemotherapeutic agent administered to a subject having a CDK4/6 replication independent proliferation disorder comprising administering to the subject Compound 1 and subsequently administering to the subject the chemotherapeutic agent. In one embodiment, the therapeutically effective dose of the chemotherapeutic agent is from about 10% to about 50% less than the therapeutically effective dose of the chemotherapeutic agent when administered without prior administration of Compound 1. In one embodiment, the chemotherapeutic agent is topotecan. In one embodiment, the therapeutically effective dose of topotecan administered subsequent to the administration of Compound 1 is about 10%, about 25%, about 35%, or about 50% less than when administered without prior administration of Compound 1. In one embodiment, the therapeutically effective dose of topotecan administered subsequent to administration of Compound 1 is about 25% less than when administered without prior administration of Compound 1. In one embodiment, the therapeutically effective dose of topotecan is about 1.25 mg/m2. In an alternative embodiment, the therapeutically effective dose of topotecan administered subsequently to the administration of Compound 1 is about 1.25 mg/m2±0.25 mg/m2 or about 1.25 mg/m2±about 20%. In one embodiment, the therapeutically effective dose of topotecan administered to the subject following administration of Compound 1 is about 0.75 mg/m2. In an alternative embodiment, the therapeutically effective dose of topotecan administered following administration of Compound 1 is about 0.75 mg/m2±0.15 mg/m2 or about 0.75 mg/m2±about 20%.

In one aspect, provided herein is a method to protect immune system cells wherein Compound 1 is administered at a dosage described herein prior to chemotherapy to protect immune system function from chemotherapy damage. In one embodiment Compound 1 is administered at a dosage described herein prior to chemotherapy to preserve bone marrow, lymphoid, progenitors, and lymphocytes from damage by chemotherapy, allowing for faster hematopoietic recovery, preserving long term bone marrow function, and enhancing the anti-tumor activity of chemotherapy. Non-limiting examples of chemotherapy include 5-flourouracil, temozolomide, paclitaxel, cisplatin, carboplatin, topotecan, vincristine, and etoposide.

### BRIEF DESCRIPTION OF THE FIGURES

FIG. 1 depicts the chemical structure of Compound 1.

FIG. 2A is a graph plotting the linear relationship between individual subject plasma concentrations versus time plots for study subjects in Cohort 1 who received 6 mg/m² of Compound 1. Cohort=Cohort 1, Treatment=6 mg/m², Subject=1.

FIG. 2B is a graph plotting the linear relationship between individual subject plasma concentrations versus time plots for study subjects in Cohort 1 who received 6 mg/m² of Compound 1. Cohort=Cohort 1, Treatment=6 mg/m², Subject=2.

FIG. 2C is a graph plotting the linear relationship between individual subject plasma concentrations versus time plots for study subjects in Cohort 1 who received 6 mg/m² of Compound 1. Cohort=Cohort 1, Treatment=6 mg/m², Subject=3.

FIG. 3A is a graph plotting the log-linear relationship between individual subject plasma concentrations versus time plots for study subjects in Cohort 1 who received 6 mg/m² of Compound 1. Cohort=Cohort 1, Treatment=6 mg/m², Subject=1.

FIG. 3B is a graph plotting the log-linear relationship between individual subject plasma concentrations versus time plots for study subjects in Cohort 1 who received 6 mg/m² of Compound 1. Cohort=Cohort 1, Treatment=6 mg/m², Subject=2.

FIG. 3C is a graph plotting the log-linear relationship between individual subject plasma concentrations versus time plots for study subjects in Cohort 1 who received 6 mg/m² of Compound 1. Cohort=Cohort 1, Treatment=6 mg/m², Subject=3.

FIG. 4A reflects WinNonlin Plots from Noncompartmental Analysis in subjects in Cohort 1 who received 6 mg/m² of Compound 1. Cohort=Cohort 1, Treatment=6 mg/m², Subject=1, Rsq=0.902, Rsq_adjusted=0.8824, HL_Lambda_z=6.358, 7 points used in calculation, Uniform Weighting.

FIG. 4B reflects WinNonlin Plots from Noncompartmental Analysis in subjects in Cohort 1 who received 6 mg/m² of Compound 1. Cohort=Cohort 1, Treatment=6 mg/m², Subject=2, Rsq=0.9819, Rsq_adjusted=0.9783, HL_Lambda_z=3.5207, 7 points used in calculation, Uniform Weighting.

FIG. 4C reflects WinNonlin Plots from Noncompartmental Analysis in subjects in Cohort 1 who received 6 mg/m² of Compound 1. Cohort=Cohort 1, Treatment=6 mg/m², Subject=3, Rsq=0.9986, Rsq_adjusted=0.9972, HL_Lambda_z=4.7366, 3 points used in calculation, Uniform Weighting.

FIG. 5A is a graph plotting the linear relationship between individual subject plasma concentrations versus time plots for study subjects in Cohort 2 who received 12 mg/m² of Compound 1. Cohort=Cohort 2, Treatment=12 mg/m², Subject=1.

FIG. 5B is a graph plotting the linear relationship between individual subject plasma concentrations versus time plots for study subjects in Cohort 2 who received 12 mg/m² of Compound 1. Cohort=Cohort 2, Treatment=12 mg/m², Subject=2.

FIG. 5C is a graph plotting the linear relationship between individual subject plasma concentrations versus time plots for study subjects in Cohort 2 who received 12 mg/m² of Compound 1. Cohort=Cohort 2, Treatment=12 mg/m², Subject=3.

FIG. 6A is a graph plotting the log-linear relationship between individual subject plasma concentrations versus time plots for study subjects in Cohort 2 who received 12 mg/m² of Compound 1. Cohort=Cohort 2, Treatment=12 mg/m², Subject=1.

FIG. 6B is a graph plotting the log-linear relationship between individual subject plasma concentrations versus time plots for study subjects in Cohort 2 who received 12 mg/m² of Compound 1. Cohort=Cohort 2, Treatment=12 mg/m², Subject=2.

FIG. 6C is a graph plotting the log-linear relationship between individual subject plasma concentrations versus time plots for study subjects in Cohort 2 who received 12 mg/m² of Compound 1. Cohort=Cohort 2, Treatment=12 mg/m², Subject=3.

FIG. 7A reflects WinNonlin Plots from Noncompartmental Analysis in subjects in Cohort 2 who received 12 mg/m² of Compound 1. Cohort=Cohort 2, Treatment=12 mg/m², Subject=1, Rsq=0.9859, Rsq_adjusted=0.9717, HL_Lambda_z=7.5562, 3 points used in calculation, Uniform Weighting.

FIG. 7B reflects WinNonlin Plots from Noncompartmental Analysis in subjects in Cohort 2 who received 12 mg/m² of Compound 1. Cohort=Cohort 2, Treatment=12 mg/m², Subject=2, Rsq=0.9856, Rsq_adjusted=0.9711, HL_Lambda_z=8.8853, 3 points used in calculation, Uniform Weighting.

FIG. 7C reflects WinNonlin Plots from Noncompartmental Analysis in subjects in Cohort 2 who received 12 mg/m² of Compound 1. Cohort=Cohort 2, Treatment=12 mg/m², Subject=3, Rsq=0.9516, Rsq_adjusted=0.9275, HL_Lambda_z=8.2114, 4 points used in calculation, Uniform Weighting.

FIG. 8A is a graph plotting the linear relationship between individual subject plasma concentrations versus time plots for study subjects in Cohort 3 who received 24 mg/m² of Compound 1. Cohort=Cohort 3, Treatment=24 mg/m², Subject=1.

FIG. 8B is a graph plotting the linear relationship between individual subject plasma concentrations versus time plots for study subjects in Cohort 3 who received 24 mg/m² of Compound 1. Cohort=Cohort 3, Treatment=24 mg/m², Subject=2.

FIG. 8C is a graph plotting the linear relationship between individual subject plasma concentrations versus time plots for study subjects in Cohort 3 who received 24 mg/m² of Compound 1. Cohort=Cohort 3, Treatment=24 mg/m², Subject=3.

FIG. 9A is a graph plotting the log-linear relationship between individual subject plasma concentrations versus time plots for study subjects in Cohort 3 who received 24 mg/m2 of Compound 1. Cohort=Cohort 3, Treatment=24 mg/m², Subject=1.

FIG. 9B is a graph plotting the log-linear relationship between individual subject plasma concentrations versus time plots for study subjects in Cohort 3 who received 24 mg/m2 of Compound 1. Cohort=Cohort 3, Treatment=24 mg/m², Subject=2.

FIG. 9C is a graph plotting the log-linear relationship between individual subject plasma concentrations versus time plots for study subjects in Cohort 3 who received 24 mg/m2 of Compound 1. Cohort=Cohort 3, Treatment=24 mg/m², Subject=3.

FIG. 10A reflects WinNonlin Plots from Noncompartmental Analysis in subjects in Cohort 3 who received 24 mg/m² of Compound 1. Cohort=Cohort 3, Treatment=24 mg/m², Subject=1, Rsq=0.9663, Rsq_adjusted=0.9326, HL_Lambda_z=13.1458, 3 points used in calculation, Uniform Weighting.

FIG. 10B reflects WinNonlin Plots from Noncompartmental Analysis in subjects in Cohort 3 who received 24 mg/m² of Compound 1. Cohort=Cohort 3, Treatment=24 mg/m², Subject=2, Rsq=0.9904, Rsq_adjusted=0.9809, HL_Lambda_z=8.6328, 3 points used in calculation, Uniform Weighting.

FIG. 10C reflects WinNonlin Plots from Noncompartmental Analysis in subjects in Cohort 3 who received 24 mg/m² of Compound 1. Cohort=Cohort 3, Treatment=24 mg/m², Subject=3, Rsq=0.998, Rsq_adjusted=0.9961, HL_Lambda_z=7.8478, 3 points used in calculation, Uniform Weighting.

FIG. 11 plots the relationship between Cmax and Dose over the range of 6 to 24 mg/m². Rsq=0.7401, Intercept=−14.2, Slope=11.14.

FIG. 12 plots the relationship between AUCinf and Dose over the range of 6 to 24 mg/m². Rsq=0.7675, Intercept=−1.749, Slope=12.08.

FIG. 13 plots the relationship between CL and Dose over the range of 6 to 24 mg/m². Rsq=0.0003929, Intercept=87.82, Slope=0.05016.

FIG. 14A is a graph plotting the linear relationship between individual subject plasma concentrations versus time plots for study subjects in Cohort 4 who received 48 mg/m² of Compound 1. Cohort=Cohort 4, Treatment—48 mg/m², Subject=1.

FIG. 14B is a graph plotting the linear relationship between individual subject plasma concentrations versus time plots for study subjects in Cohort 4 who received 48 mg/m² of Compound 1. Cohort=Cohort 4, Treatment—48 mg/m², Subject=2.

FIG. 14C is a graph plotting the linear relationship between individual subject plasma concentrations versus time plots for study subjects in Cohort 4 who received 48 mg/m² of Compound 1. Cohort=Cohort 4, Treatment—48 mg/m², Subject=3.

FIG. 15A is a graph plotting the log-linear relationship between individual subject plasma concentrations versus time plots for study subjects in Cohort 4 who received 48 mg/m² of Compound 1. Cohort=Cohort 4, Treatment=48 mg/m², Subject=1.

FIG. 15B is a graph plotting the log-linear relationship between individual subject plasma concentrations versus time plots for study subjects in Cohort 4 who received 48 mg/m² of Compound 1. Cohort=Cohort 4, Treatment=48 mg/m², Subject=2.

FIG. 15C is a graph plotting the log-linear relationship between individual subject plasma concentrations versus time plots for study subjects in Cohort 4 who received 48 mg/m² of Compound 1. Cohort=Cohort 4, Treatment=48 mg/m², Subject=3.

FIG. 16A reflects WinNonlin Plots from Noncompartmental Analysis in subjects in Cohort 4 who received 48 mg/m² of Compound 1. Cohort=Cohort 4, Treatment=48 mg/m², Subject=1, Rsq=0.9987, Rsq_adjusted=0.9981, HL_Lambda_z=10.9028, 4 points used in calculation, Uniform Weighting.

FIG. 16B reflects WinNonlin Plots from Noncompartmental Analysis in subjects in Cohort 4 who received 48 mg/m² of Compound 1. Cohort=Cohort 4, Treatment=48 mg/m², Subject=2, Rsq=0.9957, Rsq_adjusted=0.9913, HL_Lambda_z=7.2383, 3 points used in calculation, Uniform Weighting.

FIG. 16C reflects WinNonlin Plots from Noncompartmental Analysis in subjects in Cohort 4 who received 48 mg/m² of Compound 1. Cohort=Cohort 4, Treatment=48 mg/m², Subject=3, Rsq=0.9818, Rsq_adjusted=0.9637, HL_Lambda_z=8.5525, 3 points used in calculation, Uniform Weighting.

FIG. 17 reflects the mean Compound 1 plasma concentration-time plots (Linear) for Cohorts 1-4.

FIG. 18 reflects the mean Compound 1 plasma concentration-time plots (Log-Linear) for Cohorts 1-4.

FIG. 19 plots the relationship between Cmax and Dose over the range of 6 to 48 mg/m². Rsq=0.463, Intercept=78.68, Slope=3.851.

FIG. 20 plots the relationship between AUCinf and Dose over the range of 6 to 24 mg/m². Rsq=0.9268, Intercept=23.5, Slope=10.14.

FIG. 21 plots the relationship between CL and Dose over the range of 6 to 24 mg/m². Rsq=0.04472, Intercept=85.19, Slope=0.2002.

FIG. 22A is a graph plotting the linear relationship between individual subject plasma concentrations versus time plots for study subjects in Cohort 5 who received 96 mg/m² of Compound 1. Cohort=Cohort 5, Treatment=96 mg/m², Subject=1.

FIG. 22B is a graph plotting the linear relationship between individual subject plasma concentrations versus time plots for study subjects in Cohort 5 who received 96 mg/m² of Compound 1. Cohort=Cohort 5, Treatment=96 mg/m², Subject=2.

FIG. 22C is a graph plotting the linear relationship between individual subject plasma concentrations versus time plots for study subjects in Cohort 5 who received 96 mg/m² of Compound 1. Cohort=Cohort 5, Treatment=96 mg/m², Subject=3.

FIG. 22D is a graph plotting the linear relationship between individual subject plasma concentrations versus time plots for study subjects in Cohort 5 who received 96 mg/m² of Compound 1. Cohort=Cohort 5, Treatment=96 mg/m², Subject=4.

FIG. 22E is a graph plotting the linear relationship between individual subject plasma concentrations versus time plots for study subjects in Cohort 5 who received 96 mg/m² of Compound 1. Cohort=Cohort 5, Treatment=96 mg/m², Subject=5.

FIG. 22F is a graph plotting the linear relationship between individual subject plasma concentrations versus time plots for study subjects in Cohort 5 who received 96 mg/m² of Compound 1. Cohort=Cohort 5, Treatment=96 mg/m², Subject=6.

FIG. 23A is a graph plotting the log-linear relationship between individual subject plasma concentrations versus time plots for study subjects in Cohort 5 who received 48 mg/m² of Compound 1. Cohort=Cohort 5, Treatment=96 mg/m², Subject=1.

FIG. 23B is a graph plotting the log-linear relationship between individual subject plasma concentrations versus time plots for study subjects in Cohort 5 who received 48 mg/m² of Compound 1. Cohort=Cohort 5, Treatment=96 mg/m², Subject=2.

FIG. 23C is a graph plotting the log-linear relationship between individual subject plasma concentrations versus time plots for study subjects in Cohort 5 who received 48 mg/m² of Compound 1. Cohort=Cohort 5, Treatment=96 mg/m², Subject=3.

FIG. 23D is a graph plotting the log-linear relationship between individual subject plasma concentrations versus time plots for study subjects in Cohort 5 who received 48 mg/m² of Compound 1. Cohort=Cohort 5, Treatment=96 mg/m², Subject=4.

FIG. 23E is a graph plotting the log-linear relationship between individual subject plasma concentrations versus time plots for study subjects in Cohort 5 who received 48 mg/m² of Compound 1. Cohort=Cohort 5, Treatment=96 mg/m², Subject=5.

FIG. 23F is a graph plotting the log-linear relationship between individual subject plasma concentrations versus time plots for study subjects in Cohort 5 who received 48 mg/m² of Compound 1. Cohort=Cohort 5, Treatment=96 mg/m², Subject=6.

FIG. 24A reflects WinNonlin Plots from Noncompartmental Analysis in subjects in Cohort 5 who received 96 mg/m² of Compound 1. Cohort=Cohort 5, Treatment=48 mg/m², Subject=1, Rsq=0.9485, Rsq_adjusted=0.9227, HL_Lambda_z=8.4971, 4 points used in calculation, Uniform Weighting.

FIG. 24B reflects WinNonlin Plots from Noncompartmental Analysis in subjects in Cohort 5 who received 96 mg/m² of Compound 1. Cohort=Cohort 5, Treatment=48 mg/m², Subject=2, Rsq=0.9877, Rsq_adjusted=0.9754, HL_Lambda_z=10.9427, 3 points used in calculation, Uniform Weighting.

FIG. 24C reflects WinNonlin Plots from Noncompartmental Analysis in subjects in Cohort 5 who received 96 mg/m² of Compound 1. Cohort=Cohort 5, Treatment=48 mg/m², Subject=3, Rsq=0.9974, Rsq_adjusted=0.9947, HL_Lambda_z=7.5378, 3 points used in calculation, Uniform Weighting.

FIG. 24D reflects WinNonlin Plots from Noncompartmental Analysis in subjects in Cohort 5 who received 96 mg/m² of Compound 1. Cohort=Cohort 5, Treatment=48 mg/m², Subject=4, Rsq=0.9806, Rsq_adjusted=0.9611, HL_Lambda_z=10.6557, 3 points used in calculation, Uniform Weighting.

FIG. 24E reflects WinNonlin Plots from Noncompartmental Analysis in subjects in Cohort 5 who received 96 mg/m² of Compound 1. Cohort=Cohort 5, Treatment=48 mg/m², Subject=5, Rsq=0.9848, Rsq_adjusted=0.9696, HL_Lambda_z=7.1128, 3 points used in calculation, Uniform Weighting.

FIG. 24F reflects WinNonlin Plots from Noncompartmental Analysis in subjects in Cohort 5 who received 96 mg/m of Compound 1. Cohort=Cohort 5, Treatment=48 mg/m², Subject=6, Rsq=0.9863, Rsq_adjusted=0.9726, HL_Lambda_z=6.9746, 3 points used in calculation, Uniform Weighting.

FIG. 25 reflects the mean Compound 1 plasma concentration-time plots (Linear) for Cohorts 1-5.

FIG. 26 reflects the mean Compound 1 plasma concentration-time plots (Log-Linear) for Cohorts 1-5.

FIG. 27 plots the relationship between Cmax and Dose over the range of 6 to 96 mg/m². Rsq=0.8778, Intercept=−23.31, Slope=9.225.

FIG. 28 plots the relationship between AUCinf and Dose over the range of 6 to 96 mg/m². Rsq=0.9473, Intercept=−46.86, Slope=14.02.

FIG. 29 plots the relationship between CL and Dose over the range of 6 to 96 mg/m². Rsq=0.1558, Intercept=91.45, Slope=−0.1623.

FIG. 30A reflects the linear relationship between individual subject plasma concentration vs time plots from 0-24.5 hours for subjects in Cohort 6 receiving 192 mg/m². Cohort=Cohort 6, Treatment=192 mg/m², Subject=1.

FIG. 30B reflects the linear relationship between individual subject plasma concentration vs time plots from 0-24.5 hours for subjects in Cohort 6 receiving 192 mg/m². Cohort=Cohort 6, Treatment=192 mg/m², Subject=2.

FIG. 30C reflects the linear relationship between individual subject plasma concentration vs time plots from 0-24.5 hours for subjects in Cohort 6 receiving 192 mg/m². Cohort=Cohort 6, Treatment=192 mg/m², Subject=3.

FIG. 30D reflects the linear relationship between individual subject plasma concentration vs time plots from 0-24.5 hours for subjects in Cohort 6 receiving 192 mg/m². Cohort=Cohort 6, Treatment=192 mg/m², Subject=4.

FIG. 30E reflects the linear relationship between individual subject plasma concentration vs time plots from 0-24.5 hours for subjects in Cohort 6 receiving 192 mg/m². Cohort=Cohort 6, Treatment=192 mg/m², Subject=5.

FIG. 30F reflects the linear relationship between individual subject plasma concentration vs time plots from 0-24.5 hours for subjects in Cohort 6 receiving 192 mg/m². Cohort=Cohort 6, Treatment=192 mg/m², Subject=6.

FIG. 31A reflects the log-linear relationship between individual subject plasma concentration vs time plots from 0-725 hours for subjects in Cohort 6 receiving 192 mg/m². Cohort=Cohort 6, Treatment=192 mg/m², Subject 1.

FIG. 31B reflects the log-linear relationship between individual subject plasma concentration vs time plots from 0-725 hours for subjects in Cohort 6 receiving 192 mg/m². Cohort=Cohort 6, Treatment=192 mg/m², Subject 2.

FIG. 31C reflects the log-linear relationship between individual subject plasma concentration vs time plots from 0-725 hours for subjects in Cohort 6 receiving 192 mg/m². Cohort=Cohort 6, Treatment=192 mg/m², Subject 3.

FIG. 31D reflects the log-linear relationship between individual subject plasma concentration vs time plots from 0-725 hours for subjects in Cohort 6 receiving 192 mg/m². Cohort=Cohort 6, Treatment=192 mg/m², Subject 4.

FIG. 31E reflects the log-linear relationship between individual subject plasma concentration vs time plots from 0-725 hours for subjects in Cohort 6 receiving 192 mg/m². Cohort=Cohort 6, Treatment=192 mg/m², Subject 5.

FIG. 31F reflects the log-linear relationship between individual subject plasma concentration vs time plots from 0-725 hours for subjects in Cohort 6 receiving 192 mg/m². Cohort=Cohort 6, Treatment=192 mg/m², Subject 6.

FIG. 32A reflects WinNonlin Plots from Noncompartmental Analysis in subjects in Cohort 6 who received 192 mg/m². Cohort=Cohort 6, Treatment=192 mg/m², Subject=1, Rsq=0.9998, Rsq_adjusted=0.9996, HL_Lambda_z=17.4892, 3 points used in calculation, Uniform Weighting.

FIG. 32B reflects WinNonlin Plots from Noncompartmental Analysis in subjects in Cohort 6 who received 192 mg/m². Cohort=Cohort 6, Treatment=192 mg/m², Subject=2, Rsq=0.9873, Rsq_adjusted=0.9746, HL_Lambda_z=16.4466, 3 points used in calculation, Uniform Weighting.

FIG. 32C reflects WinNonlin Plots from Noncompartmental Analysis in subjects in Cohort 6 who received 192 mg/m². Cohort=Cohort 6, Treatment=192 mg/m², Subject=3, Rsq=0.9969, Rsq_adjusted=0.9939, HL_Lambda_z=16.1036, 3 points used in calculation, Uniform Weighting.

FIG. 32D reflects WinNonlin Plots from Noncompartmental Analysis in subjects in Cohort 6 who received 192 mg/m². Cohort=Cohort 6, Treatment=192 mg/m², Subject=4, Rsq=0.9997, Rsq_adjusted=0.9994, HL_Lambda_z=17.104, 3 points used in calculation, Uniform Weighting.

FIG. 32E reflects WinNonlin Plots from Noncompartmental Analysis in subjects in Cohort 6 who received 192 mg/m². Cohort=Cohort 6, Treatment=192 mg/m², Subject=5, Rsq=0.998, Rsq_adjusted=0.9961, HL_Lambda_z=20.8967, 3 points used in calculation, Uniform Weighting.

FIG. 32F reflects WinNonlin Plots from Noncompartmental Analysis in subjects in Cohort 6 who received 192 mg/m². Cohort=Cohort 6, Treatment=192 mg/m², Subject=6, Rsq=0.9854, Rsq_adjusted=0.9708, HL_Lambda_z=19.7235, 3 points used in calculation, Uniform Weighting.

FIG. 33 reflects the mean Compound 1 plasma concentration-time plots (Linear) for Cohorts 1-6.

FIG. 34 reflects the mean Compound 1 plasma concentration-time plots (Log-Linear) for Cohorts 1-6.

FIG. 35 plots the relationship between Cmax and Dose over the range of 6 to 192 mg/m². Rsq=0.8736, Intercept=−74.62, Slope=10.78.

FIG. 36 plots the relationship between AUCinf and Dose over the range of 6 to 192 mg/m². Rsq=0.9485, Intercept=−142, Slope=16.88.

FIG. 37 plots the relationship between CL and Dose over the range of 6 to 192 mg/m². Rsq=0.4031, Intercept=90.53, Slope=−0.154.

FIG. 38 depicts the three compartment model for the PK parameters of Compound 1. K31 is the rate of transport from compartment 3 to compartment 1, K13 is the rate of transport from compartment 1 to compartment 3, K12 is the rate of transport from compartment 1 to compartment 2, K21 is the rate of transport from compartment 2 to compartment 1, and K10 is the rate of transport from compartment 1 out of the system.

FIG. 39A reflects the 3-Compartment Predicted vs Observed Concentration-Time Profiles for Cohort 5. Cohort=Cohort 5, Treatment=96 mg/m², Subject=1.

FIG. 39B reflects the 3-Compartment Predicted vs Observed Concentration-Time Profiles for Cohort 5. Cohort=Cohort 5, Treatment=96 mg/m², Subject=2.

FIG. 39C reflects the 3-Compartment Predicted vs Observed Concentration-Time Profiles for Cohort 5. Cohort=Cohort 5, Treatment=96 mg/m², Subject=3.

FIG. 39D reflects the 3-Compartment Predicted vs Observed Concentration-Time Profiles for Cohort 5. Cohort=Cohort 5, Treatment=96 mg/m², Subject=4.

FIG. 39E reflects the 3-Compartment Predicted vs Observed Concentration-Time Profiles for Cohort 5. Cohort=Cohort 5, Treatment=96 mg/m², Subject=5.

FIG. 39F reflects the 3-Compartment Predicted vs Observed Concentration-Time Profiles for Cohort 5. Cohort=Cohort 5, Treatment=96 mg/m², Subject=6.

FIG. 40A reflects the 3-Compartment Predicted vs Observed Concentration-Time Profiles for Cohort 6. Cohort=Cohort 6, Treatment=192 mg/m², Subject=1.

FIG. 40B reflects the 3-Compartment Predicted vs Observed Concentration-Time Profiles for Cohort 6. Cohort=Cohort 6, Treatment=192 mg/m², Subject=2.

FIG. 40C reflects the 3-Compartment Predicted vs Observed Concentration-Time Profiles for Cohort 6. Cohort=Cohort 6, Treatment=192 mg/m², Subject=3.

FIG. 40D reflects the 3-Compartment Predicted vs Observed Concentration-Time Profiles for Cohort 6. Cohort=Cohort 6, Treatment=192 mg/m², Subject=4.

FIG. 40E reflects the 3-Compartment Predicted vs Observed Concentration-Time Profiles for Cohort 6. Cohort=Cohort 6, Treatment=192 mg/m², Subject=5.

FIG. 40F reflects the 3-Compartment Predicted vs Observed Concentration-Time Profiles for Cohort 6. Cohort=Cohort 6, Treatment=192 mg/m², Subject=6.

FIG. 41 depicts the difference in cell cycling pre dosing Compound 1 and post dosing Compound 1. Compound 1 stops the interaction of EdU and thus causes G1 cell cycle arrest.

FIG. 42 shows the PHA induced stimulation of Cohort 5 & 6, wherein the data has been normalized to all placebo cohorts.

FIG. 43 shows the PHA induced stimulation of Cohort 5 & 6, wherein the data has been normalized to each separate placebo cohort.

FIG. 44 shows decrease in percentage of Hematopoietic Stem and Progenitor Cells (HSPCs) cycling cells at 24 hours post exposure to 192 mg/m² of Compound 1.

FIG. 45 shows decrease in percentage of oligopotent progenitor cells cycling cells at 24 hours post exposure to 192 mg/m² of Compound 1.

FIG. 46 shows decrease in percentage of monocytes cycling cells at 24 hours post exposure to 192 mg/m² of Compound 1.

FIG. 47 shows decrease in percentage of platelet lineage cycling cells at 24 hours post exposure to 192 mg/m² of Compound 1.

FIG. 48A depicts mean Compound 1 plasma concentrations in male and female rats following oral administration on day 1.

FIG. 48B depicts mean Compound 1 plasma concentrations in male and female rats following oral administration on day 14.

FIG. 49A depicts change in Compound 1 Cmax with increasing dose following oral administration in rats on study day 1.

FIG. 49B depicts change in Compound 1 Cmax with increasing dose following oral administration in rats on study day 14.

FIG. 50A depicts Change in Compound 1 AUCₗₐₛₜ with increasing dose following oral administration in rats on study day 1.

FIG. 50B depicts Change in Compound 1 AUCₗₐₛₜ with increasing dose following oral administration in rats on study day 14.

FIG. 51 shows plasma concentration over time of Compound 1 delivered to beagle dogs at 15 mpk at Day 1 of treatment.

FIG. 52 shows plasma concentration over time of Compound 1 delivered to beagle dogs at 15 mpk at Day 14 of treatment.

FIG. 53 shows plasma concentration over time of Compound 1 delivered to beagle dogs at 45 mpk at Day 1 of treatment.

FIG. 54 shows plasma concentration over time of Compound 1 delivered to beagle dogs at 45 mpk at Day 14 of treatment.

FIG. 55A is a graph of Compound 1 concentration (ng/mL) vs. time after administration of Compound 1 (hours) in dogs from toxicology studies (solid lines) and spot pharmacokinetic verification from bone marrow EdU experiments (symbols). Compound 1 was administered at 1 mg/kg (black line), 5 mg/kg (gray line), or 15 mg/kg (light gray line) in toxicology studies. Compound 1 was administered at 1 mg/kg (triangles), 5 mg/kg (diamonds), or 15 mg/kg (circles) in bone marrow EdU experiments.

FIG. 55B is a graph of the relative percentage of bone marrow cells in S-Phase (%) vs. time after administration of Compound 1 (hours). Cells in S-phase were determined based on EdU incorporation in dog whole bone marrow following a single dose of Compound 1 at 0 mg/kg (circles), 1 mg/kg (squares), 5 mg/kg (triangles) and 15 mg/kg (upside down triangles).

FIG. 55C is a graph of red blood cells (1000 cells/μL) vs. time after administration of Compound 1 (days). Red blood cells (RBCs) were counted from dogs who received a single dose of Compound 1 at 0 mg/kg (circles), 1 mg/kg (squares), 5 mg/kg (triangles) and 15 mg/kg (upside down triangles).

FIG. 55D is a graph of neutrophils (1000 cells/μL) vs. time after administration of Compound 1 (days). Neutrophils were counted from dogs who received a single dose of Compound 1 at 0 mg/kg (circles), 1 mg/kg (squares), 5 mg/kg (triangles) and 15 mg/kg (upside down triangles).

FIG. 56A is a graph of the percentage of hematopoietic stem cells and multipotent progenitor cells in G1(%) vs. time after administration of Compound 1 (hours). A single dose of Compound 1 was given at a concentration of 192 mg/m².

FIG. 56B is a graph of the percentage of human oligopotent progenitor cells in G1(%) vs. time after administration of Compound 1 (hours). A single dose of Compound 1 was given at a concentration of 192 mg/m².

FIG. 56C is a graph of the percentage of human monocytes in G1(%) vs. time after administration of Compound 1 (hours). A single dose of Compound 1 was given at a concentration of 192 mg/m².

FIG. 56D is a graph of the percentage of human granulocytes in G1(%) vs. time after administration of Compound 1 (hours). A single dose of Compound 1 was given at a concentration of 192 mg/m².

FIG. 56E is a graph of the percentage of human erythrocytes in G1(%) vs. time after administration of Compound 1 (hours). A single dose of Compound 1 was given at a concentration of 192 mg/m².

FIG. 56F is a graph of the percentage of human megakaryocytes in G1(%) vs. time after administration of Compound 1 (hours). A single dose of Compound 1 was given at a concentration of 192 mg/m².

FIG. 57A is a graph of Compound 1 concentration (ng/mL) in bone marrow plasma or blood plasma at 24 or 32 hours after administration of Compound 1 (192 mg/m²).

FIG. 57B is a graph of neutrophils (1000 cells/μL) vs. time after administration of Compound 1 (days). Neutrophils were counted from human patients who received a single dose of Compound 1 at 192 mg/m².

FIG. 57C is a graph of lymphocytes (1000 cells/μL) vs. time after administration of Compound 1 (days). Lymphocytes were counted from human patients who received a single dose of Compound 1 at 192 mg/m².

FIG. 57D is a graph of red blood cells (1000 cells/μL) vs. time after administration of Compound 1 (days). Red blood cells (RBCs) were counted from human patients who received a single dose of Compound 1 at 192 mg/m².

FIG. 57E is a graph of platelets (1000 cells/μL) vs. time after administration of Compound 1 (days). Platelets were counted from human patients who received a single dose of Compound 1 at 192 mg/m².

FIG. 58A is a graph of neutrophils (1000 cells/μL) in mice vs. compound dosed. When challenged with 5-Fluorouracil (5FU) neutrophil count diminished. In mice dosed with 100 mg/kg of Compound 1 before being challenged with 5FU, neutrophil count did not significantly decrease.

FIG. 58B is a graph of lymphocytes (1000 cells/μL) in mice vs. compound dosed. When challenged with 5-Fluorouracil (5FU) lymphocytes count diminished. In mice dosed with 100 mg/kg of Compound 1 before being challenged with 5FU, lymphocyte count decreased significantly less.

FIG. 58C is a graph of red blood cells (1000 cells/μL) in mice vs. compound dosed. When challenged with 5-Fluorouracil (5FU) red blood cell count diminished. In mice dosed with 100 mg/kg of Compound 1 before being challenged with 5FU, red blood cell count decreased significantly less.

FIG. 58D is a graph of platelets (1000 cells/μL) in mice vs. compound dosed. When challenged with 5-Fluorouracil (5FU) platelet count diminished. In mice dosed with 100 mg/kg of Compound 1 before being challenged with 5FU, platelet count decreased significantly less.

FIG. 59 is a bar graph of interferon gamma concentration (pg/ml) at day 2 and 7 for mouse splenocytes that were isolated and stimulated ex vivo with anti-CD3/antiCD28. In mice challenged with 5-Fluorouracil (5FU) (50 mg/kg) interferon gamma levels decreased significantly. In mice pretreated with 100 mg/kg of Compound 1 interferon gamma levels did not decrease significantly.

FIG. 60 is a time line displaying the dosing protocol used in the measurement of interferon gamma concentration in mice after being challenged with 5-Fluorouracil (5FU) either in the presence or absence of Compound 1.

FIG. 61 is a graph of tumor size (cubic millimeters) vs time (days) for a small cell lung cancer xenograft model with various dosing regimens. The solid square denotes control tumor size, the transparent square denotes tumor size in mice treated with 100 mg/kg doses of Compound 1 (qdx5dx4), the transparent circle denotes tumor size in mice treated with 0.6 mg/kg doses of Topotecan (qdx5dx4), the solid circle denotes tumor size in mice treated with 10 mg/kg Compound 1 and 0.6 mg/kg Topotecan, the transparent triangle denotes tumor size in mice treated with 50 mg/kg Compound 1 and 0.6 mg/kg Topotecan, and the solid triangle denotes tumor size in mice treated with 100 mg/kg Compound 1 and 0.6 mg/kg Topotecan.

FIG. 62 is a graph of plasma concentration (ng/mL) vs time (h) at various doses of Compound 1. The solid circle denotes 6 mg/m² dosing, the solid square denotes 12 mg/m² dosing, the solid triangle pointing up denotes 24 mg/m² dosing, the solid triangle pointing down denotes 48 mg/m² dosing, the solid diamond denotes 96 mg/m² dosing, the transparent circle and square denote 192 mg/m² dosing.

FIG. 63A is a graph of absolute neutrophil count (ANC, 1000 cells/μL) vs time (days) during the dosing cycle. The solid line depicts the group mean, the dashed line represents the standard error of the mean (SEM), the vertical dotted line depicts the planned start date of cycles 2 and 3 (cycle 2 was delayed by 1 day for one patient).

FIG. 63B is a graph of lymphocyte count (1000 cells/μL) vs time (days) during the dosing cycle. The solid line depicts the group mean, the dashed line represents the standard error of the mean (SEM), the vertical dotted line depicts the planned start date of cycles 2 and 3 (cycle 2 was delayed by 1 day for one patient).

FIG. 63C is a graph of hemoglobin concentration in (g/dL) vs time (days) during the dosing cycle. The solid line depicts the group mean, the dashed line represents the standard error of the mean (SEM), the vertical dotted line depicts the planned start date of cycles 2 and 3 (cycle 2 was delayed by 1 day for one patient).

FIG. 63D is a graph of platelet cell count (1000 cells/μL) vs time (days) during the cycle. The solid line depicts the group mean, the dashed line represents the standard error of the mean (SEM), the vertical dotted line depicts the planned start date of cycles 2 and 3 (cycle 2 was delayed by 1 day for one patient).

FIG. 64 is a bar graph of percent change of tumor from baseline (%) per patient. The patients were subjects of the Compound 1/Etoposide/Carboplatin dosing regimen described below. Compound 1 was dosed at 200 mg/m² prior to chemotherapy, Etoposide was dosed at 100 mg/m², and Carboplatin was dosed at a target AUC of 5 mg*min/ml.

FIG. 65 is a bar graph of percent change of tumor from baseline (%) per patient. The patients were subjects were second line patients dosed with Compound 1 and Topotecan. Compound 1 was dosed at 200 mg/m² prior to chemotherapy.

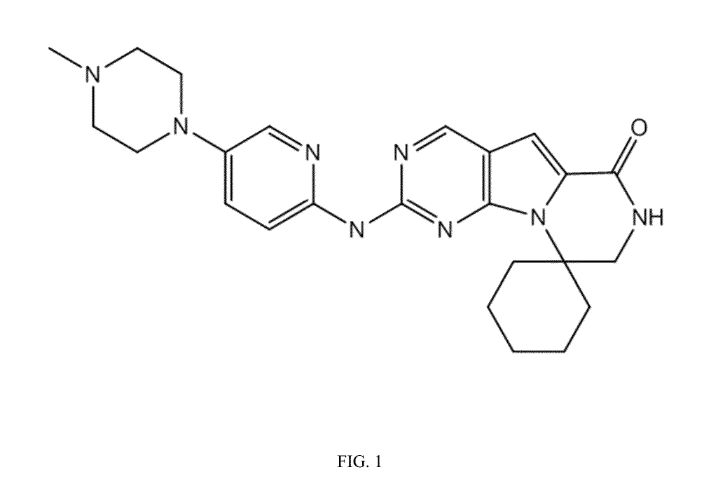

### DETAILED DESCRIPTION

Chemotherapy-induced myelosuppression continues to represent the major dose-limiting toxicity of cytotoxic chemotherapy and can be manifested as neutropenia, lymphopenia, anemia, and thrombocytopenia. As such, myelosuppression is the source of many of the adverse side effects of cancer treatment such as infection, sepsis, bleeding, and fatigue, leading to the need for hospitalizations, hematopoietic growth factor support, and transfusions (red blood cells and/or platelets). Moreover, clinical concerns raised by myelosuppression commonly lead to chemotherapy dose reductions, limit therapeutic dose-intensity, and the implementation of forced “off-cycle” or treatment holidays in order to allow a subject's hematopoietic cell lineages time to recover.

Compound 1 is a highly potent and selective, reversible, cyclin-dependent kinase (CDK)4/6 inhibitor that transiently produces a G0/G1 cell cycle arrest of hematopoietic stem and progenitor cells (HSPCs) in the bone marrow when properly dosed. These cells are dependent upon CDK4/6 for proliferation and enter the G0/G1 phase of the cell cycle upon exposure to Compound 1, termed pharmacological quiescence. When the HSPCs are transiently arrested in G0/G1, they are more resistant to the DNA damaging effects of chemotherapy, thus reducing subsequent myelosuppression and the downstream effects in treatment reduction or cessation associated with such myelosuppression.

It has been discovered that dosing human subjects with Compound 1 to achieve the PK and PD profiles described herein provides a sufficiently long arrest of HSPCs in the G0/G1 phase of the cell cycle to provide protection from chemotherapy-induced DNA damage followed by re-initiation of hematopoiesis following chemotherapy exposure, while reducing the risk associated with the use of other chemoprotectants, such as off-target effects or toxicity or hematological deficiencies. Together these characteristics provide for a clean, transient, and reversible G0/G1 arrest of HSPCs following drug exposure, and make Compound 1 dosed to achieve the PK and PD profiles described herein an ideal chemoprotectant.

The timely resumption of cellular proliferation is necessary for tissue repair, and therefore an overly long period of PQ, for example as demonstrated by the CDK 4/6 inhibitor palbociclib, is undesirable. The characteristics of a PQ compound that dictate its control of the cell cycle are its PK and enzymatic half-lives. Once initiated, a G1-arrest in vivo will be maintained as long as circulating compound remains at an inhibitory level, and as long as the compound engages the enzyme. Both in vitro and in vivo analyses have demonstrated the rapid resumption of cellular proliferation following cessation of Compound 1 drug exposure, consistent with a rapid enzymatic off-rate.

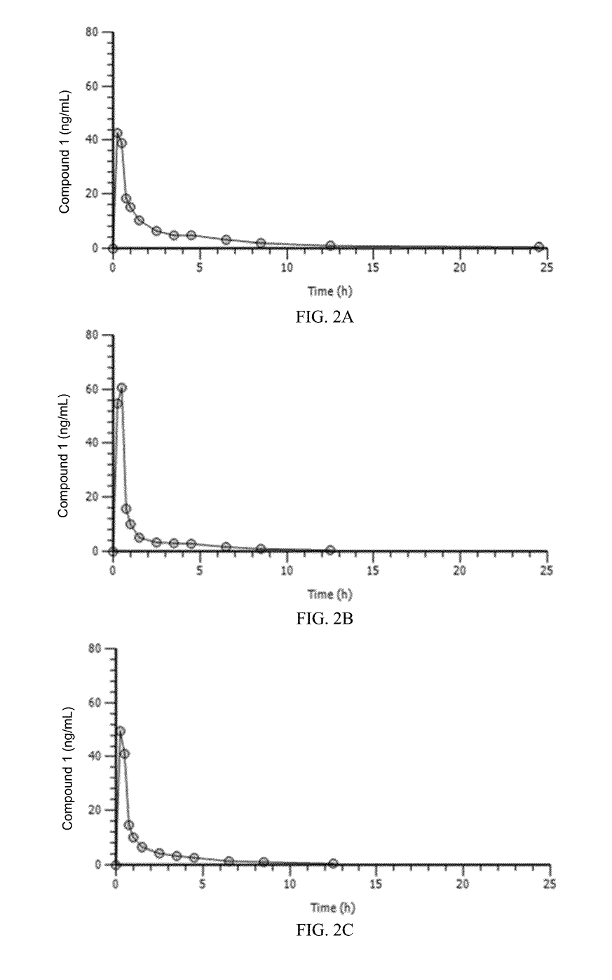

Despite reports using known CDK4/6 inhibitors such as 2BrIC and palbociclib to demonstrate chemoprotection, it has been discovered that these inhibitors may not be the most ideal compounds for use in pharmacological quiescence (PQ) strategies. For example, the use of 2BrIC in vivo is limited by its restricted bioavailability, and despite the relative selectivity for CDK4/6 exhibited by palbociclib, the compound has a relatively long-acting intra-cellular effect (see Roberts et al. Multiple Roles of Cyclin-Dependent Kinase 4/6 Inhibitors in Cancer Therapy. JCNI 2012; 104(6):476-487 (FIG. 2A)), extending the transiency of G1 arrest beyond what may be necessary for sufficient protection from chemotherapeutic treatments. Such a long acting effect delays, for example, the proliferation of HSPC cell lineages necessary to reconstitute the hematological cell lines that are adversely affected by chemotherapeutic agents or are cycled out during their natural life-cycle. The long-acting G1 arrest provided by palbociclib may limit its use as a potential chemoprotectant in subjects whose chemotherapeutic treatment regimen requires a rapid reentry into the cell cycle by HSPCs in order to reconstitute the erythroid, platelet, and myeloid cells (monocyte and granulocyte) adversely effected by chemotherapeutic agents or acute HSPC G1-arrest in order to limit myelosuppressive or hematologic toxicity effects. With respect to other affected tissues, for example renal cells, the timely resumption of proliferation is critical to tissue repair, for example renal tubular epithelium repair, due to nephrotoxic agents, and therefore, an overly long period of PQ is undesirable. In addition, palbociclib has been shown to accumulate in the blood plasma with repeated dosing schedules. Such an accumulation may be undesirable in a day-to-day dosing regimen due to the potential of exceeding an ideal therapeutic range, resulting in increased toxicities, drug-drug interactions, and off-target effects. This undesirable accumulation may also extend the re-entry of hematopoietic stem cells back into the cell-cycle, a severe and significant disadvantage in subjects whose hematologic cell lineages may have previously been adversely affected by prior chemotherapeutic treatments.

The principal component of the therapeutic use of Compound 1 as described herein is to transiently arrest HSPCs in G0/G1 while chemotherapy is administered. However, it is also very important to not impact anti-tumor efficacy of chemotherapy by promoting G0/G1 arrest of the tumor cells during chemotherapy, thus rendering them less sensitive to the intended cytotoxic effects. The downstream target of CDK4/6 is the Rb protein, which is phosphorylated upon CDK4/6 activation, allowing the cell to enter into the S phase of the cell cycle. In order to promote G0/G1 cell cycle arrest by utilizing a CDK4/6 inhibitor, a functional Rb protein (pRb) is required.

The Rb-protein is functionally inactivated in a number of cancers where highly myelosuppressive chemotherapy is used. These include, among others, small cell lung cancer, triple negative breast cancer, bladder cancer, ovarian cancer, and human papillomavirus-associated head and neck cancer. Utilizing the PQ approach with Compound 1 dosed as described herein to reduce chemotherapy-induced myelosuppression in these settings represents a significant advance for subjects, where survival is often longer for subjects able to receive multiple cycles and lines of chemotherapy without myelosuppresive induced chemotherapeutic holidays or dose reduction.

As described herein, the use of Compound 1 may also provide longer term effects by protecting HSPCs from DNA damage and thus maintaining a more robust replicative potential for these HSPCs compared to those that have been damaged by cytotoxic therapy in the absence of Compound 1. This may result in an improved ability for patients to tolerate chemotherapy, including subsequent lines of treatment.

Secondary malignancies can develop in patients who have previously received cytotoxic chemotherapy. The presumed mechanism for this effect is thought to be DNA damage to the HSPCs during exposure to chemotherapy resulting in cytogenetic changes that are associated with the development of myelodysplastic syndromes and leukemias. Reducing the damage to DNA of HSPCs by utilizing Compound 1 administered with chemotherapy could also reduce the incidence of these secondary myelodysplastic syndromes and leukemias.

### DEFINITIONS

AUC (Amount*time/volume) as used herein means the area under the plasma concentration-time curve.

AUC_{inf} (Amount*time/volume) as used herein means the area under the plasma concentration-time curve from time zero to infinity.

AUCₜ (Amount*time/volume) as used herein means the area under the plasma concentration time curve from time zero to time t.

AUCτ (Amount*time/volume) as used herein means the area under the plasma concentration-time curve during a dosage interval (τ).

AUCₗₐₛₜ (Amount*time/volume) as used herein means the area under the plasma concentration-time curve from time zero to time of the last measurable concentration.

Cₘₐₓ (Amount/volume) as used herein means the maximum (peak) plasma drug concentration.

CL (Volume/time or volume/time/kg) as used herein means the apparent total body clearance of the drug from plasma.

CL/F (Volume/time or volume/time/kg) as used herein means the apparent total clearance of the drug from plasma after administration.

κ (Time⁻¹) as used herein means first-order rate constant.

κ₁₂ (Time⁻¹) as used herein means the transfer rate constant (first-order) from the central (1) to the peripheral (2) compartment.

κ₂₁ (Time⁻¹⁾ as used herein means the transfer rate constant (first-order) from the peripheral (2) to the central (1) compartment.

κ₃₁ (Time⁻¹) as used herein means the transfer rate constant (first-order) from the deep peripheral (3) to the central (1) compartment.

κ_{Z} (Time−1) as used herein means the terminal disposition rate constant/terminal rate constant.

MRT_{inf} (Time) as used herein means mean residence time.

Tₘₐₓ (Time) as used herein means time to reach maximum (peak) plasma concentration following drug administration.

T1/2 (Time) as used herein means the elimination half-life as used in one or non-compartmental models.

T1/2β (Time) as used herein means the terminal elimination half-life as used in two-compartmental models.

T1/2γ (Time) as used herein means the terminal or elimination half-life as used in three compartmental models.

Vₛₛ (Volume or volume/kg) as used herein means the apparent volume of distribution at steady state.

As used herein, the term “prodrug” means a compound which when administered to a host in vivo is converted into the parent drug. As used herein, the term “parent drug” means any of the presently described chemical compounds that are useful to treat any of the disorders described herein, or to control or improve the underlying cause or symptoms associated with any physiological or pathological disorder described herein in a host, typically a human. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Nonlimiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

Throughout the specification and claims, a given chemical formula or name shall encompass all optical and stereoisomers, as well as racemic mixtures where such isomers and mixtures exist, unless otherwise noted.

The term “selective CDK4/6 inhibitor” used in the context of Compound 1 described herein indicates an ability to inhibit CDK4 activity, CDK6 activity, or both CDK4 and CDK6 activity at an IC₅₀ molar concentration at least about 2000 times less than the IC₅₀ molar concentration necessary to inhibit to the same degree CDK2 activity in a standard phosphorylation assay.

By “induces G1-arrest” is meant that Compound 1 induces a quiescent state in a substantial portion of a cell population at the G1 phase of the cell cycle.

By “hematological deficiency” is meant reduced hematological cell lineage counts or the insufficient production of blood cells (i.e., myelodysplasia) and/or lymphocytes (i.e., lymphopenia, the reduction in the number of circulating lymphocytes, such as B- and T-cells). Hematological deficiency can be observed, for example, as myelosuppression in form of anemia, reduction in platelet count (i.e., thrombocytopenia), reduction in white blood cell count (i.e., leukopenia), or the reduction in granulocytes (e.g., neutropenia).

By “synchronous reentry into the cell cycle” is meant that CDK4/6-replication dependent healthy cells, for example HSPCs, in G1-arrest due to the effect of Compound 1 reenter the cell-cycle within relatively the same collective timeframe or at relatively the same rate upon dissipation of the compound's effect. Comparatively, by “asynchronous reentry into the cell cycle” is meant that the healthy cells, for example HSPCs, in G1 arrest due to the effect of a CDK4/6 inhibitor compound within relatively different collective timeframes or at relatively different rates upon dissipation of the compound's effect such as pablociclib.

By “off-cycle” or “drug holiday” is meant a time period during which the subject is not administered or exposed to a chemotherapeutic. For example, in a treatment regime wherein the subject is administered the chemotherapeutic in a repeated 21 day cycle, and is not administered the chemotherapeutic at the start of the next 21-day cycle due to hematologic deficiencies, the delayed period of non-administration is considered the “off-cycle” or “drug holiday.” Off-target and drug holiday may also refer to an interruption in a treatment regime wherein the subject is not administered the chemotherapeutic for a time due to a deleterious side effect, for example, myelosuppression or other hematological deficiencies.

The term “pharmaceutically acceptable salt” as used herein refers to those salts which are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the compounds of the presently disclosed subject matter.

Thus, the term “salt” refers to the relatively non-toxic, inorganic and organic acid addition salts of compounds of the presently disclosed subject matter. These salts can be prepared in situ during the final isolation and purification of the compounds or by separately reacting the purified compound in its free base form with a suitable organic or inorganic acid and isolating the salt thus formed. Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

When used in the context of a dosage amount, e.g., mg/m², the numerical weight refers to the weight of Compound 1, exclusive of any salt, counterion, and so on. Therefore, to obtain the equivalent of 192 mg/m² of Compound 1, it would be necessary to utilize more than 192 mg/m² of its salt, due to the additional weight of the salt.

The present invention includes Compound 1 with desired isotopic substitutions of atoms, at amounts above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (²H) and tritium (³H) may be used anywhere in described structures. Alternatively or in addition, isotopes of carbon, e.g., ¹³C and ¹⁴C, may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect).

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break down can reduce the rate of or eliminate the metabolism at that bond. At any position of the compound that a hydrogen atom may be present, the hydrogen atom can be any isotope of hydrogen, including protium (¹H), deuterium (²H) and tritium (³H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby a H-isotope, i.e., hydrogen/protium (¹H), is substituted by a H-isotope, i.e., deuterium (²H). Deuterium substitution can be partial or complete. Partial deuterium substitution means that at least one hydrogen is substituted by at least one deuterium. In certain embodiments, the isotope is 90, 95 or 99% or more enriched in an isotope at any location of interest. In some embodiments it is deuterium that is 90, 95 or 99% enriched at a desired location.

The subject treated is typically a human subject, although it is to be understood the methods described herein are effective with respect to other animals, such as mammals and vertebrate species. More particularly, the term subject can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like.

By “substantial portion” or “significant portion” is meant at least 80%. In alternative embodiments, the portion may be at least 85%, 90% or 95% or greater.

In some embodiments, the term “CDK4/6-replication independent cancer” refers to a cancer that does not significantly require the activity of CDK4/6 for replication. Cancers of such type are often, but not always, characterized by (e.g., has cells that exhibit) an increased level of CDK2 activity or by reduced expression of retinoblastoma tumor suppressor protein or retinoblastoma family member protein(s), such as, but not limited to p107 and p130. The increased level of CDK2 activity or reduced or deficient expression of retinoblastoma tumor suppressor protein or retinoblastoma family member protein(s) can be increased or reduced, for example, compared to normal cells. In some embodiments, the increased level of CDK2 activity can be associated with (e.g., can result from or be observed along with) MYC proto-oncogene amplification or overexpression. In some embodiments, the increased level of CDK2 activity can be associated with overexpression of Cyclin E1, Cyclin E2, or Cyclin A.

By “long-term hematological toxicity” is meant hematological toxicity affecting a subject for a period lasting more than one or more weeks, months, or years following administration of a chemotherapeutic agent. Long-term hematological toxicity can result in bone marrow disorders that can cause the ineffective production of blood cells (i.e., myelodysplasia) and/or lymphocytes (i.e., lymphopenia, the reduction in the number of circulating lymphocytes, such as B- and T-cells). Hematological toxicity can be observed, for example, as anemia, reduction in platelet count (i.e., thrombocytopenia) or reduction in white blood cell count (i.e., neutropenia). In some cases, myelodysplasia can result in the development of leukemia. Long-term toxicity related to chemotherapeutic agents can also damage other self-renewing cells in a subject, in addition to hematological cells. Thus, long-term toxicity can also lead to graying and frailty.

### Chemotherapeutic Agents

As contemplated herein, Compound 1 is intravenously administered to a subject undergoing an anti-cancer therapeutic treatment regimen, for example a chemotherapeutic treatment regimen, prior to the subject receiving the anti-cancer therapy. As used herein the term “chemotherapy” or “chemotherapeutic agent” refers to treatment with a cytostatic or cytotoxic agent (i.e., a compound) to reduce or eliminate the growth or proliferation of undesirable cells, for example cancer cells. Thus, as used herein, “chemotherapy” or “chemotherapeutic agent” refers to a cytotoxic or cytostatic agent used to treat a proliferative disorder, for example cancer. The cytotoxic effect of the agent can be, but is not required to be, the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect. In one embodiment, the chemotherapeutic agent is selected from etoposide, carboplatin, cisplatin, and topotecan, or a combination thereof. In one embodiment, the chemotherapeutic agent is topotecan. In one embodiment, the chemotherapeutic agent is cisplatin. In one embodiment, the chemotherapeutic agent is carboplatin. In one embodiment, the chemotherapeutic agent is etoposide.

Thus, a “cytotoxic agent” can be any one or any combination of compounds also described as “antineoplastic” agents or “chemotherapeutic agents.” Such compounds include, but are not limited to, DNA damaging compounds and other chemicals that can kill cells. “DNA damaging chemotherapeutic agents” include, but are not limited to, alkylating agents, DNA intercalators, protein synthesis inhibitors, inhibitors of DNA or RNA synthesis, DNA base analogs, topoisomerase inhibitors, and telomerase inhibitors or telomeric DNA binding compounds. For example, alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylolmelamine; nitrogen mustards such as chlorambucil, chlornaphazine, cyclophosphamide, estramustine, iphosphamide, mechlorethamine, mechlorethamine oxide hydrochloride, melphalan, novembichine, phenesterine, prednimustine, trofosfamide, and uracil mustard; and nitroso ureas, such as carmustine, chlorozotocin, fotemustine, lomustine, nimustine, and ranimustine.

Antibiotics used in the treatment of cancer include dactinomycin, daunorubicin, doxorubicin, idarubicin, bleomycin sulfate, mytomycin, plicamycin, and streptozocin. Chemotherapeutic antimetabolites include mercaptopurine, thioguanine, cladribine, fludarabine phosphate, fluorouracil (5-FU), floxuridine, cytarabine, pentostatin, methotrexate, and azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine β-1-D-arabinoside, diazooxynorleucine, dideoxynucleosides, 5-fluorodeoxycytidine, 5-fluorodeoxyuridine, and hydroxyurea.

Chemotherapeutic protein synthesis inhibitors include abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim. Inhibitors of DNA synthesis, include alkylating agents such as dimethyl sulfate, mitomycin C, nitrogen and sulfur mustards; intercalating agents, such as acridine dyes, actinomycins, adriamycin, anthracenes, benzopyrene, ethidium bromide, propidium diiodide-intertwining; and other agents, such as distamycin and netropsin. Topoisomerase inhibitors, such as coumermycin, nalidixic acid, novobiocin, and oxolinic acid; inhibitors of cell division, including colcemide, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be used as the DNA damaging compound.

Current chemotherapeutic agents whose toxic effects can be mitigated by the presently disclosed dosages of Compound 1 include, but are not limited to, adrimycin, 5-fluorouracil (5FU), 6-mercaptopurine, gemcitabine, melphalan, chlorambucil, mitomycin, irinotecan, mitoxantrone, etoposide, camptothecin, topotecan, irinotecan, exatecan, lurtotecan, actinomycin-D, mitomycin, cisplatin, hydrogen peroxide, carboplatin, procarbazine, mechlorethamine, cyclophosphamide, ifosfamide, melphalan, chlorambucil, busulfan, nitrosurea, dactinomycin, daunorubicin, doxorubicin, bleomycin, plicomycin, tamoxifen, taxol, transplatinum, vinblastine, vinblastin, carmustine, cytarabine, mechlorethamine, chlorambucil, streptozocin, lomustine, temozolomide, thiotepa, altretamine, oxaliplatin, campothecin, topotecan, and methotrexate, and the like, and similar acting-type agents. In one embodiment, the DNA damaging chemotherapeutic agent is selected from the group consisting of cisplatin, carboplatin, campothecin, doxorubicin, and etoposide. In one embodiment, the DNA damaging chemotherapeutic agent is topotecan. In one embodiment, the DNA-damaging chemotherapeutic agent is etoposide. In one embodiment, the DNA damaging chemotherapeutic agent is carboplatin. In one embodiment, the DNA damaging chemotherapeutic agent is a combination of etoposide and carboplatin.

In certain alternative embodiments, Compound 1 in a dosage described herein is also used for an anti-cancer or anti-proliferative effect in combination with a chemotherapeutic to treat a CDK4/6 replication independent, such as an Rb-negative cancer or proliferative disorder. Compound 1, under certain conditions, may provide an additive or synergistic effect to the chemotherapeutic, resulting in a greater anti-cancer effect than seen with the use of the chemotherapeutic alone. In one embodiment, Compound 1 can be combined with one or more of the chemotherapeutic compounds described above. In one embodiment, Compound 1 can be combined with a chemotherapeutic selected from, but not limited to, tamoxifen, midazolam, letrozole, bortezomib, anastrozole, goserelin, an mTOR inhibitor, a PI3 kinase inhibitor, a dual mTOR-PI3K inhibitor, a Bruton's tyrosine kinase (BTK) inhibitor, a spleen tyrosine kinase (Syk) inhibitor, a MEK inhibitor, a RAS inhibitor, an ALK inhibitor, an HSP inhibitor (for example, an HSP70 or an HSP 90 inhibitor, or a combination thereof), a BCL-2 inhibitor, an apopototic inducing compound, an AKT inhibitor, including but not limited to, MK-2206, GSK690693, Perifosine, (KRX-0401), GDC-0068, Triciribine, AZD5363, Honokiol, PF-04691502, and Miltefosine, a PD-1 inhibitor including but not limited to, Nivolumab, CT-011, MK-3475, BMS936558, and AMP-514 or a FLT-3 inhibitor, including but not limited to, P406, Dovitinib, Quizartinib (AC220), Amuvatinib (MP-470), Tandutinib (MLN518), ENMD-2076, and KW-2449, or combinations thereof. Examples of mTOR inhibitors include but are not limited to rapamycin and its analogs, everolimus (Afinitor), temsirolimus, ridaforolimus, sirolimus, and deforolimus.

PI3k inhibitors that may be used in the present invention are well known. Examples of PI3 kinase inhibitors include but are not limited to Wortmannin, demethoxyviridin, perifosine, idelalisib, Pictilisib, Palomid 529, ZSTK474, PWT33597, CUDC-907, and AEZS-136, duvelisib, GS-9820, GDC-0032 (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2 S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3 aR,6E,9 S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl]acetate (also known as sonolisib)), and the structure described in WO2014/071109 having the formula:

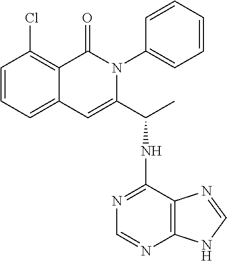 [SMILES: C[C@H](Nc1ncnc2[nH]cnc12)c1cc2cccc(Cl)c2c(=O)n1-c1ccccc1]

Compound 292. In one embodiment, Compound 1 is combined in a single dosage form with the PIk3 inhibitor.

BTK inhibitors for use in the present invention are well known. Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference. In one embodiment, Compound 1 is combined in a single dosage form with the BTK inhibitor.

Syk inhibitors for use in the present invention are well known, and include, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium(6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein). In one embodiment, Compound 1 is combined in a single dosage form with the Syk inhibitor.

MEK inhibitors for use in the present invention are well known, and include, for example, tametinib/GSK1 120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl)}phenyl)acetamide), selumetinob (6-(4-bromo-2-chloroanilin)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2 S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamin)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide). In one embodiment, Compound 1 is combined in a single dosage form with the MEK inhibitor.

Raf inhibitors for use in the present invention are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), and Sorafenib N-Oxide (4-[4-[[[[4-Chloro-3 (trifluoroMethyl)phenyl]aMino]carbonyl]aMino]phenoxy]-N-Methyl-2pyridinecarboxaMide 1-Oxide). In one embodiment, Compound 1 is combined in a single dosage form with the Raf inhibitor. In one embodiment, the at least one additional chemotherapeutic agent combined or alternated with Compound 1 is a protein cell death-1 (PD-1) inhibitor. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech). In one embodiment, Compound 1 is combined in a single dosage form with the PD-1 inhibitor.

In one embodiment, the at least one additional chemotherapeutic agent combined or alternated with a selected compound disclosed herein is a B-cell lymphoma 2 (Bcl-2) protein inhibitor. BCL-2 inhibitors are known in the art, and include, for example, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), or G3139 (Oblimersen). In one embodiment, Compound 1 is administered in the dosage described herein and combined in a single dosage form with the at least one BCL-2 inhibitor.

Examples of RAS inhibitors include but are not limited to Reolysin and siGl2D LODER. Examples of ALK inhibitors include but are not limited to Crizotinib, AP26113, and LDK378. HSP inhibitors include but are not limited to Geldanamycin or 17-N-Allylamino-17-demethoxygeldanamycin (17AAG), and Radicicol.

In one embodiment of the invention, Compound 1 is administered in the dosage described herein in combination with a topoisomerase inhibitor. In one aspect, an advantageous treatment of select Rb-negative cancers is disclosed using Compound 1 in combination with a topoisomerase inhibitor. In one embodiment, the topoisomerase inhibitor is a topoisomerase I inhibitor or a topoisomerase I and II dual inhibitor. In one embodiment, the topoisomerase inhibitor is a topoisomerase II inhibitor.

In one embodiment, the topoisomerase inhibitor is selected from a topoisomerase I inhibitor. Known topoisomerase I inhibitors useful in the present invention include (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride (topotecan), (S)-4-ethyl-4-hydroxy-1H-pyrano[3′,4′:6,7]indolizino[1,2-b]quinoline-3,14-(4H,12H)-dione (camptothecin), (1 S,9 S)-1-Amino-9-ethyl-5-fluoro-1,2,3,9,12,15-hexahydro-9-hydroxy-4-methyl-10H,13H-benzo(de)pyrano(3′,4′:6,7)indolizino(1,2-b)quinoline-10,13-dione (exatecan), (7-(4-methylpiperazinomethylene)-10,11-ethylenedioxy-20(S)-camptothecin (lurtotecan), or (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate (irinotecan), (R)-5-ethyl-9,10-difluoro-5-hydroxy-4,5-dihydrooxepino[3′,4′:6,7]indolizino[1,2-b]quinoline-3,15(1H,13H)-dione (diflomotecan), (4S)-11-((E)-((1-Dimethylethoxy)imino)methyl)-4-ethyl-4-hydroxy-1,12-dihydro-14H-pyrano(3′,4′:6,7)indolizino(1,2-b)quinoline-3,14(4H)-dione (gimatecan), (S)-8-ethyl-8-hydroxy-15-((4-methylpiperazin-1-yl)methyl)-11,14-dihydro-2H-[1,4]dioxino[2,3-g]pyrano[3′,4′:6,7]indolizino[1,2-b]quinoline-9,12(3H,8H)-dione (lurtotecan), (4S)-4-Ethyl-4-hydroxy-11-[2-[(1-methylethyl)amino]ethyl]-1H-pyrano[3 ?,4?:6,7]indolizino[1,2-b]quinoline-3,14(4H,12H)-dione (belotecan), 6-((1,3-dihydroxypropan-2-yl)amino)-2,10-dihydroxy-12-((2R,3R,4S,5 S,6R)-3,4,5-trihydroxy-6-(hydroxymethyl)tetrahydro-2H-pyran-2-yl)-12,13-dihydro-5H-indolo[2,3-a]pyrrolo[3,4-c]carbazole-5,7(6H)-dione (edotecarin), 8,9-dimethoxy-5-(2-N,N-dimethylaminoethyl)-2,3-methylenedioxy-5H-dibenzo(c,h)(1,6)naphthyridin-6-one (topovale), benzo[6,7]indolizino[1,2-b]quinolin-11(13H)-one (rosettacin), (S)-4-ethyl-4-hydroxy-11-(2-(trimethylsilyl)ethyl)-1H-pyrano[3′,4′:6,7]indolizino[1,2-b]quinoline-3,14(4H, 12H)-dione (cositecan), tetrakis{(4S)-9-[([1,4′-bipiperidinyl]-1′-carbonyl)oxy]-4,11-diethyl-3,14-dioxo-3,4,12,14-tetrahydro-1H-pyrano[3′,4′:6,7]indolizino[1,2-b]quinolin-4-yl}N,N′,N″,N′″-{{methanetetrayltetrakis[methylenepoly(oxyethylene)oxy(1-oxoethylene)]}tetraglycinate tetrahydrochloride (etirinotecan pegol), 10-hydroxy-camptothecin (HOCPT), 9-nitrocamptothecin (rubitecan), SN38 (7-ethyl-10-hydroxycamptothecin), and 10-hydroxy-9-nitrocamptothecin (CPT109), (R)-9-chloro-5-ethyl-5-hydroxy-10-methyl-12-((4-methylpiperidin-1-yl)methyl)-4,5-dihydrooxepino[3′,4′:6,7]indolizino[1,2-b]quinoline-3,15(1H, 13H)-dione (elmotecan).

In a particular embodiment, the topoisomerase inhibitor is the topoisomerase I inhibitor (S)-10-[(dimethylamino)methyl]-4-ethyl-4,9-dihydroxy-1H pyrano[3′,4′:6,7]indolizino[1,2-b]quinoline-3,14(4H,12H)-dione monohydrochloride (topotecan hydrochloride). In one non-limiting example, Compound 1 is administered in the dosage described herein in combination with a topoisomerase I inhibitor selected from the group consisting of (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride (topotecan), (S)-4-ethyl-4-hydroxy-1H-pyrano[3′,4′:6,7]indolizino[1,2-b]quinoline-3,14-(4H, 12H)-dione (camptothecin), (1S,9S)-1-Amino-9-ethyl-5-fluoro-1,2,3,9,12,15-hexahydro-9-hydroxy-4-methyl-10H,13H-benzo(de)pyrano(3′,4′:6,7)indolizino(1,2-b)quinoline-10,13-dione (exatecan), (7-(4-methylpiperazinomethylene)-10,11-ethylenedioxy-20(S)-camptothecin (lurtotecan), or (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate (irinotecan). In one embodiment, the topoisomerase I inhibitor is topotecan.

### CDK-Replication Dependent Cells and Cyclin-Dependent Kinase Inhibitors

Tissue-specific stem cells and subsets of other resident proliferating cells are capable of self-renewal, meaning that they are capable of replacing themselves throughout the adult mammalian lifespan through regulated replication. Additionally, stem cells divide asymmetrically to produce “progeny” or “progenitor” cells that in turn produce various components of a given organ. For example, in the hematopoietic system, the hematopoietic stem cells give rise to progenitor cells which in turn give rise to all the differentiated components of blood (e.g., white blood cells, red blood cells, and platelets).

Certain proliferating cells, such as HSPCs, require the enzymatic activity of the proliferative kinases cyclin-dependent kinase 4 (CDK4) and/or cyclin-dependent kinase 6 (CDK6) for cellular replication. In contrast, the majority of proliferating cells in adult mammals (e.g., the more differentiated blood-forming cells in the bone marrow) do not require the activity of CDK4 and/or CDK6 (i.e., CDK4/6). These differentiated cells can proliferate in the absence of CDK4/6 activity by using other proliferative kinases, such as cyclin-dependent kinase 2 (CDK2) or cyclin-dependent kinase 1 (CDK1).

Compound 1 shows a marked selectivity for the inhibition of CDK4 and/or CDK6 in comparison to other CDKs, for example CDK2. For example, the present invention provide for a dose-dependent G1-arresting effect on a subject's CDK4/6-replication dependent healthy cells, for example HSPCs or renal epithelial cells, and the methods provided for herein are sufficient to afford chemoprotection to targeted CDK4/6-replication dependent healthy cells during chemotherapeutic agent exposure, for example, during the time period that a DNA-damaging chemotherapeutic agent is capable of DNA-damaging effects on CDK4/6-replication dependent healthy cells in the subject, while allowing for the synchronous and rapid reentry into the cell-cycle by these cells shortly after the chemotherapeutic agent dissipates due to the time-limited CDK4/6 inhibitory effect provided by the compound compared to, for example, palbociclib.

In some embodiments, a CDK4/6-replication dependent healthy cell is a hematopoietic stem progenitor cell. Hematopoietic stem and progenitor cells include, but are not limited to, long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs). Administration of Compound 1 provides temporary, transient pharmacologic quiescence of hematopoietic stem and/or hematopoietic progenitor cells in the subject.

In some embodiments, the CDK4/6-replication dependent healthy cell may be a cell in a non-hematopoietic tissue, such as, but not limited to, the liver, kidney, pancreas, brain, lung, adrenals, intestine, gut, stomach, skin, auditory system, bone, bladder, ovaries, uterus, testicles, gallbladder, thyroid, heart, pancreatic islets, blood vessels, and the like. In some embodiments, the CDK4/6-replication dependent healthy cell is a renal cell, and in particular a renal epithelial cell, for example, a renal proximal tubule epithelial cells. In some embodiments, a CDK4/6-replication dependent healthy cell is a hematopoietic stem progenitor cell. In some embodiments, the CDK4/6-replication dependent healthy cell may be a cell in a non-hematopoietic tissue, such as, but not limited to, the liver, kidney, pancreas, brain, lung, adrenals, intestine, gut, stomach, skin, auditory system, bone, bladder, ovaries, uterus, testicles, gallbladder, thyroid, heart, pancreatic islets, blood vessels, and the like.

Likewise, the present invention provides for a dose-dependent mitigating effect on CDK4/6-replication dependent healthy cells that have been exposed to toxic levels of chemotherapeutic agents, allowing for repair of DNA damage associated with chemotherapeutic agent exposure and synchronous, rapid reentry into the cell-cycle following dissipation of the CDK4/6 inhibitory effect compared to, for example, palbociclib. In one embodiment, the use of Compound 1 results in the G1-arresting effect on the subject's CDK4/6-replication dependent healthy cells dissipating following administration of Compound 1 so that the subject's healthy cells return to or approach their pre-administration baseline cell-cycle activity within less than about 24 hours, 30 hours, 36 hours, or 40 hours, of administration. In one embodiment, the G1-arresting effect dissipates such that the subject's CDK4/6-replication dependent healthy cells return to their pre-administration baseline cell-cycle activity within less than about 24 hours, 30 hours, 36 hours, or 40 hours.

In one embodiment, the use of Compound 1 described herein results in the G1-arresting effect dissipating such that the subject's CDK4/6-dependent healthy cells return to or approach their pre-administration baseline cell-cycle activity within less than about 24 hours, 30 hours, 36 hours, or 40 hours of the chemotherapeutic agent effect. In one embodiment, the G1-arresting effect dissipates such that the subject's CDK4/6-replication dependent cells return to their pre-administration baseline cell-cycle activity within less than about 24 hours, 30 hours, 36 hours, or 40 hours, or within about 48 hours of the cessation of the chemotherapeutic agent administration. In one embodiment, the CDK4/6-replication dependent healthy cells are HSPCs. In one embodiment, the CDK4/6-dependent healthy cells are renal epithelial cells.

In one embodiment, the use of Compound 1 as described herein results in the G1-arresting effect dissipating so that the subject's CDK4/6-replication dependent healthy cells return to or approach their pre-administration baseline cell-cycle activity within less than about 24 hours, 30 hours, 36 hours, 40 hours, or within less than about 48 hours from the point in which the CDK4/6 inhibitor's concentration level in the subject's blood drops below a therapeutic effective concentration.

In one embodiment, Compound 1 is used to protect renal epithelium cells during exposure to a chemotherapeutic agent, for example, a DNA damaging chemotherapeutic agent, wherein the renal epithelial cells are transiently prevented from entering S-phase in response to chemotherapeutic agent induced renal tubular epithelium damage for no more than about 24 hours, about 30 hours, about 36 hours, about 40 hours, or about 48 hours from the point in which the Compound 1's concentration level in the subject's blood drops below a therapeutic effective concentration or biological effective concentration, from the cessation of the chemotherapeutic agent effect, or from administration of Compound 1.

Compound 1 may be synchronous in its off-effect, that is, upon dissipation of the G1 arresting effect, CDK4/6-replication dependent healthy cells exposed to Compound 1 in the concentrations described herein reenter the cell-cycle in a similarly timed fashion. CDK4/6-replication dependent healthy cells that reenter the cell-cycle do so such that the normal proportion of cells in G1 and S are reestablished quickly and efficiently, within less than about 24 hours, 30 hours, 36 hours, 40 hours, or within about 48 hours of the from the point in which Compound 1's concentration level in the subject's blood drops below a therapeutic effective concentration. This advantageously allows for a larger number of healthy cells to begin replicating upon dissipation of the G1 arrest compared with asynchronous CDK4/6 inhibitors such as palbociclib.

In addition, synchronous cell-cycle reentry following G1 arrest using Compound 1 in concentrations and dosages described herein provides for the ability to time the administration of hematopoietic growth factors to assist in the reconstitution of hematopoietic cell lines to maximize the growth factor effect. In one embodiment of the invention, Compound 1 can be administered in a concerted regimen with a blood growth factor agent. As such, in one embodiment, the use of Compound 1 and methods described herein are combined with the use of hematopoietic growth factors including, but not limited to, granulocyte colony stimulating factor (G-CSF, for example, sold as Neupogen (filgrastin), Neulasta (peg-filgrastin), or lenograstin), granulocyte-macrophage colony stimulating factor (GM-CSF, for example sold as molgramostim and sargramostim (Leukine)), M-CSF (macrophage colony stimulating factor), thrombopoietin (megakaryocyte growth development factor (MGDF), for example sold as Romiplostim and Eltrombopag) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (sold as for example epoetin-α as Darbopoetin, Epocept, Nanokine, Epofit, Epogin, Eprex and Procrit; epoetin-β sold as for example NeoRecormon, Recormon and Micera), epoetin-delta (sold as for example Dynepo), epoetin-omega (sold as for example Epomax), epoetin zeta (sold as for example Silapo and Reacrit) as well as for example Epocept, EPOTrust, Erypro Safe, Repoeitin, Vintor, Epofit, Erykine, Wepox, Espogen, Relipoeitin, Shanpoietin, Zyrop and EPIAO). In one embodiment, Compound 1 is administered prior to administration of the hematopoietic growth factor. In one embodiment, the hematopoietic growth factor administration is timed so that Compound 1's inhibitory effect on HSPCs has dissipated.

### CDK 4/6-Replication Independent Proliferative Disorders

In one aspect of the present invention, provided herein is a dosing regimen comprising the administration of Compound 1 that provides a specific PK and/or PD blood profile followed by the administration of a chemotherapeutic agent for the treatment of the CDK 4/6-replication independent cellular proliferation disorder. The subject treated according to the present invention may be undergoing therapeutic chemotherapy for the treatment of a proliferative disorder that is CDK4/6 replication independent.

CDK 4/6-replication independent cellular proliferation disorders, for example as seen in certain types of cancer, can be characterized by one or a combination of increased activity of cyclin-dependent kinase 1 (CDK1), increased activity of cyclin-dependent kinase 2 (CDK2), loss, deficiency, or absence of retinoblastoma tumor suppressor protein (Rb)(Rb-null), high levels of MYC expression, increased cyclin E1, E2, and increased cyclin A. The cancer may be characterized by reduced expression of the retinoblastoma tumor suppressor protein or a retinoblastoma family member protein or proteins (such as, but not limited to p107 and p130). In one embodiment, the subject is undergoing chemotherapeutic treatment for the treatment of an Rb-null or Rb-deficient cancer, including but not limited to small cell lung cancer, triple-negative breast cancer, HPV-positive head and neck cancer, retinoblastoma, Rb-negative bladder cancer, Rb negative prostate cancer, osteosarcoma, or cervical cancer. Administration of Compound 1 may allow for a higher dose of a chemotherapeutic agent to be used to treat the disease than the standard dose that would be safely used in the absence of administration of Compound 1.

The host or subject, including a human, may be undergoing chemotherapeutic treatment of a non-malignant proliferative disorder, or other abnormal cellular proliferation, such as a tumor, multiple sclerosis, lupus, or arthritis.

Proliferative disorders that are treated with chemotherapy include cancerous and non-cancer diseases. In a typical embodiment, the proliferative disorder is a CDK4/6-replication independent disorder. Compound 1 is effective in protecting healthy CDK4/6-replication dependent cells, for example HSPCs, during chemotherapeutic treatment of a broad range of tumor types, including but not limited to the following: breast, prostate, ovarian, skin, lung, colorectal, brain (i.e., glioma) and renal. Preferably, Compound 1 should not compromise the efficacy of the chemotherapeutic agent or arrest G1 arrest the cancer cells. Many cancers do not depend on the activities of CDK4/6 for proliferation as they can use the proliferative kinases promiscuously (e.g., can use CDK 1/2/4/or 6) or lack the function of the retinoblastoma tumor suppressor protein (Rb), which is inactivated by the CDKs. The potential sensitivity of certain tumors to CDK4/6 inhibition can be deduced based on tumor type and molecular genetics using standard techniques. Cancers that are not typically affected by the inhibition of CDK4/6 are those that can be characterized by one or more of the group including, but not limited to, increased activity of CDK1 or CDK2, loss, deficiency, or absence of retinoblastoma tumor suppressor protein (Rb), high levels of MYC expression, increased cyclin E (e.g., E1 or E2) and increased cyclin A, or expression of a Rb-inactivating protein (such as HPV-encoded E7). Such cancers can include, but are not limited to, small cell lung cancer, retinoblastoma, HPV positive malignancies like cervical cancer and certain head and neck cancers, MYC amplified tumors such as Burkitts' Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancers, and certain classes of testicular cancers.

The loss or absence of retinoblastoma (Rb) tumor suppressor protein (Rb-null) can be determined through any of the standard assays known to one of ordinary skill in the art, including but not limited to Western Blot, ELISA (enzyme linked immunoadsorbent assay), IHC (immunohistochemistry), and FACS (fluorescent activated cell sorting). The selection of the assay will depend upon the tissue, cell line or surrogate tissue sample that is utilized e.g., for example Western Blot and ELISA may be used with any or all types of tissues, cell lines or surrogate tissues, whereas the IHC method would be more appropriate wherein the tissue utilized in the methods of the present invention was a tumor biopsy. FACs analysis would be most applicable to samples that were single cell suspensions such as cell lines and isolated peripheral blood mononuclear cells. See for example, US 20070212736 “Functional Immunohistochemical Cell Cycle Analysis as a Prognostic Indicator for Cancer”.

Alternatively, molecular genetic testing may be used for determination of retinoblastoma gene status. Molecular genetic testing for retinoblastoma includes the following as described in Lohmann and Gallie “Retinoblastoma. Gene Reviews” (2010) or Parsam et al. “A comprehensive, sensitive and economical approach for the detection of mutations in the RB 1 gene in retinoblastoma” Journal of Genetics, 88(4), 517-527 (2009).

Increased activity of CDK1 or CDK2, high levels of MYC expression, increased cyclin E and increased cyclin A can be determined through any of the standard assays known to one of ordinary skill in the art, including but not limited to Western Blot, ELISA (enzyme linked immunoadsorbent assay), IHC (immunohistochemistry), and FACS (fluorescent activated cell sorting). The selection of the assay will depend upon the tissue, cell line, or surrogate tissue sample that is utilized e.g., for example Western Blot and ELISA may be used with any or all types of tissues, cell lines, or surrogate tissues, whereas the IHC method would be more appropriate wherein the tissue utilized in the methods of the present invention was a tumor biopsy. FACs analysis would be most applicable to samples that were single cell suspensions such as cell lines and isolated peripheral blood mononuclear cells.

In some embodiments, the cancer is selected from a small cell lung cancer, retinoblastoma, and triple negative (ER/PR/Her2 negative) or “basal-like” breast cancer, which almost always have inactivate retinoblastoma tumor suppressor proteins (Rb), and therefore do not require CDK4/6 activity to proliferate. Triple negative (basal-like) breast cancer is also almost always genetically or functionally Rb-null. Also, certain virally induced cancers (e.g. cervical cancer and subsets of Head and Neck cancer) express a viral protein (E7) which inactivates Rb making these tumors functionally Rb-null. Some lung cancers are also believed to be caused by HPV. In one particular embodiment, the cancer is small cell lung cancer, and the patient is treated with a DNA-damaging agent selected from the group consisting of etoposide, carboplatin, and cisplatin, or a combination thereof.

Compound 1 can also be used in protecting healthy CDK4/6-replication dependent cells during chemotherapeutic treatments of abnormal tissues in non-cancer proliferative diseases, including but not limited to: psoriasis, lupus, arthritis (notably rheumatoid arthritis), hemangiomatosis in infants, multiple sclerosis, myelodegenerative disease, neurofibromatosis, ganglioneuromatosis, keloid formation, Paget's Disease of the bone, fibrocystic disease of the breast, Peyronie's and Duputren's fibrosis, restenosis, and cirrhosis. Further, Compound 1 can be used to ameliorate the effects of chemotherapeutic agents in the event of accidental exposure or overdose (e.g., methotrexate overdose).

### Chemoprotective Regimes

In certain embodiments, Compound 1 or a pharmaceutically acceptable composition, salt, isotopic analog, or prodrug thereof, is administered at a dose described herein so that the protection afforded by the compound is short term and transient in nature, allowing a significant portion of the cells to synchronously renter the cell-cycle quickly following the cessation of the chemotherapeutic agent's effect, for example within less than about 24, 30, 36, or 40 hours. Cells that are quiescent within the G1 phase of the cell cycle are more resistant to the damaging effect of chemotherapeutic agents than proliferating cells.

As described herein, Compound 1 can be administered to the subject prior to treatment with a chemotherapeutic agent, during treatment with a chemotherapeutic agent, after exposure to a chemotherapeutic agent, or a combination thereof. As contemplated herein, Compound 1 is typically administered in a manner that allows the drug facile access to the blood stream, for example via intravenous injection. In one embodiment, the compound is administered to the subject less than about 24 hours, 20 hours, 16 hours, 12 hours, 8 hours, or 4 hours, 2.5 hours, 2 hours, 1 hour, 1/2 hour or less prior to treatment with the chemotherapeutic agent. In an alternative embodiment, the compound is administered to the subject less than about 48 hours, 40 hours, 36 hours, or 32 hours or less prior to treatment with the chemotherapeutic agent.

Typically, the compound described herein is administered to the subject prior to treatment with the chemotherapeutic agent such that the compound reaches peak serum levels before or during treatment with the chemotherapeutic agent. In one embodiment, Compound 1 is administered to the subject about 30 minutes prior to administration of the chemotherapeutic agent. In one embodiment, Compound 1 is administered to the subject over about a 30 minute period and then the subject is administered a chemotherapeutic agent. In one embodiment, the Compound is administered concomitantly, or closely thereto, with the chemotherapeutic agent exposure. If desired, the compound can be administered multiple times during the chemotherapeutic agent treatment to maximize inhibition, especially when the chemotherapeutic drug is administered over a long period or has a long half-life. In an alternative embodiment, Compound 1 can be administered following exposure to the chemotherapeutic agent if desired to mitigate healthy cell damage associated with chemotherapeutic agent exposure. In certain embodiments, the compound is administered up to about ½ Hour, Up to about 1 Hour, Up to about 2 Hours, Up to about 4 Hours, Up to about 8 Hours, Up to about 10 hours, up to about 12 hours, up to about 14 hours, up to about 16 hours, or up to about 20 hours or greater following the chemotherapeutic agent exposure. In a particular embodiment, the compound is administered up to between about 12 hours and 20 hours following exposure to the chemotherapeutic agent.

Importantly, at the dosing ranges provided herein, Compound 1 can be used in a multi-day chemotherapeutic regimen without concomitant accumulation in the subject. Accordingly, the PK and/or PD levels provided herein are not significantly altered, that is, by no more than about 10%, across a multi-day dosing regimen. Because of this, Compound 1 is an ideal chemoprotectant in chemotherapeutic treatment regimens that require multi-day chemotherapeutic agent administration, for example as seen in small cell lung cancer, triple negative breast cancer, bladder cancer, and HPV-positive head and neck and cervical cancer.

In one aspect, the use of Compound 1 at the PK and PD parameters described herein allows for a chemo-protective regimen for use during standard chemotherapeutic dosing schedules or regimens common in many anti-cancer treatments. For example, Compound 1 can be administered so that CDK4/6-replication dependent healthy cells are G1 arrested during chemotherapeutic agent exposure wherein, due to the rapid dissipation of the G1-arresting effect of the compounds, a significant number of healthy cells reenter the cell-cycle and are capable of replicating shortly after chemotherapeutic agent exposure, for example, within less than about 24, 30, 40, or 48 hours, and continue to replicate until administration of Compound 1 in anticipation of the next chemotherapeutic treatment. In one embodiment, Compound 1 is administered to allow for the cycling of the CDK4/6-replication dependent healthy cells between G1-arrest and reentry into the cell-cycle to accommodate a repeated-dosing chemotherapeutic treatment regimen, for example including but not limited to a treatment regimen wherein the chemotherapeutic agent is administered: on day 1-3 every 21 days; on days 1-3 every 28 days; on day 1 every 3 weeks; on day 1, day 8, and day 15 every 28 days, on day 1 and day 8 every 28 days; on days 1 and 8 every 21 days; on days 1-5 every 21 days; 1 day a week for 6-8 weeks; on days 1, 22, and 43; days 1 and 2 weekly; days 1-4 and 22-25; 1-4; 22-25, and 43-46; and similar type-regimens, wherein the CDK4/6-replication dependent cells are G1 arrested during chemotherapeutic agent exposure. In one embodiment, Compound 1 can be administered so that the subject's CDK4/6-replication dependent cells are G1-arrested during daily chemotherapeutic agent exposure, for example a contiguous multi-day chemotherapeutic regimen. In one embodiment, Compound 1 can be administered so that the subject's CDK4/6-replication dependent cells are G1-arrested during chemotherapeutic agent exposure, for example a contiguous multi-day regimen, but a significant portion of healthy cells reenter the cell-cycle and replicate during the off periods before starting the next cycle of chemotherapeutic agent exposure, for example cycle 2, cycle 3, cycle 4, etc. In one embodiment, Compound 1 is administered so that a subject's CDK4/6-replication dependent cells' G1-arrest is provided during a daily chemotherapeutic agent treatment regimen, for example, a contiguous multi-day treatment regimen, and the arrested cells are capable of reentering the cell-cycle shortly after the multi-day regimen ends.

In one embodiment, the subject has small cell lung cancer and Compound 1 is administered intravenously over about a 30 minute period about 30 minutes prior to administration of either etoposide or carboplatin on day 1, and etoposide on days 2 and 3 during a 21-day treatment cycle, wherein the subject is administered both etoposide and carboplatin on day 1 and etoposide on day 2 and 3 during a 21-day cycle first line treatment protocol. In one embodiment, the dose of etoposide administered is 100 mg/m² administered intravenously over about 60 minutes daily on days 1, 2, and 3 of each 21-day cycle. In one embodiment, the dose of carboplatin administered to the subject is calculated using the Calvert formula with a target AUC of 5 (maximum dose of 750 mg) administered intravenously over 30 minutes on day1 of each 21-day cycle.

In one embodiment, the subject has small cell lung cancer and Compound 1 is administered intravenously over about a 30 minute period about 30 minutes prior to administration of topotecan during a 21-day treatment cycle, wherein the subject is administered topotecan on days 1, 2, 3, 4, and 5 during a 21-day cycle second or third line treatment protocol. In one embodiment, the dose of topotecan administered is 1.5 mg/m² administered intravenously over about 30 minutes daily on days 1, 2, 3, 4, and 5 of each 21-day cycle. In one embodiment, the dose of topotecan administered is 1.25 mg/m² administered intravenously over about 30 minutes daily on days 1, 2, 3, 4, and 5 of each 21-day cycle. In one embodiment, the dose of topotecan administered is 0.75 mg/m² administered intravenously over about 30 minutes daily on days 1, 2, 3, 4, and 5 of each 21-day cycle.

In one embodiment, the subject has small cell lung cancer and Compound 1 is administered intravenously over about a 30 minute period about 30 minutes prior to administration of topotecan during a 21-day treatment cycle, wherein the subject is administered topotecan on days 1, 2, and 3 during a 21-day cycle second or third line treatment protocol. In one embodiment, the dose of topotecan administered is 1.25 mg/m² administered intravenously over about 30 minutes daily on days 1, 2, and 3 of each 21-day cycle.

Administration of Compound 1 in the doses described herein can result in reduced anemia, reduced lymphopenia, reduced thrombocytopenia, or reduced neutropenia compared to that typically expected after, common after, or associated with treatment with chemotherapeutic agents in the absence of administration of Compound 1. The use of Compound 1 as described herein results in a faster recovery from bone marrow suppression associated with long-term use of CDK4/6 inhibitors, such as myelosuppression, anemia, lymphopenia, thrombocytopenia, or neutropenia, following the cessation of use of Compound 1. In some embodiments, the use of Compound 1 as described herein results in reduced or limited bone marrow suppression associated with long-term use of CDK4/6 inhibitors, such as myelosuppression, anemia, lymphopenia, thrombocytopenia, or neutropenia.

In one embodiment, Compound 1, at the concentrations and doses described herein, is used in a CDK4/6-replication dependent healthy cell cycling strategy wherein a subject is exposed to regular, repeated chemotherapeutic treatments, wherein the healthy cells are G1-arrested when chemotherapeutic agent exposed and allowed to reenter the cell-cycle before the subject's next chemotherapeutic treatment. Such cycling allows CDK4/6-replication dependent cells to regenerate damaged blood cell lineages between regular, repeated treatments, for example those associated with standard chemotherapeutic treatments for cancer, and reduces the risk associated with long term CDK4/6 inhibition. This cycling between a state of G1-arrest and a state of replication is not feasible in limited time-spaced, repeated chemotherapeutic agent exposures using longer acting CDK4/6 inhibitors such as palbociclib, as the lingering G1-arresting effects of the compound prohibit significant and meaningful reentry into the cell-cycle before the next chemotherapeutic agent exposure or delay the healthy cells from entering the cell cycle and reconstituting damaged tissues or cells following treatment cessation.

According to the present invention, Compound 1 can be administered to a subject on any chemotherapeutic treatment schedule and in any dose consistent with the prescribed course of treatment. Compound 1 can be administered prior to, during, or following the administration of the chemotherapeutic agent. In one embodiment, Compound 1 can be administered to the subject during the time period ranging from 24 hours prior to chemotherapeutic treatment until 24 hours following exposure. This time period, however, can be extended to time earlier that 24 hour prior to exposure to the agent (e.g., based upon the time it takes the chemotherapeutic agent used to achieve suitable plasma concentrations and/or the compound's plasma half-life). Further, the time period can be extended longer than 24 hours following exposure to the chemotherapeutic agent so long as later administration of Compound 1 leads to at least some protective effect. Such post-exposure treatment can be especially useful in cases of accidental exposure or overdose. In an alternative embodiment, Compound 1 can be administered to the subject during the time period ranging from 48 hours prior to chemotherapeutic treatment until 48 hours following exposure.

In some embodiments, Compound 1 can be administered to the subject at a time period prior to the administration of the chemotherapeutic agent, so that plasma levels of Compound 1 are peaking at the time of administration of the chemotherapeutic agent. If convenient, Compound 1 can be administered at the same time as the chemotherapeutic agent, in order to simplify the treatment regimen. In some embodiments, the chemoprotectant and chemotherapeutic can be provided in a single formulation.

In some embodiments, Compound 1 can be administered to the subject such that the chemotherapeutic agent can be administered either at higher doses (increased chemotherapeutic dose intensity) or more frequently (increased chemotherapeutic dose density) or at a dose that achieves equivalent AUC therapeutic levels as seen when the chemotherapeutic agent is administered alone.

Dose-dense chemotherapy is a chemotherapy treatment plan in which drugs are given with less time between treatments than in a standard chemotherapy treatment plan. Chemotherapy dose intensity represents unit dose of chemotherapy administered per unit time. Dose intensity can be increased or decreased through altering dose administered, time interval of administration, or both. Myelosuppression continues to represent the major dose-limiting toxicity of cancer chemotherapy, resulting in considerable morbidity and mortality along with frequent reductions in chemotherapy dose intensity, which may compromise disease control and survival. The compounds and their use as described herein represent a way of increasing chemotherapy dose density and/or dose intensity while mitigating adverse events such as, but not limited to, myelosuppression.

If desired, multiple doses of Compound 1 can be administered to the subject. Alternatively, the subject can be given a single dose of Compound 1. For example, Compound 1 can be administered so that CDK4/6-replication dependent healthy cells are G1 arrested during chemotherapeutic agent exposure wherein, due to the rapid dissipation of the G1-arresting effect of the compounds, a significant number of healthy cells reenter the cell-cycle and are capable of replicating shortly after chemotherapeutic agent exposure, for example, within about 24-48 hours or less, and continue to replicate until administration of the CDK4/6-inhibitor in anticipation of the next chemotherapeutic treatment. In one embodiment, Compound 1 is administered to allow for the cycling of the CDK4/6-replication dependent healthy cells between G1-arrest and reentry into the cell-cycle to accommodate a repeated-dosing chemotherapeutic treatment regimen, for example, including but not limited to a treatment regimen wherein the chemotherapeutic agent is administered: on day 1-3 every 21 days; on days 1-3 every 28 days; on day 1 every 3 weeks; on day 1, day 8, and day 15 every 28 days, on day 1 and day 8 every 28 days; on days 1 and 8 every 21 days; on days 1-5 every 21 days; 1 day a week for 6-8 weeks; on days 1, 22, and 43; days 1 and 2 weekly; days 1-4 and 22-25; 1-4; 22-25, and 43-46; and similar type-regimens, wherein the CDK4/6-replication dependent cells are G1 arrested during chemotherapeutic agent exposure and a significant portion of the cells reenter the cell-cycle in between chemotherapeutic agent exposure.

As contemplated herein, Compound 1 can be used as a chemoprotectant in conjunction with a number of standard of care chemotherapeutic treatment regimens used to provide chemoprotection to a subject's CDK4/6-replication dependent healthy cells during a CDK4/6-replication independent cancer treatment protocol. In alternative embodiments, for example, Compound 1 can be administered to provide chemoprotection in a small cell lung cancer therapy protocol such as, but not limited to: cisplatin 60 mg/m² IV on day 1 plus etoposide 120 mg/m² IV on days 1-3 every 21 d for 4 cycles; cisplatin 80 mg/m² IV on day 1 plus etoposide 100 mg/m² IV on days 1-3 every 28 d for 4 cycles; cisplatin 60-80 mg/m² IV on day 1 plus etoposide 80-120 mg/m² IV on days 1-3 every 21-28 d (maximum of 4 cycles); carboplatin AUC 5-6 min*mg/mL IV on day 1 plus etoposide 80-100 mg/m² IV on days 1-3 every 28 d (maximum of 4 cycles); Cisplatin 60-80 mg/m² IV on day 1 plus etoposide 80-120 mg/m² IV on days 1-3 every 21-28 d; carboplatin AUC 5-6 min*mg/mL IV on day 1 plus etoposide 80-100 mg/m² IV on days 1-3 every 28 d (maximum 6 cycles); cisplatin 60 mg/m² IV on day 1 plus irinotecan 60 mg/m² IV on days 1, 8, and 15 every 28 d (maximum 6 cycles); cisplatin 30 mg/m² IV on days 1 and 8 or 80 mg/m² IV on day 1 plus irinotecan 65 mg/m² IV on days 1 and 8 every 21 d (maximum 6 cycles); carboplatin AUC 5 min*mg/mL IV on day 1 plus irinotecan 50 mg/m² IV on days 1, 8, and 15 every 28 d (maximum 6 cycles); carboplatin AUC 4-5 IV on day 1 plus irinotecan 150-200 mg/m² IV on day 1 every 21 d (maximum 6 cycles); cyclophosphamide 800-1000 mg/m² IV on day 1 plus doxorubicin 40-50 mg/m² IV on day 1 plus vincristine 1-1.4 mg/m² IV on day 1 every 21-28 d (maximum 6 cycles); Etoposide 50 mg/m² PO daily for 3 wk every 4 wk; topotecan 2.3 mg/m² PO on days 1-5 every 21 d; topotecan 1.5 mg/m² IV on days 1-5 every 21 d; carboplatin AUC 5 min*mg/mL IV on day 1 plus irinotecan 50 mg/m² IV on days 1, 8, and 15 every 28 d; carboplatin AUC 4-5 IV on day 1 plus irinotecan 150-200 mg/m² IV on day 1 every 21 d; cisplatin 30 mg/m² IV on days 1, 8, and 15 plus irinotecan 60 mg/m² IV on days 1, 8, and 15 every 28 d; cisplatin 60 mg/m² IV on day 1 plus irinotecan 60 mg/m² IV on days 1, 8, and 15 every 28 d; cisplatin 30 mg/m² IV on days 1 and 8 or 80 mg/m² IV on day 1 plus irinotecan 65 mg/m² IV on days 1 and 8 every 21 d; paclitaxel 80 mg/m² IV weekly for 6 wk every 8 wk; paclitaxel 175 mg/m² IV on day 1 every 3 wk; etoposide 50 mg/m² PO daily for 3 wk every 4 wk; topotecan 2.3 mg/m² PO on days 1-5 every 21 d; topotecan 1.5 mg/m² IV on days 1-5 every 21 d; carboplatin AUC 5 min*mg/mL IV on day 1 plus irinotecan 50 mg/m² IV on days 1, 8, and 15 every 28 d; carboplatin AUC 4-5 IV on day 1 plus irinotecan 150-200 mg/m² IV on day 1 every 21 d; cisplatin 30 mg/m² IV on days 1, 8, and 15 plus irinotecan 60 mg/m² IV on days 1, 8, and 15 every 28 d; cisplatin 60 mg/m² IV on day 1 plus irinotecan 60 mg/m² IV on days 1, 8, and 15 every 28 d; cisplatin 30 mg/m² IV on days 1 and 8 or 80 mg/m² IV on day 1 plus irinotecan 65 mg/m² IV on days 1 and 8 every 21 d; paclitaxel 80 mg/m² IV weekly for 6 wk every 8 wk; and paclitaxel 175 mg/m² IV on day 1 every 3 wk. In alternative embodiments, Compound 1 is administered to provide chemoprotection in a small cell lung cancer therapy protocol such as, but not limited to: topotecan 2.0 mg/m² PO on days 1-5 every 21 d; topotecan 1.5-2.3 mg/m² PO on days 1-5 every 21 d; etoposide 100 mg/m² intravenously (IV) on days 1 through 3 plus cisplatin 50 mg/m² IV on days 1 and 2 (treatment cycles administered every 3 weeks to a maximum of six cycles); etoposide 100 mg/m² intravenously (IV) on days 1 through 3 plus carboplatin 300 mg/m² IV on day 1 (treatment cycles administered every 3 weeks to a maximum of six cycles); carboplatin (300 mg/m² IV on day 1) and escalating doses of etoposide starting with 80 mg/m² IV on days 1-3; carboplatin 125 mg/m²/day combined with etoposide 200 mg/m²/day administered for 3 days; etoposide 80-200 mg/m² intravenously (IV) on days 1 through 3 plus carboplatin 125-450 mg/m² IV on day 1 (treatment cycles administered every 21-28 days); carboplatin AUC 5-6 min*mg/mL IV on day 1 plus etoposide 80-200 mg/m² IV on days 1-3 every 28 d (maximum of 4 cycles).

In one embodiment, Compound 1 is administered in a dosage describe herein to a subject with small cell lung cancer on days 1, 2, and 3 of a treatment protocol wherein the DNA damaging agent selected from the group consisting of carboplatin, etoposide, and cisplatin, or a combination thereof, is administered on days 1, 2, and 3 every 21 days.

In one embodiment, Compound 1 is used to provide chemoprotection to a subject's CDK4/6-replication dependent healthy cells during a CDK4/6-replication independent head and neck cancer treatment protocol. In one embodiment, Compound 1 is administered to provide chemoprotection in a CDK4/6-replication independent head and neck cancer therapy protocol such as, but not limited to: cisplatin 100 mg/m² IV on days 1, 22, and 43 or 40-50 mg/m² IV weekly for 6-7 wk; cetuximab 400 mg/m² IV loading dose 1 wk before the start of radiation therapy, then 250 mg/m² weekly (premedicate with dexamethasone, diphenhydramine, and ranitidine); cisplatin 20 mg/m² IV on day 2 weekly for up to 7 wk plus paclitaxel 30 mg/m² IV on day 1 weekly for up to 7 wk; cisplatin 20 mg/m²/day IV on days 1-4 and 22-25 plus 5-FU 1000 mg/m²/day by continuous IV infusion on days 1-4 and 22-25; 5-FU 800 mg/m² by continuous IV infusion on days 1-5 given on the days of radiation plus hydroxyurea 1 g PO q12h (11 doses per cycle); chemotherapy and radiation given every other week for a total of 13 wk; carboplatin 70 mg/m²/day IV on days 1-4, 22-25, and 43-46 plus 5-FU 600 mg/m²/day by continuous IV infusion on days 1-4, 22-25, and 43-46; carboplatin AUC 1.5 IV on day 1 weekly plus paclitaxel 45 mg/m² IV on day 1 weekly; cisplatin 100 mg/m² IV on days 1, 22, and 43 or 40-50 mg/m² IV weekly for 6-7 wk; docetaxel 75 mg/m² IV on day 1 plus cisplatin 100 mg/m² IV on day 1 plus 5-FU 100 mg/m²/day by continuous IV infusion on days 1-4 every 3 wk for 3 cycles, then 3-8 wk later, carboplatin AUC 1.5 IV weekly for up to 7 wk during radiation therapy; docetaxel 75 mg/m² IV on day 1 plus cisplatin 75 mg/m² IV on day 1 plus 5-FU 750 mg/m²/day by continuous IV infusion on days 1-4 every 3 wk for 4 cycles; cisplatin 100 mg/m² IV on day 1 every 3 wk for 6 cycles plus 5-FU 1000 mg/m²/day by continuous IV infusion on days 1-4 every 3 wk for 6 cycles plus cetuximab 400 mg/m² IV loading dose on day 1, then 250 mg/m² IV weekly until disease progression (premedicate with dexamethasone, diphenhydramine, and ranitidine); carboplatin AUC 5 min*mg/mL IV on day 1 every 3 wk for 6 cycles plus 5-FU 1000 mg/m²/day by continuous IV infusion on days 1-4 every 3 wk for 6 cycles plus cetuximab 400 mg/m² IV loading dose on day 1, then 250 mg/m² IV weekly until disease progression (premedicate with dexamethasone, diphenhydramine, and ranitidine); cisplatin 75 mg/m² IV on day 1 plus docetaxel 75 mg/m² IV on day 1 every 3 wk; cisplatin 75 mg/m² IV on day 1 plus paclitaxel 175 mg/m² IV on day 1 every 3 wk; carboplatin AUC 6 IV on day 1 plus docetaxel 65 mg/m² IV on day 1 every 3 wk; carboplatin AUC 6 IV on day 1 plus paclitaxel 200 mg/m² IV on day 1 every 3 wk; cisplatin 75-100 mg/m² IV on day 1 every 3-4 wk plus cetuximab 400 mg/m² IV loading dose on day 1, then 250 mg/m² IV weekly (premedicate with dexamethasone, diphenhydramine, and ranitidine); cisplatin 100 mg/m² IV on day 1 plus 5-FU 1000 mg/m²/day by continuous IV infusion on days 1-4 every 3 wk; methotrexate 40 mg/m² IV weekly (3 wk equals 1 cycle); paclitaxel 200 mg/m² IV every 3 wk; docetaxel 75 mg/m² IV every 3 wk; cetuximab 400 mg/m² IV loading dose on day 1, then 250 mg/m² IV weekly until disease progression (premedicate with dexamethasone, diphenhydramine, and ranitidine); cisplatin 100 mg/m² IV on day 1 every 3 wk for 6 cycles plus 5-FU 1000 mg/m²/day by continuous IV infusion on days 1-4 every 3 wk for 6 cycles plus cetuximab 400 mg/m² IV loading dose on day 1, then 250 mg/m² IV weekly (premedicate with dexamethasone, diphenhydramine, and ranitidine); carboplatin AUC 5 min*mg/mL IV on day 1 every 3 wk for 6 cycles plus 5-FU 1000 mg/m²/day by continuous IV infusion on days 1-4 every 3 wk for 6 cycles plus cetuximab 400 mg/m² IV loading dose on day 1, then 250 mg/m² IV weekly (premedicate with dexamethasone, diphenhydramine, and ranitidine); cisplatin 75 mg/m² IV on day 1 plus docetaxel 75 mg/m² IV on day 1 every 3 wk; cisplatin 75 mg/m² IV on day 1 plus paclitaxel 175 mg/m² IV on day 1 every 3 wk; carboplatin AUC 6 IV on day 1 plus docetaxel 65 mg/m² IV on day 1 every 3 wk; carboplatin AUC 6 IV on day 1 plus paclitaxel 200 mg/m² IV on day 1 every 3 wk; cisplatin 75-100 mg/m² IV on day 1 every 3-4 wk plus cetuximab 400 mg/m² IV loading dose on day 1, then 250 mg/m² IV weekly (premedicate with dexamethasone, diphenhydramine, and ranitidine); cisplatin 100 mg/m² IV on day 1 plus 5-FU 1000 mg/m²/day by continuous IV infusion on days 1-4 every 3 wk; methotrexate 40 mg/m² IV weekly (3 wk equals 1 cycle); paclitaxel 200 mg/m² IV every 3 wk; docetaxel 75 mg/m² IV every 3 wk; cetuximab 400 mg/m² IV loading dose on day 1, then 250 mg/m² IV weekly until disease progression (premedicate with dexamethasone, diphenhydramine, and ranitidine); cisplatin 100 mg/m² IV on days 1, 22, and 43 with radiation, then cisplatin 80 mg/m² IV on day 1 plus 5-FU 1000 mg/m²/day by continuous IV infusion on days 1-4 every 4 wk for 3 cycles; cisplatin 75 mg/m² IV on day 1 plus docetaxel 75 mg/m² IV on day 1 every 3 wk; cisplatin 75 mg/m² IV on day 1 plus paclitaxel 175 mg/m² IV on day 1 every 3 wk; carboplatin AUC 6 IV on day 1 plus docetaxel 65 mg/m² IV on day 1 every 3 wk; carboplatin AUC 6 IV on day 1 plus paclitaxel 200 mg/m² IV on day 1 every 3 wk; cisplatin 100 mg/m² IV on day 1 plus 5-FU 1000 mg/m²/day by continuous IV infusion on days 1-4 every 3 wk; cisplatin 50-70 mg/m² IV on day 1 plus gemcitabine 1000 mg/m² IV on days 1, 8, and 15 every 4 wk; gemcitabine 1000 mg/m² IV on days 1, 8, and 15 every 4 wk or gemcitabine 1250 mg/m² IV on days 1 and 8 every 3 wk; methotrexate 40 mg/m² IV weekly (3 wk equals 1 cycle); paclitaxel 200 mg/m² IV every 3 wk; docetaxel 75 mg/m² IV every 3 wk; cisplatin 75 mg/m² IV on day 1 plus docetaxel 75 mg/m² IV on day 1 every 3 wk; cisplatin 75 mg/m² IV on day 1 plus paclitaxel 175 mg/m² IV on day 1 every 3 wk; carboplatin AUC 6 IV on day 1 plus docetaxel 65 mg/m² IV on day 1 every 3 wk; carboplatin AUC 6 IV on day 1 plus paclitaxel 200 mg/m² IV on day 1 every 3 wk; cisplatin 100 mg/m² IV on day 1 plus 5-FU 1000 mg/m²/day by continuous IV infusion on days 1-4 every 3 wk; cisplatin 50-70 mg/m² IV on day 1 plus gemcitabine 1000 mg/m² IV on days 1, 8, and 15 every 4 wk; gemcitabine 1000 mg/m² IV on days 1, 8, and 15 every 4 wk or gemcitabine 1250 mg/m² IV on days 1 and 8 every 3 wk; methotrexate 40 mg/m² IV weekly (3 wk equals 1 cycle); paclitaxel 200 mg/m² IV every 3 wk; and docetaxel 75 mg/m² IV every 3 wk.

In one embodiment, Compound 1 is used to provide chemoprotection to a subject's CDK4/6-replication dependent healthy cells during a CDK4/6-replication independent triple negative breast cancer treatment protocol. In one embodiment, Compound 1 is administered to provide a blood plasma concentration described herein to provide chemoprotection in a CDK4/6-replication independent triple negative breast cancer therapy protocol such as, but not limited to: dose-dense doxorubicin (adriamycin) and cyclophosphamide (cytoxan) every two weeks for four cycles followed by dose-dense paclitaxel (Taxol) every two weeks for four cycles; adriamycin/paclitaxel/cyclophosphomide every three weeks for a total of four cycles; adriamycin/paclitaxel/cyclophosphomide every two weeks for a total of four cycles; adriamycin/cyclophosphomide followed by paclitaxel (Taxol) every three weeks for four cycles each; and adriamycin/cyclophosphomide followed by paclitaxel (Taxol) every two weeks for four cycles each.

In one embodiment, Compound 1 is used to provide chemoprotection to a subject's CDK4/6-replication dependent healthy cells during a CDK4/6-replication independent bladder cancer treatment protocol. In one embodiment, Compound 1 is administered to provide a blood plasma concentration described herein to provide chemoprotection in a CDK4/6-replication independent bladder cancer therapy protocol such as, but not limited to: postoperative adjuvant intravesical chemotherapy for non-muscle invasive bladder cancer, first-line chemotherapy for muscle-invasive bladder cancer, and second-line chemotherapy for muscle invasive bladder cancer. Non-limiting examples of postoperative chemotherapy for bladder cancer include one dose or mitomycin (40 mg), epirubicin (80 mg), thiotepa (30 mg), or doxorubicin (50 mg). Non-limiting examples of first-line chemotherapy for bladder cancer include: gemcitabine 1000 mg/m² on days 1, 8, and 15 plus cisplatin 70 mg/m² on day 1 or 2 repeating cycle every 28 days for a total of four cycles; dosing methotrexate 30 mg/m² IV on days 1, 15, and 22 plus vinblastine 3 mg/m² IV on days 2, 15, and 22 plus doxorubicin 30 mg/m² IV on day 2 plus cisplatin 70 mg/m² IV on day 2, repeat cycle every 28 d for a total of 3 cycles; and dose-dense regimens of the above administered along with doses of growth factor stimulants.

In one embodiment, Compound 1 is used to provide chemoprotection to a subject's CDK4/6-replication dependent healthy cells during a CDK4/6-replication independent retinoblastoma treatment protocol. In one embodiment, Compound 1 is administered to provide a blood plasma concentration described herein to provide chemoprotection in a CDK4/6-replication independent retinoblastoma therapy protocol such as, but not limited to the administration of carboplatin, vincristine, or etoposide in conjunction with surgery, radiotherapy, cryotherapy, thermotherapy, or other local therapy techniques.

In one embodiment, Compound 1 is used to provide chemoprotection to a subject's CDK4/6-replication dependent healthy cells during a CDK4/6-replication independent cervical cancer treatment protocol. In one embodiment, Compound 1 is administered to provide a blood plasma concentration described herein to provide chemoprotection in a CDK4/6-replication independent cervical cancer therapy protocol such as, but not limited to the administration of cisplatin 40 mg/m² IV once weekly, cisplatin 50-75 mg/m² IV on day 1 plus 5-fluorouracil (5-FU) 1000 mg/m² continuous IV infusion on days 2-5 and days 30-33, cisplatin 50-75 mg/m² IV on day 1 plus 5-FU 1000 mg/m² IV infusion over 24 hour on days 1-4 every 3 weeks for 3-4 cycles, bevacizumab 15 mg/kg IV over 30-90 minutes plus cisplatin on day 1 or 2 plus paclitaxel on day 1 every 3 weeks, bevacizumab plus paclitaxel on day 1 plus topotecan on days 1-3 every 3 weeks, paclitaxel followed by cisplatin on day 1 every 3 weeks, topotecan on days 1-3 followed by cisplatin on day 1 every 3 weeks, and paclitaxel on day 1 every 3 weeks. In another embodiment the cervical cancer therapy protocol is as above in addition to radiation, surgery, or another procedure.

Triple-negative breast cancer (TNBC) is defined as the absence of staining for estrogen receptor, progesterone receptor, and HER2/neu. TNBC is insensitive to some of the most effective therapies available for breast cancer treatment including HER2-directed therapy such as trastuzumab and endocrine therapies such as tamoxifen or the aromatase inhibitors. Combination cytotoxic chemotherapy administered in a dose-dense or metronomic schedule remains the standard therapy for early-stage TNBC. Platinum agents have recently emerged as drugs of interest for the treatment of TNBC with carboplatin added to paclitaxel and adriamycin plus cyclophosphamide chemotherapy in the neoadjuvant setting. The poly (ADP-ribose) polymerase (PARP) inhibitors are emerging as promising therapeutics for the treatment of TNBC. PARPs are a family of enzymes involved in multiple cellular processes, including DNA repair.

As a nonlimiting illustration, the subject is exposed to chemotherapeutic agent at least 5 times a week, at least 4 times a week, at least 3 times a week, at least 2 times a week, at least 1 time a week, at least 3 times a month, at least 2 times a month, or at least 1 time a month, wherein the subject's CDK4/6-replication dependent healthy cells are G1 arrested during treatment and allowed to cycle in between chemotherapeutic agent exposure, for example during a treatment break. In one embodiment, the subject is undergoing 5 times a week chemotherapeutic treatment, wherein the subject's CDK4/6-replication dependent healthy cells are G1 arrested during the chemotherapeutic agent exposure and allowed to reenter the cell-cycle during the 2 day break, for example, over the weekend.

In one embodiment, using Compound 1 at the dosage described herein, the subject's CDK4/6-replicaton dependent healthy cells are arrested during the entirety of the chemotherapeutic agent exposure time-period, for example, during a contiguous multi-day regimens, the cells are arrested over the time period that is required to complete the contiguous multi-day course, and then allowed to recycle at the end of the contiguous multi-day course. In one embodiment, using Compound 1 at the dosage described herein, the subject's CDK4/6-replication dependent healthy cells are arrested during the entirety of the chemotherapeutic regimen, for example, in a daily chemotherapeutic exposure for three weeks, and rapidly reenter the cell-cycle following the completion of the therapeutic regimen.

In one embodiment, the subject has been exposed to a chemotherapeutic agent, and, using Compound 1 at the dosage described herein, the subject's CDK4/6-replication dependent healthy cells are placed in G1 arrest following exposure in order to mitigate, for example, DNA damage. In one embodiment, Compound 1 at the dosage described herein is administered at least 1/2 hour, at least 1 hour, at least 2 hours, at least 3 hours, at least 4 hours, at least 5 hours, at least 6 hours, at least 7 hours, at least 8 hours, at least 10 hours, at least 12 hours, at least 14 hours, at least 16 hours, at least 18 hours, at least 20 hours or more post chemotherapeutic agent exposure.

In some embodiments, the CDK4/6-replication dependent healthy cells can be arrested for longer periods to allow for intensified chemotherapeutic treatment, for example, over a period of hours, days, and/or weeks, through multiple, time separated administrations of a CDK4/6 inhibitor described herein. Because of the rapid and synchronous reentry into the cell cycle by CDK4/6-replication dependent healthy cells, for example HSPCs, upon dissipation of the CDK4/6 inhibitors intra-cellular effects, the cells are capable of reconstituting the cell lineages faster than CDK4/6 inhibitors with longer G1 arresting profiles, for example palbociclib.

The reduction in chemotoxicity afforded by Compound 1 at the dosage described herein can allow for dose intensification (e.g., more therapy can be given in a fixed period of time) in medically related chemotherapies, which will translate to better efficacy. Therefore, the presently disclosed methods can result in chemotherapy regimens that are less toxic and more effective.

The use of a Compound 1 at the dosage described herein can induce selective G1 arrest in CDK4/6-dependent cells (e.g., as measured in a cell-based in vitro assay). In one embodiment, Compound 1 at the dosage described herein is capable of increasing the percentage of CDK4/6-dependent cells in the G1 phase, while decreasing the percentage of CDK4/6-dependent cells in the G2/M phase and S phase. In one embodiment, Compound 1 at the dosage described herein induces substantially pure (i.e., “clean”) G1 cell cycle arrest in the CDK4/6-dependent cells (e.g., wherein treatment with Compound 1 induces cell cycle arrest such that the majority of cells are arrested in G1 as defined by standard methods (e.g. propidium iodide (PI) staining or others) with the population of cells in the G2/M and S phases combined being less than about 30%, about 25%, about 20%, about 15%, about 10%, about 5%, about 3% or less of the total cell population. Methods of assessing the cell phase of a population of cells are known in the art (see, for example, in U.S. Patent Application Publication No. 2002/0224522) and include cytometric analysis, microscopic analysis, gradient centrifugation, elutriation, fluorescence techniques including immunofluorescence, and combinations thereof. Cytometric techniques include exposing the cell to a labeling agent or stain, such as DNA-binding dyes, e.g., PI, and analyzing cellular DNA content by flow cytometry. Immunofluorescence techniques include detection of specific cell cycle indicators such as, for example, thymidine analogs (e.g., 5-bromo-2-deoxyuridine (BrdU) or an iododeoxyuridine), with fluorescent antibodies.

In some embodiments, the use of Compound 1 at the dosage described herein results in reduced or substantially free off-target effects, particularly related to inhibition of kinases other than CDK4 and or CDK6 such as CDK2, as Compound 1 at the dosage described herein is a poor inhibitor (e.g., >1 uM IC₅₀) of CDK2. Furthermore, because of the high selectivity for CDK4/6, the use of Compound 1 should not induce cell cycle arrest in CDK4/6-independent cells. In addition, because of the short transient nature of the G1-arrest effect, the CDK4/6-replication dependent cells more quickly reenter the cell-cycle than, comparatively, use of palbociclib provides, resulting in the reduced risk of, in one embodiment, hematological toxicity development during long term treatment regimens due to the ability of HSPCs to replicate between chemotherapeutic treatments.

In some embodiments, the use of Compound 1 at the dosage described herein reduces the risk of undesirable off-target effects including, but not limited to, long term toxicity, anti-oxidant effects, and estrogenic effects. Anti-oxidant effects can be determined by standard assays known in the art. For example, a compound with no significant anti-oxidant effects is a compound that does not significantly scavenge free-radicals, such as oxygen radicals. The anti-oxidant effects of a compound can be compared to a compound with known anti-oxidant activity, such as genistein. Thus, a compound with no significant anti-oxidant activity can be one that has less than about 2, 3, 5, 10, 30, or 100 fold anti-oxidant activity relative to genistein. Estrogenic activities can also be determined via known assays. For instance, a non-estrogenic compound is one that does not significantly bind and activate the estrogen receptor. A compound that is substantially free of estrogenic effects can be one that has less than about 2, 3, 5, 10, 20, or 100 fold estrogenic activity relative to a compound with estrogenic activity, e.g., genistein.

### Dosing Profiles of Compound 1

The invention provides particular dosing and blood profile ranges of the CDK4/6 inhibitor compound 2′-((5-(4-methylpiperazin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one (Compound 1), and methods using said dosages, for treating a subject undergoing DNA-damaging chemotherapeutic therapy for the treatment of a CDK 4/6-replication independent cellular proliferation disorder.

As contemplated herein and for purposes of the disclosed ranges herein, all ranges described herein include any and all numerical values occurring within the identified ranges. For example, a range of 1 to 10, as contemplated herein, would include the numerical values 1, 2, 3, 4, 5, 6, 7, 8, 9, 10, as well as fractions thereof.

In one aspect, Compound 1 is intravenously administered to a subject prior to administration of a chemotherapeutic agent so that a single dose of Compound 1 provides a specific PK and/or PD blood profile as described herein. In one embodiment, the dose administered to the subject is between about 180 and about 215 mg/m². In one embodiment, the dose is between about 180 and about 280 mg/m². In one embodiment, the dose administered is between about 170 to about 215 mg/m². In one embodiment, the dose administered is between about 170 to about 280 mg/m². For example, the dose is about 170, 175, 180, 185, 190, 195, 200, 205, 210, 215, 220, 225, 230, 235, 240, 245, 250, 255, 260, 265, 270, 275, or 280 mg/m². In one embodiment, the dose is about 192 mg/m². In one embodiment, the dose is about 200 mg/m². In one embodiment, the dose is about 240 mg/m². In one embodiment, the dose administered provides for a mean AUC₍ₗₐₛₜ₎ measured at 24.5 hours or a mean Cmax as described below.

In one embodiment, Compound 1 is intravenously administered to a subject prior to administration of a chemotherapeutic agent so that a single dose of Compound 1 provides a blood plasma level profile dosage-corrected mean Cmax ((ng/ml)/(mg/m²)) of between about 1 (ng/ml)/(mg/m²) and 20 (ng/ml)/(mg/m²), between about 2.5 (ng/ml)/(mg/m²) and 15 (ng/ml)/(mg/m²), or of between about 4 (ng/ml)/(mg/m²) and 12 (ng/ml)/(mg/m²). In one embodiment, the dosage-corrected mean Cmax ((ng/ml)/(mg/m²) is about 1, 2, 3, 4, 5, 6, 7, 8, 9, 10, 11, 12, 13, 14, 15, 16, 17, 18, 19, or 20 ((ng/ml)/(mg/m²). In one embodiment, the dosage-corrected mean Cmax ((ng/ml)/(mg/m²)) is about 8.0 (ng/ml)/(mg/m²)±3.5 (ng/ml)/(mg/m²), about 8.5 (ng/ml)/(mg/m²)±2.5 (ng/ml)/(mg/m²), about 9.5 (ng/ml)/(mg/m²)±2.0 (ng/ml)/(mg/m²), or about 10.2 (ng/ml)/(mg/m²)±1.5 (ng/ml)/(mg/m²). In one embodiment, the dosage-corrected mean Cmax is about 6.0±20%. The dosage corrected mean Cmax is mean Cmax divided by the number of milligrams/m² of Compound 1 in the formulation. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m².

In an alternative, provided is a method of treating a subject undergoing chemotherapy for the treatment of an CDK 4/6-replication independent cellular proliferation disorder by providing an intravenously administered formulation of Compound 1 wherein a single-dose provides a blood plasma level profile dosage-corrected mean Cmax ((ng/ml)/(mg/m²)) of between about 4.6 (ng/ml)/(mg/m²) and about 17.1 (ng/ml)/(mg/m²) or about 1.8 (ng/ml)/(mg/m²) to about 16.8 (ng/ml)/(mg/m²). In one embodiment, the dosage-corrected mean Cmax ((ng/ml)/(mg/m²)) is about at least 8.5 (ng/ml)/(mg/m²) or about at least 3.8 (ng/ml)/(mg/m²).

In one alternative, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a dosage-corrected mean Cmax (ng/ml)/(mg/m²) of between of between about 1 (ng/ml)/(mg/m²) and 20 (ng/ml)/(mg/m²), between about 2.5 (ng/ml)/(mg/m²) and 15 (ng/ml)/(mg/m²), or of between about 4 (ng/ml)/(mg/m²) and 14 (ng/ml)/(mg/m²). In one embodiment, the dosage-corrected mean Cmax ((ng/ml)/(mg/m²) is about 1, 2, 3, 4, 5, 6, 7, 8, 9, 10, 11, 12, 13, 14, 15, 16, 17, 18, 19, or 20 ((ng/ml)/(mg/m²). In one embodiment, the dosage-corrected mean Cmax ((ng/ml)/(mg/m²)) is about 9.5 (ng/ml)/(mg/m²)±1.5 (ng/ml)/(mg/m²). In an alternative embodiment the dosage-corrected mean Cmax is about 9.5 (ng/ml)/(mg/m²)±1.9 (ng/ml)/(mg/m²) or 9.5 (ng/ml)/(mg/m²)±about 20%. In one embodiment, the mean dose-corrected Cmax ((ng/ml)/(mg/m²)) is about 10.45 (ng/ml)/(mg/m²)±about 20%. In one embodiment, the dosage-corrected mean Cmax is about 6.0 ((ng/ml)/(mg/m²))±20%. In one embodiment, the dosage-corrected mean Cmax is about 6.5 ((ng/ml)/(mg/m²))±20%. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one embodiment, Compound 1 is intravenously administered to a subject prior to administration of a chemotherapeutic agent so that a single dose of Compound 1 provides a blood plasma level profile dosage-corrected with a mean Cmax (ng/ml) of between about 1000 ng/ml and about 3500 ng/ml, or between about 1400 ng/ml and about 3100 ng/ml, or between about 1700 ng/ml and about 2500 ng/ml, or between about 1900 ng/ml and about 2150 ng/ml. In one embodiment, the mean Cmax (ng/ml) is about 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, or 3500 (ng/ml). In one embodiment, the mean Cmax (ng/ml) is about 2030 ng/ml±555 ng/ml. In one embodiment, the mean Cmax (ng/ml) is about 1900 ng/ml, about 1950 ng/ml, about 1975 ng/ml, about 2000 ng/ml, about 2025 ng/ml, about 2030 ng/ml, about 2040 ng/ml, about 2050 ng/ml. about 2075 ng/ml, or about 2100 ng/ml. In one embodiment, the maximum mean concentration occurs at the end of the infusion period of the formulation. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m².

In an alternative embodiment, Compound 1 is intravenously administered to a subject prior to administration of a chemotherapeutic agent so that a single dose of Compound 1 provides a blood plasma level profile dosage-corrected a mean Cmax (ng/ml) of between about 885 ng/ml and about 3280 ng/ml, or between about 355 ng/ml and about 3360 ng/ml. In one embodiment, the mean Cmax (ng/ml) is about at least 1705 ng/ml. In one embodiment, the Cmax (ng/ml) is about at least 752 ng/ml.

In one alternative, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a mean Cmax (ng/ml) of between about 1000 ng/ml and 3500 ng/ml. In one embodiment, the mean Cmax (ng/ml) is between about 1400 ng/ml and about 3100 ng/ml. In one embodiment, the mean Cmax (ng/ml) of about 1000, 1100, 1200, 1300, 1400, 1450, 1500, 1550, 1600, 1650, 1700, 1750, 1800, 1850, 1900, 1950, 2000, 2050, 2100, 2150, 2200, 2250, 2300, 2400, 2450, 2500, 2600, 2650, 2700, 2750, 2800, 2850, 2900, 2950, 3000, 3050, 3100, 3200, 3300, 3400, 3500 (ng/ml). In one embodiment, the mean Cmax (ng/ml) is about 2030 ng/ml±555 ng/ml. In an alternative embodiment the mean Cmax is about 2030 ng/ml±406 ng/ml or about 2030 ng/ml about 20%. In an alternative embodiment the mean Cmax is about 2230 ng/ml±about 20%. In one embodiment the mean Cmax is at least about 1020 ng/ml. In one embodiment, the maximum mean concentration occurs at the end of the infusion period of Compound 1. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one embodiment, provided is a method of treating a subject undergoing chemotherapy for the treatment of a CDK 4/6-replication independent cellular proliferation disorder by providing an intravenous administered formulation of Compound 1 wherein a single-dose provides a blood plasma level profile with a mean Tmax (h) of between about 0.10 hrs and about 1.0 hrs, of between about 0.20 hrs and about 0.6 hrs, or of between about 0.30 hrs and about 0.5 hrs. In one embodiment, the Tmax(h) is about 0.10, 0.15, 0.20, 0.25, 0.30, 0.35, 0.40, 0.45, 0.50, 0.55, 0.60, 0.65, 0.70. 0.85, 0.90, 0.95, or 1.0 (h). In one embodiment, the mean Tmax (h) is about 0.417 hrs 0.129 hrs. In one embodiment, the mean Tmax (h) is about 0.3 hrs, about 0.35 hrs, about 0.375 hrs, about 0.40 hrs, about 0.415 hrs, about 0.425 hrs, about 0.45 hrs, about 0.475 hrs, or about 0.5 hrs. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a Tmax(h) as described above.

In an alternative, provided is a method of treating a subject undergoing chemotherapy for the treatment of a CDK 4/6-replication independent cellular proliferation disorder by providing an intravenous administered formulation of Compound 1 wherein a single-dose provides a blood plasma level profile with a mean Tmax (h) of between about 0.25 hrs and about 0.48 hrs. In one embodiment, the mean Tmax (h) is about at least 0.47 hrs.

In one embodiment, Compound 1 is intravenously administered to a subject prior to administration of a chemotherapeutic agent so that a single dose of Compound 1 provides a blood plasma level profile mean AUC_{inf} (h*ng/ml) measured over 24.5 hours after administration of between about 2000 h*ng/ml to about 4500 h*ng/ml, of between about 2300 h*ng/ml to about 4000 h*ng/ml, of between about 2500 h*ng/ml to about 3500 h*ng/ml, or of between about 2700 h*ng/ml to about 3200 h*ng/ml. In one embodiment, the mean AUC_{inf} (h*ng/ml) is about 2000, 2100, 2200, 2300, 2400, 2500, 2600, 2700, 2800, 2900, 3000, 3100, 3200, 3300, 3400, 3500, 3600, 3700, 3800, 3900, or 4000 (h*ng/ml). In one embodiment, the mean AUC_{inf} (h*ng/ml) measured over 24.5 hours after administration is about 3050 h*ng/ml±513 h*ng/ml. In one embodiment, the mean AUC_{inf} (h*ng/ml) measured over 24.5 hours after administration is about 2500 h*ng/ml, is about 2750 h*ng/ml, about 2900 h*ng/ml, about 3000 h*ng/ml, about 3050 h*ng/ml, about 3100 h*ng/ml, about 3250 h*ng/ml, about 3300 h*ng/ml. In one embodiment, the mean AUC_{inf} (h*ng/ml) measured over 72.5 hours after administration is between about 2000 h*ng/ml to about 4500 h*ng/ml, of between about 2300 h*ng/ml to about 4000 h*ng/ml, of between about 2500 h*ng/ml to about 3500 h*ng/ml, or of between about 2700 h*ng/ml to about 3200 h*ng/ml. In one embodiment, the mean AUC_{inf} (h*ng/ml) measured over 72.5 hours after administration is about 2000, 2100, 2200, 2300, 2400, 2500, 2600, 2700, 2800, 2900, 3000, 3100, 3200, 3300, 3400, 3500, 3600, 3700, 3800, 3900, or 4000 (h*ng/ml). In one embodiment, the mean AUC_{inf} (h*ng/ml) measured over 72.5 hours after administration is about 3160 h*ng/ml±522 h*ng/ml. In one embodiment, the mean AUC_{inf} (h*ng/ml) measured over 72.5 hours after administration is about 2500 h*ng/ml, is about 2600 h*ng/ml, about 2900 h*ng/ml, about 3000 h*ng/ml, about 3050 h*ng/ml, about 3100 h*ng/ml, about 3250 h*ng/ml, about 3300 h*ng/ml, about 3500 h*ng/ml, about 3600 h*ng/ml, about 3700 h*ng/ml, or about 3800 h*ng/ml. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a AUC_{inf} (h*ng/ml) as described above.

In an alternative embodiment, Compound 1 is intravenously administered to a subject prior to administration of a chemotherapeutic agent so that a single dose of Compound 1 provides a blood plasma level profile with a mean AUC_{inf} (h*ng/ml) measured over 24.5 hours after administration of between about 2379 h*ng/ml to about 3762 h*ng/ml or about 1530 h*ng/ml to about 3300 h*ng/ml. In one embodiment, the mean AUC_{inf} h*ng/ml measured over 24.5 hours after administration is about at least 2991 h*ng/ml or about at least 2140 h*ng/ml. In one embodiment, the mean AUC_{inf} h*ng/ml measured over 72.5 hours after administration of between about 2379 h*ng/ml to about 3762 h*ng/ml or about 1530 h*ng/ml to about 3300 h*ng/ml. In one embodiment, the mean AUC_{inf} h*ng/ml measured over 72.5 hours after administration is about at least 2991 h*ng/ml or about at least 2140 h*ng/ml.

In one embodiment, Compound 1 is intravenously administered to a subject prior to administration of a chemotherapeutic agent so that a single dose of Compound 1 provides a blood plasma level profile with a mean AUCₜ (ng*hr/ml) measured over 24.5 hours after administration of between about 2000 h*ng/ml to about 4500 h*ng/ml, between about 2600 h*ng/ml to about 3700 h*ng/ml, between about 2800 h*ng/ml to about 3500 h*ng/ml, or between about 3000 h*ng/ml to about 3200 h*ng/ml. In one embodiment, the mean AUCₜ (ng*hr/ml) measured over 24.5 hours after administration is about 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 (h*ng/ml). In one embodiment, the mean AUCₜ (ng*hr/ml) measured over 24.5 hours after administration is about 2830 (ng*hr/ml)±474 (ng*hr/ml). In one embodiment, the mean AUCₜ (ng*hr/ml) measured over 72.5 hours after administration is between about 2000 h*ng/ml to about 4500 h*ng/ml, between about 2600 h*ng/ml to about 3700 h*ng/ml, between about 2800 h*ng/ml to about 3500 h*ng/ml, or between about 3000 h*ng/ml to about 3200 h*ng/ml. In one embodiment, the mean AUCₜ (ng*hr/ml) measured over about 72.5 hours after administration is about 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 (ng*hr/ml). In one embodiment, the mean AUCₜ (ng*hr/ml) measured over 72.5 hours after administration is about 3110 (ng*hr/ml)±515 (ng*hr/ml). In one embodiment, the mean AUCₜ (ng*hr/ml) measured over 72.5 hours after administration is about 3000 (ng*hr/ml), is about 3050 (ng*hr/ml), is about 3100 (ng*hr/ml), is about 3110 (ng*hr/ml), is about 3150 (ng*hr/ml), is about 3200 (ng*hr/ml), or is about 3250 (ng*hr/ml). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m².

In an alternative embodiment, Compound 1 is intravenously administered to a subject prior to administration of a chemotherapeutic agent so that a single dose of Compound 1 provides a blood plasma level profile with a mean AUCₜ (ng*hr/ml) of between about 2360 h*ng/ml to about 3750 h*ng/ml or about 1530 h*ng/ml to about 3300 h*ng/ml. In one embodiment, the mean AUCₜ (ng*hr/ml) is about at least 2991 h*ng/ml or about at least 2140 h*ng/ml.

In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a mean AUCₜ (ng*hr/ml) measured over about 24.5 hours after administration of between about 2300 h*ng/ml to about 4000 h*ng/ml. In one embodiment, the mean AUCₜ (ng*hr/ml) measured over about 24.5 hours after administration is about 2300, 2400, 2500, 2600, 2700, 2800, 2900, 3000, 3100, 3200, 3300, 3400, 3500, 3600, 3700, 3800, 3900, or 4000 (ng*hr/ml). In one embodiment, the mean AUCₜ (ng*hr/ml) measured over about 24.5 hours after administration is about 2830 (ng*hr/ml)±550 (ng*hr/ml). In an alternative embodiment, the mean AUCₜ (ng*hr/ml) measured over about 24.5 hours after administration is about 2830 (ng*hr/ml)±560 (ng*hr/ml) or about 2830 (ng*hr/ml)±about 20%. In one embodiment, the mean AUCₜ (ng*hr/ml) measured over about 24.5 hours after administration is about 3020 (ng*hr/ml) about 20%. In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a mean AUCₜ (ng*hr/ml) measured over about 24.5 hours after administration of at least about 2040 ng*hr/ml. In one embodiment, the mean AUCₜ (ng*hr/ml) measured over about 72.5 hours after administration is between about 2300 h*ng/ml to about 4100 h*ng/ml. In one embodiment, the mean AUCₜ (ng*hr/ml) measured over about 72.5 hours after administration is about 2300, 2400, 2500, 2600, 2700, 2800, 2900, 3000, 3100, 3200, 3300, 3400, 3500, 3600, 3700, 3800, 3900, 4000, or 4100 (ng*hr/ml). In an alternative embodiment, the mean AUCₜ (ng*hr/ml) measured over about 72.5 hours after administration is about 3100 (ng*hr/ml) 620 (ng*hr/ml) or about 3100 (ng*hr/ml)±about 20%. In one embodiment, the mean AUCₜ (ng*hr/ml) measured over about 72.5 hours after administration is about 3410 (ng*hr/ml)±about 20%. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one embodiment, Compound 1 is intravenously administered to a subject prior to administration of a chemotherapeutic agent so that a single dose of Compound 1 provides a blood plasma level profile with a dosage-corrected mean AUCₜ (h*ng/ml)/(mg/m²) of between about 6 (h*ng/ml)/(mg/m²) and 20 (h*ng/ml)/(mg/m²), of between about 8 (h*ng/ml)/(mg/m²) and 15 (h*ng/ml)/(mg/m²), of between about 10 (h*ng/ml)/(mg/m²) and 13 (h*ng/ml)/(mg/m²). In one embodiment, the dosage-corrected mean AUCₜ (h*ng/ml)/(mg/m²) is about 6, 7, 8, 9, 10, 11, 12, 13, 14, 15, 16, 17, 18, 19, or 20 (h*ng/ml)/(mg/m²). In one embodiment, the dosage-corrected mean AUCₜ (h*ng/ml)/(mg/m²) is about 12.5 (h*ng/ml)/(mg/m²)±2.2 (h*ng/ml)/(mg/m²). In one embodiment, the dosage-corrected mean AUCₜ (h*ng/ml)/(mg/m²) is about 10.5 (h*ng/ml)/(mg/m²), about 11.0 (h*ng/ml)/(mg/m²), about 11.5 (h*ng/ml)/(mg/m²), about 12.0 (h*ng/ml)/(mg/m²), about 12.5 (h*ng/ml)/(mg/m²), or about 13.0 (h*ng/ml)/(mg/m²). The dosage corrected mean AUCₜ is mean AUCₜ divided by the number of milligrams/m² of Compound 1 in the formulation. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m².

In an alternative embodiment, Compound 1 is intravenously administered to a subject prior to administration of a chemotherapeutic agent so that a single dose of Compound 1 provides a blood plasma level profile with a dosage-corrected mean AUCₜ (h*ng/ml)/(mg/m²) of between about 12.3 (h*ng/ml)/(mg/m²) to about 19.5 (h*ng/ml)/(mg/m²) or about 7.6 (h*ng/ml)/(mg/m²) to about 16.5 (h*ng/ml)/(mg/m²). In one embodiment, the dosage-corrected mean AUCₜ (h*ng/ml)/(mg/m²) is about at least 15.6 (h*ng/ml)/(mg/m²) or about at least 10.7 (h*ng/ml)/(mg/m²).

In one aspect, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a dosage-corrected mean AUCₜ (h*ng/ml)/(mg/m²) of between about 6 (h*ng/ml)/(mg/m²) and 20 (h*ng/ml)/(mg/m²). In one embodiment, the dosage-corrected mean AUCₜ (h*ng/ml)/(mg/m²) is about 15.0 (h*ng/ml)/(mg/m²)±3.0 (h*ng/ml)/(mg/m²) or about 15.0 (h*ng/ml)/(mg/m²)±about 20%. In one embodiment, the dosage-corrected mean AUCₜ (h*ng/ml)/(mg/m²) is at least about 8.35 (h*ng/ml)/(mg/m²). In one embodiment, the dosage-corrected mean AUCₜ (h*ng/ml)/(mg/m²) is about 16.5 (h*ng/ml)/(mg/m²)±about 20%. In one embodiment, the dosage-corrected mean AUCₜ (h*ng/ml)/(mg/m²) is at least about 10.0 (h*ng/ml)/(m g/m²). The dosage corrected AUCₜ is AUCₜ divided by the number of milligrams/m² of Compound 1 in the formulation. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one embodiment, Compound 1 is intravenously administered to a subject prior to administration of a chemotherapeutic agent so that a single dose of Compound 1 provides a blood plasma level profile with a dosage-corrected mean AUC_{inf} (h*ng/ml)/(mg/m²) of between about 6 (h*ng/ml)/(mg/m²) and 20 (h*ng/ml)/(mg/m²), of between about 8 (h*ng/ml)/(mg/m²) and 15 (h*ng/ml)/(mg/m²), of between about 10 (h*ng/ml)/(mg/m²) and 13 (h*ng/ml)/(mg/m²). In one embodiment, the dosage-corrected mean AUC_{inf} (h*ng/ml)/(mg/m²) is about 6, 7, 8, 9, 10, 11, 12, 13, 14, 15, 16, 17, 18, 19, or 20 (h*ng/ml)/(mg/m²). In one embodiment, the dosage-corrected mean AUC_{inf} (h*ng/ml)/(mg/m²) is about 12.7 (h*ng/ml)/(mg/m²)±2.5 (h*ng/ml)/(mg/m²). In one embodiment, the dosage-corrected mean AUC_{inf} (h*ng/ml)/(mg/m²) is about 10.5 (h*ng/ml)/(mg/m²), about 11.0 (h*ng/ml)/(mg/m²), about 11.5 (h*ng/ml)/(mg/m²), about 12.0 (h*ng/ml)/(mg/m²), about 12.5 (h*ng/ml)/(mg/m²), about 13.0 (h*ng/ml)/(mg/m²), or about 13.5 (h*ng/ml)/(mg/m²). The dosage corrected mean AUC_{inf} is mean AUC_{inf} divided by the number of milligrams/m² of Compound 1 in the formulation. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m².

In an alternative embodiment, Compound 1 is intravenously administered to a subject prior to administration of a chemotherapeutic agent so that a single dose of Compound 1 provides a blood plasma level profile with a dosage-corrected mean AUC_{inf} (h*ng/ml)/(mg/m²) measured over 24.5 hours after administration of between about 12.4 (h*ng/ml)/(mg/m²) to about 19.6 (h*ng/ml)/(mg/m²) or about 7.6 (h*ng/ml)/(mg/m²) to about 16.5 (h*ng/ml)/(mg/m²). In one embodiment, the mean AUC_{inf} (h*ng/ml)/(mg/m²) measured over 24.5 hours after administration is about at least 15. (h*ng/ml)/(mg/m²) or about at least 10.7 (h*ng/ml)/(mg/m²). In one embodiment, the mean AUC_{inf} (h*ng/ml)/(mg/m²) measured over 72.5 hours after administration of between about 12.4 (h*ng/ml)/(mg/m²) to about 19.6 (h*ng/ml)/(mg/m²) or about 7.6 (h*ng/ml)/(mg/m²) to about 16.5 (h*ng/ml)/(mg/m²). In one embodiment, the mean AUC_{inf} (h*ng/ml)/(mg/m²) measured over 72.5 hours after administration is about at least 15.6 h*(h*ng/ml)/(mg/m²) or about at least 10.7 (h*ng/ml)/(mg/m²).

In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a dosage-corrected mean AUCₜ (h*ng/ml)/(mg/m²) of between about 6 (h*ng/ml)/(mg/m²) and 20 (h*ng/ml)/(mg/m²). In one embodiment, the dosage-corrected mean AUCₜ (h*ng/ml)/(mg/m²) is 6, 7, 8, 9, 10, 11, 12, 13, 14, 15, 16, 17, 18, 19, or 20 (h*ng/ml)(mg/m²). In one embodiment, the dosage-corrected mean AUCₜ (h*ng/ml)/(mg/m²) is about 15.0 (h*ng/ml)/(mg/m²)±3.0 (h*ng/ml)/(mg/m²) or about 15.0 (h*ng/ml)/(mg/m²)±about 20%. In one embodiment, the dosage-corrected mean AUCₜ (h*ng/ml)/(mg/m²) is about 8.35 (h*ng/ml)/(mg/m²)±about 20%. In one embodiment, the dosage-corrected mean AUCₜ (h*ng/ml)/(mg/m²) is about 16.5 (h*ng/ml)/(mg/m²)±about 20%. In one embodiment, the dosage-corrected mean AUCₜ (h*ng/ml)/(mg/m²) is at least about 10.0 (h*ng/ml)/(mg/m²). The dosage corrected AUCₜ is AUCₜ divided by the number of milligrams/m² of Compound 1 in the formulation. In one embodiment, Compound 1 is administered on days 1 and 2 of the treatment regime. In one embodiment, Compound 1 is administered on days 1, 2, and 3 of the treatment regime. In one embodiment, Compound 1 is administered on days 1, 2, 3, and 4 of the treatment regime. In one embodiment, Compound 1 is administered on days 1, 2, 3, 4, and 5 of the treatment regime.

In one embodiment, Compound 1 is intravenously administered to a subject prior to administration of a chemotherapeutic agent so that a single dose of Compound 1 provides a blood plasma level profile with a with a mean CL (L/h/m²) measured over 24.5 hours after administration of between about 45 L/h/m² and about 85 L/h/m². In one embodiment, the mean CL (L/h/m²) measured over 24.5 hours after administration is 45, 50, 55, 60, 65, 70, 75, 80, or 85 (L/h/m²). In one embodiment, the mean CL (L/h/m²) measured over 24.5 hours after administration is about 65 (L/h/m²)±15 (L/h/m²). In one embodiment, the mean CL (L/h/m²) measured over 24.5 hours after administration is about 64.4 (L/h/m²)±10.6 (L/h/m²). In one embodiment, the mean CL (L/h/m²) measured over 72.5 hours after administration is between about 45 (L/h/m²) to about 80 (L/h/m²). In one embodiment, the mean CL (L/h/m²) measured over 72.5 hours after administration is about 60 (L/h/m²)±15 (L/h/m²). In one embodiment, the mean CL (L/h/m2) measured over 72.5 hours after administration is about 62.1 (L/h/m²)±10.3 (L/h/m²). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a CL (L/h/m²) as described above.

In one embodiment, provided is a method of treating a subject undergoing chemotherapy for the treatment of a CDK 4/6-replication independent cellular proliferation disorder by providing an intravenous administered formulation of Compound 1 wherein a single-dose provides a blood plasma level profile with a mean Vss (L/m²) measured over 24.5 hours after final administration of between about 320 (L/m²) and about 630 (L/m²). In one embodiment, the mean Vss (L/m²) measured over 24.5 hours is 320, 370, 400, 420, 470, 500, 520, 570, 600, or 630 (L/m²). In one embodiment, the mean Vss (L/m²) measured over 24.5 hours is about 425 (L/m²)±150 (L/m²). In one embodiment, the mean Vss (L/m²) measured over 24.5 hours in about 421 (L/m²)±101 (L/m²). In one embodiment, the mean Vss (L/m²) measured over 72.5 hours after final administration of between about 390 (L/m²) and about 825 (L/m²). In one embodiment, the mean Vss (L/m²) measured over 72.5 hours after final administration is 400, 450, 500, 550, 600, 650, 700, 750, 800, or 825 (L/m²). In one embodiment, the mean Vss (L/m²) measured over 72.5 hours after final administration is about 550 (L/m²)±175 (L/m²). In one embodiment, the mean Vss (L/m²) measured over 72.5 hours after final administration is about 547 (L/m²)±147 (L/m²). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a Vss (L/m²) as described above.

In one embodiment, Compound 1 is intravenously administered to a subject prior to administration of a chemotherapeutic agent so that a single dose of Compound 1 provides a blood plasma level profile with a mean MRTs (h) measured over 24.5 hours of between about 4.75 (h) and about 9.25 (h). In one embodiment, the mean MRT_{inf} (h) measured over 24.5 hours is 4.75, 5.25, 5.75, 6.25, 6.75, 7.25, 7.75, 8.25, 8.75, or 9.25 (h). In one embodiment, the mean MRT_{inf} (h) measured over 24.5 hours is about 6.5 (h)±1.75 (h). In one embodiment, the mean MRT_{inf} (h) measured over 24.5 hours is about 6.59 (h)±1.33 (h). In one embodiment, the mean MRT_{inf} (h) measured over 72.5 hours is between about 6 (h) and about 13 (h). In one embodiment, the mean MRT_{inf} (h) measured over 72.5 hours is about 9 (h)±2.5 (h). In one embodiment, the mean MRT_{inf} (h) measured over 72.5 hours is about 8.86 (h)±2.12 (h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a Vss (L/m2) as described above.

In one embodiment, Compound 1 is intravenously administered to a subject prior to administration of a chemotherapeutic agent so that a single dose of Compound 1 provides a blood plasma level profile with a mean λ_{z} (1/h) measured over 24.5 hours of between about 0.07 and 0.15. In one embodiment, the mean λz (1/h) measured over 24.5 hours is 0.07, 0.08, 0.09, 0.10, 0.11, 0.12, 0.13, 0.14, or 0.15 (1/h). In one embodiment, the mean λ_{z} (1/h) measured over 24.5 hours is about 0.09±0.025. In one embodiment, the λ_{z} mean (1/h) measured over 24.5 hours is about 0.0899±0.0157. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a λz (1/h) measured over 24.5 hours as described above.

In one embodiment, Compound 1 is intravenously administered to a subject prior to administration of a chemotherapeutic agent so that a single dose of Compound 1 provides a blood plasma level profile with a mean t_{1/2} (h) measured over 24.5 hours of between about 5 h and 9.5 h. In one embodiment, the mean t_{1/2} (h) measured over 24.5 hours is 5, 5.5, 6, 6.5, 7, 7.5, 8, 8.5, 9, or 9.5 h. In one embodiment, the mean t_{1/2} (h) measured over 24.5 hours is about 8±1.5 (h). In one embodiment, the mean t_{1/2} (h) measured over 24.5 hours is about 7.87±1.14 (h). In one embodiment, the mean t_{1/2}β (h) measured over 72.5 hours is between about 5.5 (h) and about 9 (h). In one embodiment, the mean t_{1/2β} (h) measured over 24.5 hours is 5, 5.5, 6, 6.5, 7, 7.5, 8, 8.5, or 9 h. In one embodiment, the mean t_{1/2β} (h) measured over 72.5 hours is about 8 (h)±1.5 (h). In one embodiment, the mean t_{1/2β} (h) measured over 72.5 hours is about 7.87 (h)±1.14 (h). In one embodiment, the mean t_{1/2}γ (h) measured over 72.5 hours is between about 15 (h) and about 22 (h). In one embodiment, the mean t_{1/2}γ (h) measured over 72.5 hours is 15, 16, 17, 18, 19, 20, 21, or 22 (h). In one embodiment, the mean t_{1/2}γ (h) measured over 72.5 hours is about 18 (h)±2.25 (h). In one embodiment, the mean t_{1/2}γ (h) measured over 72.5 hours is about 18.0 (h)±1.92 (h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a t1_{/2} (h), t_{1/2}β (h), and/or t_{1/2}γ (h) measured over 24.5 hours and/or 72.5 hours as described above.

In an alternative embodiment, Compound 1 is intravenously administered to a subject prior to administration of a chemotherapeutic agent so that a single dose of Compound 1 provides a blood plasma level profile with a mean t_{1/2} (h) of between about 11.9 h and 17.3 h.

In one embodiment, Compound 1 is intravenously administered to a subject prior to administration of a chemotherapeutic agent so that a single dose of Compound 1 provides a blood plasma level profile with a mean concentration (ng/ml) at 24.5 hours after the end of administration of between about 5 (ng/ml) and about 35 (ng/ml). In one embodiment, the mean concentration at 24.5 hours after the end of administration is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 (ng/ml). In one embodiment, the mean concentration at 24.5 hours after the end of administration is about 19 (ng/ml)±5.24 (ng/ml). In one embodiment, the mean concentration at 24.5 hours after the end of administration is about 20 (ng/ml)±7.5 (ng/ml). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean concentration (ng/ml) at 24.5 hours after the end of administration as described above.

In one embodiment, Compound 1 is intravenously administered to a subject prior to administration of a chemotherapeutic agent so that a single dose of Compound 1 provides a blood plasma level profile with a mean concentration (ng/ml) at 72.5 hours after the end of administration of between about 0.7 (ng/ml) and about 3 (ng/ml). In one embodiment, the mean concentration at 72.5 hours after the end of administration is about 0.7, 0.9, 1.0, 1.2, 1.4, 1.6, 1.8, 2.0, 2.2, 2.4, 2.6, 2.8, or 3 (ng/ml). In one embodiment, the mean concentration at 72.5 hours after the end of administration is about 2.25 (ng/ml)±1.5 (ng/ml). In one embodiment, the mean concentration at 72.5 hours after the end of administration is about 1.79 (ng/ml)±0.731 (ng/ml). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean concentration (ng/ml) at 72.5 hours after the end of administration as described above.

In one embodiment, Compound 1 is intravenously administered to a subject prior to administration of a chemotherapeutic agent so that a single dose of Compound 1 provides a primary pharmacokinetic blood plasma level profile with a mean α (1/h) of between about 1 (1/h) and 15 (1/h). In one embodiment, a single-dose provides a primary pharmacokinetic blood plasma level profile with a mean α (1/h) of 1, 2, 3, 4, 5, 6, 7, 8, 9, 10, 11, 12, 13, 14, 15 (1/h). In one embodiment, a single-dose provides a primary pharmacokinetic blood plasma level profile with a mean α (1/h) of about 11 (1/h)±9 (1/h). In one embodiment, a single-dose provides a primary pharmacokinetic blood plasma level profile with a mean α (1/h) of about 11.3 (1/h)±7.06 (1/h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean α (1/h) as described above.

In one embodiment, Compound 1 is intravenously administered to a subject prior to administration of a chemotherapeutic agent so that a single dose of Compound 1 provides a primary pharmacokinetic blood plasma level profile with a mean β (1/h) of about 0.4 (1/h)±0.3 (1/h). In one embodiment, provided is a primary pharmacokinetic blood plasma level profile with a mean β (1/h) of about 0.362 (1/h)±0.110 (1/h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean β (1/h) as described above.

In one embodiment, Compound 1 is intravenously administered to a subject prior to administration of a chemotherapeutic agent so that a single dose of Compound 1 provides a primary pharmacokinetic blood plasma level profile with a mean γ (1/h) of about 0.05 (1/h)±0.01 (1/h). In one embodiment, provided is a primary pharmacokinetic blood plasma level profile with a mean γ (1/h) of about 0.0497 (1/h)±0.00442 (1/h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean γ (1/h) as described above.

In one embodiment, Compound 1 is intravenously administered to a subject prior to administration of a chemotherapeutic agent so that a single dose of Compound 1 provides a 3-compartment primary pharmacokinetic blood plasma level profile with a mean K₂₁ (1/h) of about 1 (1/h)±0.6. In one embodiment, a single-dose provides a 3-compartment primary pharmacokinetic blood plasma level profile with a mean K₂₁ (1/h) of about 0.993 (1/h)±0.439. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean K₂₁ (1/h) as described above.

In one embodiment, Compound 1 is intravenously administered to a subject prior to administration of a chemotherapeutic agent so that a single dose of Compound 1 provides a 3-compartment primary pharmacokinetic blood plasma level profile with a mean K₃₁ (1/h) of about 0.08 (1/h)±0.03 (1/h). In one embodiment, a single-dose provides a 3-compartment primary pharmacokinetic blood plasma level profile with a mean K31 (1/h) of about 0.0750 (1/h)±0.0160 (1/h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean K31 (1/h) as described above.

In one embodiment, Compound 1 is intravenously administered to a subject prior to administration of a chemotherapeutic agent so that a single dose of Compound 1 provides a 3-compartment primary pharmacokinetic blood plasma level profile with a mean V1 (L/m²) of about 25±15. In one embodiment, a single-dose provides a 3-compartment primary pharmacokinetic blood plasma level profile with a mean V1 (L/m²) of about 25.6±9.51. In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean V1 (L/m²) as described above.

In one embodiment, Compound 1 is intravenously administered to a subject prior to administration of a chemotherapeutic agent so that a single dose of Compound 1 provides a 3-compartment primary pharmacokinetic blood plasma level profile with a mean Cmax (ng/ml) of about 2000 (ng/ml)±650 (ng/ml). In one embodiment, a single-dose provides a 3-compartment primary pharmacokinetic blood plasma level profile with a mean Cmax (ng/ml) of about 2020 (ng/ml)±505 (ng/ml). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean Cmax (ng/ml) as described above.

In one embodiment, Compound 1 is intravenously administered to a subject prior to administration of a chemotherapeutic agent so that a single dose of Compound 1 provides a primary pharmacokinetic blood plasma level profile with a mean t_{1/2α} of about 0.1 (h)±0.05 (h). In one embodiment, a single-dose provides a primary pharmacokinetic blood plasma level profile with a mean t_{1/2α} of about 0.0776 (h)±0.0329 (h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, the single dose is about 192 mg/m2. In one embodiment, the single dose is about 200 mg/m2. In one embodiment, the single dose is about 240 mg/m2. In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean t_{1/2α} (h) as described above.

In one embodiment, Compound 1 is intravenously administered to a subject prior to administration of a chemotherapeutic agent so that a single dose of Compound 1 provides a 2-compartment primary pharmacokinetic blood plasma level profile with a mean t_{1/2β} of about 2 (h)±0.75 (h). In one embodiment, a single-dose provides a 2-compartment primary pharmacokinetic blood plasma level profile with a mean t_{1/2β} of about 2.03 (h)±0.444 (h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean t_{1/2β} (h) as described above.

In one embodiment, Compound 1 is intravenously administered to a subject prior to administration of a chemotherapeutic agent so that a single dose of Compound 1 provides a 3-compartment primary pharmacokinetic blood plasma level profile with a mean t_{1/2γ} of about 15 (h)±3 (h). In one embodiment, a single-dose provides a 3-compartment primary pharmacokinetic blood plasma level profile with a mean t_{1/2γ} of about 14.0 (h)±1.35 (h). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean t_{1/2γ} (h) as described above.

In one embodiment, Compound 1 is intravenously administered to a subject prior to administration of a chemotherapeutic agent so that a single dose of Compound 1 provides a 3-compartment primary pharmacokinetic blood plasma level profile with a mean AUC (h*ng/ml) of about 3200(h*ng/ml)±750 (h*ng/ml). In one embodiment, a single-dose provides a 3-compartment primary pharmacokinetic blood plasma level profile with a mean AUC (h*ng/ml) of about 3220(h*ng/ml)±559 (h*ng/ml). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean AUC (h*ng/ml) as described above.

In one embodiment, Compound 1 is intravenously administered to a subject prior to administration of a chemotherapeutic agent so that a single dose of Compound 1 provides a 3-compartment primary pharmacokinetic blood plasma level profile with a mean CL (L/h/m²) of about 60 (L/h/m²)±15 (L/h/m²). In one embodiment, a 3-compartment primary pharmacokinetic blood plasma level profile with a mean CL (L/h/m²) of about 61.1 (L/h/m²)±10.6 (L/h/m²). In one embodiment, the single dose is between about 170 mg/m² and 240 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean CL (L/h/m²) as described above.

In one embodiment, Compound 1 is intravenously administered to a subject prior to administration of a chemotherapeutic agent so that a single dose of Compound 1 provides a 3-compartment primary pharmacokinetic blood plasma level profile with a mean Vss (L/m²) of about 500 (L/m²)±200 (L/m²). In one embodiment, a 3-compartment primary pharmacokinetic blood plasma level profile with a mean Vss (L/m²) of about 508 (L/m²)±131 (L/m²). In one embodiment, the single dose is between about 170 mg/m² and 280 mg/m² or 170 mg/m² and 215 mg/m². In one embodiment, the single dose is about 192 mg/m². In one embodiment, the single dose is about 200 mg/m². In one embodiment, the single dose is about 240 mg/m². In one embodiment, Compound 1 is administered to a subject prior to administration of a chemotherapeutic agent in a multi-day chemotherapeutic treatment regime, for example, 2 days, 3 days, 4 days, or 5 days, wherein Compound 1, following administration on any day of the multi-day chemotherapeutic treatment regime, for example day 2, 3, 4, or 5, provides a blood plasma level profile of Compound 1 with a with a mean Vss (L/m²) as described above.

In certain embodiments, Compound 1 at the dosages described about is administered daily for more than 1, more than 2, more than 3, more than 4, more than 5, more than 6, more than 7, more than 8, more than 9, more than 10, more than 11, more than 12, more than 13, more than 14, more than 15, more than 16, more than 17, more than 18, more than 19, more than 20, more than 21, more than 22, more than 23, more than 24, more than 25 more than 26, more than 27, or more than 28 days. In one embodiment, provided is a method of treating a subject undergoing chemotherapy for the treatment of a CDK 4/6-replication independent cellular proliferation disorder by providing an intravenous administered formulation of Compound 1 at a dosage described above daily for 1, 2, 3, 4, 5, 6, 7, 8, 9, 10, 11, 12, 13, 14, 15, 16, 17, 18, 19, 20, 21, 22, 23, 24, 25, 26, 27, or 28 days or more.

In one embodiment, provided is a method of treating a subject undergoing chemotherapy for the treatment of a CDK 4/6-replication independent cellular proliferation disorder by providing an intravenous administered formulation of Compound 1 wherein a single-dose of Compound 1 followed by a single-dose of Topotecan at 1.5 mg/m² provides a mean Cmax (ng/ml) of Topotecan between about 30 ng/ml to about 150 ng/ml. In one embodiment, the mean Cmax (ng/ml) of Topotecan at 1.5 mg/m² is about 30 ng/ml, about 40 ng/ml, about 50 ng/ml, about 60 ng/ml, about 70 ng/ml, about 80 ng/ml, about 90 ng/ml, about 100 ng/ml. about 110 ng/ml, about 120 ng/ml, about 130 ng/ml, about 140 ng/ml or about 150 ng/ml. In one embodiment, a single-dose of Compound 1 followed by a single-dose of Topotecan at 1.25 mg/m² provides a mean Cmax (ng/ml) of Topotecan between about 20 ng/ml and 120 ng/ml. In one embodiment, the mean Cmax (ng/ml) of Topotecan at 1.25 mg/m² is about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 110 ng/ml, or about 120 ng/ml. In one embodiment, a single-dose of Compound 1 followed by a single-dose of Topotecan at 0.75 mg/m² provides a mean Cmax (ng/ml) of Topotecan between about 10 ng/ml and 70 ng/ml. In one embodiment, the mean Cmax (ng/ml) of Topotecan at 0.75 mg/m² is about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, or about 70 ng/ml.

In an alternative embodiment, provided is a method of treating a subject undergoing chemotherapy for the treatment of a CDK 4/6-replication independent cellular proliferation disorder by providing an intravenous administered formulation of Compound 1 wherein a single-dose of Compound 1 followed by a single-dose of Topotecan at 1.5 mg/m² provides a mean Cmax (ng/ml) of Topotecan between about 40.2 ng/ml and about 122 ng/ml. In one embodiment, a single-dose of Compound 1 followed by a single-dose of Topotecan at 1.25 mg/m² provides a mean Cmax (ng/ml) of Topotecan between about 33.1 ng/ml and 104 ng/ml. In one embodiment, a single-dose of Compound 1 followed by a single-dose of Topotecan at 0.75 mg/m² provides a mean Cmax (ng/ml) of Topotecan between about 17.9 ng/ml and 38.5 ng/ml.

In one embodiment, provided is a method of treating a subject undergoing chemotherapy for the treatment of a CDK 4/6-replication independent cellular proliferation disorder by providing an intravenous administered formulation of Compound 1 wherein a single-dose of Compound 1 followed by a single-dose of Topotecan at 1.5 mg/m² provides a mean AUCτ (h*ng/ml) of Topotecan between about 100 h*ng/ml and about 300 h*ng/ml. In one embodiment, the mean AUCτ (h*ng/ml) of Topotecan at 1.5 mg/m² is about 100 h*ng/ml, about 110 h*ng/ml, about 120 h*ng/ml, about 130 h*ng/ml, about 140 h*ng/ml, about 150 h*ng/ml, about 160 h*ng/ml, about 170 h*ng/ml, about 180 h*ng/ml, about 190 h*ng/ml, about 200 h*ng/ml, about 220 h*ng/ml, about 240 h*ng/ml, about 260 h*ng/ml, about 280 h*ng/ml, or about 300 h*ng/ml. In one embodiment, a single-dose of Compound 1 followed by a single-dose of Topotecan at 1.25 mg/m² provides a mean AUCτ (h*ng/ml) of Topotecan between about 80 h*ng/ml and about 300 h*ng/ml. In one embodiment, the mean AUCτ (h*ng/ml) of Topotecan at 1.25 mg/m² is about 80 h*ng/ml, about 90 h*ng/ml, about 100 h*ng/ml, about 110 h*ng/ml, about 120 h*ng/ml, about 130 h*ng/ml, about 140 h*ng/ml, about 150 h*ng/ml, about 160 h*ng/ml, about 170 h*ng/ml, about 180 h*ng/ml, about 190 h*ng/ml, about 200 h*ng/ml, about 220 h*ng/ml, about 240 h*ng/ml, about 260 h*ng/ml, about 280 h*ng/ml, or about 300 h*ng/ml. In one embodiment, a single-dose of Compound 1 followed by a single-dose of Topotecan at 0.75 mg/m² provides a mean AUCτ (h*ng/ml) of Topotecan between about 50 h*ng/ml and about 200 h*ng/ml. In one embodiment, the mean AUCτ (h*ng/ml) of Topotecan at 0.75 mg/m² is about 50 h*ng/ml, about 60 h*ng/ml, 70 h*ng/ml, about 80 h*ng/ml, about 90 h*ng/ml, about 100 h*ng/ml, about 110 h*ng/ml, about 120 h*ng/ml, about 130 h*ng/ml, about 140 h*ng/ml, about 150 h*ng/ml, about 160 h*ng/ml, about 170 h*ng/ml, about 180 h*ng/ml, about 190 h*ng/ml, or about 200 h*ng/ml.

In one embodiment, provided is a method of treating a subject undergoing chemotherapy for the treatment of a CDK 4/6-replication independent cellular proliferation disorder by providing an intravenous administered formulation of Compound 1 wherein a single-dose of Compound 1 followed by a single-dose of Topotecan at 1.5 mg/m² provides a mean AUCτ (h*ng/ml) of Topotecan between about 132 h*ng/ml and about 181 h*ng/ml. In one embodiment, a single-dose of Compound 1 followed by a single-dose of Topotecan at 1.25 mg/m² provides a mean AUCτ (h*ng/ml) of Topotecan between about 121 h*ng/ml and about 254 h*ng/ml. In one embodiment, a single-dose of Compound 1 followed by a single-dose of Topotecan at 0.75 mg/m² provides a mean AUCτ (h*ng/ml) of Topotecan between about 74.4 h*ng/ml and about 120 h*ng/ml.

As provided herein, Compound 1 can be administered wherein any one or more of the above described PK or PD blood profile parameters described herein is reached to treat a subject undergoing chemotherapy for the treatment of a CDK 4/6-replication independent cellular proliferation disorder.

In one embodiment, provided is a method of treating a subject having a CDK 4/6-replication dependent cellular proliferation disorder by providing an intravenous administered formulation of Compound 1 in a dosage providing a blood plasma profile as described above.

In one embodiment, provided is method of treating a subject having a CDK 4/6-replication independent cellular proliferation disorder by providing an intravenous administered formulation of Compound 1 in a dosage providing a combination of two or more blood plasma parameters at levels described above. Non-limiting examples of parameters that can be provided in combinations of two or more at levels described above include: mean Cmax, mean dosage-corrected Cmax, mean Tmax, mean AUC_{inf}, dosage-corrected mean AUC_{inf}, mean AUCₜ, dosage-corrected mean AUCₜ, and mean t_{1/2}.

TABLE 1
PK Parameter Ranges Measured Over 24.5 hours

| | | dosage-corrected | | dosage-corrected | | dosage-corrected | |
|---|---|---|---|---|---|---|---|
| Dose | Cmax | Cmax | AUC_{inf} | AUC_{inf} | AUCₜ | AUCₜ | t₁/₂ |
| (mg/m²) | (ng/ml) | (ng/ml)/(mg/m²) | (ng * h/ml) | (ng * h/ml)/(mg/m²) | (ng * h/ml) | (ng * h/ml)/(mg/m²) | (h) |
| 170-280 | 1000-3500 | 1-20 | 2000-4500 | 6-20 | 2300-4000 | 6-20 | 5-9.5 |

In one embodiment, provided is method of treating a subject having a CDK 4/6-replication independent cellular proliferation disorder by providing an intravenous administered formulation of Compound 1 in a single dosage providing at least two parameters in the ranges specified in Table 1. In an alternative embodiment, the single dosage of Compound 1 provides at least three parameters in the ranges specified in Table 1. In an alternative embodiment, the single dosage of Compound 1 provides at least four parameters in the ranges specified in Table 1. In an alternative embodiment, the single dosage of Compound 1 provides at least five parameters in the ranges specified in Table 1. In an alternative embodiment, the single dosage of Compound 1 provides at least six parameters in the ranges specified in Table 1. In an alternative embodiment, the single dosage of Compound 1 provides at least seven parameters in the ranges specified in Table 1. In an alternative embodiment, the single dosage of Compound 1 provides at least eight parameters in the ranges specified in Table 1.

### EXAMPLES

### Example 1

### Compound 1 Demonstrates a Good Drug Profile

Compound 1 was determined to be a highly potent and selective CDK4/6 inhibitor in in vitro and in vivo studies. Treatment of animals with Compound 1 produced a clean and transient G1 arrest in bone marrow stem and progenitor cells, which induced subtle changes in the complete blood count (CBC) following multiple daily doses. Additionally, it was demonstrated that Compound 1 treatment can protect normal cells in vitro and in vivo from the cytotoxic effects of chemotherapy and radiation. Additionally, Compound 1 is considered to have a low potential for producing adverse effects due to off-target pharmacodynamic (PD) activity.

Pharmacokinetic (PK) parameters studied in rats and dogs following IV administration showed that the relationship between dose level and plasma exposure to Compound 1 was generally similar between males and females and did not change with repeated daily dosing. Exposure to Compound 1 increased with dose level, but not always proportionally. Plasma half-life values for Compound 1 after IV administration were ˜4 hours in rats and dogs.

Following oral administration, Compound 1 was well absorbed in the rat, based both upon rapid appearance in plasma and high oral bioavailability (˜60-70%). Systemic exposure in rats as measured by Cmax and AUClast was dose-dependent. In 14-day GLP toxicity studies in rats given oral doses of Compound 1, there were no marked changes in Compound 1 systemic exposure at any dose level when comparing Day 1 vs Day 14. In dogs, Compound 1 exhibited a moderate rate and extent of absorption, with oral bioavailability of 30.2%, 39.9%, and 17.1% at 10, 30 and 90 mg/kg respectively. Systemic exposure in dogs as measured by Cmax and AUCₗₐₛₜ was dose-proportional from 10 to 30 mg/kg, and less than proportional from 30 to 90 mg/kg.

When administered by IV daily for 7 days, Compound 1 was well tolerated in rats at up to 50 mg/kg (≈300 mg/m²) and in dogs at up to 15 mg/kg (≈300 mg/m²) with toxicity characterized chiefly by reduced hematopoiesis that involved all cell lines and was a reflection of the drug's intended PD activity. The magnitude and/or duration of this effect differed among cell lineages but was dose-related in all lineages. Effects on hematopoiesis were readily monitored by peripheral blood cell counts and were reversible when dosing stopped. Clinically significant leukopenia occurred in rats and dogs given Compound 1 for 7 days at ≧150 and 300 mg/m², respectively, and it led to morbidity and mortality in dogs given daily doses for 6 days at 900 mg/m².

These studies indicate that rats and dogs were appropriate species for evaluating the toxicity of Compound 1. In both species, the toxicity profile was similar and the repeat-dose toxicity studies identified no observed adverse effect levels (NOAELs). The lower NOAEL for IV administration was 10 mg/kg (60 mg/m²) in rats, based on the occurrence of adverse hematopoietic effects at ≧150 mg/m² that reflected an exaggeration of the intended PD activity of Compound 1. This IV NOAEL was the basis for selecting a starting dose of 6 mg/m² for the first clinical trial, which is 1/10th of the lowest safe dose level in any animal study.

In rats, the toxicity profile with oral administration was similar to that with IV administration, except that pulmonary macrophage accumulation occurred with oral administration for 14 days at ≧5 mg/kg (≧30 mg/m²) but not with IV administration for 7 days at up to 25 mg/kg (≧150 mg/m²). Based on this finding, the oral NOAEL was 2 mg/kg (≧12 mg/m²).

Human cohorts have received IV doses of Compound 1 of 6, 12, 24, 48, 96, and 192 mg/m². Results have shown that Compound 1 is well tolerated, with no serious adverse events observed. The pharmacokinetic results indicate that both Cmax and AUC increase proportionally to the increasing dose and that CL is independent of dose over the dose range of 6 to 192 mg/m².

### Example 2

### IV Formulation

The synthesis of Compound 1 is described in WO 2012/061156, incorporated in its entirety herein.

An IV formulation for use in the experiments described herein can be a sterile powder, 40 mg of Compound 1 per 10 mL vial. D-mannitol, USP can be added as a cake forming agent and citrate buffer is added to maintain the reconstituted pH at 4.0-4.5. The sterile powder can be reconstituted with 5% sterile dextrose and diluted with water to provide a final concentration between 0.2 mg/mL and 8.0 mg/mL of Compound 1. The reconstituted and diluted product exhibits a final pH of 4.0-4.5 and can be delivered, where indicated, by IV infusion.

### Example 3

### Oral Formulation

An oral formulation for use in the experiments described herein can be a sterile powder, 40 mg Compound 1 per 10 mL vial. D-mannitol, USP can be added as a cake forming agent and citrate buffer can be added to maintain the reconstituted pH at 4.0-4.5. The sterile powder can be reconstituted with apple juice (Brand Name: “Goudappel.” Supplied by Appelsientje) to provide a final concentration between 3 to 12 mg/mL. The reconstituted and diluted product can be delivered by oral administration where indicated.

### Example 4

### IV Administration

Compound 1 sterile powder, 40 mg/vial can be stored in the refrigerator at 2° C.-8° C. After reconstitution and dilution the solution may be stored in a plastic syringe for up to 24 hours at ambient temperature and ambient lighting prior to administration.

### Example 5

### Oral Administration 2

Compound 1 Sterile Powder, 40 mg/vial can be reconstituted with apple juice (Brand Name: “Goudappel.” Supplied by Appelsientje) to a final concentration of 3-12 mg Compound 1 per mL. The product can be stored at ambient conditions for up to 4 hours.

### Example 6

### Pharmacokinetic Analysis

2′-((5-(4-methylpiperazin-1-yl)pyridin-2-yl)amino)-7′,8′-dihydro-6′H-spiro[cyclohexane-1,9′-pyrazino[1′,2′:1,5]pyrrolo[2,3-d]pyrimidin]-6′-one (Compound 1) is a highly potent, selective, CDK4/6 inhibitor useful for chemoprotection to reduce chemotherapy-induced myelosuppression (CIM). The CDK 4/6 pathway is important in regulating cell proliferation of certain tumors. In addition, hematopoietic stem and progenitor cells (HSPC) are dependent upon CDK4/6 for proliferation. Transient Compound 1 induced G0/G1 cell cycle arrest of HSPCs renders them resistant to the cytotoxic effects of chemotherapy.

The safety and tolerability of Compound 1, as well as its PK and PD profile, were assessed in a double blind, placebo-controlled, single escalating dose study in healthy volunteers of both sexes, where subjects were randomized (3:1) to receive Compound 1 or placebo as a single 30-minute IV infusion. Pharmacodynamic assessments included evaluation of ex vivo stimulation of lymphocytes and bone marrow cell cycle analysis.

Forty-five subjects have enrolled in the study. Compound 1 was administered at doses of 6, 12, 24, 48, 96 and 192 mg/m². Compound 1 was well tolerated, with no dose limiting toxicity or serious adverse events (SAEs) reported. Compound 1 exposure (Cₘₐₓ and AUC) increased proportionally with dose, while CL was relatively constant. Compound 1 at 96 and 192 mg/m² produced a dose dependent decrease in PHA-stimulated lymphocyte proliferation 4 h post-dosing, with recovery starting at 8 h and approaching baseline at 24 h.

i) Cohort 1: 6 mg/m²

Five subjects were enrolled into Cohort 1 of Study Compound 1. Three subjects received active treatment and 2 received placebo. Single intravenous infusion doses of 6 mg/m² Compound 1 were administered over a 30-minute duration. Blood samples were obtained over at nominal times of 15 minutes into the infusion, at the end of the 30-minute infusion (EOI), and at 0.25, 0.5, 1, 2, 3, 4, 6, 8, 12, 24, 48, and 72 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times. Random subject numbers were assigned by the bioanalytical laboratory to retain the blind.

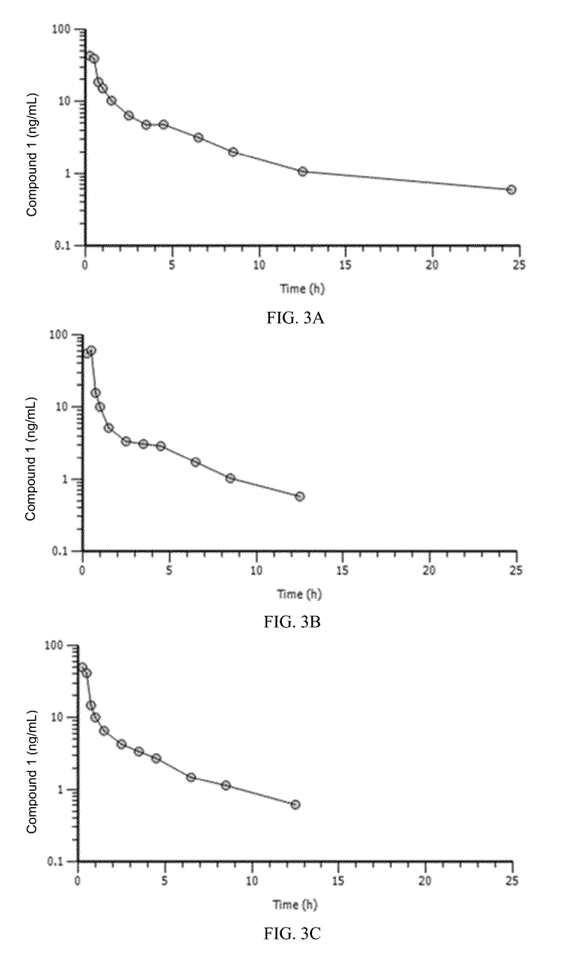

Table 2 contains a summary of the Compound 1 concentration-time data for individual subjects with descriptive statistics for the 3 subjects in Cohort 1. FIG. 2 displays individual subject plots of the Compound 1 concentration-time data on a linear scale and FIG. 3 contains plots on log-linear axes. The highest concentrations for 2 of the 3 subjects occurred at the 15 minute sample during infusion (42.7 and 42.9 ng/mL). For the 3rd subject the peak concentration of 60.5 ng/mL occurred at the end of infusion. Concentrations subsequently decayed with a multi-phase disposition profile as shown on the log-linear plots. At 24.5 hours after the start of infusion, 1 of 3 subjects had a measurable Compound 1 concentration of 0.595 ng/mL. No subjects had measurable concentrations at 48.5 hours after the start of infusion.

TABLE 2
Compound 1 Concentration (ng/mL) Summary Table - Cohort 1 (6 mg/m²)

| | | | Time (h) | | | | | | | | | | | | | |
|---|---|---|---|---|---|---|---|---|---|---|---|---|---|---|---|---|
| | | | 0.00 | 0.25 | 0.50 | 0.75 | 1.00 | 1.50 | 2.50 | 3.50 | 4.50 | 6.50 | 8.50 | 12.50 | 24.50 | 48.50 |
| Cohort | Treatment | Subject | Compound I (ng/mL) | | | | | | | | | | | | | |
| Cohort 1 | 6 mg/m² | 1 | 0.00 | 42.7 | 39.0 | 18.4 | 15.2 | 10.3 | 6.40 | 4.77 | 4.81 | 3.17 | 1.99 | 1.06 | 0.595 | BLQ |
| | | 2 | 0.00 | 54.7 | 60.5 | 15.8 | 10.1 | 5.17 | 3.37 | 3.10 | 2.90 | 1.73 | 1.03 | 0.576 | BLQ | NS |
| | | 3 | 0.00 | 49.5 | 41.1 | 14.7 | 10.1 | 6.58 | 4.30 | 3.39 | 2.73 | 1.48 | 1.14 | 0.619 | BLQ | NS |
| | | N | 3 | 3 | 3 | 3 | 3 | 3 | 3 | 3 | 3 | 3 | 3 | 3 | 1 | 0 |
| | | Mean | 0.00 | 49.0 | 46.9 | 16.3 | 11.8 | 7.35 | 4.69 | 3.75 | 3.48 | 2.13 | 1.39 | 0.752 | 0.595 | NC |
| | | SD | 0.00 | 6.02 | 11.9 | 1.90 | 2.94 | 2.65 | 1.55 | 0.892 | 1.15 | 0.912 | 0.525 | 0.268 | NC | NC |
| | | Min | 0.00 | 42.7 | 39.0 | 14.7 | 10.1 | 5.17 | 3.37 | 3.10 | 2.73 | 1.48 | 1.03 | 0.576 | NC | NC |
| | | Median | 0.00 | 49.5 | 41.1 | 15.8 | 10.1 | 6.58 | 4.30 | 3.39 | 2.90 | 1.73 | 1.14 | 0.619 | NC | NC |
| | | Max | 0.00 | 54.7 | 60.5 | 18.4 | 15.2 | 10.3 | 6.40 | 4.77 | 4.81 | 3.17 | 1.99 | 1.06 | NC | NC |
| | | CV % | NC | 12.3 | 25.3 | 11.7 | 25.0 | 36.1 | 33.1 | 23.8 | 33.2 | 42.9 | 37.9 | 35.6 | NC | NC |
| | | Geometric | NC | 48.7 | 45.9 | 16.2 | 11.6 | 7.05 | 4.53 | 3.69 | 3.36 | 2.01 | 1.33 | 0.723 | NC | NC |
| | | Mean | | | | | | | | | | | | | | |

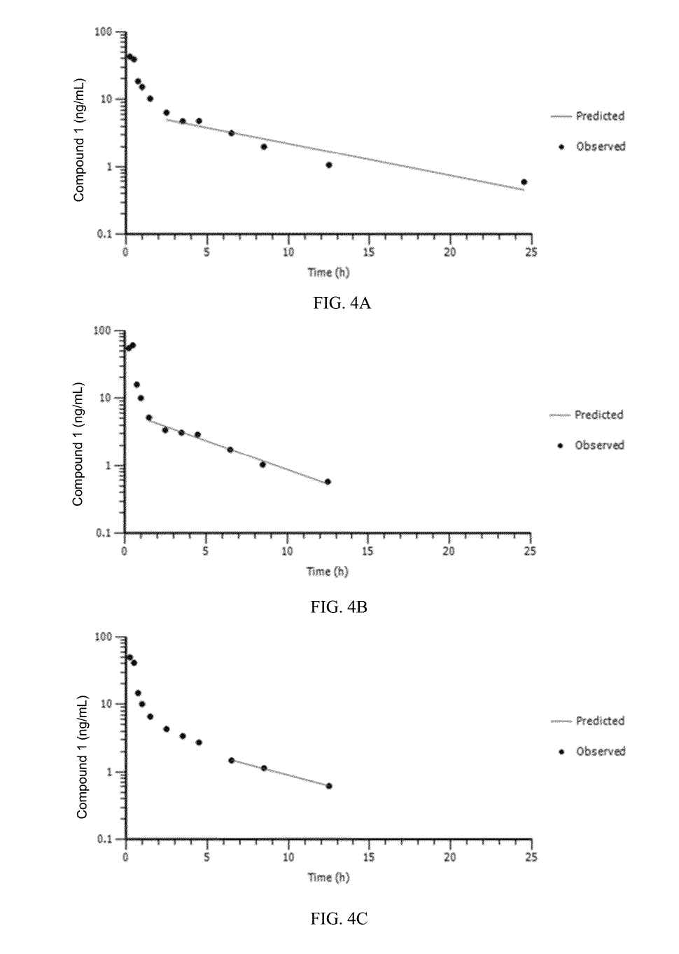

Noncompartmental PK parameters for Compound 1 are summarized in Table 3. Plots of the linear regression analysis for determination of the terminal phase rate constant (λz) and half-life are displayed in FIG. 4. The Cmax averaged 50.9 ng/mL and the Tmax occurred at 0.33 hour. AUCinf values averaged 67.7 h*ng/mL and with low variability, with a CV % of 22.7%. The calculated volume of distribution at steady state (Vss) was large, averaging 358 L/m². Clearance (CL) was high at 91.4 L/h/m², ranging from 70.3 to 104 L/h/m². The variability in the CL values was low, with CV % of 20.1%.

TABLE 3
Pharmacokinetic Parameters for Compound 1 - Cohort 1 (6 mg/m²)

| | | Cmax | Tmax | λz | t½ | AUCinf | CL | Vss | MRTinf |
|---|---|---|---|---|---|---|---|---|---|
| Cohort | Subject | (ng/mL) | (h) | (1/h) | (h) | (h * ng/mL) | (L/h/m²) | (L/m²) | (h) |
| Cohort 1 | 1 | 42.7 | 0.25 | 0.109 | 6.36 | 85.4 | 70.3 | 452 | 6.43 |
| | 2 | 60.5 | 0.50 | 0.197 | 3.52 | 60.1 | 99.8 | 270 | 2.70 |
| | 3 | 49.5 | 0.25 | 0.146 | 4.74 | 57.6 | 104 | 353 | 3.39 |
| | N | 3 | 3 | 3 | 3 | 3 | 3 | 3 | 3 |
| | Mean | 50.9 | 0.333 | 0.151 | 4.87 | 67.7 | 91.4 | 358 | 4.18 |
| | SD | 8.98 | 0.144 | 0.0441 | 1.42 | 15.3 | 18.4 | 91.4 | 1.99 |
| | Min | 42.7 | 0.250 | 0.109 | 3.52 | 57.6 | 70.3 | 270 | 2.70 |
| | Median | 49.5 | 0.250 | 0.146 | 4.74 | 60.1 | 99.8 | 353 | 3.39 |
| | Max | 60.5 | 0.500 | 0.197 | 6.36 | 85.4 | 104 | 452 | 6.43 |
| | CV % | 17.6 | 43.3 | 29.3 | 29.2 | 22.7 | 20.1 | 25.5 | 47.6 |
| | Geometric | 50.4 | 0.315 | 0.146 | 4.73 | 66.6 | 90.0 | 350 | 3.89 |
| | Mean | | | | | | | | |

ii) Cohort 2: 12 mg/m2

Three subjects received 12 mg/m² of Compound 1 administered over a 30-minute duration. Blood samples were obtained at nominal times of 15 minutes into the infusion, at the end of the 30-minute infusion (EOI), and at 0.25, 0.5, 1, 2, 3, 4, 6, 8, 12, 24, 48, and 72 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times.

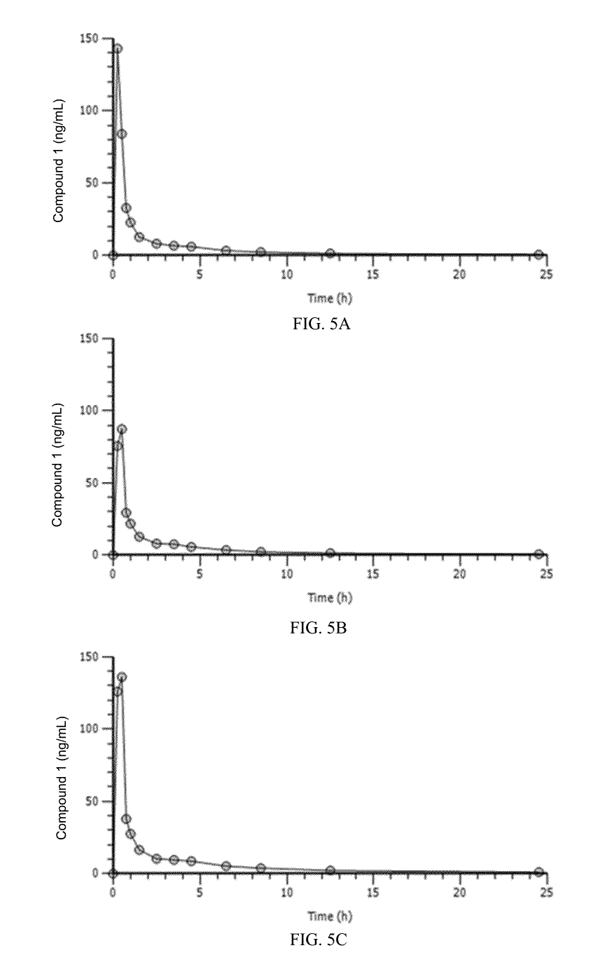

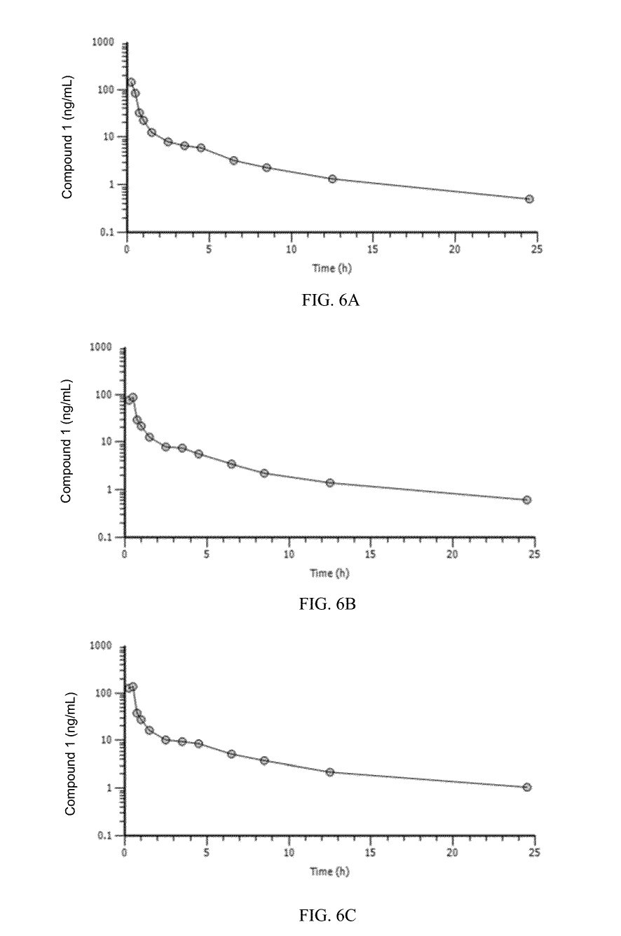

Table 4 contains a summary of Compound 1 concentration-time data for individual subjects with descriptive statistics for the 3 subjects in Cohort 2. FIG. 5 displays individual subject plots of the Compound 1 concentration-time data on a linear scale and FIG. 6 contains plots on log-linear axes. The highest concentrations for 2 of the 3 subjects occurred at the end of the infusion (87.4 and 136 ng/mL). For the 3rd subject the peak concentration of 143 ng/mL occurred at the 15-minute sampling time. Concentrations subsequently decayed with a multi-phase disposition profile as shown on the log-linear plots. At 24.5 hours after the start of infusion, all 3 subjects had low but measurable Compound 1 concentrations ranging from 0.506 to 1.06 ng/mL.

TABLE 4
Compound 1 Concentration (ng/mL) Summary Table - Cohort 2 (12 mg/m²)

| | | | Time (h) | | | | | | | | | | | | |
|---|---|---|---|---|---|---|---|---|---|---|---|---|---|---|---|
| | | | 0.0 | 0.25 | 0.50 | 0.75 | 1.0 | 1.5 | 2.5 | 3.5 | 4.5 | 6.5 | 8.5 | 12.5 | 24.5 |
| Cohort | Treatment | Subject | Compound (ng/mL) | | | | | | | | | | | | |
| Cohort 2 | 12 mg/m² | 1 | 0.00 | 143 | 84.0 | 32.6 | 22.6 | 12.6 | 8.03 | 6.65 | 6.02 | 3.26 | 2.31 | 1.34 | 0.506 |
| | | 2 | 0.00 | 75.7 | 87.4 | 29.3 | 21.7 | 12.7 | 7.92 | 7.51 | 5.65 | 3.49 | 2.26 | 1.42 | 0.621 |
| | | 3 | 0.00 | 126 | 136 | 37.8 | 27.5 | 16.4 | 10.3 | 9.50 | 8.63 | 5.21 | 3.80 | 2.20 | 1.06 |
| | | N | 3 | 3 | 3 | 3 | 3 | 3 | 3 | 3 | 3 | 3 | 3 | 3 | 3 |
| | | Mean | 0.00 | 115 | 102 | 33.2 | 23.9 | 13.9 | 8.75 | 7.89 | 6.77 | 3.99 | 2.79 | 1.65 | 0.729 |
| | | SD | 0.00 | 35.0 | 29.1 | 4.29 | 3.12 | 2.17 | 1.34 | 1.46 | 1.62 | 1.07 | 0.875 | 0.475 | 0.292 |
| | | Min | 0.00 | 75.7 | 84.0 | 29.3 | 21.7 | 12.6 | 7.92 | 6.65 | 5.65 | 3.26 | 2.26 | 1.34 | 0.506 |
| | | Median | 0.00 | 126 | 87.4 | 32.6 | 22.6 | 12.7 | 8.03 | 7.51 | 6.02 | 3.49 | 2.31 | 1.42 | 0.621 |
| | | Max | 0.00 | 143 | 136 | 37.8 | 27.5 | 16.4 | 10.3 | 9.50 | 8.63 | 5.21 | 3.80 | 2.20 | 1.06 |
| | | CV % | NC | 30.5 | 28.4 | 12.9 | 13.0 | 15.6 | 15.4 | 18.5 | 24.0 | 26.7 | 31.4 | 28.7 | 40.1 |
| | | Geometric | NC | 111 | 99.9 | 33.1 | 23.8 | 13.8 | 8.68 | 7.80 | 6.65 | 3.90 | 2.71 | 1.61 | 0.693 |
| | | Mean | | | | | | | | | | | | | |

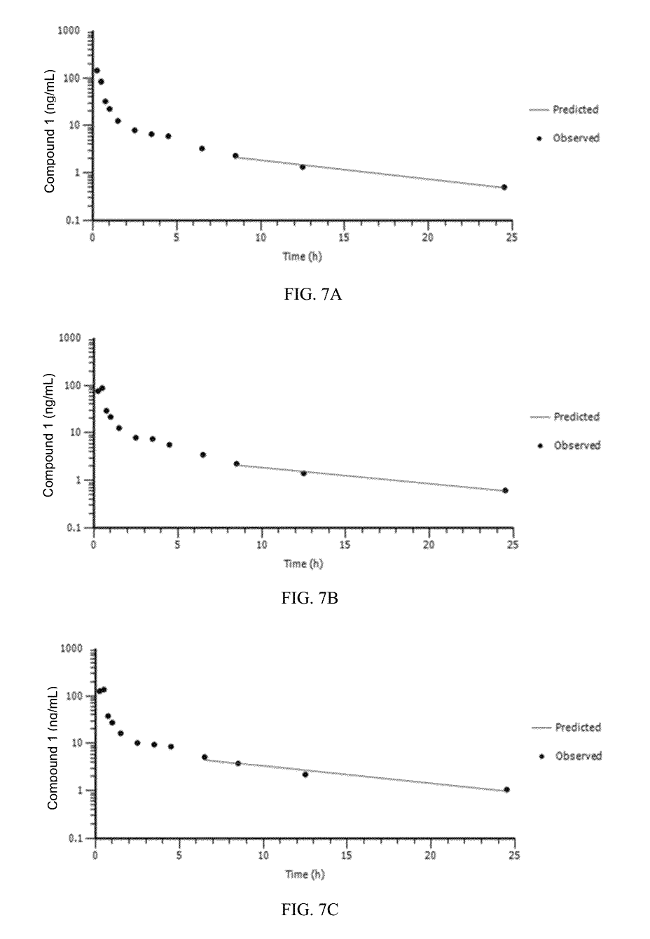

Noncompartmental PK parameters for Compound 1 for Cohort 2 are summarized in Table 5 and previously derived PK parameters for Cohort 1 are displayed above for comparative purposes. Plots of the linear regression analysis for determination of the terminal phase rate constant (λz) and half-life for Cohort 2 subjects are displayed in FIG. 7. The Cmax averaged 122 ng/mL and the median Tmax was 0.5 hour. Half-lives averaged 8.22 hours with a narrow range of 7.56 to 8.89 hours. AUCinf values averaged 148 h*ng/mL with low variability, as reflected by a CV % of 21.6%. The calculated volume of distribution at steady state (Vss) was large, averaging 461 L/m². Clearance (CL) was high at 83.5 L/h/m², ranging from 65.2 to 97 L/h/m². The variability in the CL values was low, with CV % of 19.7%.

TABLE 5
Pharmacokinetic Parameters for Compound 1 - Cohort 2 (12 mg/m²)

| | | Cmax | Tmax | λz | t½ | AUCinf | CL | Vss | MRTinf |
|---|---|---|---|---|---|---|---|---|---|
| Cohort | Subject | (ng/mL) | (h) | (1/h) | (h) | (h * ng/mL) | (L/h/m²) | (L/m²) | (h) |
| Cohort 2 | 1 | 143 | 0.25 | 0.0917 | 7.56 | 136 | 88.4 | 398 | 4.50 |
| | 2 | 87.4 | 0.50 | 0.0780 | 8.89 | 124 | 97.0 | 580 | 5.98 |
| | 3 | 136 | 0.50 | 0.0844 | 8.21 | 184 | 65.2 | 405 | 6.21 |
| | N | 3 | 3 | 3 | 3 | 3 | 3 | 3 | 3 |
| | Mean | 122 | 0.417 | 0.0847 | 8.22 | 148 | 83.5 | 461 | 5.56 |
| | SD | 30.3 | 0.144 | 0.00687 | 0.665 | 32.0 | 16.5 | 103 | 0.930 |
| | Min | 87.4 | 0.250 | 0.0780 | 7.56 | 124 | 65.2 | 398 | 4.50 |
| | Median | 136 | 0.500 | 0.0844 | 8.21 | 136 | 88.4 | 405 | 5.98 |
| | Max | 143 | 0.500 | 0.0917 | 8.89 | 184 | 97.0 | 580 | 6.21 |
| | CV % | 24.8 | 34.6 | 8.1 | 8.1 | 21.6 | 19.7 | 22.4 | 16.7 |
| | Geometric | 119 | 0.397 | 0.0845 | 8.20 | 146 | 82.4 | 454 | 5.51 |
| | Mean | | | | | | | | |

By comparison, the Cmax for Cohort 1 averaged 50.9 ng/mL, and the AUC_{inf} values averaged 67.7 h*ng/mL. Thus, a doubling of the Compound 1 dose resulted in an approximate doubling of the exposure. The calculated (Vss) was slightly larger and the CL values were slightly lower for Cohort 2. As a result the average half-life of 8.22 hours was longer than the average of 4.87 hours observed for Cohort 1.

iii) Cohort 3: 24 mg/m² Compound 1

Three subjects received 24 mg/m² of Compound 1 administered over a 30-minute duration. Blood samples were obtained at nominal times of 15 minutes into the infusion, at the end of the 30-minute infusion (EOI), and at 0.25, 0.5, 1, 2, 3, 4, 6, 8, 12, 24, 48, and 72 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times.

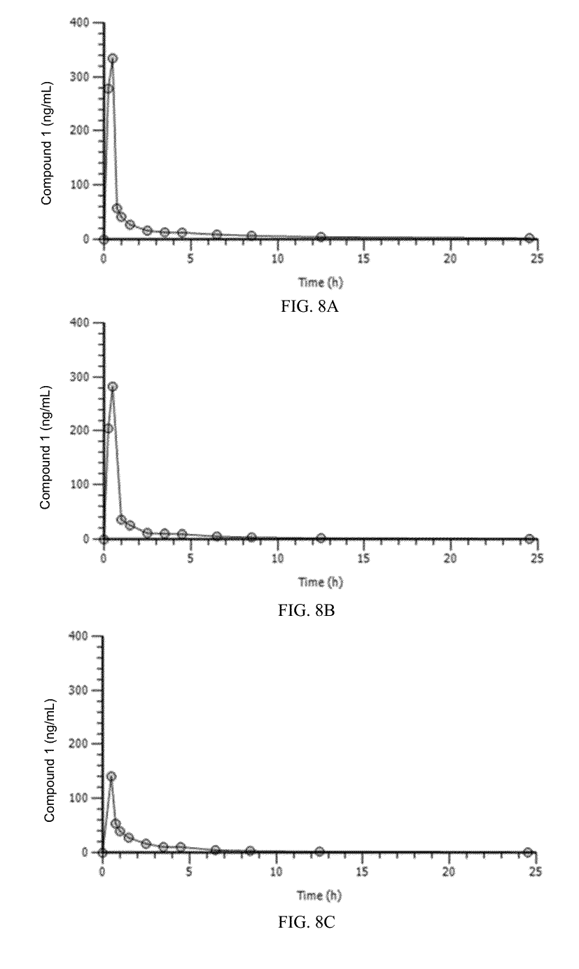

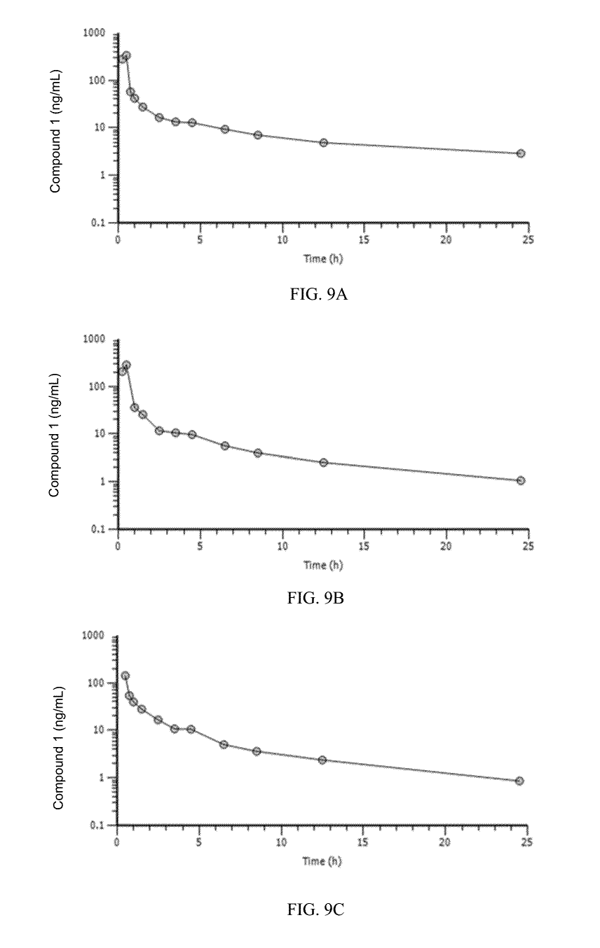

Table 6 contains a summary of the Compound 1 concentration-time data for individual subjects with descriptive statistics for the 3 subjects who received Compound 1 in Cohort 3. Results for single concentration-time values were not available for 2 subjects due to instrument failure and these samples will be repeated during the next run. Subject 2 is missing the 0.75 hour result and Subject 3 is missing the 0.25 hour result. FIG. 8 displays individual subject plots of the Compound 1 concentration-time data on a linear scale and FIG. 9 contains plots on log-linear axes. The highest concentrations for all 3 subjects occurred at the end of the infusion. Concentrations subsequently decayed with a multi-phase disposition profile as shown on the log-linear plots. At 24.5 hours after the start of infusion, all 3 subjects had low but measurable Compound 1 concentrations ranging from 0.870 to 2.91 ng/mL. It should be noted that concentration-time data were also provided for samples obtained at 48.5 and 72.5 hours after the start of the infusion for Cohorts 1 and 2 and the concentrations in all of these were below the lower limit of quantification (BLQ).

TABLE 6
Compound 1 Concentration (ng/mL) Summary Table - Cohort 3 (24 mg/m²)

| | | | Time (h) | | | | | | | | | | | | |
|---|---|---|---|---|---|---|---|---|---|---|---|---|---|---|---|
| | | | 0.0 | 0.25 | 0.50 | 0.75 | 1.0 | 1.5 | 2.5 | 3.5 | 4.5 | 6.5 | 8.5 | 12.5 | 24.5 |
| Cohort | Treatment | Subject | Compound (ng/ml) | | | | | | | | | | | | |
| Cohort 3 | 24 mg/m² | 1 | 0.00 | 278 | 334 | 57.5 | 41.8 | 27.5 | 16.5 | 13.4 | 12.9 | 9.38 | 7.08 | 4.89 | 2.91 |
| | | 2 | 0.00 | 205 | 282 | NR | 36.0 | 25.6 | 11.7 | 10.6 | 9.74 | 5.65 | 4.01 | 2.56 | 1.07 |
| | | 3 | 0.00 | NR | 141 | 53.9 | 39.7 | 28.0 | 16.7 | 10.8 | 10.6 | 5.06 | 3.64 | 2.40 | 0.870 |
| | | N | 3 | 2 | 3 | 2 | 3 | 3 | 3 | 3 | 3 | 3 | 3 | 3 | 3 |
| | | Mean | 0.00 | 242 | 252 | 55.7 | 39.2 | 27.0 | 15.0 | 11.6 | 11.1 | 6.70 | 4.91 | 3.28 | 1.62 |
| | | SD | 0.00 | 51.6 | 99.9 | 2.55 | 2.94 | 1.27 | 2.83 | 1.56 | 1.63 | 2.34 | 1.89 | 1.39 | 1.12 |
| | | Min | 0.00 | 205 | 141 | 53.9 | 36.0 | 25.6 | 11.7 | 10.6 | 9.74 | 5.06 | 3.64 | 2.40 | 0.870 |
| | | Median | 0.00 | 242 | 282 | 55.7 | 39.7 | 27.5 | 16.5 | 10.8 | 10.6 | 5.65 | 4.01 | 2.56 | 1.07 |
| | | Max | 0.00 | 278 | 334 | 57.5 | 41.8 | 28.0 | 16.7 | 13.4 | 12.9 | 9.38 | 7.08 | 4.89 | 2.91 |
| | | CV % | NC | 21.4 | 39.6 | 4.6 | 7.5 | 4.7 | 18.9 | 13.5 | 14.7 | 35.0 | 38.5 | 42.4 | 69.6 |
| | | Geometric | NC | 239 | 237 | 55.7 | 39.1 | 27.0 | 14.8 | 11.5 | 11.0 | 6.45 | 4.69 | 3.11 | 1.39 |
| | | Mean | | | | | | | | | | | | | |

NR = Not reported
NC = Not calculated

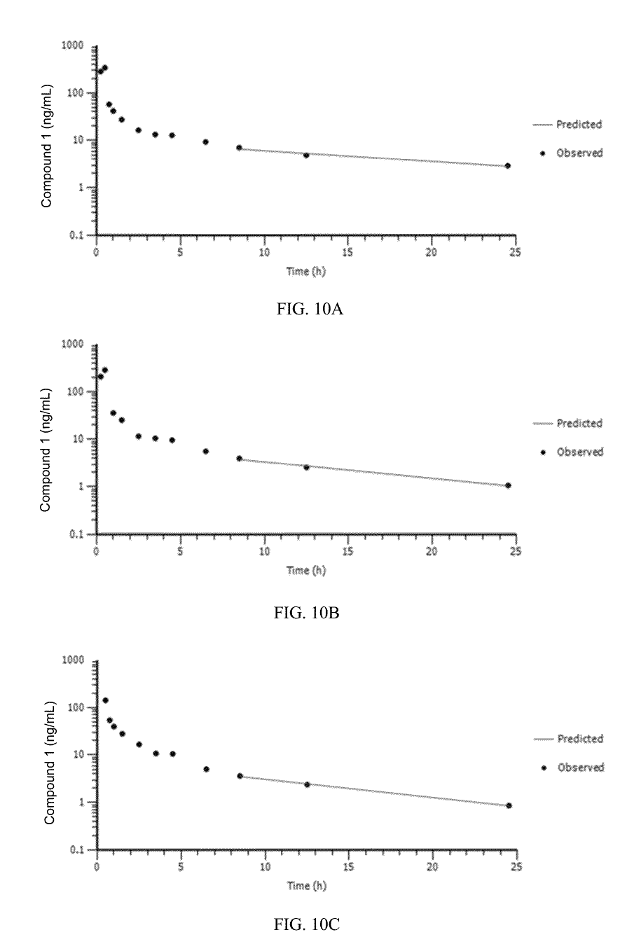

Noncompartmental PK parameters for Compound 1 for Cohort 3 are summarized in Table 7 and previously derived PK parameters for Cohorts 1 and 2 are above for comparative purposes. Plots of the linear regression analysis for determination of the terminal phase rate constant (λz) and half-life for Cohort 3 subjects are displayed in FIG. 10. The Cmax averaged 252 ng/mL and the median Tmax was 0.5 hour. Half-lives averaged 9.88 hours with a range of 7.85 to 13.1 hours. AUCinf values averaged 287 h*ng/mL with relatively low variability, as reflected by a CV % of 34.6%. The calculated volume of distribution at steady state (Vss) was large, averaging 562 L/m². Clearance (CL) was high at 90.7 L/h/m², ranging from 61.1 to 123 L/h/m². The variability in the CL values was relatively low, with CV % of 34.0%.

TABLE 7
Preliminary Pharmacokinetic Parameters for Compound 1 - Cohort 3 (24 mg/m²)

| | | Cmax | Tmax | λz | t½ | AUClast | AUCinf | CL | Vss | MRTinf |
|---|---|---|---|---|---|---|---|---|---|---|
| Cohort | Subject | (ng/mL) | (h) | (1/h) | (h) | (h * ng/mL) | (h * ng/mL) | (L/h/m²) | (L/m²) | (h) |
| Cohort 3 | 1 | 334 | 0.50 | 0.0527 | 13.1 | 337 | 393 | 61.1 | 599 | 9.79 |
| | 2 | 282 | 0.50 | 0.0803 | 8.63 | 259 | 272 | 88.3 | 412 | 4.67 |
| | 3 | 141 | 0.50 | 0.0883 | 7.85 | 186 | 196 | 123 | 674 | 5.50 |
| | N | 3 | 3 | 3 | 3 | 3 | 3 | 3 | 3 | 3 |
| | Mean | 252 | 0.50 | 0.0738 | 9.88 | 261 | 287 | 90.7 | 562 | 6.65 |
| | SD | 99.9 | 0.00 | 0.0187 | 2.86 | 75.7 | 99.2 | 30.8 | 135 | 2.75 |
| | Min | 141 | 0.50 | 0.0527 | 7.85 | 186 | 196 | 61.1 | 412 | 4.67 |
| | Median | 282 | 0.50 | 0.0803 | 8.63 | 259 | 272 | 88.3 | 599 | 5.50 |
| | Max | 334 | 0.50 | 0.0883 | 13.1 | 337 | 393 | 123 | 674 | 9.79 |
| | CV % | 39.6 | 0.0 | 25.3 | 29.0 | 29.1 | 34.6 | 34.0 | 24.0 | 41.3 |
| | Geometric | 237 | 0.50 | 0.0720 | 9.62 | 253 | 275 | 87.1 | 550 | 6.31 |
| | Mean | | | | | | | | | |

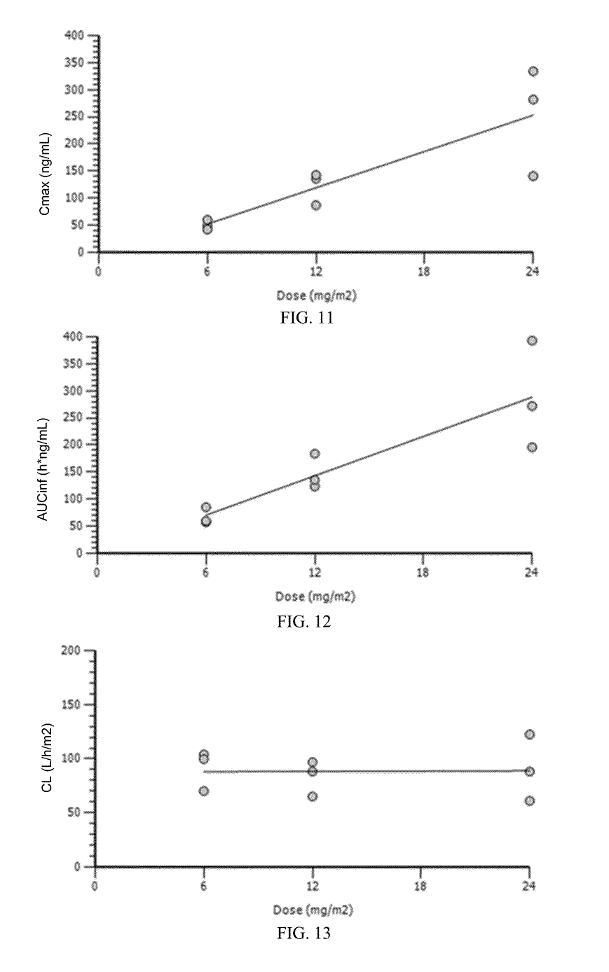

Plots of the relationship between dose of Compound 1 and Cmax, AUC_{inf}, and CL are displayed in FIG. 11 to FIG. 13, respectively. The results indicate that both Cmax and AUC_{inf} are increasing proportionally to the increase in dose and that CL is independent of dose over the dose range of 6 to 24 mg/m².

iv) Cohort 4: 48 mg/m²

Three subjects received 48 mg/m² of Compound 1 administered over a 30-minute duration. Blood samples were obtained at nominal times of 15 minutes into the infusion, at the end of the 30-minute infusion (EOI), and at 0.25, 0.5, 1, 2, 3, 4, 6, 8, 12, 24, 48, and 72 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times.

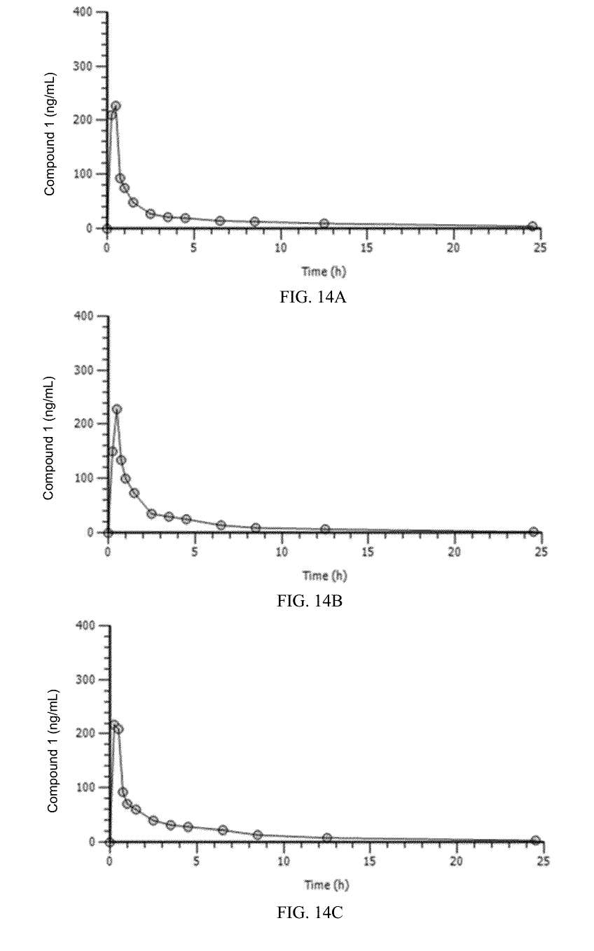

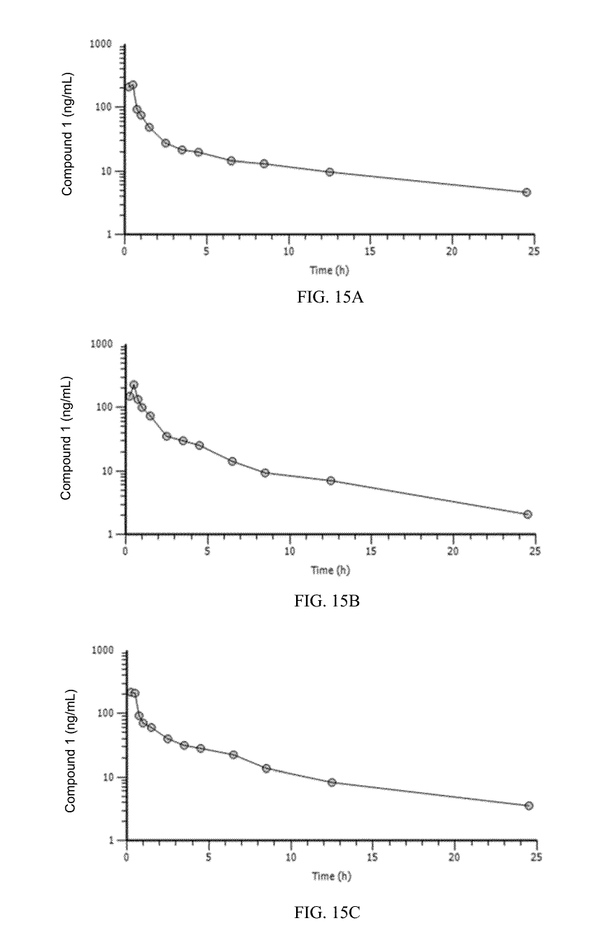

Table 8 contains a summary of the Compound 1 concentration-time data for individual subjects with descriptive statistics for the 3 subjects who received Compound 1 in Cohort 4. FIG. 14 displays individual subject plots of the Compound 1 concentration-time data on a linear scale and FIG. 15 contains plots on log-linear axes. The highest concentrations for 2 subjects occurred at the end of the infusion and for 1 subject at 0.25 hour. Concentrations subsequently decayed with a multi-phase disposition profile as shown on the log-linear plots. At 24.5 hours after the start of infusion, all 3 subjects had low but measurable Compound 1 concentrations ranging from 2.08 to 4.66 ng/mL. Additional concentration-time data were provided for the subjects in Cohort 3. The additional data provide complete profiles for all 3 subjects and a measurable concentration (0.889 ng/mL) for one subject at 48.5 hours.

TABLE 8
Compound 1 Concentration (ng/mL) Summary Table - Cohort 4 (48 mg/m²)

| | | | Time (h) | | | | | | | | | | | | |
|---|---|---|---|---|---|---|---|---|---|---|---|---|---|---|---|
| | | | 0.00 | 0.25 | 0.50 | 0.75 | 1.00 | 1.50 | 2.50 | 3.50 | 4.50 | 6.50 | 8.50 | 12.50 | 24.50 |
| Cohort | Treatment | Subject | Compound (ng/mL) | | | | | | | | | | | | |
| Cohort 4 | 48 mg/m² | 1 | 0.00 | 210 | 227 | 93.1 | 75.2 | 48.5 | 27.4 | 21.4 | 19.7 | 14.5 | 13.1 | 9.72 | 4.66 |
| | | 2 | 0.00 | 150 | 228 | 134 | 100 | 73.4 | 35.2 | 29.9 | 25.3 | 14.2 | 9.35 | 7.06 | 2.08 |
| | | 3 | 0.00 | 217 | 209 | 92.4 | 70.6 | 60.2 | 39.9 | 31.5 | 28.2 | 22.4 | 13.7 | 8.29 | 3.56 |
| | | N | 3 | 3 | 3 | 3 | 3 | 3 | 3 | 3 | 3 | 3 | 3 | 3 | 3 |
| | | Mean | 0.00 | 192 | 221 | 107 | 81.9 | 60.7 | 34.2 | 27.6 | 24.4 | 17.0 | 12.1 | 8.36 | 3.43 |
| | | SD | 0.00 | 36.8 | 10.7 | 23.8 | 15.8 | 12.5 | 6.31 | 5.43 | 4.32 | 4.65 | 2.36 | 1.33 | 1.29 |
| | | Min | 0.00 | 150 | 209 | 92.4 | 70.6 | 48.5 | 27.4 | 21.4 | 19.7 | 14.2 | 9.35 | 7.06 | 2.08 |
| | | Median | 0.00 | 210 | 227 | 93.1 | 75.2 | 60.2 | 35.2 | 29.9 | 25.3 | 14.5 | 13.1 | 8.29 | 3.56 |
| | | Max | 0.00 | 217 | 228 | 134 | 100 | 73.4 | 39.9 | 31.5 | 28.2 | 22.4 | 13.7 | 9.72 | 4.66 |
| | | CV % | NC | 19.1 | 4.8 | 22.4 | 19.3 | 20.5 | 18.5 | 19.7 | 17.7 | 27.3 | 19.6 | 15.9 | 37.7 |
| | | Geometric | NC | 190 | 221 | 105 | 81.0 | 59.8 | 33.8 | 27.2 | 24.1 | 16.6 | 11.9 | 8.29 | 3.26 |
| | | Mean | | | | | | | | | | | | | |

NC = Not calculated

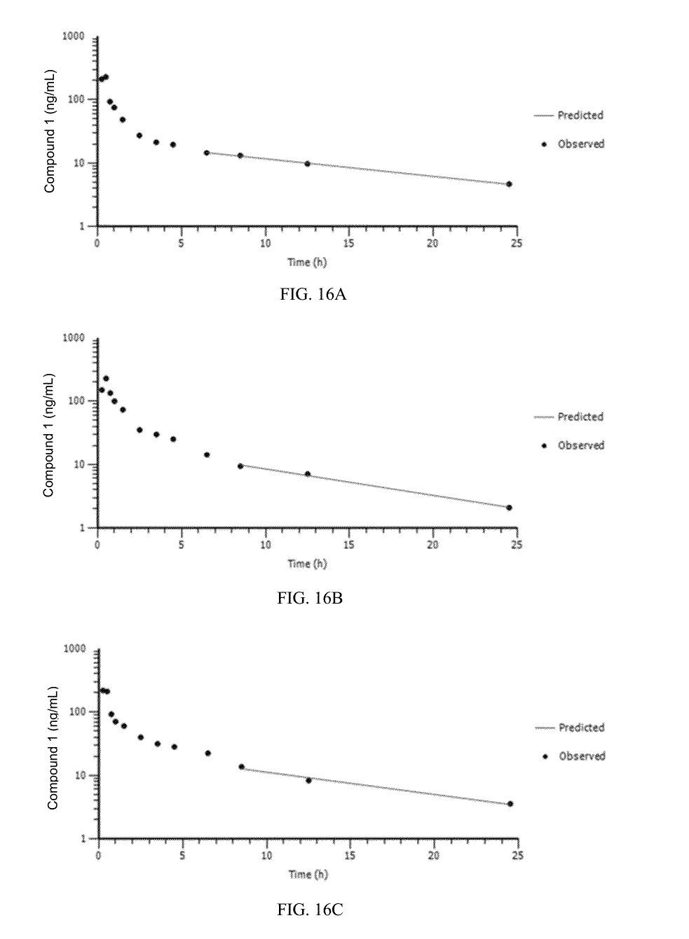

Noncompartmental PK parameters for Compound 1 for Cohort 4 are summarized in Table 9 and previously derived PK parameters for Cohorts 1 to 3 are described above for comparative purposes. Plots of the linear regression analysis for determination of the terminal phase rate constant (λz) and half-life for Cohort 3 subjects are displayed in FIG. 16. For Cohort 4, the Cmax averaged 224 ng/mL and the median Tmax was 0.5 hour. Half-lives averaged 8.90 hours with a range of 7.24 to 10.9 hours. AUCinf values averaged 499 h*ng/mL with very low variability, as reflected by a CV % of 7.0%. The calculated volume of distribution at steady state (Vss) was large, averaging 771 L/m². Clearance (CL) was high at 96.5 L/h/m², ranging from 91.7 to 105 L/h/m². The variability in the CL values was low, with CV % of 7.3%.

TABLE 9
Preliminary Pharmacokinetic Parameters for Compound 1 - Cohort 4 (48 mg/m²)

| | | Cmax | Tmax | λz | t½ | AUClast | AUCinf | CL | Vss | MRTinf |
|---|---|---|---|---|---|---|---|---|---|---|
| Cohort | Subject | (ng/mL) | (h) | (1/h) | (h) | (h * ng/mL) | (h * ng/mL) | (L/h/m²) | (L/m²) | (h) |
| Cohort 4 | 1 | 227 | 0.50 | 0.0636 | 10.9 | 441 | 514 | 93.3 | 995 | 10.7 |
| | 2 | 228 | 0.50 | 0.0958 | 7.24 | 437 | 459 | 105 | 603 | 5.76 |
| | 3 | 217 | 0.25 | 0.0810 | 8.55 | 480 | 524 | 91.7 | 715 | 7.80 |
| | N | 3 | 3 | 3 | 3 | 3 | 3 | 3 | 3 | 3 |
| | Mean | 224 | 0.417 | 0.0801 | 8.90 | 453 | 499 | 96.5 | 771 | 8.08 |
| | SD | 6.08 | 0.144 | 0.0161 | 1.86 | 23.4 | 34.8 | 7.00 | 202 | 2.46 |
| | Min | 217 | 0.250 | 0.0636 | 7.24 | 437 | 459 | 91.7 | 603 | 5.76 |
| | Median | 227 | 0.500 | 0.0810 | 8.55 | 441 | 514 | 93.3 | 715 | 7.80 |
| | Max | 228 | 0.500 | 0.0958 | 10.9 | 480 | 524 | 105 | 995 | 10.7 |
| | CV % | 2.7 | 34.6 | 20.1 | 20.9 | 5.2 | 7.0 | 7.3 | 26.2 | 30.5 |
| | Geometric | 224 | 0.397 | 0.0790 | 8.77 | 452 | 498 | 96.4 | 754 | 7.83 |
| | Mean | | | | | | | | | |

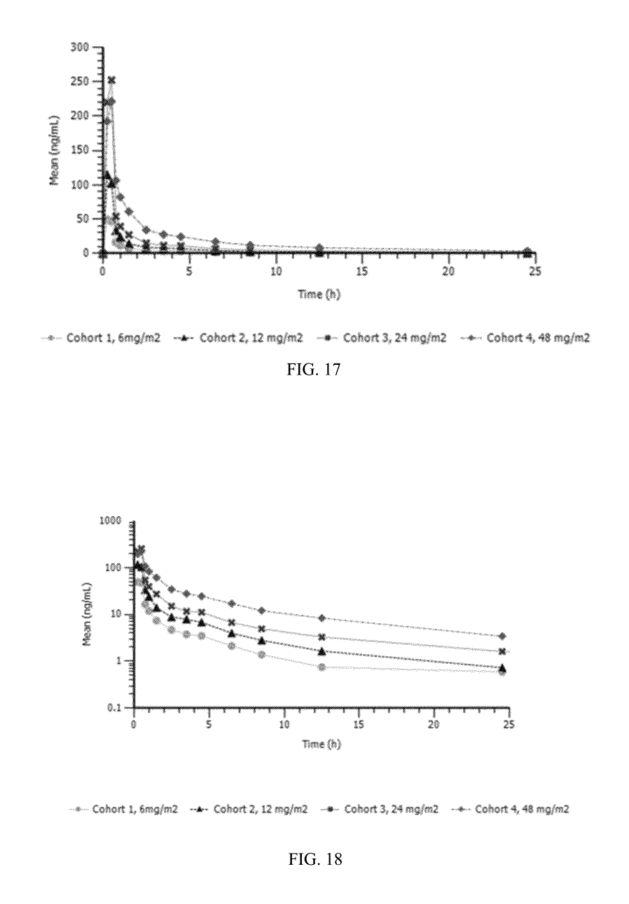

Mean concentration-time plots by cohort are displayed in FIG. 17 and FIG. 18, respectively. As shown in the figures, Compound 1 concentrations decay rapidly following the end of infusion, but as shown in FIG. 18, tend to decay in parallel during the terminal phase.

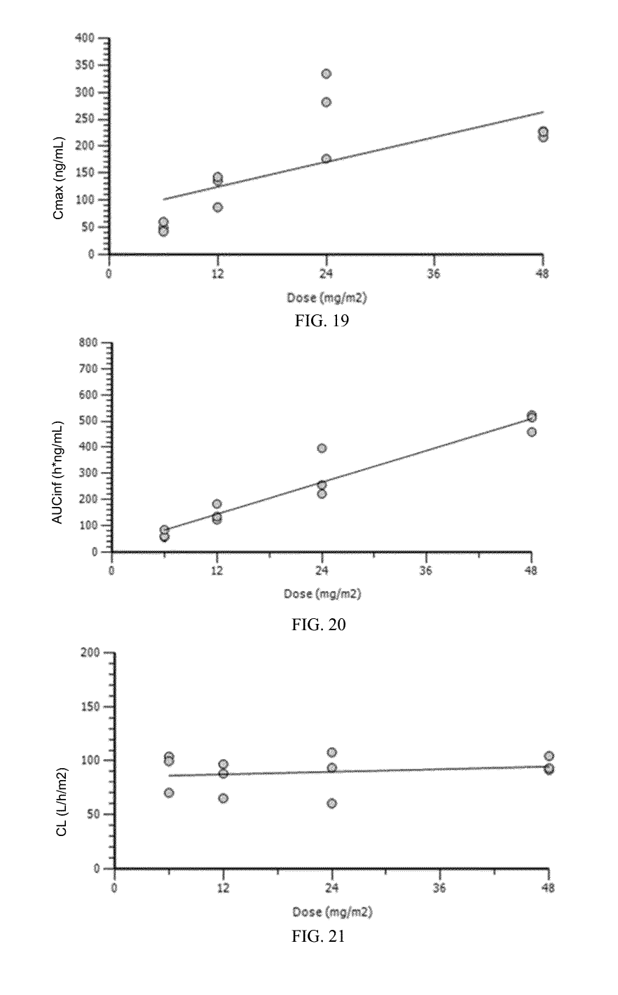

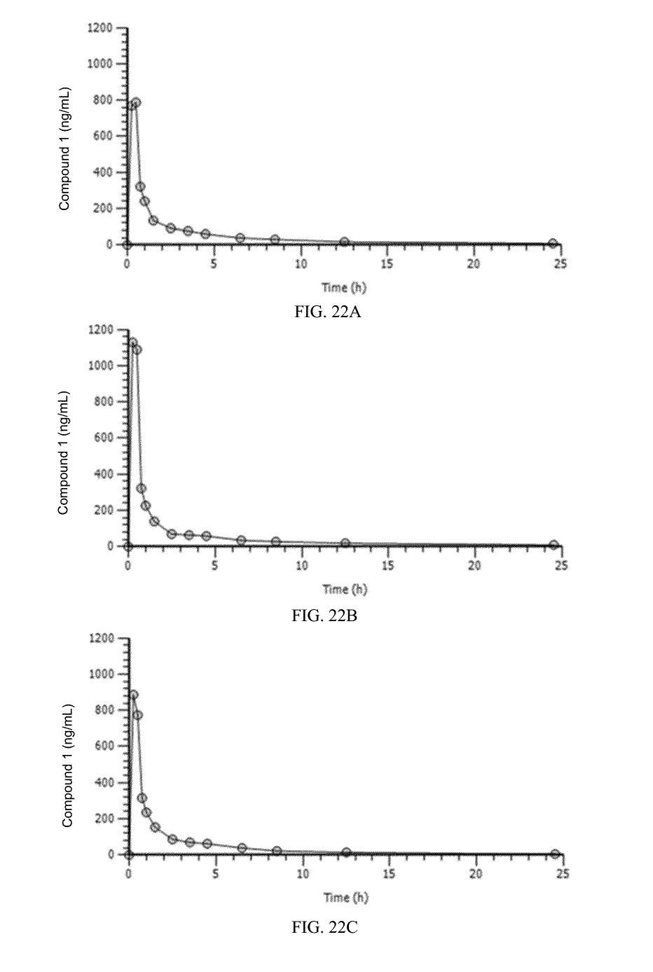

Plots of the relationship between dose of Compound 1 and Cmax, AUC_{inf}, and CL are displayed in FIG. 19 to FIG. 21. The results indicate that AUC_{inf} is increasing proportionally to the increase in dose and that CL is independent of dose over the dose range of 6 to 48 mg/m². However, there was virtually no increase in Cmax values between the 24 and 48 mg/m² dose levels.

v) Cohort 5: 96 mg/m²

In an expanded cohort, 6 subjects received 96 mg/m² of Compound 1 administered over a 30-minute infusion duration. Blood samples were obtained at nominal times of 15 minutes into the infusion, at the end of the 30-minute infusion (EOI), and at 0.25, 0.5, 1, 2, 3, 4, 6, 8, 12, 24, 48, 36, and 72 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times.

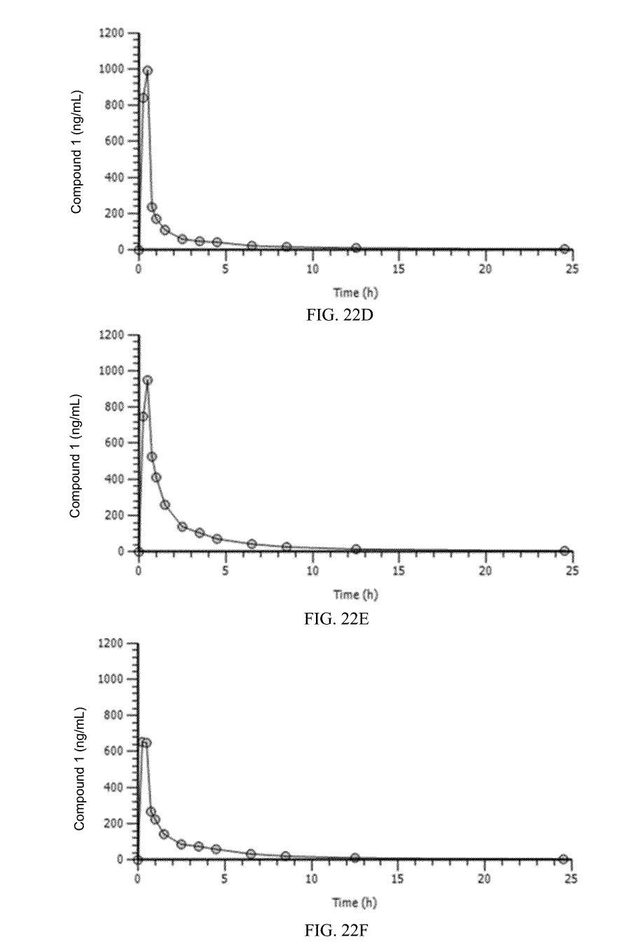

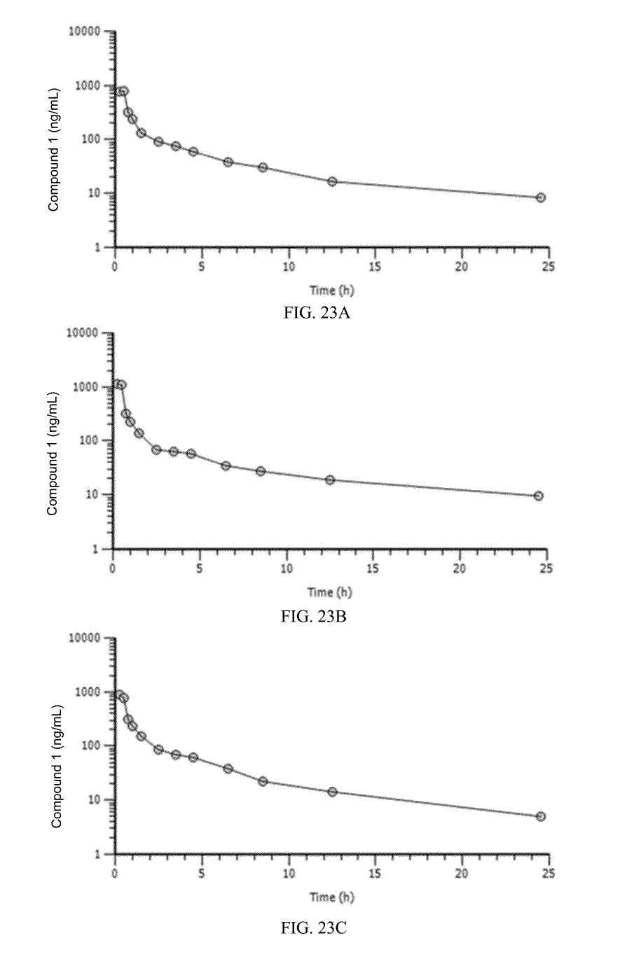

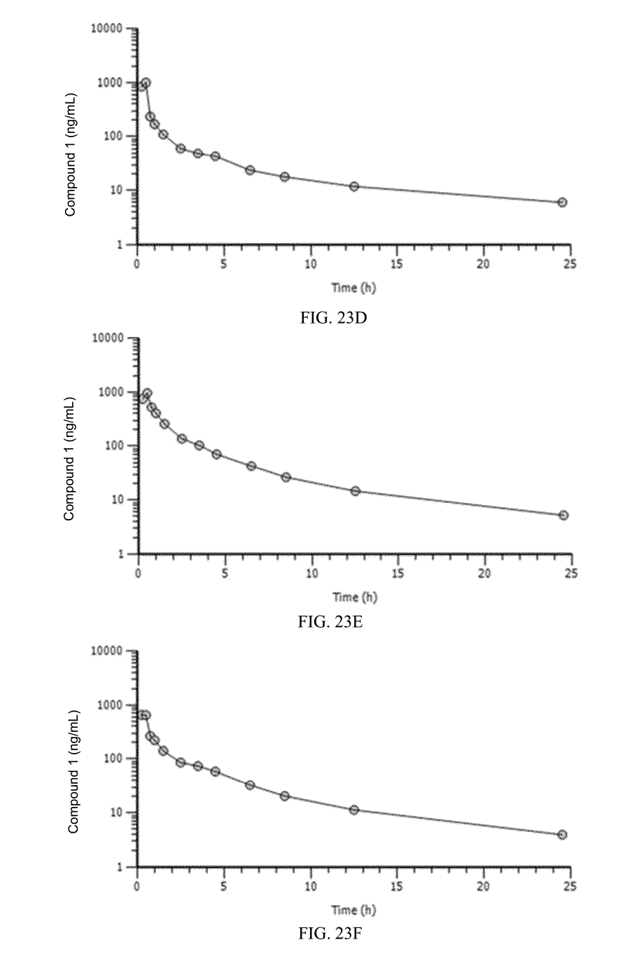

Table 10 contains a summary of the Compound 1 concentration-time data for individual subjects with descriptive statistics for the 6 subjects who received Compound 1 in Cohort 5. FIG. 22 displays individual subject plots of the Compound 1 concentration-time data on a linear scale and FIG. 23 contains plots on log-linear axes. The highest concentrations for 3 subjects occurred at the end of the infusion and for 3 subjects at 0.25 hour. The maximum mean concentration of 874 ng/mL occurred at the end of infusion (EOI). Concentrations subsequently decayed with a multi-phase disposition profile as shown on the log-linear plots. At 24.5 hours after the start of infusion, all 3 subjects had measurable Compound 1 concentrations ranging from 3.96 to 9.59 ng/mL. Additional concentration-time data were provided for the subjects in Cohort 4 (described above). The additional data provided measurable concentration-time values for all 3 subjects at 36 and 48 hours after the EOI and one subject had a measurable concentration (0.760 ng/mL) at 72 hours after the EOI.

TABLE 10
Compound 1 Concentration (ng/mL) Summary Table - Cohort 5 (96 mg/m²)

| | | | Time (h) | | | | | | | | | | | | |
|---|---|---|---|---|---|---|---|---|---|---|---|---|---|---|---|
| | | | 0.00 | 0.25 | 0.50 | 0.75 | 1.00 | 1.50 | 2.50 | 3.50 | 4.50 | 6.50 | 8.50 | 12.50 | 24.50 |
| Cohort | Treatment | Subject | Compound (ng/mL) | | | | | | | | | | | | |
| Cohort 5 | 96 mg/m² | 1 | 0.00 | 770 | 789 | 321 | 240 | 133 | 91.8 | 76.2 | 60.0 | 38.2 | 30.3 | 16.7 | 8.44 |
| | | 2 | 0.00 | 1130 | 1090 | 322 | 226 | 139 | 69.1 | 63.8 | 58.4 | 34.6 | 27.3 | 18.9 | 9.59 |
| | | 3 | 0.00 | 887 | 774 | 315 | 235 | 153 | 86.5 | 69.7 | 62.0 | 38.4 | 22.3 | 14.3 | 5.01 |
| | | 4 | 0.00 | 843 | 992 | 236 | 171 | 110 | 60.5 | 48.8 | 43.4 | 23.7 | 17.9 | 11.9 | 6.06 |
| | | 5 | 0.00 | 749 | 949 | 526 | 410 | 259 | 138 | 104 | 71.2 | 42.8 | 26.4 | 14.7 | 5.25 |
| | | 6 | 0.00 | 654 | 649 | 267 | 223 | 142 | 86.2 | 74.3 | 59.0 | 32.9 | 20.5 | 11.4 | 3.96 |
| | | N | 6 | 6 | 6 | 6 | 6 | 6 | 6 | 6 | 6 | 6 | 6 | 6 | 6 |
| | | Mean | 0.00 | 839 | 874 | 331 | 251 | 156 | 88.7 | 72.8 | 59.0 | 35.1 | 24.1 | 14.7 | 6.39 |
| | | SD | 0.00 | 164 | 164 | 102 | 81.8 | 52.4 | 26.9 | 18.2 | 8.97 | 6.55 | 4.66 | 2.85 | 2.18 |
| | | Min | 0.00 | 654 | 649 | 236 | 171 | 110 | 60.5 | 48.8 | 43.4 | 23.7 | 17.9 | 11.4 | 3.96 |
| | | Median | 0.00 | 807 | 869 | 318 | 231 | 141 | 86.4 | 72.0 | 59.5 | 36.4 | 24.4 | 14.5 | 5.66 |
| | | Max | 0.00 | 1130 | 1090 | 526 | 410 | 259 | 138 | 104 | 71.2 | 42.8 | 30.3 | 18.9 | 9.59 |
| | | CV % | NC | 19.5 | 18.7 | 30.7 | 32.6 | 33.6 | 30.4 | 25.0 | 15.2 | 18.7 | 19.3 | 19.4 | 34.1 |
| | | Geometric | NC | 826 | 861 | 320 | 242 | 150 | 85.7 | 71.0 | 58.4 | 34.5 | 23.7 | 14.4 | 6.09 |
| | | Mean | | | | | | | | | | | | | |

NC = Not calculated

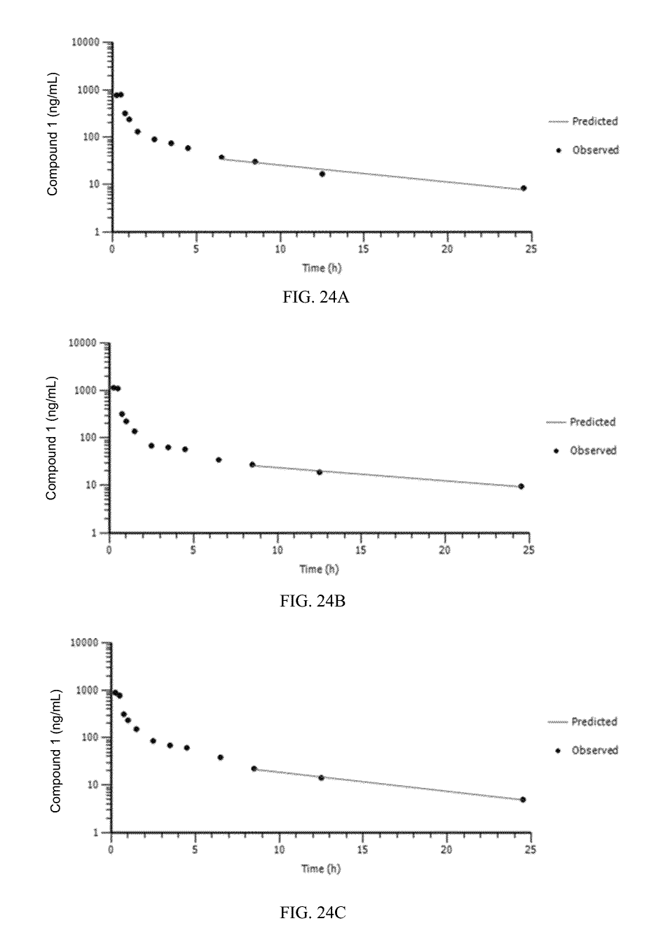

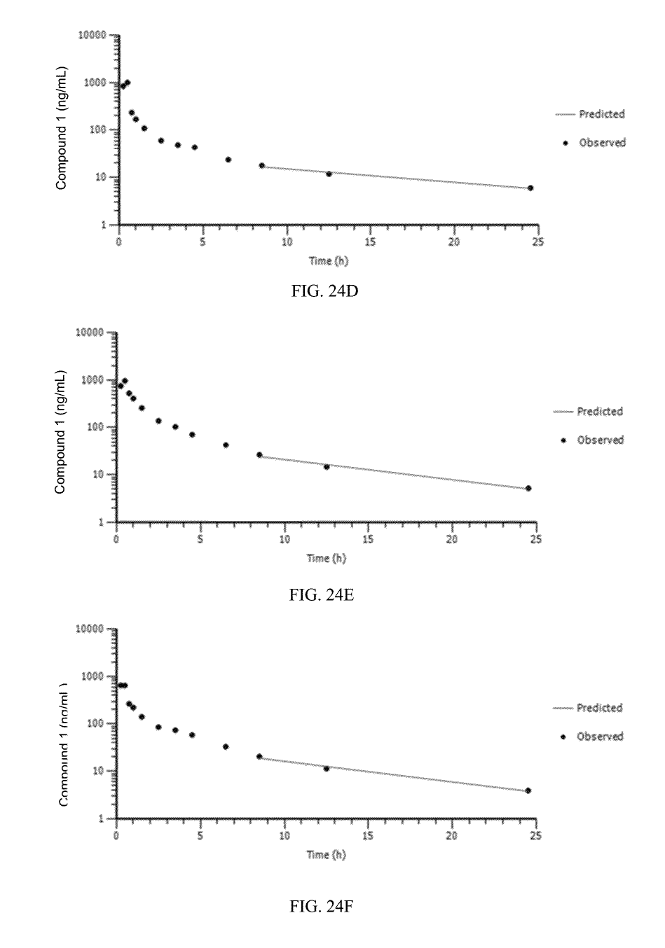

Noncompartmental PK parameters for Compound 1 for Cohort 5 are summarized in Table 11 and previously derived PK parameters for Cohorts 1 to 4 are provided above for comparative purposes. Plots of the linear regression analysis for determination of the terminal phase rate constant (λz) and half-life for Cohort 5 subjects are displayed in FIG. 24. For Cohort 5, the Cmax averaged 900 ng/mL and the median Tmax was 0.375 hour. Half-lives averaged 8.62 hours with a range of 6.97 to 10.9 hours. AUCinf values averaged 1320 h*ng/mL with low variability, as reflected by a CV % of 15.3%. The calculated volume of distribution at steady state (Vss) was large, averaging 429 L/m². Clearance (CL) was high at 74 L/h/m², ranging from 60.4 to 88.1 L/h/m². The variability in the CL values was low, with CV % of 15.2%.

TABLE 11
Preliminary Pharmacokinetic Parameters for Compound 1 - Cohort 5 (96 mg/m²)

| | | Cmax | Tmax | λz | t½ | AUClast | AUCinf | CL | Vss | MRTinf |
|---|---|---|---|---|---|---|---|---|---|---|
| Cohort | Subject | (ng/mL) | (h) | (1/h) | (h) | (h * ng/mL) | (h * ng/mL) | (L/h/m²) | (L/m²) | (h) |
| Cohort 5 | 1 | 789 | 0.50 | 0.0816 | 8.50 | 1250 | 1350 | 71.2 | 481 | 6.76 |
| | 2 | 1130 | 0.25 | 0.0633 | 10.9 | 1370 | 1520 | 63.1 | 481 | 7.62 |
| | 3 | 887 | 0.25 | 0.0920 | 7.54 | 1210 | 1260 | 76.2 | 379 | 4.97 |
| | 4 | 992 | 0.50 | 0.0650 | 10.7 | 1040 | 1130 | 84.8 | 554 | 6.52 |
| | 5 | 949 | 0.50 | 0.0975 | 7.11 | 1530 | 1590 | 60.4 | 264 | 4.36 |
| | 6 | 654 | 0.25 | 0.0994 | 6.97 | 1050 | 1090 | 88.1 | 414 | 4.70 |
| | N | 6 | 6 | 6 | 6 | 6 | 6 | 6 | 6 | 6 |
| | Mean | 900 | 0.375 | 0.0831 | 8.62 | 1240 | 1320 | 74.0 | 429 | 5.82 |
| | SD | 165 | 0.137 | 0.0159 | 1.77 | 191 | 203 | 11.2 | 101 | 1.32 |
| | Min | 654 | 0.250 | 0.0633 | 6.97 | 1040 | 1090 | 60.4 | 264 | 4.36 |
| | Median | 918 | 0.375 | 0.0868 | 8.02 | 1230 | 1300 | 73.7 | 448 | 5.75 |
| | Max | 1130 | 0.500 | 0.0994 | 10.9 | 1530 | 1590 | 88.1 | 554 | 7.62 |
| | CV % | 18.4 | 36.5 | 19.2 | 20.6 | 15.4 | 15.3 | 15.2 | 23.5 | 22.7 |
| | Geometric | 887 | 0.354 | 0.0818 | 8.47 | 1230 | 1310 | 73.3 | 418 | 5.70 |
| | Mean | | | | | | | | | |

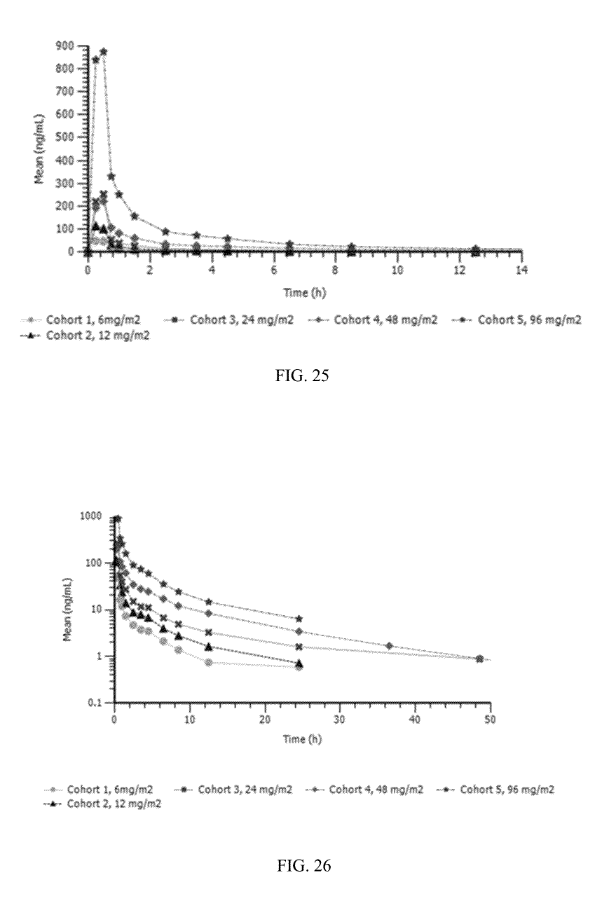

Mean concentration-time plots by cohort are displayed in FIGS. 25 and 26 respectively. As shown in the figures, Compound 1 concentrations decay rapidly following the end of infusion, but as shown in FIG. 26, tend to decay in parallel during the terminal phase.

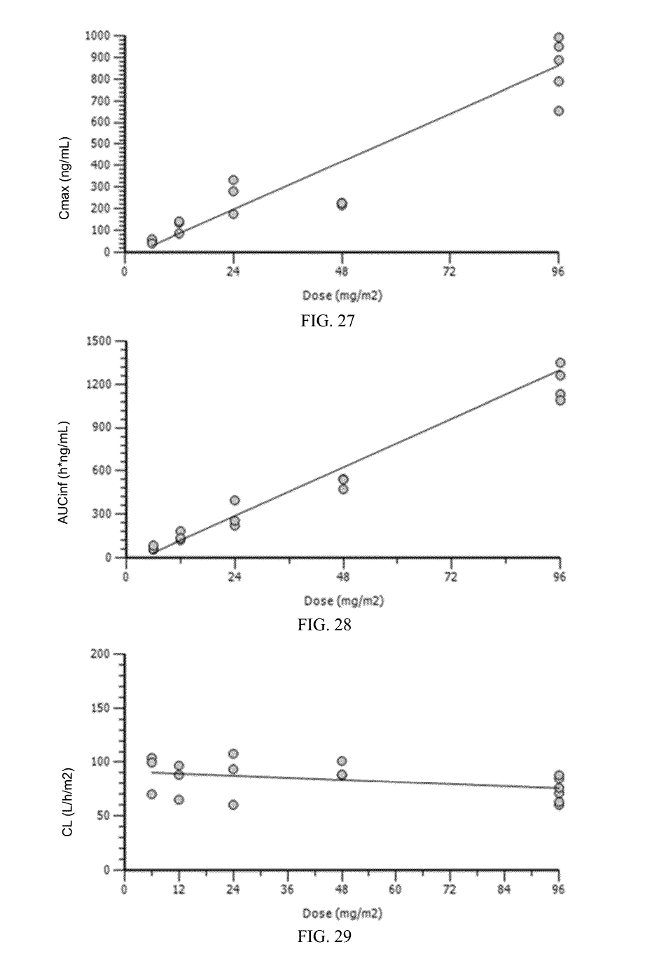

Plots of the relationship between dose of Compound 1 and Cmax, AUC_{inf}, and CL are displayed in FIG. 27 to FIG. 29. The results indicate that both Cmax and AUC_{inf} are increasing proportionally to the increase in dose (with the exception of the 48 mg/m² cohort) and that CL is independent of dose over the dose range of 6 to 96 mg/m².

vi) Cohort 6: 192 mg/m²

In an expanded cohort, 6 subjects received 192 mg/m² of Compound 1 administered over a 30-minute infusion duration. Blood samples were obtained at nominal times of 15 minutes into the infusion, at the end of the 30-minute infusion (EOI), and at 0.25, 0.5, 1, 2, 3, 4, 6, 8, 12, 24, 48, 36, and 72 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times.

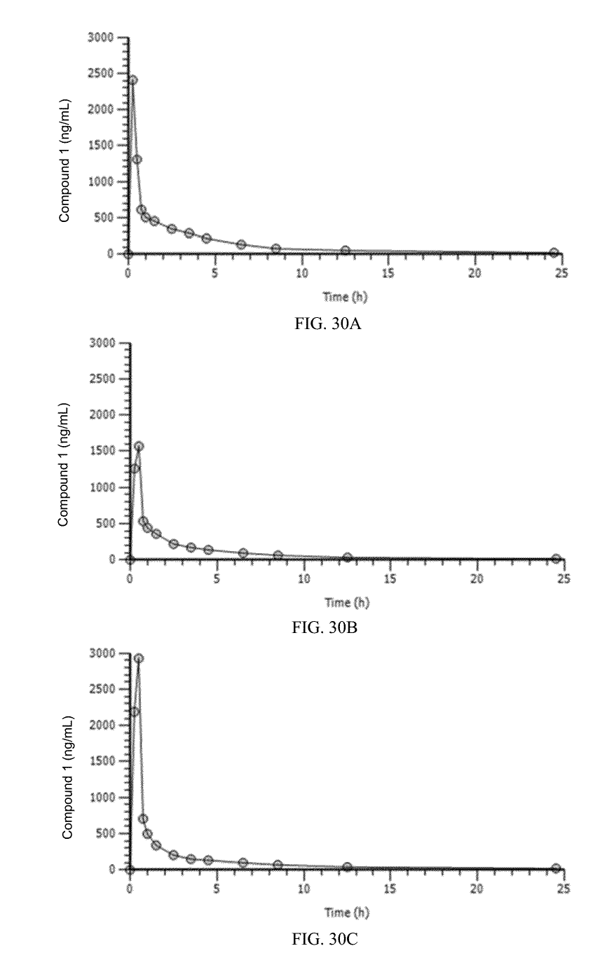

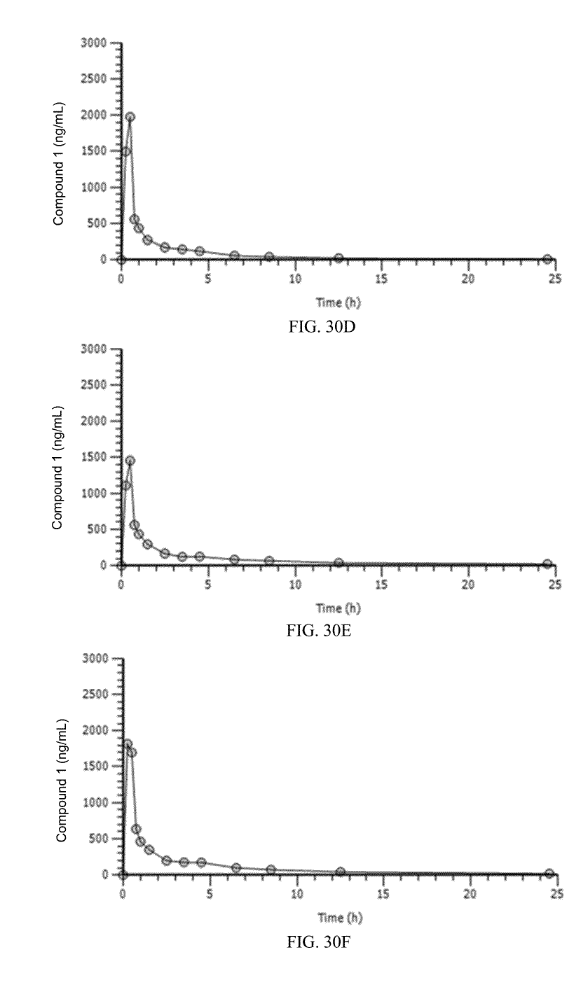

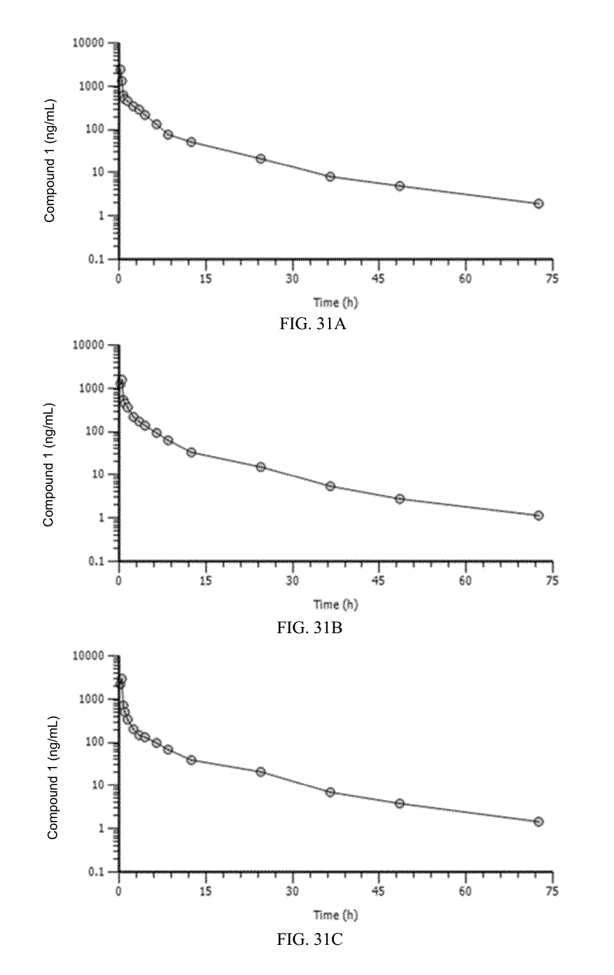

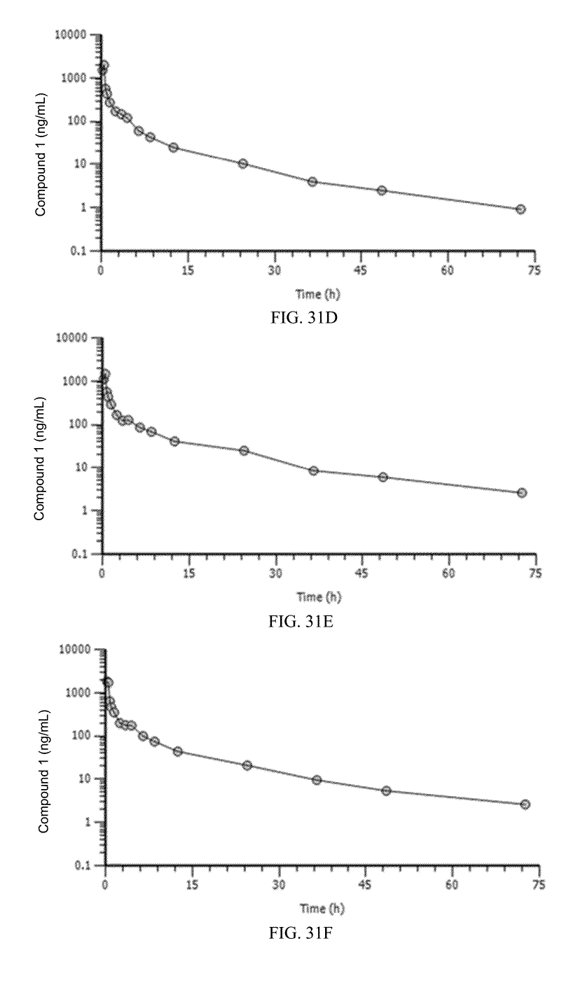

Table 12 contains a summary of the Compound 1 concentration-time data for individual subjects with descriptive statistics for the 6 subjects who received Compound 1 in Cohort 6. FIG. 30 displays individual subject plots of the Compound 1 concentration-time data on a linear scale and FIG. 31 contains plots on log-linear axes. The highest concentrations for 4 subjects occurred at the end of the infusion and for 2 subjects at 0.25 hour. The maximum mean concentration of 1830 ng/mL occurred at the end of infusion (EOI). Concentrations subsequently decayed with a multi-phase disposition profile as shown on the log-linear plots. At 72.5 hours after the start of infusion, all 6 subjects had measurable Compound 1 concentrations ranging from 0.93 to 2.63 ng/mL.

TABLE 12
Compound 1 Concentration (ng/mL) Summary Table - Cohort 6 (192 mg/m²)

| | | Time (h) | | | | | | | | | | | | | | | |
|---|---|---|---|---|---|---|---|---|---|---|---|---|---|---|---|---|---|
| | | 0.00 | 0.25 | 0.50 | 0.75 | 1.00 | 1.50 | 2.50 | 3.50 | 4.50 | 6.50 | 8.50 | 12.50 | 24.50 | 36.50 | 48.50 | 72.50 |
| Treatment | Subject | Compound (ng/mL) | | | | | | | | | | | | | | | |
| 192 mg/m² | 1 | 0.00 | 2410 | 1310 | 612 | 506 | 454 | 346 | 289 | 217 | 133 | 76.6 | 51.8 | 21.2 | 8.07 | 4.92 | 1.93 |
| | 2 | 0.00 | 1260 | 1570 | 534 | 442 | 359 | 219 | 171 | 138 | 93.8 | 63.2 | 33.3 | 15.3 | 5.42 | 2.77 | 1.15 |
| | 3 | 0.00 | 2190 | 2930 | 704 | 497 | 338 | 204 | 148 | 132 | 97.7 | 68.6 | 39.4 | 21.0 | 6.99 | 3.84 | 1.46 |
| | 4 | 0.00 | 1500 | 1980 | 557 | 434 | 272 | 171 | 146 | 120 | 60.0 | 43.1 | 24.9 | 10.4 | 3.98 | 2.51 | 0.930 |
| | 5 | 0.00 | 1110 | 1460 | 561 | 433 | 293 | 167 | 124 | 128 | 86.2 | 68.6 | 41.2 | 25.1 | 8.56 | 6.05 | 2.62 |
| | 6 | 0.00 | 1820 | 1700 | 635 | 461 | 349 | 198 | 177 | 174 | 99.5 | 74.1 | 43.8 | 20.9 | 9.60 | 5.43 | 2.63 |
| | N | 6 | 6 | 6 | 6 | 6 | 6 | 6 | 6 | 6 | 6 | 6 | 6 | 6 | 6 | 6 | 6 |
| | Mean | 0.00 | 1720 | 1830 | 601 | 462 | 344 | 218 | 176 | 152 | 95.0 | 65.7 | 39.1 | 19.0 | 7.10 | 4.25 | 1.79 |
| | SD | 0.00 | 517 | 587 | 63.1 | 32.2 | 63.5 | 66.0 | 58.6 | 37.2 | 23.6 | 12.0 | 9.20 | 5.24 | 2.09 | 1.45 | 0.731 |
| | Min | 0.00 | 1110 | 1310 | 534 | 433 | 272 | 167 | 124 | 120 | 60.0 | 43.1 | 24.9 | 10.4 | 3.98 | 2.51 | 0.930 |
| | Median | 0.00 | 1660 | 1640 | 587 | 452 | 344 | 201 | 160 | 135 | 95.8 | 68.6 | 40.3 | 21.0 | 7.53 | 4.38 | 1.70 |
| | Max | 0.00 | 2410 | 2930 | 704 | 506 | 454 | 346 | 289 | 217 | 133 | 76.6 | 51.8 | 25.1 | 9.60 | 6.05 | 2.63 |
| | CV % | NC | 30.2 | 32.2 | 10.5 | 7.0 | 18.5 | 30.4 | 33.3 | 24.5 | 24.8 | 18.3 | 23.5 | 27.6 | 29.5 | 34.0 | 40.9 |
| | Geometric | NC | 1650 | 1760 | 598 | 461 | 340 | 211 | 169 | 148 | 92.5 | 64.6 | 38.1 | 18.3 | 6.81 | 4.03 | 1.66 |
| | Mean | | | | | | | | | | | | | | | | |

NC = Not calculated

Noncompartmental PK parameters for Compound 1 for Cohort 6 are summarized in Table 13 (0-24.5 hr data) and in Table 14 (0-72.5 hr data) and previously derived PK parameters for Cohorts 1 to 5 are provided above for comparative purposes. Plots of the linear regression analysis for determination of the terminal phase rate constant (λz) and half-life through the 72.5 hour sample for Cohort 6 subjects are displayed in FIG. 31. For Cohort 6, the Cmax averaged 2030 ng/mL and the median Tmax was 0.50 hour. Half-lives determined from the 0-24.5 hr data averaged 7.87 hours with a range of 5.78 to 8.79 hours and half-lives for the 0-72.5 hr data were considerably longer, averaging 18.0 hours and ranging from 16.9 to 20.1 hours. AUC_{inf} values averaged 3160 h*ng/mL with low variability, as reflected by a CV % of 16.5%. The calculated volume of distribution at steady state (Vss) was large, averaging 547 L/m². Clearance (CL) was high at 62.1 L/h/m², ranging from 49.2 to 77.0 L/h/m². The variability in the CL values was low, with CV % of 16.6%.

TABLE 13
Preliminary Pharmacokinetic Parameters for Compound 1 - Cohort 6 (192 mg/m²) (0-24.5 hr data)

| | | Cmax | Tmax | λz | t½ | AUClast | AUCinf | CL | Vss | MRTinf |
|---|---|---|---|---|---|---|---|---|---|---|
| Cohort | Subject | (ng/mL) | (h) | (1/h) | (h) | (h * ng/mL) | (h * ng/mL) | (L/h/m²) | (L/m²) | (h) |
| Cohort 6 | 1 | 2410 | 0.25 | 0.0789 | 8.78 | 3540 | 3810 | 50.4 | 342 | 6.78 |
| | 2 | 1570 | 0.50 | 0.120 | 5.78 | 2550 | 2680 | 71.7 | 406 | 5.67 |
| | 3 | 2930 | 0.50 | 0.0935 | 7.41 | 3190 | 3420 | 56.2 | 337 | 6.00 |
| | 4 | 1980 | 0.50 | 0.0851 | 8.14 | 2320 | 2440 | 78.8 | 405 | 5.14 |
| | 5 | 1460 | 0.50 | 0.0789 | 8.79 | 2460 | 2780 | 69.0 | 613 | 8.88 |
| | 6 | 1820 | 0.25 | 0.0831 | 8.34 | 2940 | 3200 | 60.1 | 425 | 7.08 |
| | N | 6 | 6 | 6 | 6 | 6 | 6 | 6 | 6 | 6 |
| | Mean | 2030 | 0.417 | 0.0899 | 7.87 | 2830 | 3050 | 64.4 | 421 | 6.59 |
| | SD | 555 | 0.129 | 0.0157 | 1.14 | 474 | 513 | 10.6 | 101 | 1.33 |
| | Min | 1460 | 0.250 | 0.0789 | 5.78 | 2320 | 2440 | 50.4 | 337 | 5.14 |
| | Median | 1900 | 0.500 | 0.0841 | 8.24 | 2750 | 2990 | 64.6 | 405 | 6.39 |
| | Max | 2930 | 0.500 | 0.120 | 8.79 | 3540 | 3810 | 78.8 | 613 | 8.88 |
| | CV % | 27.3 | 31.0 | 17.4 | 14.5 | 16.7 | 16.8 | 16.5 | 23.9 | 20.1 |
| | Geometric | 1970 | 0.397 | 0.0889 | 7.80 | 2800 | 3020 | 63.6 | 413 | 6.48 |
| | Mean | | | | | | | | | |

TABLE 14
Preliminary Pharmacokinetic Parameters for Compound 1 - Cohort 6 (192 mg/m²) (0-72.5 hr data)

| | | Cmax | Tmax | t½β | t½γ | AUClast | AUCinf | CL | Vss | MRTinf |
|---|---|---|---|---|---|---|---|---|---|---|
| Cohort | Subject | (ng/mL) | (h) | (h) | (h) | (h * ng/mL) | (h * ng/mL) | (L/h/m2) | (L/m2) | (h) |
| Cohort 6 | 1 | 2410 | 0.25 | 8.78 | 17.5 | 3850 | 3900 | 49.2 | 417 | 8.48 |
| | 2 | 1570 | 0.50 | 5.78 | 16.4 | 2760 | 2790 | 68.9 | 533 | 7.73 |
| | 3 | 2930 | 0.50 | 7.41 | 16.1 | 3470 | 3500 | 54.8 | 414 | 7.54 |
| | 4 | 1980 | 0.50 | 8.14 | 17.1 | 2470 | 2490 | 77.0 | 510 | 6.63 |
| | 5 | 1460 | 0.50 | 8.79 | 20.9 | 2830 | 2910 | 65.9 | 811 | 12.3 |
| | 6 | 1820 | 0.25 | 8.34 | 19.7 | 3300 | 3370 | 56.9 | 595 | 10.5 |
| | N | 6 | 6 | 6 | 6 | 6 | 6 | 6 | 6 | 6 |
| | Mean | 2030 | 0.417 | 7.87 | 18.0 | 3110 | 3160 | 62.1 | 547 | 8.86 |
| | SD | 555 | 0.129 | 1.14 | 1.92 | 515 | 522 | 10.3 | 147 | 2.12 |
| | Min | 1460 | 0.250 | 5.78 | 16.1 | 2470 | 2490 | 49.2 | 414 | 6.63 |
| | Median | 1900 | 0.500 | 8.24 | 17.3 | 3070 | 3140 | 61.4 | 522 | 8.10 |
| | Max | 2930 | 0.500 | 8.79 | 20.9 | 3850 | 3900 | 77.0 | 811 | 12.3 |
| | CV % | 27.3 | 31.0 | 14.5 | 10.7 | 16.5 | 16.5 | 16.6 | 26.9 | 24.0 |
| | Geometric | 1970 | 0.397 | 7.80 | 17.9 | 3080 | 3130 | 61.4 | 532 | 8.66 |
| | Mean | | | | | | | | | |

The study was further expanded to a seventh cohort maintaining the dosage of 192 mg/m² found to be safe in cohort 6. The summary statistics for cohort 7 (N=12) and cohort 6+7 (N=18) are provided below in Table 15.

TABLE 15
Summary Statistics for Additional Cohort 7
and Combined Statistics for Cohort 6 and 7

| | | Cₘₐₓ | tₘₐₓ | AUCₗₐₛₜ | AUC_{inf} | |
|---|---|---|---|---|---|---|
| | Statistic | (ng/mL) | (h) | (ng*h/mL) | (ng*h/mL) | t₁/₂ |
| Cohort 7 | Geo. Mean | 1705 | 0.36 | 2964 | 2991 | 14.5 |
| 192 mg/m² | min-max | 885-3280 | 0.25-0.48 | 2360-3750 | 2379-3762 | 11.9-17.3 |
| N = 12 | | | | | | |
| Cohort 6 + 7 | Geo. Mean | 1789 | 0.37 | 2998 | 3029 | 14.5 |
| 192 mg/m² | min-max | 885-3280 | 0.25-0.48 | 2360-3829 | 2379-3869 | 11.9-17.3 |
| N = 18 | | | | | | |

The additional terminal phase data now available through 72 hours after the end of infusion indicates that the true terminal phase half-life determined in Cohorts 5 and 6 is considerably longer than the values determined for Cohorts 1-4 where concentrations were mostly BLQ at that time.

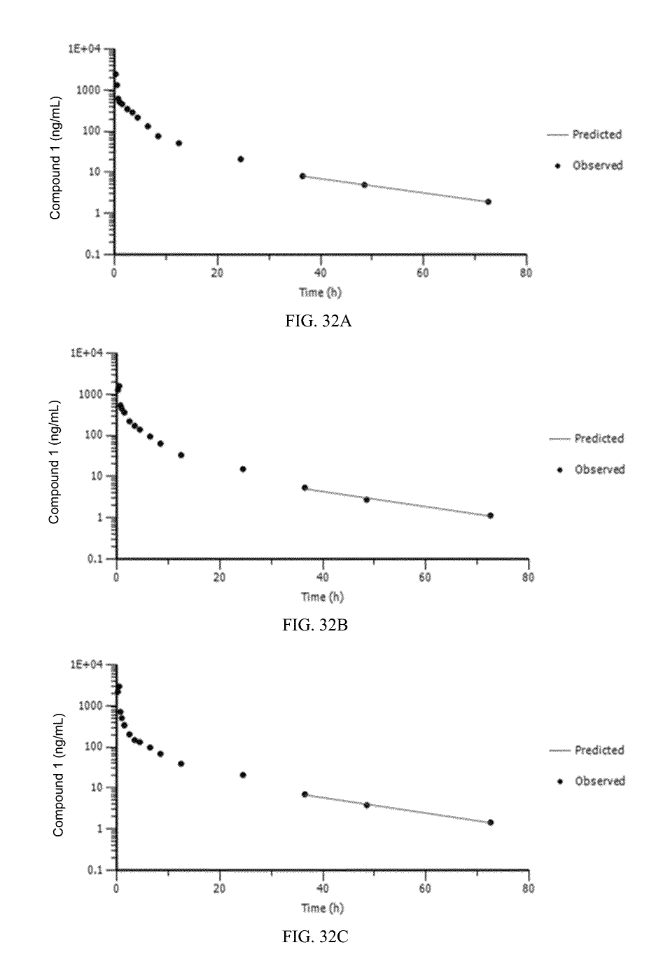

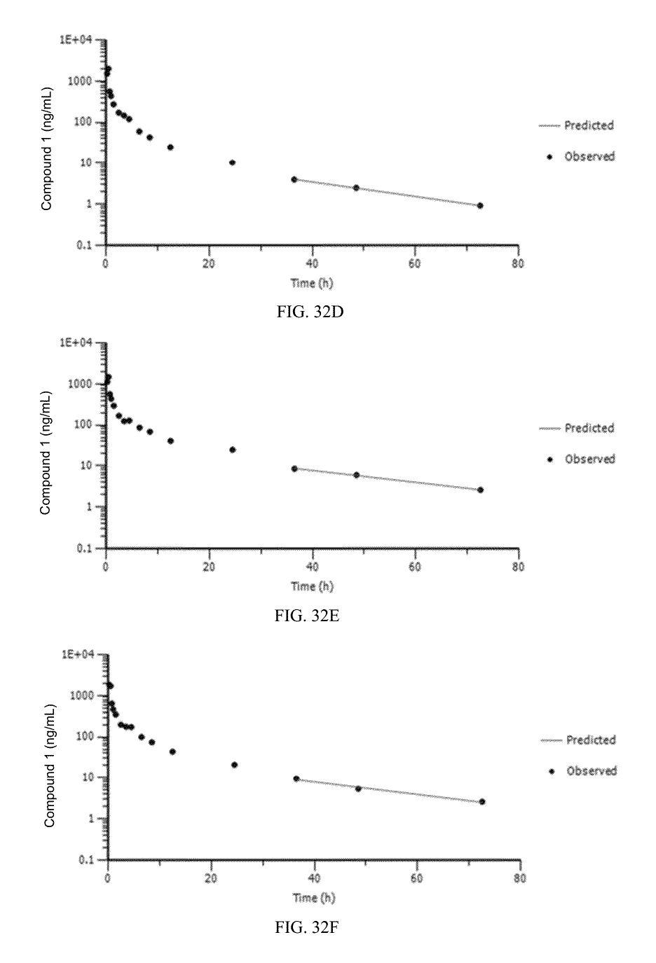

WinNonlin plots in FIG. 32 show evidence of the 3-compartment nature of the disposition of Compound 1 and the results for the intermediate half-life (β-phase) and γ-phase half-life are summarized in Table 16 and Table 17. It is highly likely that the β-phase is more important than the γ-phase since only a small percentage of the total AUC is associated with the 3^{rd} phase.

TABLE 16
3-Compartment Primary Pharmacokinetic Parameters for Compound 1 - Cohort 5

| | | | Alpha | Beta | Gamma | K21 | K31 | V1 |
|---|---|---|---|---|---|---|---|---|
| Cohort | Treatment | Subject | (1/h) | (1/h) | (1/h) | (1/h) | (1/h) | (L/m**2) |
| Cohort 5 | 96 mg/m² | 1 | 5.51 | 0.323 | 0.0521 | 0.660 | 0.0771 | 36.4 |
| | | 2 | 7.15 | 0.389 | 0.0435 | 0.647 | 0.0660 | 20.0 |
| | | 3 | 5.59 | 0.287 | 0.0451 | 0.644 | 0.0560 | 35.8 |
| | | 4 | 7.31 | 0.386 | 0.0400 | 0.700 | 0.0573 | 28.5 |
| | | 5 | 16.7 | 0.500 | 0.0552 | 2.29 | 0.0709 | 19.9 |
| | | 6 | 58.2 | 0.440 | 0.0656 | 1.57 | 0.0881 | 6.80 |
| | | N | 6 | 6 | 6 | 6 | 6 | 6 |
| | | Mean | 16.7 | 0.388 | 0.0502 | 1.09 | 0.0692 | 24.6 |
| | | SD | 20.7 | 0.0769 | 0.00940 | 0.694 | 0.0122 | 11.3 |
| | | Median | 7.23 | 0.387 | 0.0486 | 0.680 | 0.0685 | 24.2 |
| | | CV % | 123.9 | 19.8 | 18.7 | 63.9 | 17.7 | 46.0 |

TABLE 17
3-Compartment Primary Pharmacokinetic Parameters for Compound 1 - Cohort 6

| | | | Alpha | Beta | Gamma | K21 | K31 | V1 |
|---|---|---|---|---|---|---|---|---|
| Cohort | Treatment | Subject | (1/h) | (1/h) | (1/h) | (1/h) | (1/h) | (L/m**2) |
| Cohort 6 | 192 mg/m² | 1 | 15.5 | 0.295 | 0.0487 | 0.954 | 0.0662 | 13.4 |
| | | 2 | 8.11 | 0.322 | 0.0529 | 1.02 | 0.0732 | 37.0 |
| | | 3 | 6.84 | 0.355 | 0.0548 | 0.663 | 0.0809 | 21.8 |
| | | 4 | 6.31 | 0.336 | 0.0498 | 0.745 | 0.0643 | 34.7 |
| | | 5 | 24.0 | 0.580 | 0.0502 | 1.84 | 0.104 | 17.6 |
| | | 6 | 7.29 | 0.284 | 0.0420 | 0.737 | 0.0611 | 29.1 |
| | | N | 6 | 6 | 6 | 6 | 6 | 6 |
| | | Mean | 11.3 | 0.362 | 0.0497 | 0.993 | 0.0750 | 25.6 |
| | | SD | 7.06 | 0.110 | 0.00442 | 0.439 | 0.0160 | 9.51 |
| | | Median | 7.70 | 0.329 | 0.0500 | 0.850 | 0.0697 | 25.4 |
| | | CV % | 62.3 | 30.3 | 8.9 | 44.2 | 21.3 | 37.2 |

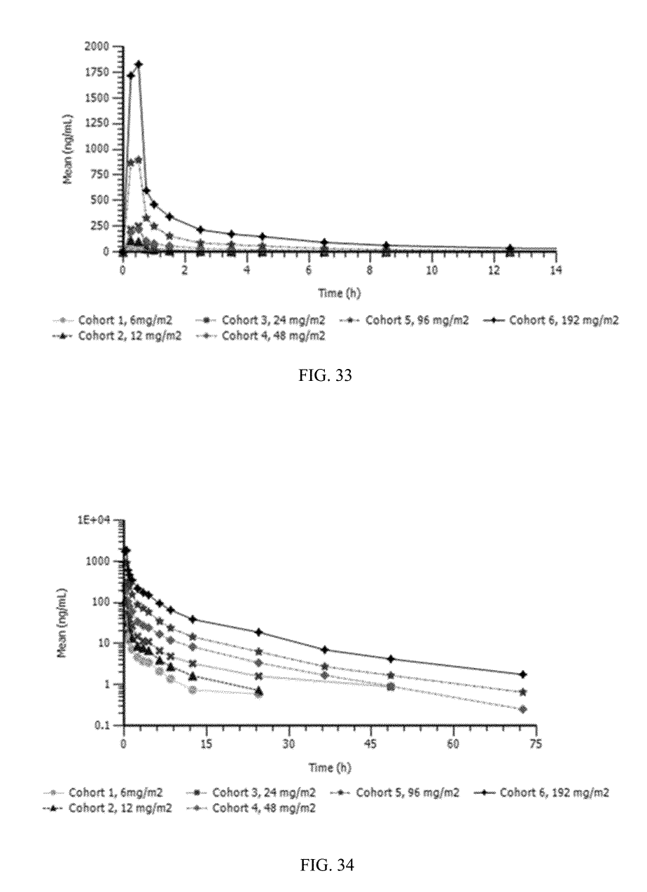

Mean concentration-time plots by cohort are displayed in FIG. 33 and FIG. 34, respectively. As shown in the figures, Compound 1 concentrations decay rapidly following the end of infusion, but as shown in FIG. 34, tend to decay in parallel during the terminal phase.

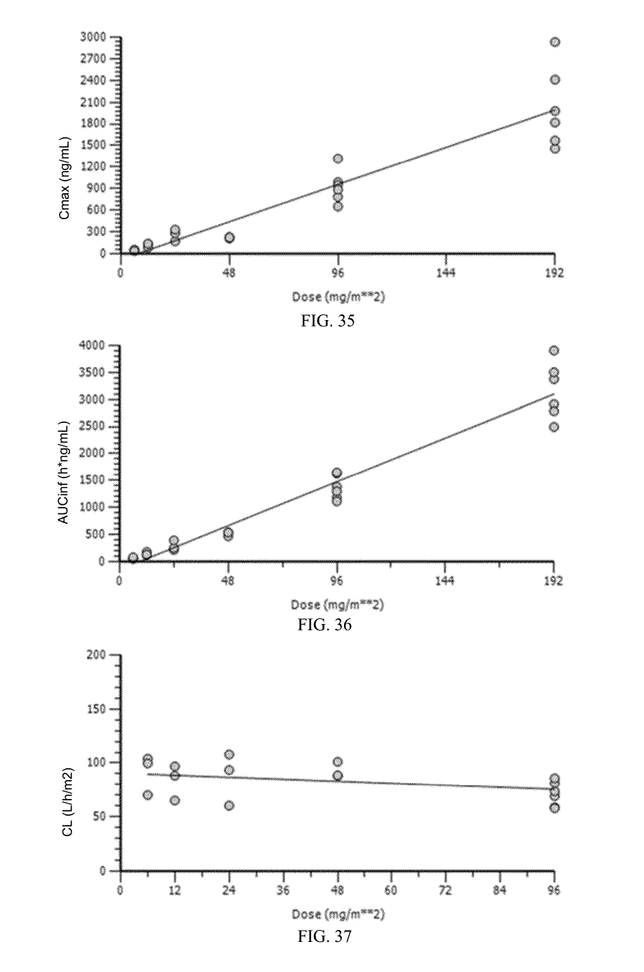

Plots of the relationship between dose of Compound 1 and Cmax, AUC_{inf}, and CL are displayed in FIG. 35 to FIG. 37. The results indicate that both Cmax and AUC_{inf} are increasing proportionally to the increase in dose (with the exception of the 48 mg/m² cohort) and that CL is independent of dose over the dose range of 6 to 192 mg/m².

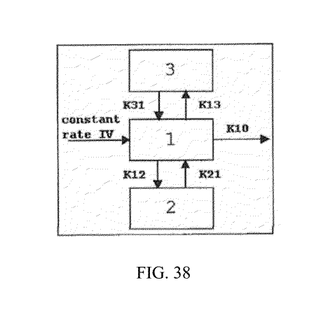

A compartmental analysis was conducted for Cohorts 5 and 6 where the majority of subjects had measurable concentrations through 72.5 hours after the start of infusion. The most appropriate model was a 3-compartment model with IV infusion is shown in FIG. 38.

The concentration-time profile is represented by a tri-exponential equation similar to the following:

C=Ae^{-αt}+Be^{-βt}+C^{-γt}

Where A, B, and C are the ‘macroconstants’ which are a function of the dose, volume of distribution and the ‘microconstants’ (K₁₂, K₂₁, K₁₃, K₃₁, and K₁₀).

The primary (estimated) parameters are summarized in Table 18 and

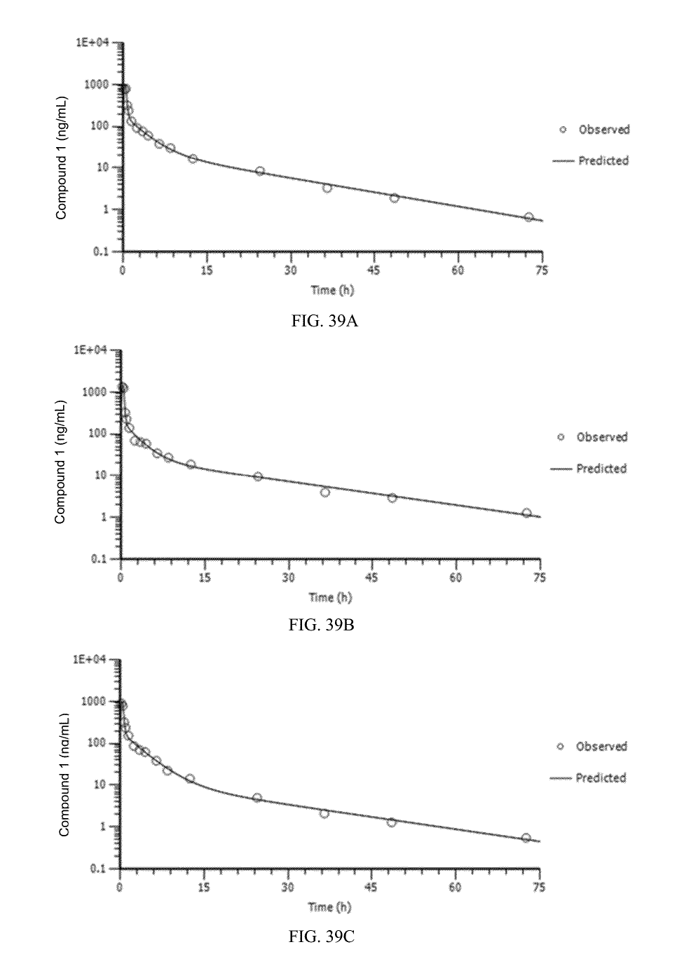

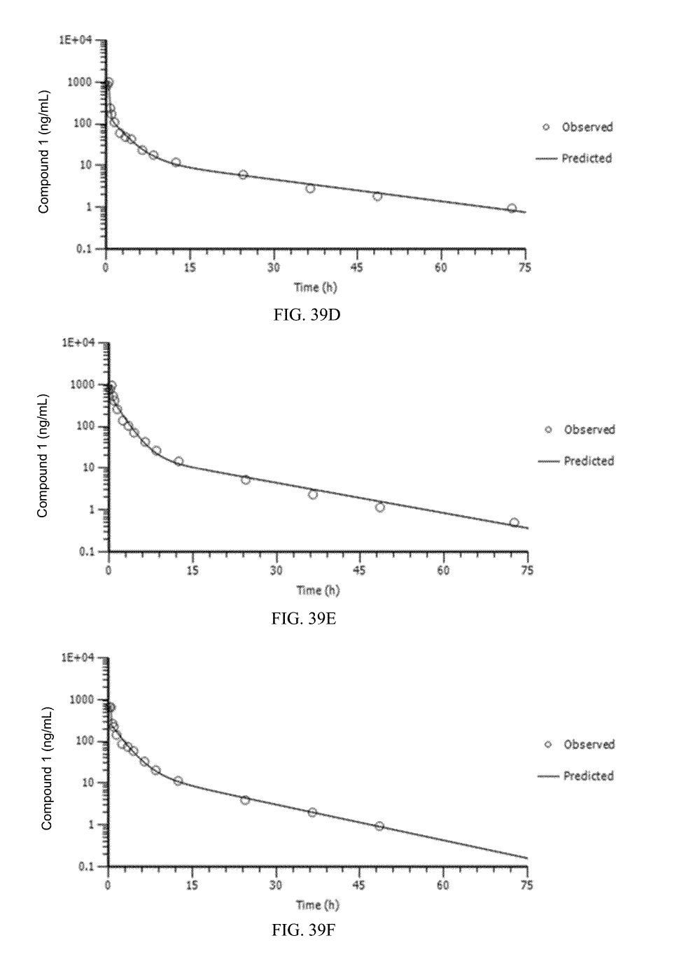

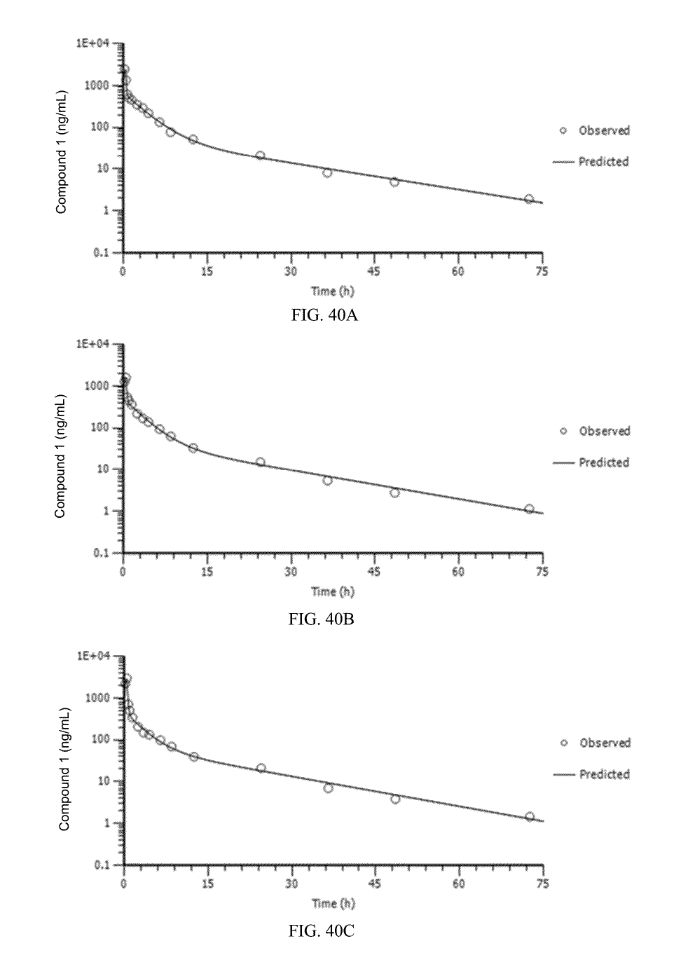

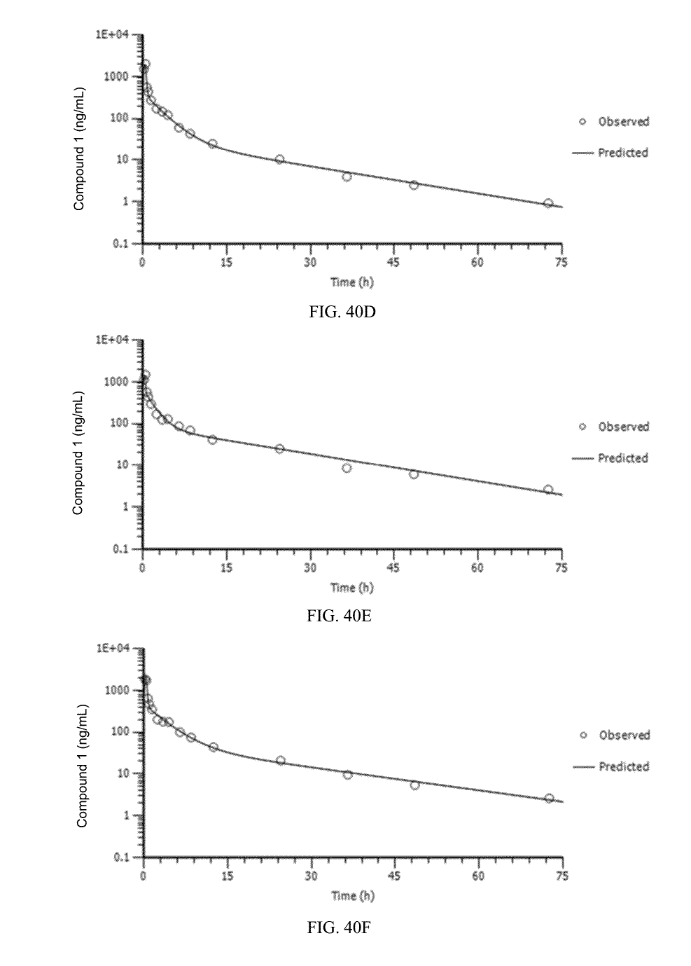

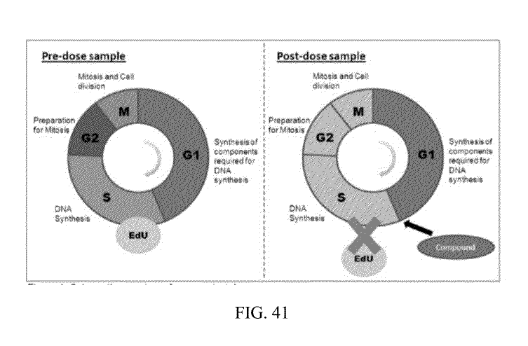

Table 19 for Cohorts 5 and 6, respectively. Five of these parameters are rate constants and the 6^{th} is the volume of distribution of the central compartment (V1). Overall, the analysis provided an excellent fit of the model to the data. The observed and predicted model fits are displayed in FIG. 39 and FIG. 40 for Cohort 5 and Cohort 6, respectively.

TABLE 18
3-Compartment Secondary Pharmacokinetic Parameters for Compound 1 - Cohort 5

| | | | Cmax | t½α | t½β | t½γ | AUC | CL | Vss |
|---|---|---|---|---|---|---|---|---|---|
| Cohort | Treatment | Subject | (ng/mL) | (h) | (h) | (h) | (h*ng/mL) | (L/h/m**2) | (L/m**2) |
| Cohort 5 | 96 mg/m² | ASD | 1000 | 0.126 | 2.15 | 13.3 | 1450 | 66.4 | 530 |
| | | ISS | 1440 | 0.0969 | 1.78 | 15.9 | 1700 | 56.5 | 510 |
| | | MLP | 1010 | 0.124 | 2.41 | 15.4 | 1340 | 71.9 | 463 |
| | | PTT | 1000 | 0.0948 | 1.80 | 17.3 | 1200 | 80.3 | 711 |
| | | QLC | 991 | 0.0416 | 1.39 | 12.6 | 1700 | 56.4 | 318 |
| | | VPQ | 729 | 0.0119 | 1.57 | 10.6 | 1160 | 82.7 | 458 |
| | | N | 6 | 6 | 6 | 6 | 6 | 6 | 6 |
| | | Mean | 1030 | 0.0825 | 1.85 | 14.2 | 1420 | 69.0 | 498 |
| | | SD | 227 | 0.0460 | 0.375 | 2.49 | 238 | 11.4 | 128 |
| | | Median | 1000 | 0.0959 | 1.79 | 14.3 | 1390 | 69.1 | 486 |
| | | CV % | 22.1 | 55.8 | 20.3 | 17.5 | 16.7 | 16.5 | 25.6 |

Secondary (derived) pharmacokinetic parameters are summarized in Table 17 and Table 18 for Cohorts 5 and 6, respectively, and contain half-lives for the 3 phases, calculated Cmax and AUC values, and CL and Vss (volume of distribution at steady state) values.

TABLE 19
3-Compartment Secondary Pharmacokinetic Parameters for Compound 1 - Cohort 6

| | | | Cmax | t½α | t½β | t½γ | AUC | CL | Vss |
|---|---|---|---|---|---|---|---|---|---|
| Cohort | Treatment | Subject | (ng/mL) | (h) | (h) | (h) | (h*ng/mL) | (L/h/m**2) | (L/m**2) |
| Cohort 6 | 192 mg/m² | BBY | 2350 | 0.0447 | 2.35 | 14.2 | 4050 | 47.4 | 371 |
| | | BJQ | 1580 | 0.0855 | 2.15 | 13.1 | 2790 | 68.7 | 515 |
| | | FFP | 2780 | 0.101 | 1.95 | 12.6 | 3540 | 54.2 | 396 |
| | | LOP | 1920 | 0.110 | 2.06 | 13.9 | 2510 | 76.5 | 483 |
| | | MSP | 1400 | 0.0289 | 1.20 | 13.8 | 3020 | 63.7 | 736 |
| | | VCL | 2060 | 0.0951 | 2.44 | 16.5 | 3420 | 56.1 | 547 |
| | | N | 6 | 6 | 6 | 6 | 6 | 6 | 6 |
| | | Mean | 2020 | 0.0776 | 2.03 | 14.0 | 3220 | 61.1 | 508 |
| | | SD | 505 | 0.0329 | 0.444 | 1.35 | 559 | 10.6 | 131 |
| | | Median | 1990 | 0.0903 | 2.11 | 13.9 | 3220 | 59.9 | 499 |
| | | CV % | 25.0 | 42.5 | 21.9 | 9.6 | 17.3 | 17.3 | 25.8 |

As shown in the tables, the γ phase half-life averaged 14.2 and 14.0 hours for the 96 and 192 mg/m² doses, respectively. These values are somewhat shorter than the terminal phase half-life estimates from the noncompartmental analysis since only the last 3 point were used in that analysis and the compartmental analysis generates the best fit of the model to all of the data points.

The other derived parameters such as Cmax, AUC, and CL are similar to the values obtained from the noncompartmental analysis.

### Example 7

### Human Ex-Vivo Stimulation and Proliferation Assays

The magnitude and duration of Compound 1 induced HSPC G0/G1 arrest in humans was assessed in order to determine utilization of the Compound in a chemoprotection strategy. PK/PD data from 3 species (mouse, rat, dog) was used to evaluate dose response relationships for HSPC G0/G1 cell cycle arrest and to construct a cross species allometrically scaled PK/PD model. Simulations from the model, in conjunction with human PK and PD, guided selection of the biologically effective dose (BED) of 192 mg/m² in humans.

Whole blood was collected from each subject in Cohorts 5 and 6 at various time points. The whole blood was stimulated in vitro with PHA (phytohemagglutinin) to initiate cell prolideration. PHA is a mitogen that triggers T-Cell proliferation. At the end of the stimulation period, EdU is added to the whole blood cells to be incorporated into the DNA during active DNA synthesis. After

PHA stimulation, the whole blood cells were stained with CD45 and CD3 to identify the T lymphocytes. To detect if DNA synthesis had taken place the EdU was stained. Proliferating cells stained positive for EdU because it was incorporated into the DNA. Flow cytometry was used to identify proliferating cells. An overview of this cytometry is provided in FIG. 41.

Upon PHA stimulation of a pre-dose sample, DNA synthesis takes place and EdU is incorporated into the DNA during active synthesis. EdU will stain during the assay and the fluorescence intensity will be measured using flow cytometry. In the presence of Compound 1 (post-dose sample), cells are arrested in G1 and active DNA synthesis does not take place in the presence of PHA.

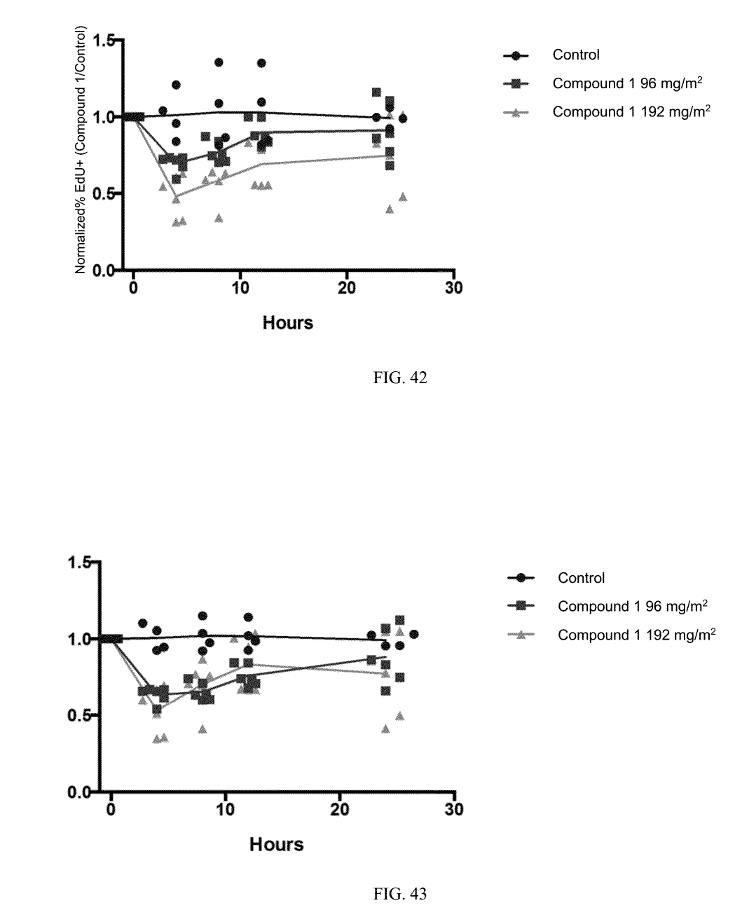

FIG. 42 and FIG. 43 show the results of peripheral blood stimulation pre-dose and post-dose of Compound 1. FIG. 42 was normalized to all placebo cohorts, while FIG. 43 was normalized to separate placebo cohorts.

Single bone marrow aspirates were obtained at the BED at various time points relative to Compound 1 administration in a Phase I trial (NCT02243150). Diluted human bone marrow was filtered by a 40 um filtered and layers on a Ficoll-Paque Premium solution and centrifuged. Differential migration during centrifugation results in the formation of layers containing different cell types. The bottom layer contains erythrocytes, which have been aggregated by the Ficoll and, therefore, sediment completely through the Ficoll-Paque Premium. The layer immediately above the erythrocyte layer contains mostly granulocytes which at the osmotic pressure of the Ficoll-Paque Premium solution attain a density great enough to migrate through the Ficoll-Paque Premium layer. Because of their lower density, the bone marrow mononuclear cells (BM MNCs) are found at the interface between the plasma and the Ficoll-Paque Premium with other slowly sedimenting particles. The BM MNCs are then recovered from the interface and subjected to short washing steps with a balances salt solution to remove any sedimenting particles and Ficoll-Paque Premium.

By flow cytometry staining, the assay determines the cell cycle status of the various cell types in human bone marrow by staining DNA content. Mononuclear cells were isolated by means of Ficoll gradient isolation. Cells were stained with 3 antibody panels:

1) CD45/CD34/CD38/Draq5;

2) CD45/CD14/CD11b/Draq5; and,

3) CD45/CD71/CD61/Draq5.

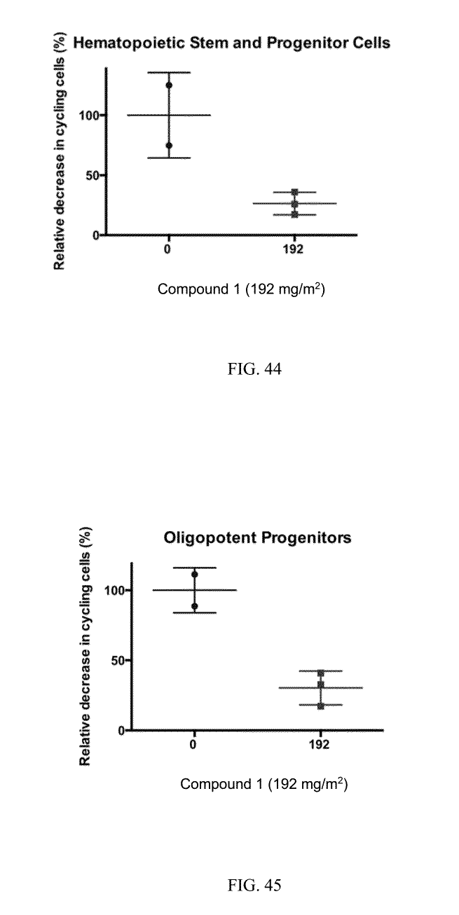

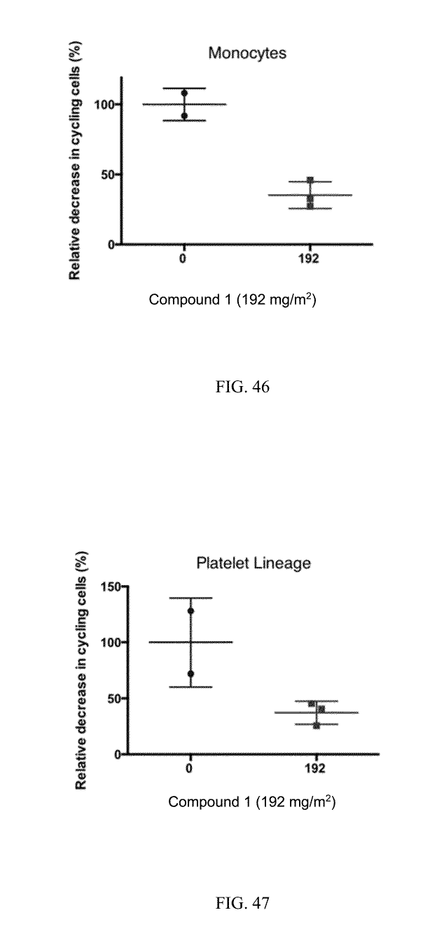

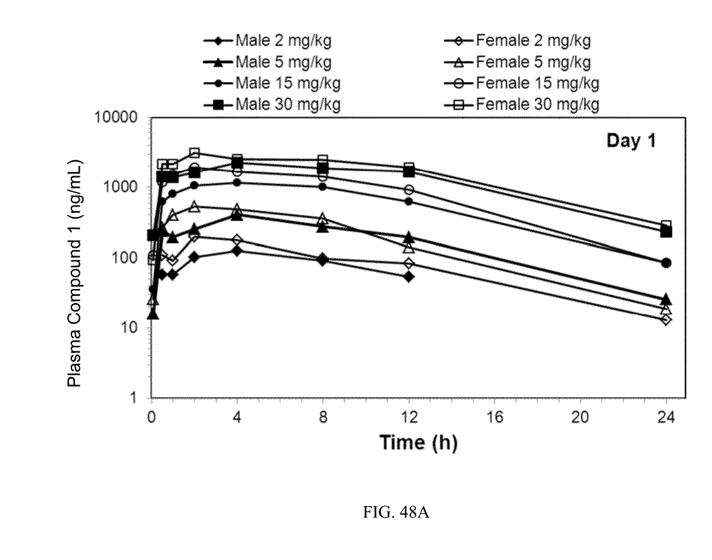

At the predicted BED of 192 mg/m², a single bone marrow aspirate was obtained from 12 subjects (baseline, n=5; 24 h post, n=3, or 32 h post, n=4). Compound 1 administration produced robust and transient inhibition of HSPC and oligopotent progenitor (OPP) proliferation at 24 h, which persisted at 32 hours. FIG. 44 shows decrease in percentage of Hematopoietic Stem and Progenitor Cells (HSPCs) cycling cells at 24 hours post exposure to 192 mg/m² of Compound 1. FIG. 45 shows decrease in percentage of oligopotent progenitor cells cycling cells at 24 hours post exposure to 192 mg/m² of Compound 1. FIG. 46 shows decrease in percentage of monocytes cycling cells at 24 hours post exposure to 192 mg/m² of Compound 1. FIG. 47 shows decrease in percentage of platelet lineage cycling cells at 24 hours post exposure to 192 mg/m² of Compound 1.

A single IV administration of Compound 1 at the BED of 192 mg/m² produced robust and transient inhibition of HSPC and OPP within the bone marrow for greater than about 24 hours.

### Example 8

### Toxicokinetic Studies in Rats

Compound 1's toxicity was evaluated in rats when administered orally via gavage daily for 14 days. Briefly, for the toxicokinetic portion of the study, rats were given daily oral gavage doses of vehicle or Compound 1 at 1, 10, or 25 mg/kg for 14 consecutive days to the study design shown in Table 20.

TABLE 20
Study Design

| | Dose Level | Dose Volume | No. of Animals | |
|---|---|---|---|---|
| Group/Treatment | (mg/kg) | (mL/kg) | Males | Females |
| 6/Vehicle Control | 0 | 5 | 3 | 3 |
| 7/Cmpd 1 | 2 | 5 | 6 | 6 |
| 8/Cmpd 1 | 5 | 5 | 6 | 6 |
| 9/Cmpd 1 | 15 | 5 | 6 | 6 |
| 10/Cmpd 1 | 30 | 5 | 6 | 6 |

*Animal 8M142 replaced Animal 8M136 on Day 1 prior to the first dose.

Plasma was obtained from blood samples collected from rats in a composite sampling scheme, with 3 samples collected per sex, per dose, at each target collection time, as illustrated in Table 21 to Table 25. Samples were collected from the vehicle control group on Day 1 and Day 14 at 3 hour after administration. For all other groups, samples were collected on Day 1 and Day 14 at 5 and 30 minutes and at 1, 2, 4, 8, 12, and 24 hours after administration. In addition, a sample was collected from all groups receiving Compound 1 on Day 14 just prior to dose administration. Blood samples were collected in tubes containing K3EDTA as an anticoagulant and placed on ice until processed to plasma via centrifugation. Samples were stored frozen at −80° C. until shipment to the bioanalytical laboratory for analysis.

TABLE 21
Individual Compound 1 Plasma Concentrations (ng/mL) 3 hours
Following Daily Oral Administration of Vehicle to Rats

| | | | Day 1 Conc | Day 14 Conc |
|---|---|---|---|---|
| Dose Group | Sex | Subject | (ng/mL) | (ng/mL) |
| 6 | Male | 1051 | BLQ | BLQ |
| Vehicle | | 1052 | BLQ | BLQ |
| 0 mg/kg | | 1053 | BLQ | BLQ |
| | Female | 1054 | BLQ | BLQ |
| | | 1055 | BLQ | BLQ |
| | | 1056 | BLQ | BLQ |

BLQ = Below the lower limit of quantitation (<10.0 ng/mL)

TABLE 22
Individual Compound 1 Plasma Concentrations (ng/mL) Following
Daily Oral Administration of 2 mg/kg Compound 1 to Rats

| Dose | | | | Compound Concentration (ng/mL) by Time (h) | | | | | | | | |
|---|---|---|---|---|---|---|---|---|---|---|---|---|
| Group | Day | Sex | Subject | Predose | 0.0833 | 0.5 | 1 | 2 | 4 | 8 | 12 | 24 |
| 7 | 1 | Male | 1057 | — | BLQ | — | 47.1 | — | 107 | — | 61.3 | — |
| 2 mg/kg | | | 1058 | — | — | 35.0 | — | 95.5 | — | 64.1 | — | BLQ |
| | | | 1059 | — | BLQ | — | 65.2 | — | 148 | — | 47.8 | — |
| | | | 1060 | — | — | 64.1 | — | 97.4 | — | 99.0 | — | BLQ |
| | | | 1061 | — | BLQ | — | 59.1 | — | 122 | — | 51.9 | — |
| | | | 1062 | — | — | 74.1 | — | 113 | — | 108 | — | BLQ |
| | | Female | 1063 | — | BLQ | — | 53.3 | — | 90.2 | — | 91.8 | — |
| | | | 1064 | — | — | 139 | — | 216 | — | 70.0 | — | BLQ |
| | | | 1065 | — | BLQ | — | 115 | — | 201 | — | 100 | — |
| | | | 1066 | — | — | 69.7 | — | 185 | — | 69.2 | — | 28.8 |
| | | | 1067 | — | BLQ | — | 106 | — | 234 | — | 54.7 | — |
| | | | 1068 | — | — | 120 | — | 187 | — | 151 | — | 10.2 |
| | 14 | Male | 1057 | — | BLQ | — | 34.9 | — | 117 | — | 65.7 | — |
| | | | 1058 | BLQ | — | 36.3 | — | 84.3 | — | 68.7 | — | BLQ |
| | | | 1059 | — | BLQ | — | 75.2 | — | 119 | — | 50.8 | — |
| | | | 1060 | BLQ | — | 49.0 | — | 109 | — | 112 | — | BLQ |
| | | | 1061 | — | BLQ | — | 61.2 | — | 112 | — | 45.6 | — |
| | | | 1062 | BLQ | — | 55.7 | — | 91.3 | — | 102 | — | BLQ |
| | | Female | 1063 | — | — | — | — | — | — | — | — | — |
| | | | 1064 | BLQ | — | 192 | — | 224 | — | 54.0 | — | 21.6 |
| | | | 1065 | — | 20.0 | — | 108 | — | 129 | — | 65.4 | — |
| | | | 1066 | — | — | — | — | — | — | — | — | — |
| | | | 1067 | — | BLQ | — | 113 | — | 119 | — | 32.0 | — |
| | | | 1068 | BLQ | — | 108 | — | 200 | — | 180 | — | BLQ |

BLQ = Below the lower limit of quantitation (<10.0 ng/mL);
— = No sample per study protocol or amendment

TABLE 23
Individual Compound 1 Plasma Concentrations (ng/mL) Following
Daily Oral Administration of 5 mg/kg Compound 1 to Rats

| Dose | | | | Compound 1 Concentration (ng/mL) by Time (h) | | | | | | | | |
|---|---|---|---|---|---|---|---|---|---|---|---|---|
| Group | Day | Sex | Subject | Predose | 0.0833 | 0.5 | 1 | 2 | 4 | 8 | 12 | 24 |
| 8 | 1 | Male | 1069 | — | 12.4 | — | 145 | — | 295 | — | 182 | — |
| 5 mg/kg | | | 1070 | — | — | 165 | — | 75.3 | — | 323 | — | 31.0 |
| | | | 1071 | — | 25.5 | — | 194 | — | 366 | — | 197 | — |
| | | | 1072 | — | — | 225 | — | 300 | — | 235 | — | 26.5 |
| | | | 1073 | — | 10.0 | — | 244 | — | 562 | — | 206 | — |
| | | | 1074 | — | — | 321 | — | 387 | — | 270 | — | 19.3 |
| | | Female | 1075 | — | 15.6 | — | 195 | — | 410 | — | 133 | — |
| | | | 1076 | — | — | 246 | — | 533 | — | 432 | — | 23.2 |
| | | | 1077 | — | 34.9 | — | 645 | — | 560 | — | 140 | — |
| | | | 1078 | — | — | 233 | — | 544 | — | 294 | — | 12.7 |
| | | | 1079 | — | 26.4 | — | 377 | — | 476 | — | 148 | — |
| | | | 1080 | — | — | 322 | — | 508 | — | 353 | — | 19.9 |
| | 14 | Male | 1069 | — | 26.0 | — | 254 | — | 512 | — | 169 | — |
| | | | 1070 | — | — | — | — | — | — | — | — | — |
| | | | 1071 | — | 25.5 | — | 183 | — | 307 | — | 261 | — |
| | | | 1072 | 16.6 | — | 205 | — | 344 | — | 282 | — | 11.1 |
| | | | 1073 | — | 30.0 | — | 337 | — | 443 | — | 164 | — |
| | | | 1074 | 23.4 | — | 390 | — | 527 | — | 263 | — | 21.2 |
| | | Female | 1075 | — | 34.3 | — | 428 | — | 423 | — | 112 | — |
| | | | 1076 | 15.3 | — | 248 | — | 431 | — | 234 | — | 22.2 |
| | | | 1077 | — | 112 | — | 660 | — | 474 | — | 95.9 | — |
| | | | 1078 | 15.0 | — | 589 | — | 716 | — | 240 | — | 16.7 |
| | | | 1079 | — | 65.9 | — | 469 | — | 421 | — | 107 | — |
| | | | 1080 | 22.7 | — | 308 | — | 332 | — | 292 | — | 18.6 |

BLQ = Below the lower limit of quantitation (<10.0 ng/mL);
— = No sample per study protocol or amendment

TABLE 24
Individual Compound 1 Plasma Concentrations (ng/mL) Following
Daily Oral Administration of 15 mg/kg Compound 1 to Rats

| Dose | | | | Compound 1 Concentration (ng/mL) by Time (h) | | | | | | | | |
|---|---|---|---|---|---|---|---|---|---|---|---|---|
| Group | Day | Sex | Subject | Predose | 0.0833 | 0.5 | 1 | 2 | 4 | 8 | 12 | 24 |
| 9 | 1 | Male | 1081 | — | 26.7 | — | 767 | — | 1010 | — | 683 | — |
| 15 mg/kg | | | 1082 | — | — | 674 | — | 1060 | — | 1170 | — | 46.9 |
| | | | 1083 | — | 30.4 | — | 1050 | — | 1320 | — | 721 | — |
| | | | 1084 | — | — | 377 | — | 759 | — | 696 | — | 134 |
| | | | 1085 | — | 49.7 | — | 614 | — | 1160 | — | 451 | — |
| | | | 1086 | — | — | 810 | — | 1370 | — | 1180 | — | 72.5 |
| | | Female | 1087 | — | 198 | — | 1670 | — | 1750 | — | 1010 | — |
| | | | 1088 | — | — | 1050 | — | 1820 | — | 1250 | — | 63.9 |
| | | | 1089 | — | 107 | — | 2010 | — | 1860 | — | 904 | — |
| | | | 1090 | — | — | 1360 | — | 2100 | — | 1670 | — | 110 |
| | | | 1091 | — | 22.9 | — | 914 | — | 1450 | — | 833 | — |
| | | | 1092 | — | — | 1120 | — | 1820 | — | 1390 | — | 79.7 |
| | 14 | Male | 1081 | — | 89.9 | — | 1020 | — | 1160 | — | 583 | — |
| | | | 1082 | 85.2 | — | 741 | — | 1350 | — | 1190 | — | 61.4 |
| | | | 1083 | — | 80.3 | — | 875 | — | 968 | — | 776 | — |
| | | | 1084 | 67.0 | — | 701 | — | 1390 | — | 1260 | — | 51.5 |
| | | | 1085 | — | 151 | — | 632 | — | 1120 | — | 519 | — |
| | | | 1086 | 81.7 | — | 778 | — | 1230 | — | 1060 | — | 68.9 |
| | | Female | 1087 | — | 117 | — | 1410 | — | 1580 | — | 1060 | — |
| | | | 1088 | 100 | — | 908 | — | 1310 | — | 996 | — | 106 |
| | | | 1089 | — | 167 | — | 1660 | — | 1380 | — | 661 | — |
| | | | 1090 | 121 | — | 1210 | — | 1750 | — | 1050 | — | 98.2 |
| | | | 1091 | — | 108 | — | 1290 | — | 1760 | — | 693 | — |
| | | | 1092 | 140 | — | 1390 | — | 1410 | — | 892 | — | 186 |

BLQ = Below the lower limit of quantitation (<10.0 ng/mL);
— = No sample per study protocol or amendment

TABLE 25
Individual Compound 1 Plasma Concentrations (ng/mL) Following
Daily Oral Administration of 30 mg/kg Compound 1 to Rats

| Dose | | | | Compound 1 Concentration (ng/mL) by Time (h) | | | | | | | | |
|---|---|---|---|---|---|---|---|---|---|---|---|---|
| Group | Day | Sex | Subject | Predose | 0.0833 | 0.5 | 1 | 2 | 4 | 8 | 12 | 24 |
| 10 | 1 | Male | 1093 | — | 122 | — | 1610 | — | 1830 | — | 1560 | — |
| 30 mg/kg | | | 1094 | — | — | 1300 | — | 1560 | — | 1930 | — | 177 |
| | | | 1095 | — | 51.5 | — | 968 | — | 2220 | — | 1500 | — |
| | | | 1096 | — | — | 1450 | — | 1810 | — | 2000 | — | 280 |
| | | | 1097 | — | 450 | — | 1620 | — | 2660 | — | 1930 | — |
| | | | 1098 | — | — | 1510 | — | 1490 | — | 1640 | — | 244 |
| | | Female | 1099 | — | 114 | — | 2500 | — | 2570 | — | 2000 | — |
| | | | 1100 | — | — | 2030 | — | 3440 | — | 2480 | — | 310 |
| | | | 1101 | — | 83.8 | — | 2060 | — | 2660 | — | 1570 | — |
| | | | 1102 | — | — | 2390 | — | 2550 | — | 2600 | — | 273 |
| | | | 1103 | — | 80.8 | — | 1870 | — | 2310 | — | 2250 | — |
| | | | 1104 | — | — | 2050 | — | 3290 | — | 2320 | — | 270 |
| | 14 | Male | 1093 | — | 197 | — | 1920 | — | 2080 | — | 1230 | — |
| | | | 1094 | 151 | — | 977 | — | 1630 | — | 1750 | — | 208 |
| | | | 1095 | — | 219 | — | 853 | — | 1940 | — | 1210 | — |
| | | | 1096 | 216 | — | 1150 | — | 1630 | — | 2240 | — | 214 |
| | | | 1097 | — | 204 | — | 1810 | — | 2070 | — | 1450 | — |
| | | | 1098 | 149 | — | 852 | — | 1520 | — | 1650 | — | 192 |
| | | Female | 1099 | — | 842 | — | 2740 | — | 2700 | — | 2290 | — |
| | | | 1100 | 332 | — | 1450 | — | 2170 | — | 1970 | — | 280 |
| | | | 1101 | — | 293 | — | 1750 | — | 2050 | — | 1860 | — |
| | | | 1102 | 352 | — | 1710 | — | 2040 | — | 2010 | — | 409 |
| | | | 1103 | — | 476 | — | 2210 | — | 2210 | — | 2730 | — |
| | | | 1104 | 323 | — | 1460 | — | 1830 | — | 1900 | — | 276 |

BLQ = Below the lower limit of quantitation (<10.0 ng/mL);
— = No sample per study protocol or amendment

Plasma samples were analyzed to measure Compound 1 concentration using a validated LC-MS/MS method.

i. Calculation of TK Parameters

Mean composite plasma Compound 1 concentration-time data were used to calculate TK parameters using standard non-compartmental pharmacokinetic methods in Watson version 7.3.0.01 (Thermo Fisher Scientific, Inc., Philadelphia, Pa.). The following parameters were determined:

Cmax: Maximum observed plasma concentration (ng/mL).

Tmax: Time to reach maximum observed plasma concentration (h), expressed in terms of time from Compound 1 administration.

AUC(0-t): Area under the plasma concentration vs. time curve from 0 to the time of the last measurable Compound 1 concentration (ng*h/mL), calculated by the linear trapezoidal method. A value of 0 ng/mL was assigned to all values below the lower limit of quantitation (BLQ, <10.0 ng/mL).

AUC(0-inf): Area under the plasma concentration vs. time curve extrapolated from 0 to infinity (ng*h/mL), calculated as AUC(0-t)+AUC(t-inf) where AUC(t-inf) is calculated as last measurable concentration (CLast) divided by ke, where ke is the elimination rate constant determined by linear regression of the last three analytically measured points on the log plasma concentration vs. time curve. The selection criteria of the data points for inclusion in the calculation of ke required that at least three data points representing the terminal phase (after Tmax) were regressed and that R2≧0.850 when rounded. AUC(0-inf) was only reported when R2≧0.850 and AUC(t-inf) was less than 20% of AUC(0-inf).

CL: Clearance (L/h/kg), calculated as the dose divided by AUC(0-t).

Vd: Volume of distribution (L/kg), calculated as dose divided by the product of ke and AUC(0-inf).

Vdss: Steady state volume of distribution (L/kg), calculated as clearance divided by mean residence time, where mean residence time is calculated as the area under the first moment curve divided by AUC(0-t).

t½: Elimination half-life (h), calculated as ln(2)/ke, where ke is the elimination rate constant determined by linear regression. Half-life was defined as not determined if regression criteria (specified in AUC(0-inf) above) were not met.

Cmax/Dose: Normalized Cmax, calculated as Cmax divided by total dose.

AUC/Dose: Normalized AUC, calculated as AUC(0-t) divided by total dose.

ii. Statistical Analysis Mean and standard deviation (SD) were calculated in Watson. Values of BLQ were assigned a value of 0 ng/mL for mean calculations. Concentration values and derived TK parameters were reported to three significant figures.

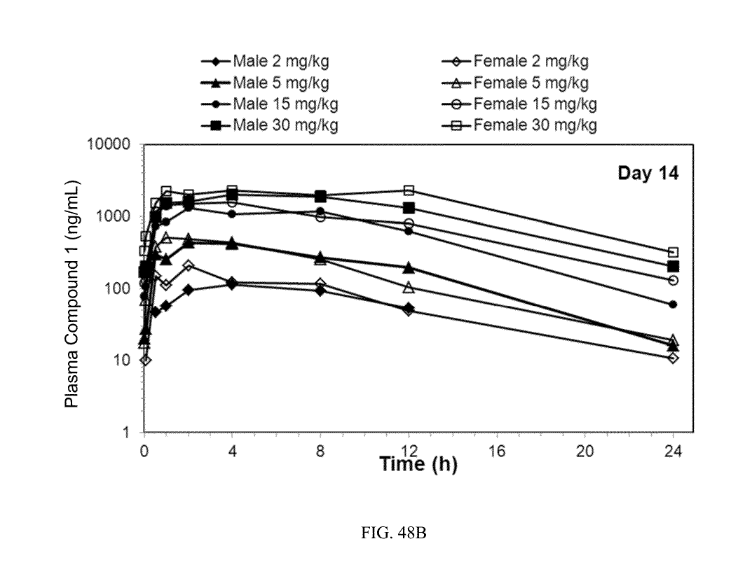

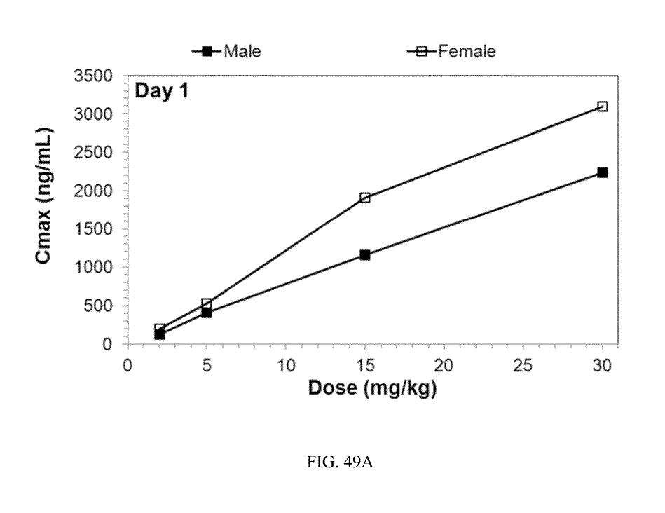

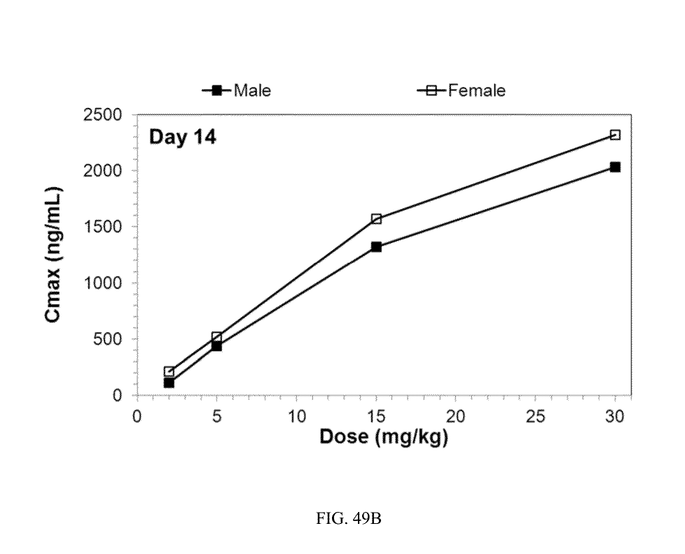

Mean plasma Compound 1 concentrations are listed in Table 26 and shown in FIG. 48. Individual concentrations are given in Table 21 through Table 25. No measurable Compound 1 was observed in samples collected from animals in the vehicle control group. Compound 1 was detectable in plasma through 24 hours post-dose at all dose levels except the 2 mg/kg dose group. Compound 1 was observed at 24 hours post-dose following administration of 2 mg/kg in only 3 samples collected from female animals.

TABLE 26
Mean Compound 1 Plasma Concentrations (ng/mL) Following
an Oral Administration to Rats on Study Days 1 and 14

| | | | Male | | | Female | | | Overall | | |
|---|---|---|---|---|---|---|---|---|---|---|---|
| Dose Group | Day | Time (h) | Mean | SD | n | Mean | SD | n | Mean | SD | n |
| 6 | 1 | 1 | BLQ | NA | 3 | BLQ | NA | 3 | BLQ | NA | 6 |
| Vehicle | 14 | 1 | BLQ | NA | 3 | BLQ | NA | 3 | BLQ | NA | 6 |
| 7 | 1 | 0.0833 | BLQ | NA | 3 | BLQ | NA | 3 | BLQ | NA | 6 |
| 2 mg/kg | | 0.5 | 57.7 | 20.3 | 3 | 110 | 35.8 | 3 | 83.7 | 38.5 | 6 |
| | | 1 | 57.1 | 9.21 | 3 | 91.4 | 33.3 | 3 | 74.3 | 28.8 | 6 |
| | | 2 | 102 | 9.60 | 3 | 196 | 17.3 | 3 | 149 | 53.0 | 6 |
| | | 4 | 126 | 20.7 | 3 | 175 | 75.3 | 3 | 150 | 56.3 | 6 |
| | | 8 | 90.4 | 23.2 | 3 | 96.7 | 47.0 | 3 | 93.6 | 33.3 | 6 |
| | | 12 | 53.7 | 6.92 | 3 | 82.2 | 24.1 | 3 | 67.9 | 22.3 | 6 |
| | | 24 | BLQ | NA | 3 | 13.0 | 14.6 | 3 | 6.50 | 11.7 | 6 |
| | 14 | PD | BLQ | NA | 3 | BLQ | NA | 2 | BLQ | NA | 5 |
| | | 0.0833 | BLQ | NA | 3 | 10.0 | NA | 2 | 4.00 | 8.94 | 5 |
| | | 0.5 | 47.0 | 9.85 | 3 | 150 | NA | 2 | 88.2 | 64.1 | 5 |
| | | 1 | 57.1 | 20.5 | 3 | 111 | NA | 2 | 78.5 | 32.7 | 5 |
| | | 2 | 94.9 | 12.7 | 3 | 212 | NA | 2 | 142 | 65.3 | 5 |
| | | 4 | 116 | 3.61 | 3 | 124 | NA | 2 | 119 | 6.18 | 5 |
| | | 8 | 94.2 | 22.7 | 3 | 117 | NA | 2 | 103 | 49.0 | 5 |
| | | 12 | 54.0 | 10.4 | 3 | 48.7 | NA | 2 | 51.9 | 14.2 | 5 |
| | | 24 | BLQ | NA | 3 | 10.8 | NA | 2 | 4.32 | 9.66 | 5 |
| 8 | 1 | 0.0833 | 16.0 | 8.34 | 3 | 25.6 | 9.67 | 3 | 20.8 | 9.66 | 6 |
| 5 mg/kg | | 0.5 | 237 | 78.7 | 3 | 267 | 48.1 | 3 | 252 | 60.6 | 6 |
| | | 1 | 194 | 49.5 | 3 | 406 | 226 | 3 | 300 | 187 | 6 |
| | | 2 | 254 | 161 | 3 | 528 | 18.4 | 3 | 391 | 182 | 6 |
| | | 4 | 408 | 138 | 3 | 482 | 75.2 | 3 | 445 | 108 | 6 |
| | | 8 | 276 | 44.3 | 3 | 360 | 69.2 | 3 | 318 | 69.3 | 6 |
| | | 12 | 195 | 12.1 | 3 | 140 | 7.51 | 3 | 168 | 31.3 | 6 |
| | | 24 | 25.6 | 5.90 | 3 | 18.6 | 5.37 | 3 | 22.1 | 6.34 | 6 |
| | 14 | PD | 20.0 | NA | 2 | 17.7 | 4.36 | 3 | 18.6 | 4.11 | 5 |
| | | 0.0833 | 27.2 | 2.47 | 3 | 70.7 | 39.1 | 3 | 49.0 | 34.4 | 6 |
| | | 0.5 | 298 | NA | 2 | 382 | 182 | 3 | 348 | 152 | 5 |
| | | 1 | 258 | 77.1 | 3 | 519 | 124 | 3 | 389 | 170 | 6 |
| | | 2 | 436 | NA | 2 | 493 | 199 | 3 | 470 | 158 | 5 |
| | | 4 | 421 | 104 | 3 | 439 | 30.0 | 3 | 430 | 69.4 | 6 |
| | | 8 | 273 | NA | 2 | 255 | 31.9 | 3 | 262 | 25.3 | 5 |
| | | 12 | 198 | 54.6 | 3 | 105 | 8.24 | 3 | 151 | 61.8 | 6 |
| | | 24 | 16.2 | NA | 2 | 19.2 | 2.79 | 3 | 18.0 | 4.40 | 5 |
| 9 | 1 | 0.0833 | 35.6 | 12.4 | 3 | 109 | 87.6 | 3 | 72.5 | 69.0 | 6 |
| 15 mg/kg | | 0.5 | 620 | 221 | 3 | 1180 | 163 | 3 | 899 | 351 | 6 |
| | | 1 | 810 | 221 | 3 | 1530 | 561 | 3 | 1170 | 549 | 6 |
| | | 2 | 1060 | 306 | 3 | 1910 | 162 | 3 | 1490 | 514 | 6 |
| | | 4 | 1160 | 155 | 3 | 1690 | 212 | 3 | 1430 | 331 | 6 |
| | | 8 | 1020 | 277 | 3 | 1440 | 214 | 3 | 1230 | 320 | 6 |
| | | 12 | 618 | 146 | 3 | 916 | 89.1 | 3 | 767 | 196 | 6 |
| | | 24 | 84.5 | 44.8 | 3 | 84.5 | 23.4 | 3 | 84.5 | 32.0 | 6 |
| | 14 | PD | 78.0 | 9.66 | 3 | 120 | 20.0 | 3 | 99.2 | 27.1 | 6 |
| | | 0.0833 | 107 | 38.3 | 3 | 131 | 31.8 | 3 | 119 | 34.1 | 6 |
| | | 0.5 | 740 | 38.5 | 3 | 1170 | 244 | 3 | 955 | 282 | 6 |
| | | 1 | 842 | 196 | 3 | 1450 | 189 | 3 | 1150 | 376 | 6 |
| | | 2 | 1320 | 83.3 | 3 | 1490 | 231 | 3 | 1410 | 180 | 6 |
| | | 4 | 1080 | 101 | 3 | 1570 | 190 | 3 | 1330 | 301 | 6 |
| | | 8 | 1170 | 101 | 3 | 979 | 80.3 | 3 | 1070 | 133 | 6 |
| | | 12 | 626 | 134 | 3 | 805 | 222 | 3 | 715 | 191 | 6 |
| | | 24 | 60.6 | 8.73 | 3 | 130 | 48.6 | 3 | 95.3 | 49.2 | 6 |
| 10 | 1 | 0.0833 | 208 | 213 | 3 | 92.9 | 18.4 | 3 | 150 | 149 | 6 |
| 30 mg/kg | | 0.5 | 1420 | 108 | 3 | 2160 | 202 | 3 | 1790 | 429 | 6 |
| | | 1 | 1400 | 374 | 3 | 2140 | 323 | 3 | 1770 | 513 | 6 |
| | | 2 | 1620 | 168 | 3 | 3090 | 476 | 3 | 2360 | 868 | 6 |
| | | 4 | 2240 | 415 | 3 | 2510 | 182 | 3 | 2380 | 324 | 6 |
| | | 8 | 1860 | 191 | 3 | 2470 | 140 | 3 | 2160 | 366 | 6 |
| | | 12 | 1660 | 233 | 3 | 1940 | 344 | 3 | 1800 | 303 | 6 |
| | | 24 | 234 | 52.3 | 3 | 284 | 22.3 | 3 | 259 | 45.4 | 6 |
| | 14 | PD | 172 | 38.1 | 3 | 336 | 14.8 | 3 | 254 | 93.3 | 6 |
| | | 0.0833 | 207 | 11.2 | 3 | 537 | 280 | 3 | 372 | 253 | 6 |
| | | 0.5 | 993 | 150 | 3 | 1540 | 147 | 3 | 1270 | 328 | 6 |
| | | 1 | 1530 | 587 | 3 | 2230 | 495 | 3 | 1880 | 621 | 6 |
| | | 2 | 1590 | 63.5 | 3 | 2010 | 172 | 3 | 1800 | 258 | 6 |
| | | 4 | 2030 | 78.1 | 3 | 2320 | 339 | 3 | 2180 | 271 | 6 |
| | | 8 | 1880 | 316 | 3 | 1960 | 55.7 | 3 | 1920 | 207 | 6 |
| | | 12 | 1300 | 133 | 3 | 2290 | 435 | 3 | 1800 | 617 | 6 |
| | | 24 | 205 | 11.4 | 3 | 322 | 75.7 | 3 | 263 | 80.3 | 6 |

BLQ = Below the lower limit of quantitation (<10.0 ng/mL);
NA = SD not calculated for BLQ or n < 3;
PD = Pre-dose

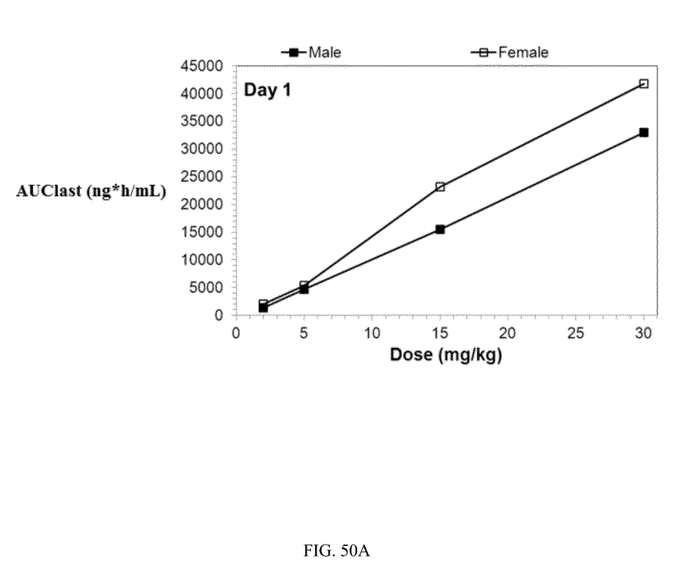

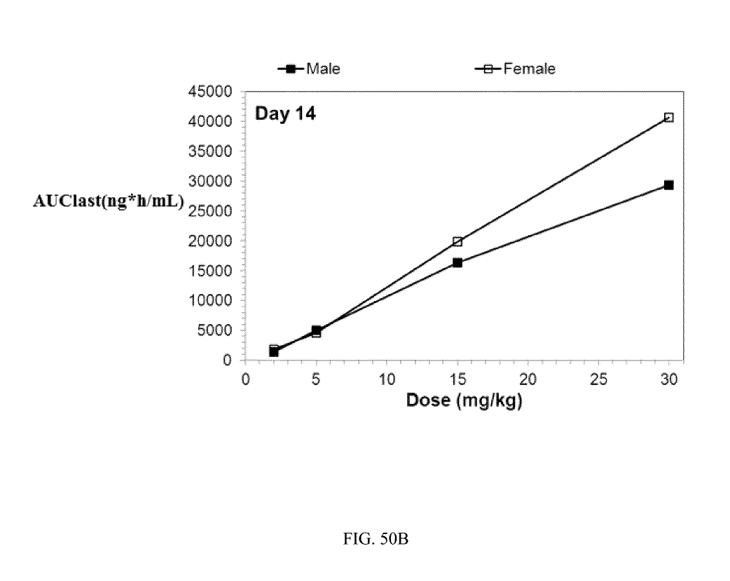

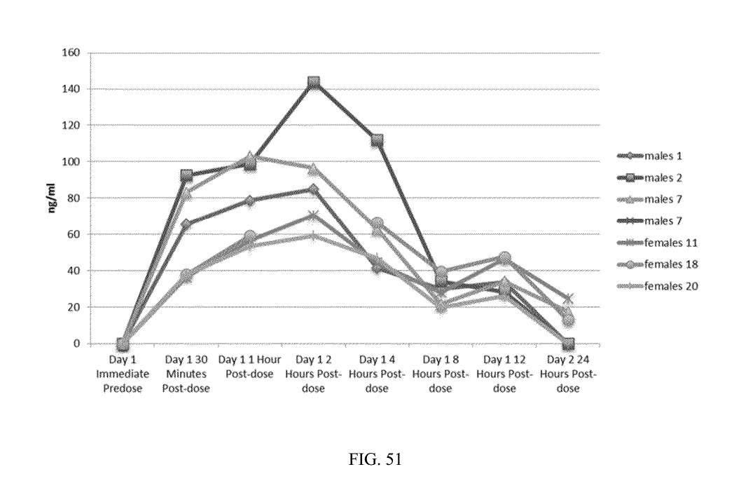

TK parameter values from mean Compound 1 concentrations are listed in Table 27. Increases in Cmax and AUC(0-t) with increasing dose are shown in FIGS. 49 and 50, respectively.

While systemic exposure, as evidenced by Cmax and AUC(0-t), tended to be higher in females than males, exposure was generally similar between males and females. Systemic Compound 1 exposure increased roughly proportionally with increasing dose level. The relationship between dose level and systemic exposure did not change with repeated daily dosing, as Cmax and AUC(0-t) were similar after the first and last doses.

TABLE 27
Mean TK Parameter Values from Compound 1 Plasma Concentrations (ng/mL)
Following Oral Administration to Rats on Study Days 1 and 14

| Dose | Study | | Cmax | Tmax | AUC(0-t) | AUC(0-inf) | t½ | CL | Vd | Vdss | Cmax/ | AUC/ | |
|---|---|---|---|---|---|---|---|---|---|---|---|---|---|
| Group | Day | Gender | (ng/mL) | (h) | (ng*h/mL) | (ng*h/mL) | (h) | (L/h/kg) | (L/kg) | (L/kg) | Dose | Dose | R2 |
| 7 | 1 | Male | 126 | 4 | 1390 | [a] | 5.32 | [a] | [a] | 10300 | 63.0 | 695 | 1.00 |
| 2 mg/kg | | Female | 196 | 2 | 2060 | 2160 | 5.25 | 926 | 7020 | 7350 | 98.0 | 1030 | 0.972 |
| | | Overall | 150 | 4 | 1730 | 1770 | 4.00 | 1130 | 6520 | 8580 | 75.0 | 865 | 0.983 |
| | 14 | Male | 116 | 4 | 1360 | [a] | 4.98 | [a] | [a] | 10700 | 58.0 | 680 | 1.00 |
| | | Female | 212 | 2 | 1770 | 1850 | 4.83 | 1080 | 7530 | 7790 | 106 | 885 | 0.983 |
| | | Overall | 142 | 2 | 1530 | 1550 | 3.46 | 1290 | 6440 | 9250 | 71.0 | 765 | 0.999 |
| 8 | 1 | Male | 408 | 4 | 4680 | 4850 | 4.52 | 1030 | 6720 | 8490 | 81.6 | 936 | 0.989 |
| 5 mg/kg | | Female | 528 | 2 | 5340 | 5440 | 3.82 | 919 | 5070 | 6210 | 106 | 1070 | 0.994 |
| | | Overall | 445 | 4 | 5020 | 5150 | 4.14 | 971 | 5810 | 7200 | 89.0 | 1000 | 1.00 |
| | 14 | Male | 436 | 2 | 5030 | 5120 | 3.77 | 977 | 5310 | 7260 | 87.2 | 1010 | 0.981 |
| | | Female | 519 | 1 | 4620 | 4740 | 4.41 | 1050 | 6720 | 6590 | 104 | 924 | 0.990 |
| | | Overall | 470 | 2 | 4820 | 4930 | 4.08 | 1010 | 5980 | 6990 | 94.0 | 964 | 0.998 |
| 9 | 1 | Male | 1160 | 4 | 15500 | 16000 | 4.39 | 938 | 5930 | 7620 | 77.3 | 1030 | 0.997 |
| 15 mg/kg | | Female | 1910 | 2 | 23200 | 23700 | 3.80 | 633 | 3470 | 4850 | 127 | 1550 | 0.991 |
| | | Overall | 1490 | 2 | 19400 | 19900 | 4.05 | 754 | 4400 | 5900 | 99.3 | 1290 | 0.994 |
| | 14 | Male | 1320 | 2 | 16300 | 16600 | 3.70 | 904 | 4830 | 7000 | 88.0 | 1090 | 0.998 |
| | | Female | 1570 | 4 | 19800 | 20800 | 5.24 | 721 | 5460 | 5820 | 105 | 1320 | 0.976 |
| | | Overall | 1410 | 2 | 18100 | 18700 | 4.47 | 802 | 5170 | 6320 | 94.0 | 1210 | 0.993 |
| 10 | 1 | Male | 2240 | 4 | 33000 | 34700 | 5.05 | 865 | 6290 | 7880 | 74.7 | 1100 | 0.963 |
| 30 mg/kg | | Female | 3090 | 2 | 41800 | 43800 | 4.92 | 685 | 4860 | 5980 | 103 | 1390 | 0.980 |
| | | Overall | 2380 | 4 | 37500 | 39400 | 4.98 | 761 | 5470 | 6760 | 79.3 | 1250 | 0.973 |
| | 14 | Male | 2030 | 4 | 29300 | 30700 | 4.88 | 977 | 6880 | 8530 | 67.7 | 977 | 0.993 |
| | | Female | 2320 | 4 | 40600 | 43200 | 5.56 | 694 | 5580 | 6640 | 77.3 | 1350 | 0.905 |
| | | Overall | 2180 | 4 | 35000 | 37000 | 5.23 | 811 | 6120 | 7460 | 72.7 | 1170 | 0.955 |

[a] = Insufficient data after Cmax to perform regression

Rats in the control group were not exposed to Compound 1 and so were a true control group. Rats given Compound 1 had detectable exposure up to 24 hours after administration at all dose levels, except for males in the 2 mg/kg dose group, where exposure was observed up to 12 hours post-dose. The relationship between dose level and systemic exposure was generally similar in both sexes, although systemic exposure at a given dose level tended to be higher in females. Systemic Compound 1 exposure increased roughly proportionally with increasing dose level. The relationship between dose level and systemic exposure did not change with repeated daily dosing, as Cmax and AUC(0-t) were similar after the first and last doses.

### Example 9

### Toxicokinetic Studies in Dogs

The toxicokinetic profile of Compound 1 in dogs (Beagle) was evaluated to determine dose tolerability. Two groups of dogs received Compound 1 at 15 and 45 mg/kg once daily for 14 consecutive days. Dogs in the control group were not exposed to Compound 1 and so were a true control group.

Compound 1 was dissolved in 5% dextrose in water, pH adjusted to 4.0-4.5. Compound 1 dose formulations were prepared once weekly by combining the appropriate weighed amount of Compound 1 di-HCl salt with the appropriate volume of vehicle control to achieve each of the required final concentrations of test article formulation. All Compound 1 concentrations in dose formulations are expressed as the free base. Therefore, a correction factor of 1.3 was be applied such that 1.3 mg of Compound 1 di-HCl salt=1.0 mg of Compound 1 (free base).

The procedure for preparing each of the concentrations of Compound 1 dose formulations was as follows. The required weight of Compound 1 di-HCl was added to an appropriate size vessel. Approximately 90-95% of the anticipated total volume of formulated vehicle control (5% dextrose in water) was added to the vessel containing Compound 1. The vessel was placed on a stirring hotplate and the formulation heated to a temperature of 30-35° C. The contents of the vessel were allowed to stir at 30-35° C. or a stir plate until all test article appeared to be dissolved, and for a period of at least 1 hour. The pH of the formulation was verified and adjusted to 4.0-4.5 using 1N HCl or 1N NaOH as required. The formulation was transferred into an appropriate size graduated cylinder. Sufficient vehicle control will be added to the cylinder to obtain the final desired concentration of formulated test article. The completed stock formulation was dispensed into several amber glass bottles as aliquots for each day of dosing. These formulations were stored at controlled room temperature (15-30° C.) until used for dose administration.

The beagle dog was chosen for this study as it is a species that is used for non-clinical toxicity and pharmacokinetic evaluations and satisfies the regulatory requirement for non-clinical safety studies in a non-rodent species. The total number of animals used is considered to be the minimum number of dogs required to assess the tolerability and the pharmacokinetic responses of Compound 1, and allowing for individual variability in responses, when administered once daily for fourteen consecutive days. Two groups of dogs, each group comprising of 3 males and 3 females, were administered Compound 1 at dose levels of 15 mg/kg and 45 mg/kg. An additional group of dogs (3 males and 3 females) received the vehicle control once daily for fourteen consecutive days. To evaluate plasma exposure, plasma samples were obtained at selected time points on each of Days 1 and 14, and analyzed to measure Compound 1 concentration. These data were used to generate toxicokinetic parameters. The study design is summarized in Table 28 below.

TABLE 28
Toxicokinetic Study Design in Dogs

| Group No./ | Dose Levels | Dose Volume | Number of Animals | |
|---|---|---|---|---|
| Treatment | (mg/kg) | (ml/kg) | Males | Females |
| 1/Vehicle Control | 0 | 5 | 3 | 3 |
| 2/Compound 1 | 15 | 5 | 3 | 3 |
| 3/Compound 1 | 45 | 5 | 3 | 3 |

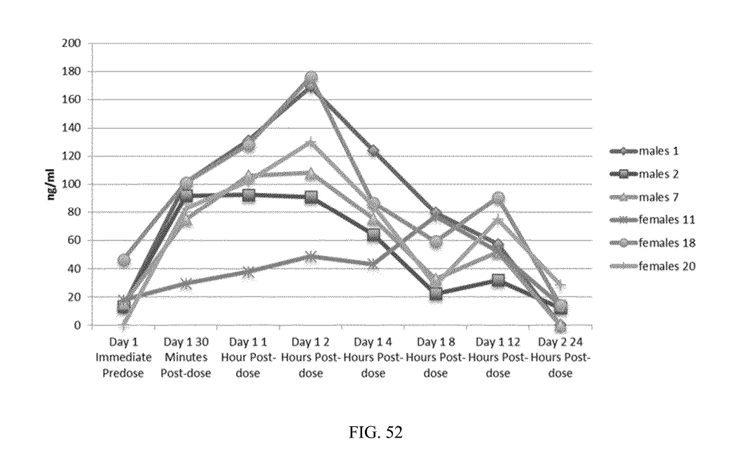

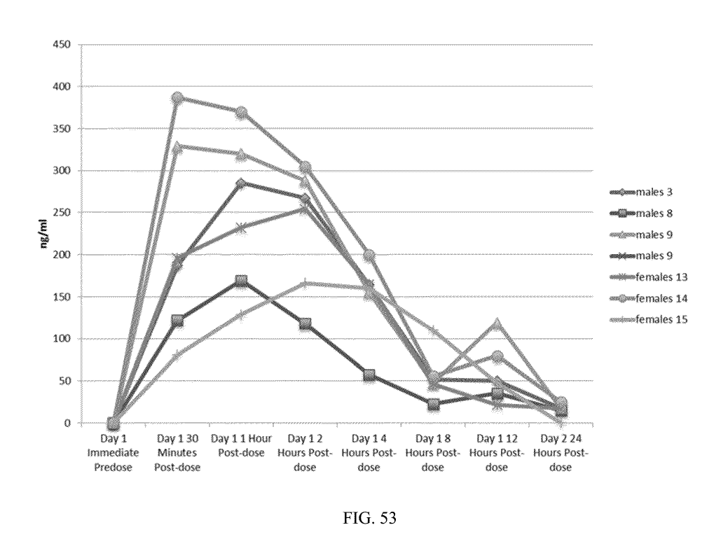

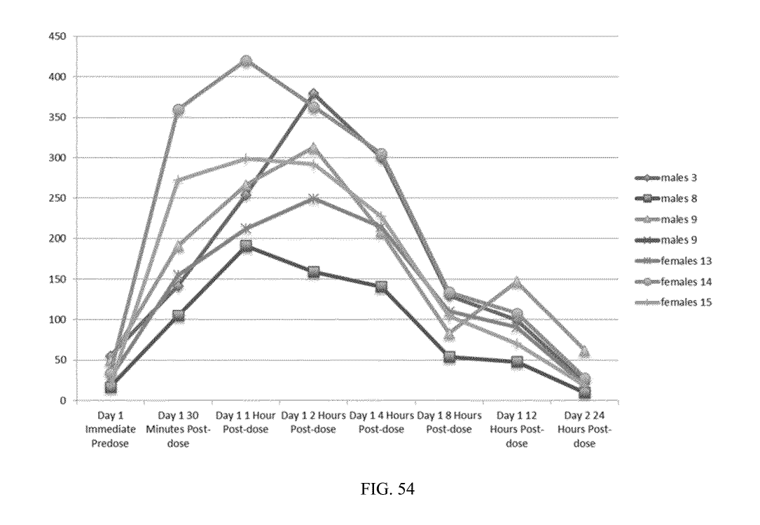

FIG. 51 shows plasma concentration over time of Compound 1 delivered to beagle dogs at 15 mpk at Day 1 of treatment. FIG. 52 shows plasma concentration over time of Compound 1 delivered to beagle dogs at 15 mpk at Day 14 of treatment. FIG. 53 shows plasma concentration over time of Compound 1 delivered to beagle dogs at 45 mpk at Day 1 of treatment. FIG. 54 shows plasma concentration over time of Compound 1 delivered to beagle dogs at 45 mpk at Day 14 of treatment. No measurable Compound 1 was observed in samples collected from animals in the vehicle control group. Compound 1 was detectable in plasma through 24 hours post-dose at the highest dose levels. Compound 1 was observed at 24 hours post-dose following administration of 10 mg/kg in only 1/3 samples collected from male animals, and 2/3 female animals. Systemic Compound 1 exposure increased roughly proportionally with increasing dose level in both male and female dogs.

### Example 10

### Pharmokinetic Comparison of Palbociclib to Compound 1

Palbociclib is a CDK4/6 inhibitor developed and marketed by Pfizer. It is currently approved as an antineoplastic for the treatment of advanced CDK4/6-replication dependent breast cancer. The use of palbociclib as a chemoprotectant, however, is problematic because of its extended efficacy period and long comparative half-life in prohibiting cell-cycle replication, leading to hematological side effects such as myelosuppression.

Table 29 compares the derived pharmacokinetic data from the Compound 1 clinical trials with literature reported PK values for palbociclib. As illustrated, Compound 1 has a higher Cmax, a higher AUC at its BED dose, a quicker clearance rate, and a much shorter half-life compared to palbociclib.

TABLE 29
PK/PD Comparison of Compound 1 with Palbociclib

| | Cmax | AUC (n*hr/ml | Vz/F | CL/F | T_{1/2} |
|---|---|---|---|---|---|
| Compound | (ng/ml) | (0-inf) | (L) | (L/hr) | (hours) |
| Palbociclib | 78 | 2002 | 3241 | 88.5 | 26.7 |
| (150 mg oral)¹ | | | | | |
| Compound 1 | 932 | 1380 | 940 | 121 | 8.6 |
| (96 mg/m² IV) | | | | | |
| Compound 1 | 2030 | 3050 | 729 | 126 | 7.9 |
| (192 mg/m² IV) | | | | | |

¹Schwartz G K, Lorusso P M, Dickson M A, et al. Phase I study of PD 0332991, a cyclin dependent kinaseinhibitor, administered in 3-week cycles (Schedule 2/1). Br J Cancer. 2011; 104(12):1862-1868.

In addition, as shown below in Table 30, palbociclib accumulates into higher blood plasma concentrations over time when administered in consecutive daily doses. Comparatively, as described above, Compound 1 does not show accumulation in blood plasma over time when administered on consecutive days.

TABLE 30
Accumulation of Palbociclib in Blood Plasma
with Consecutive Daily Dose Administration

| | 100 mg QD | | 150 mg QD | | 200 mg QD | | 225 mg QD | |
|---|---|---|---|---|---|---|---|---|
| Parameter | Day 1 | Day 8 | Day 1 | Day 8 | Day 1 | Day 8 | Day 1 | Day 8 |
| Cmax (ng/ml) | 44 | 58 | 78 | 194 | 81 | 174 | 104 | 186 |
| Tmax (h) | 4 | 4 | 6 | 7 | 5.7 | 4 | 4 | 4.5 |
| AUC (0-10) | 333 | 433 | 622 | 1599 | 525 | 1395 | 718 | 1491 |
| ng*hr/ml | | | | | | | | |

Schwartz G K, Lorusso P M, Dickson M A, et al. Phase I study of PD 0332991, a cyclin dependent kinase inhibitor, administered in 3-week cycles (Schedule 2/1). Br J Cancer. 2011; 104(12): 1862-1868.

### Example 11

### Compound 1 Induces a Dose Dependent Bone Marrow Arrest in Dog Bone Marrow

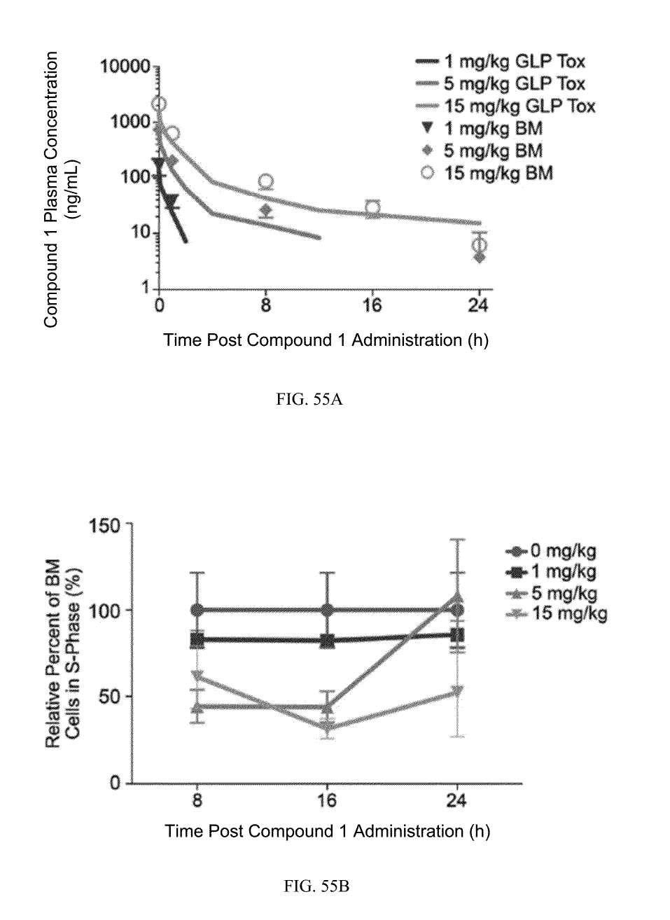

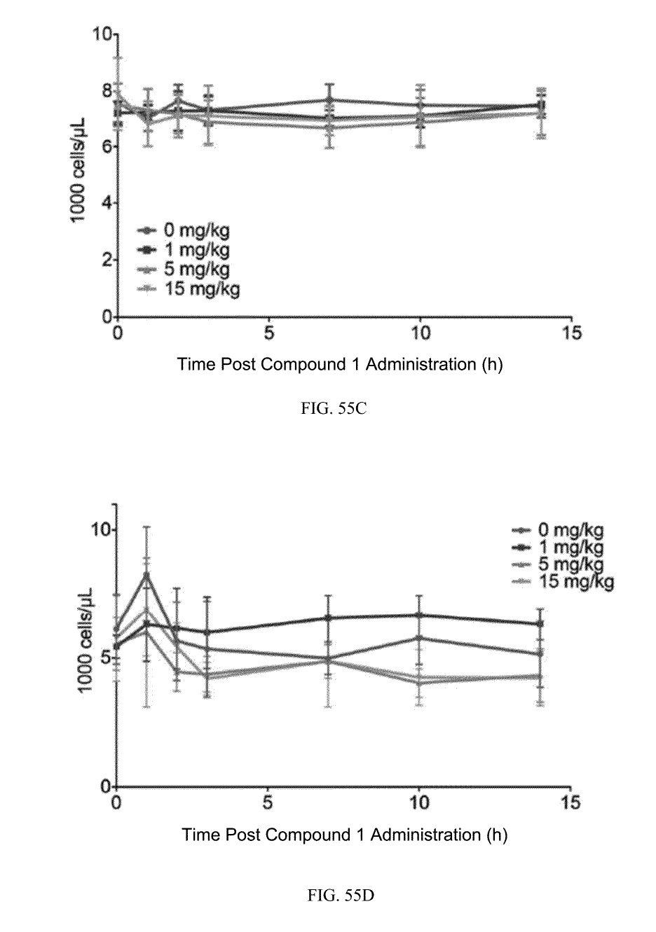

A pharmacokinetic time course was performed in canines to determine the effect of Compound 1 on bone marrow arrest. As seen in FIG. 55A, plasma concentrations of Compound 1 increased with higher doses of Compound 1 given in either GLP toxicology studies or in bone marrow EdU experiments. As seen in FIG. 55B, increasing dosages of Compound 1 led to a decrease in the number of cells in S phase at 8 and 16 hours, with bone marrow cells showing recovery at 24 hours compared to 16 hours. The effect of Compound 1 administration on red blood cells and neutrophils in dogs out to 15 days was minimal (FIGS. 55C and 55D). By day 15, red blood cell counts in dogs treated with Compound 1 were similar to untreated dogs. By day 15, neutrophil counts in dogs treated with Compound 1 were slightly lower than in untreated dogs. These results show that administration of Compound 1 produces a dose dependent bone marrow arrest in canines.

### Example 12

### Compound 1 Induces a G1 Arrest in Human Bone Marrow Hematopoietic Stem and Progenitor Cells

The effect of Compound 1 on G1 arrest was examined in various human hematopoietic cell populations. Bone marrow aspirates were drawn from subjects in the biologically effective dose (BED) Cohort at various time points (predose [n=5], and 24 hours [n=3] or 32 [n=4] hours post Compound 1 dose). One pre-dose sample and one 32 hour post-dose sample were inadequate and were excluded. White blood cells were isolated using a Ficoll gradient and stained for specific bone marrow lineage markers (CD45, CD71, CD61, CD38, CD11b, CD14). Cells were then treated with Draq5 (DNA staining dye), and cell cycle analysis was completed using flow cytometry. Phases of the cell cycle (G1 vs. S/G2M) were calculated in each lineage population at each time point. Cell surface markers were used to identify the specific HSPC populations: Hematopoietic stem and multipotent progenitor cells (HSC and MPP)=CD45^{dim}/CD34+/CD38−; Oligopotent progenitors (OPPs)=CD45^{dim}/CD34+/CD38+; Monocyte progenitors=CD45+/CD14+/CD11b+; Granulocyte progenitors=CD45+/CD14−/CD11b+; Erythroid progenitors=CD45−/CD71+; Megakaryocyte progenitors=CD45+/CD61+.

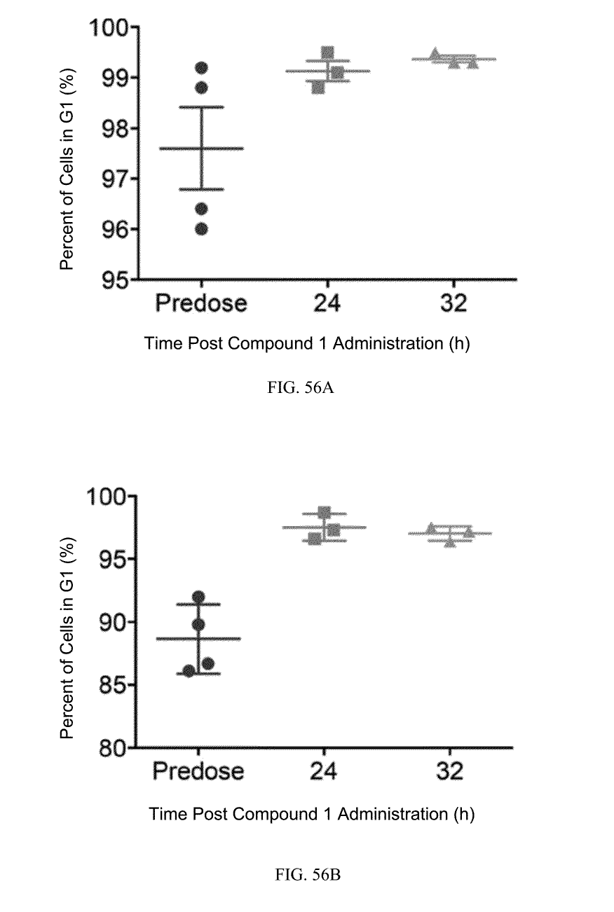

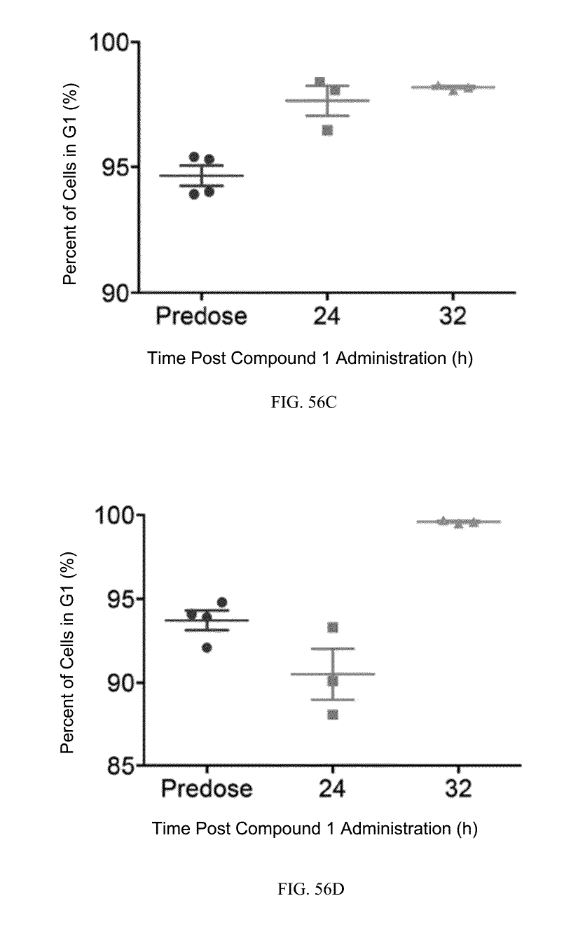

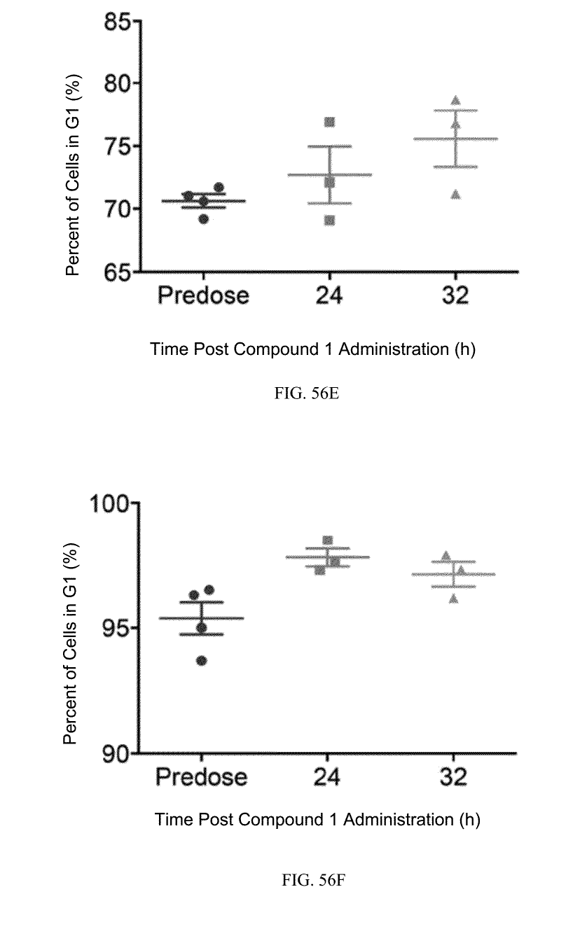

As shown in FIG. 56A, administration of Compound 1 (192 mg/m²) to human patients led to an increase in the percentage of hematopoietic stem cells and multipotent progenitor cells in G1 at 24 and 32 hours. As shown in FIG. 56B, administration of Compound 1 (192 mg/m²) to human patients led to an increase in the percentage of oligopotent progenitor cells in G1 at 24 and 32 hours. As shown in FIG. 56C, administration of Compound 1 (192 mg/m²) to human patients led to an increase in the percentage of monocytes in G1 at 24 and 32 hours.

As shown in FIG. 56D, administration of Compound 1 (192 mg/m²) to human patients led to a decrease in the percentage of granulocytes in G1 at 24 hours, but led to an increase at 32 hours. As shown in FIG. 56E, administration of Compound 1 (192 mg/m²) to human patients led to an increase in the percentage of erythrocytes in G1 at 24 and 32 hours. As shown in FIG. 56F, administration of Compound 1 (192 mg/m²) to human patients led to an increase in the percentage of megakaryocytes in G1 at 24 and 32 hours. The data obtained from experiments shown in FIG. 56 is summarized in Table 31.

TABLE 31
Percent of Bone Marrow Lineage Populations in G1 or S/G2/M
Phase of the Cell Cycle Following Compound 1 Administration

| | G1 | | | S/G2/M | | |
|---|---|---|---|---|---|---|
| | Predose | 24 hours | 32 hours | Predose | 24 hours | 32 hours |
| HSC and MPP | 97.60 | 99.13 | 99.37 | 2.55 | 0.93 | 0.63 |
| OPP | 88.65 | 97.53 | 97.03 | 11.78 | 2.57 | 2.97 |
| Granulocyte Lineage | 93.73 | 90.50 | 99.60 | 6.85 | 10.37 | 1.80 |
| Monocyte Lineage | 94.65 | 97.67 | 38.20 | 5.58 | 2.73 | 1.53 |
| Erythrocyte Lineage | 70.63 | 72.70 | 75.57 | 29.28 | 27.30 | 24.53 |
| Megakaryocyte | 95.38 | 97.80 | 97.13 | 4.75 | 2.30 | 2.93 |
| Lineage | | | | | | |

HSC, hematopoietic stem cells; MPP, multipotent progenitors; OPP, oligopotent progenitors.

### Example 13

### Compound 1 Demonstrates Prolonged Exposure in the Bone Marrow with no Impact on Peripheral Blood Cell Counts

The effect of Compound 1 on peripheral blood cell counts was examined in human subjects. Matched Compound 1 plasma concentrations (n=5) from bone marrow aspirates and peripheral blood were determined at the indicated times following Compound 1 administration (192 mg/m²).

Time course of blood cell counts (n=18) for neutrophils, lymphocytes, red blood cells (RBCs), and platelets were conducted following Compound 1 administration (192 mg/m²) for up to 14 days.

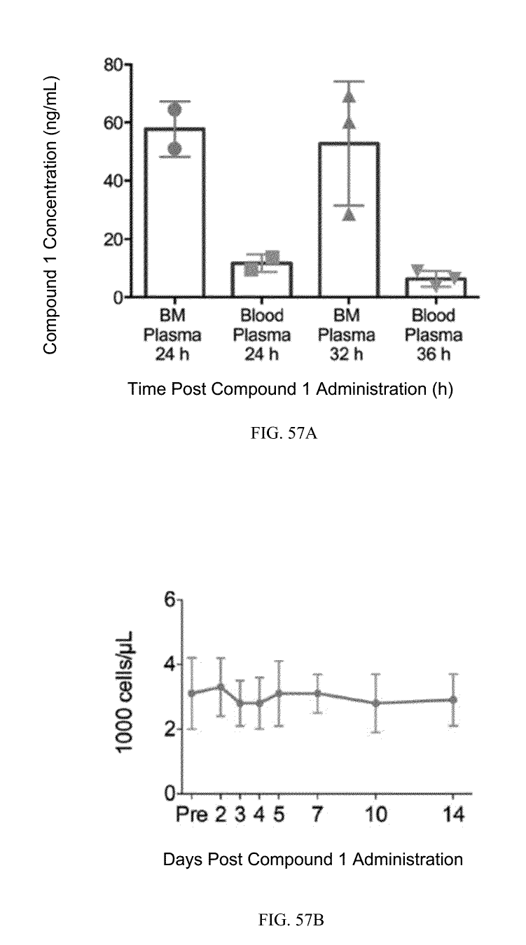

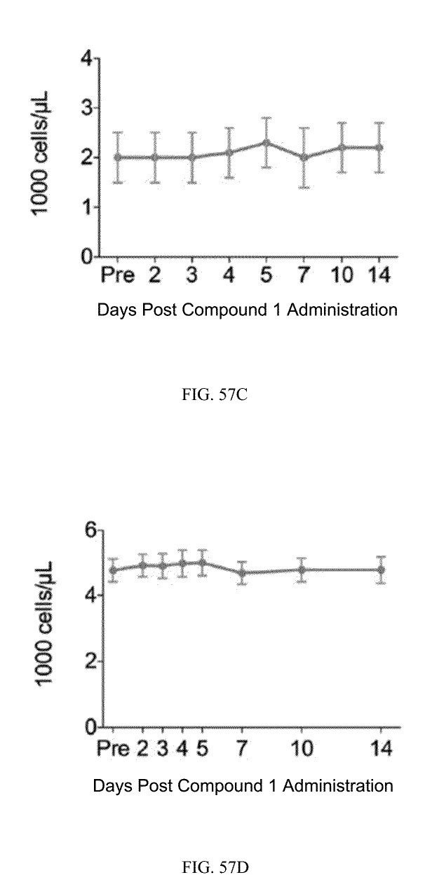

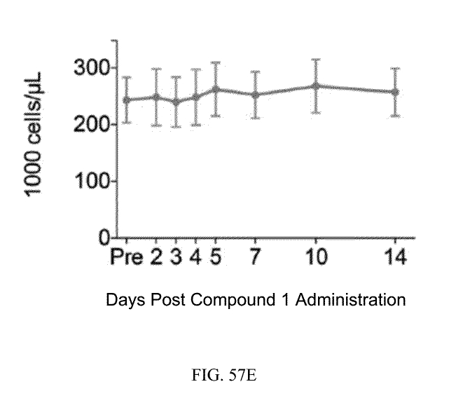

As shown in FIG. 57A, the matched samples from human subjects demonstrated significantly more Compound 1 in the bone marrow plasma compared to the peripheral blood plasma at both 24 and 32 hours after administration of Compound 1. As shown in FIG. 57B-57E, despite robust inhibition of hematopoietic stem and progenitor cell (HSPC) proliferation, a single dose of Compound 1 (192 mg/m²) did not alter peripheral blood cell counts, including neutrophils, lymphocytes, red blood cells, and platelets, when examined for up to fourteen days post-Compound 1 administration.

### Example 14

### Compound 1 Demonstrates Protection from Cell Loss Associated with Chemotherapy

The effect of Compound 1 on peripheral blood cell counts was examined in mice subjects. Compound 1 plasma concentrations were determined following the administration of 5-Fluorouacil (5FU) in the presence or absence of a prior dose of Compound 1 (100 mg/m²). Blood cell counts for neutrophils, lymphocytes, red blood cells (RBCs), and platelets were conducted.

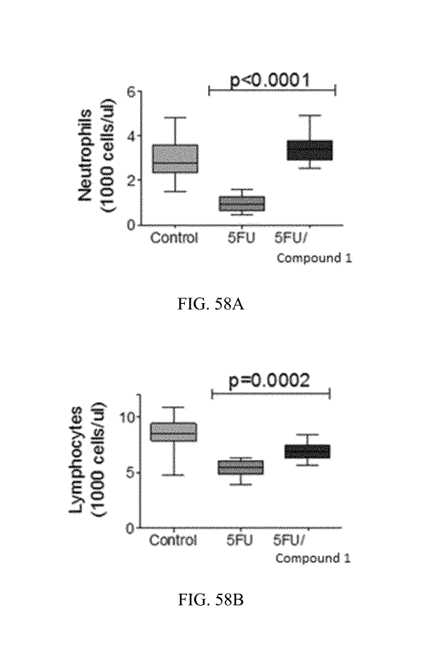

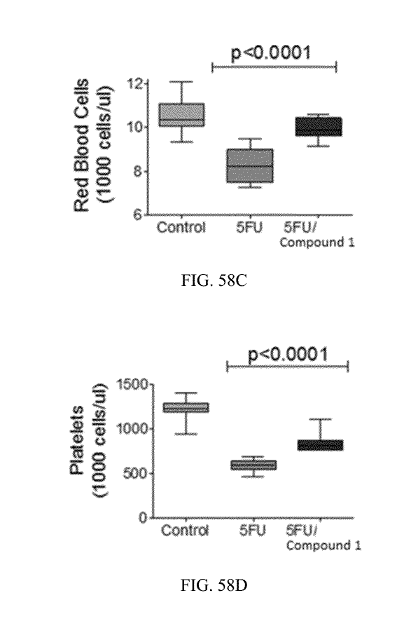

As shown in FIG. 58A, the samples from mice demonstrated significantly less loss of neutrophils upon exposure to 5FU chemotherapy when Compound 1 was previously dosed at 100 mg/kg. As shown in FIG. 58B, the samples from mice demonstrated significantly less loss of lymphocytes upon exposure to 5FU chemotherapy when Compound 1 was previously dosed at 100 mg/kg. As shown in FIG. 58C, the samples from mice demonstrated significantly less loss of red blood cells upon exposure to 5FU chemotherapy when Compound 1 was previously dosed at 100 mg/kg. As shown in FIG. 58D, the samples from mice demonstrated significantly less loss of platelets upon exposure to 5FU chemotherapy when Compound 1 was previously dosed at 100 mg/kg.

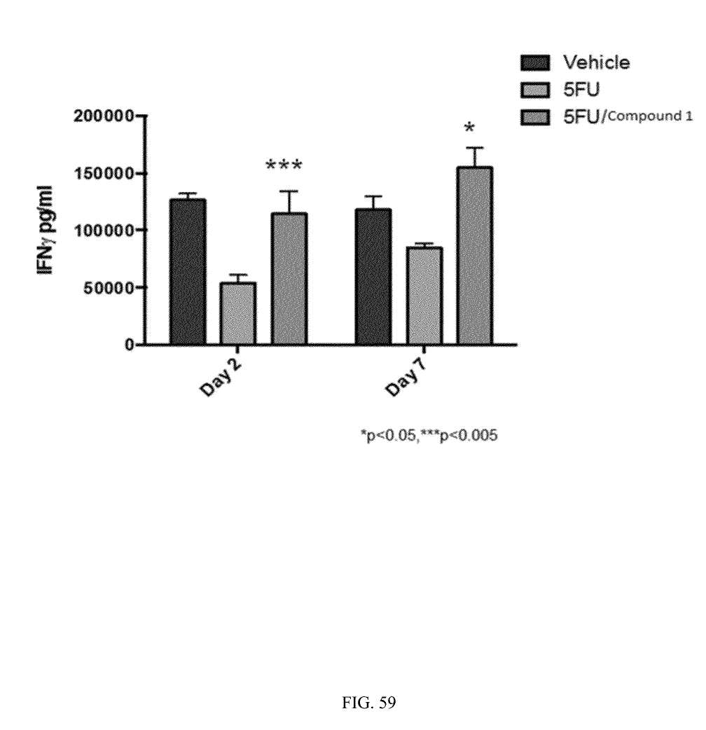

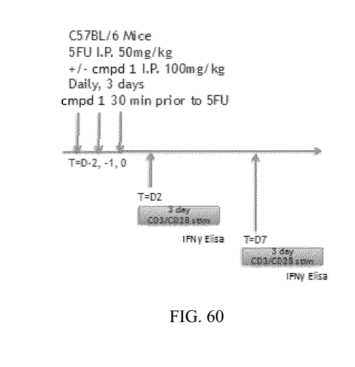

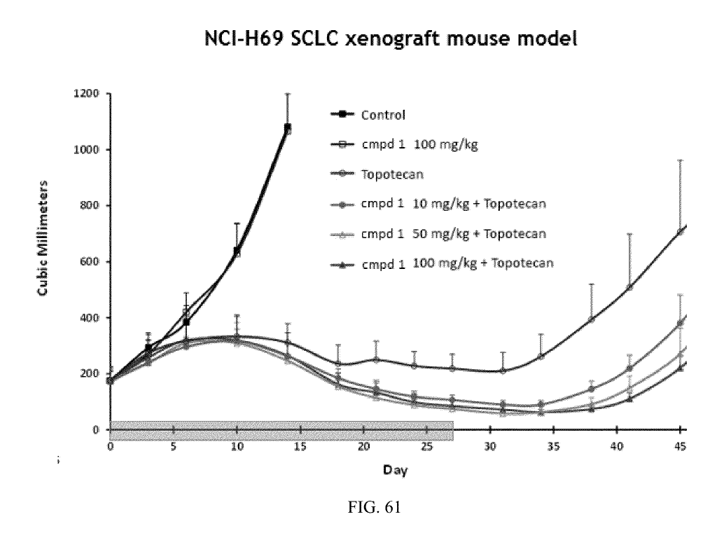

Similarly the effect of 5FU in the presence and absence of Compound 1 on interferon gamma concentration was measured 2 and 5 days post dosing in mice subjects. As shown in FIG. 59, mice challenged with 5-Fluorouracil (5FU) (50 mg/kg) interferon gamma levels decreased significantly. In mice pretreated with 100 mg/kg of Compound 1 interferon gamma levels did not decrease significantly. As shown in FIG. 60 C57BL/6 Mice were dosed with 50 mg/kg I.P. For the treatment group the mice were dosed with 100 mg/kg Compound 1 30 minutes prior to chemotherapy.

### Example 15

### Compound 1 Causes a Dose-Dependent Increase in Topotecan Efficacy in NCI-H69 SCLC Xenograft Mouse Model

Compound 1 and Topotecan were tested in a NCI-H69 mice small cell lung cancer (SCLC) xenograft study during a treatment cycle of 28 days. Mice were treated with 100 mg/kg doses of Compound 1 (qdx5dx4), 0.6 mg/kg doses of Topotecan (qdx5dx4), 10 mg/kg Compound 1 and 0.6 mg/kg Topotecan, 50 mg/kg Compound 1 and 0.6 mg/kg Topotecan, or 100 mg/kg Compound 1 and 0.6 mg/kg Topotecan. As shown in FIG. 61 in the absence of Topotecan mice treated with Compound 1 did not have tumor grown suppression. Mice treated with Topotecan and Compound 1, on the other hand had a significantly higher level of tumor suppression then those treated with Topotecan alone. Further, this higher level of suppression was Compound 1 dose dependent. These results when taken together suggest a synergistic effect of dosing Compound 1 in conjunction with Topotecan.

### Example 16

### Compound 1 Displays a Dose-Dependent Increase in Exposure with Little Accumulation

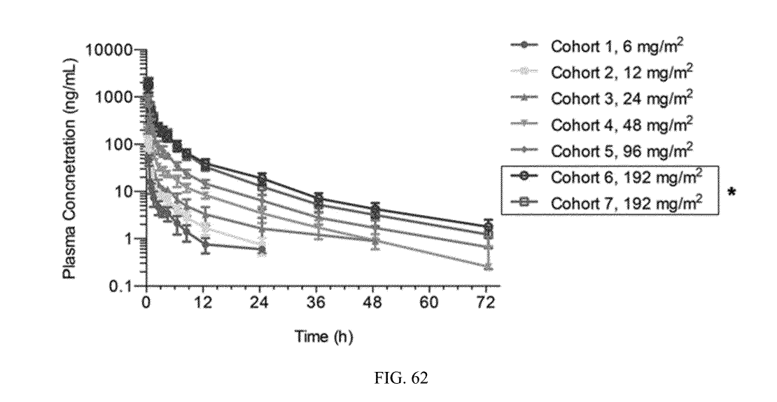

Seven cohorts of healthy subjects were given a single dose of Compound 1 by IV ranging from 6 mg/m² to 192 mg/m². As shown in FIG. 62 the concentration level of Compound 1 in the blood was dose dependent and decreased rapidly over the time course of 72 hours. Subjects receiving a 192 mg/m² or less dose of Compound 1 had less than 10 ng/mL of compound in the plasma after 72 hours, suggesting that the compound does not accumulate significantly.

### Example 17

### Compound 1 Dosed Over Multiple Days with Etoposide and Carboplatin Chemotherapy does not Significantly Accumulate

Subjects received 200 mg/m² of Compound 1 administered over a 30-minute infusion duration once daily on Days 1 to 3 of each 21-day cycle prior to chemotherapy. Carboplatin was dosed with a target AUC=5 min*mg/mL IV over 30 minutes on Day 1 and Etoposide was dosed at 100 mg/m² administered by IV over 60 minutes daily on days 1, 2, and 3 on each 21 day cycle. Blood samples were obtained prior to dosing, at the end of the 30-minute infusion (EOI), and at 0.5, 1, 1.5, 2, 2.5, 4, 6, 8, and 24 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times.

TABLE 32
Noncompartmental Pharmacokinetic Parameters for Compound
1(200 mg/m²) with Etoposide and Carboplatin Day 1

| | Cmax | t½ | AUC_{24.5} | AUCinf |
|---|---|---|---|---|
| Subject | (ng/mL) | (h) | (h*ng/mL) | (h*ng/mL) |
| 1 | 845 | 8.91 | 2010 | 2290 |
| 2 | 1840 | 7.09 | 2520 | 2680 |
| 3 | 587 | 7.28 | 1530 | 1650 |
| 4 | 2170 | 6.29 | 2860 | 2960 |
| 5 | 742 | 8.94 | 2850 | 3320 |
| 6 | 1000 | 10.6 | 1930 | 2290 |
| N | 6 | 6 | 6 | 6 |
| Mean | 1200 | 8.19 | 2290 | 2530 |
| SD | 648 | 1.60 | 543 | 585 |
| Min | 587 | 6.29 | 1530 | 1650 |
| Median | 923 | 8.09 | 2270 | 2490 |
| Max | 2170 | 10.6 | 2860 | 3320 |
| CV % | 54.2 | 19.5 | 23.7 | 23.1 |
| Geo. | 1070 | 8.07 | 2230 | 2470 |
| Mean | | | | |

Noncompartmental PK parameters of Compound 1 for the subjects on Day 1 of dosing are summarized in Table The Cmax averaged 1200 ng/mL. Half-lives averaged 8.19 hours and ranged from 6.29 to 10.6 hours. AUC_{inf} values averaged 2530 h*ng/mL with low variability, as reflected by a CV % of 23.1%.

TABLE 33
Noncompartmental Pharmacokinetic Parameters for Compound
1 (200 mg/m²) with Etoposide and Carboplatin Day 3

| | Cmax | t½ | AUCτ |
|---|---|---|---|
| Subject | (ng/mL) | (h) | (h*ng/mL) |
| 1 | 1190 | 8.20 | 2630 |
| 2 | 1630 | 11.0 | 2610 |
| 3 | 3360 | 6.99 | 2890 |
| 4 | 723 | 9.27 | 2020 |
| 5 | 1000 | 9.04 | 3300 |
| 6 | 355 | 9.75 | 2010 |
| N | 6 | 6 | 6 |
| Mean | 1380 | 9.04 | 2570 |
| SD | 1060 | 1.37 | 501 |
| Min | 355 | 6.99 | 2010 |
| Median | 1100 | 9.15 | 2620 |
| Max | 3360 | 11.0 | 3300 |
| CV % | 77.2 | 15.1 | 19.5 |
| Geo. | 1090 | 8.95 | 2530 |
| Mean | | | |

Noncompartmental PK parameters of Compound 1 for the subjects on Day 3 of dosing are summarized in Table 33. The Cmax on Day 3 was marginally higher, averaging 1380 ng/mL. Half-lives averaged 9.04 hours and ranged from 6.99 to 11.0 hours. AUCτ values averaged 2570 h*ng/mL with low variability, as reflected by a CV % of 19.5%.

At 24.5 hours after the start of infusion on Day 1, all 4 subjects had measurable compound 1 concentrations averaging 20.0 ng/mL and ranging from 11.3 to 35.9 ng/mL. On Day 3, there were measurable pre-dose concentrations averaging 20.6 ng/mL and ranging from 10.7 to 30.3 ng/mL. Concentrations at 24.5 hours after dosing on Day 3 averaged 24.6 ng/mL and ranged from 11.5 to 34.5 ng/mL.

### Example 18

### Compound 1 Displays no Clinically Relevant Myelotoxicity During Etoposide and Carboplatin Chemotherapy

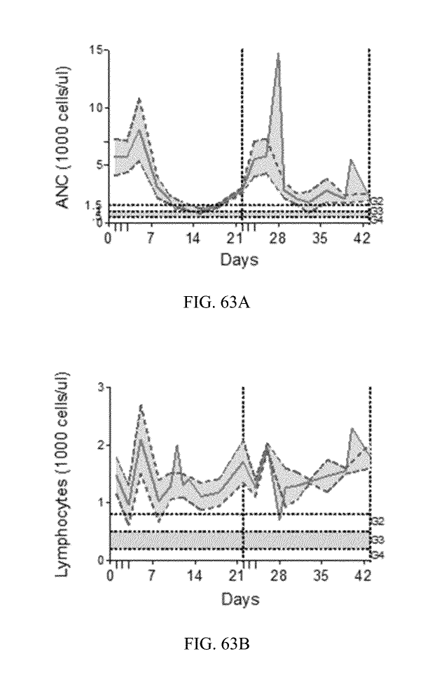

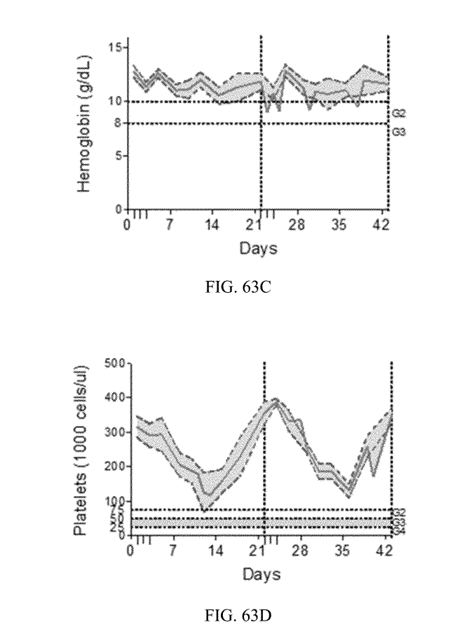

Subjects from the above study displayed no clinically relevant myelotoxicity in scans of absolute neutrophil count (ANC), lymphocyte count, hemoglobin concentration and platelet count. As shown in FIG. 63A the absolute neutrophil count though affected by chemotherapy rarely dipped below the G1 phase. As shown in FIG. 63B the lymphocyte count though affected by chemotherapy rarely dipped below the G1 phase. As shown in FIG. 63C the hemoglobin concentration though affected by chemotherapy rarely dipped below the G1 phase. As shown in FIG. 63D the platelet count though affected by chemotherapy rarely dipped below the G1 phase.

### Example 19

### Compound 1 Dosed Over Multiple Days with Topotecan Chemotherapy does not Significantly Accumulate

In Part 1, subjects in Cohorts 1, 2 and 3 received 200 mg/m² of Compound 1 administered over a 30-minute infusion duration once daily on Days 1 to 5 of each 21-day cycle prior to chemotherapy (1.5 mg/m², 1.25 mg/m², and 0.75 mg/m² of Topotecan for Cohorts 1, 2, and 3 respectively). Blood samples were obtained prior to dosing, at the end of the 30-minute infusion (EOI), and at 0.5, 1, 1.5, 2, 2.5, 4, 6, 8, and 24 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times.

TABLE 34
Noncompartmental Pharmacokinetic Parameters for Compound
1(200 mg/m²) with Topotecan Day 1

| | Cmax | t½ | AUC_{24.5} | AUCinf |
|---|---|---|---|---|
| Subject | (ng/mL) | (h) | (h*ng/mL) | (h*ng/mL) |
| 1 | 749 | 5.60 | 1770 | 1840 |
| 2 | 1160 | 6.24 | 2310 | 2430 |
| N | 2 | 2 | 2 | 2 |
| Mean | 955 | 5.92 | 2040 | 2140 |
| SD | 291 | 0.454 | 381 | 414 |
| Min | 749 | 5.60 | 1770 | 1840 |
| Median | 955 | 5.92 | 2040 | 2140 |
| Max | 1160 | 6.24 | 2310 | 2430 |
| CV % | 30.4 | 7.7 | 18.7 | 19.4 |
| Geo. Mean | 932 | 5.91 | 2030 | 2120 |
| 3 | 1000 | 6.14 | 2210 | 2320 |
| 4 | 985 | 8.67 | 2450 | 2720 |
| 5 | 727 | 8.81 | 2130 | 2430 |
| N | 3 | 3 | 3 | 3 |
| Mean | 904 | 7.87 | 2260 | 2490 |
| SD | 153 | 1.50 | 166 | 204 |
| Min | 727 | 6.14 | 2130 | 2320 |
| Median | 985 | 8.67 | 2210 | 2430 |
| Max | 1000 | 8.81 | 2450 | 2720 |
| CV % | 17.0 | 19.0 | 7.4 | 8.2 |
| Geo. Mean | 895 | 7.77 | 2260 | 2490 |
| 6 | 1390 | 7.01 | 2090 | 2190 |
| 7 | 660 | 6.64 | 1610 | 1700 |
| 8 | 835 | 9.24 | 1970 | 2240 |
| 9 | 1670 | 6.69 | 2540 | 2660 |
| N | 4 | 4 | 4 | 4 |
| Mean | 1140 | 7.40 | 2050 | 2200 |
| SD | 471 | 1.24 | 382 | 392 |
| Min | 660 | 6.64 | 1610 | 1700 |
| Median | 1110 | 6.85 | 2030 | 2220 |
| Max | 1670 | 9.24 | 2540 | 2660 |
| CV % | 41.4 | 16.8 | 18.6 | 17.8 |
| Geo. Mean | 1060 | 7.33 | 2020 | 2170 |

Noncompartmental PK parameters of Compound 1 for the subjects in Cohorts 1, 2 and 3 on Day 1 of Compound 1 dosing are summarized in Table 34. The Cmax values averaged 955 ng/mL, 904 ng/mL, and 1140 ng/mL for Cohorts 1-3, respectively. Half-lives averaged 5.92, 7.87, and 7.4 hours. AUCinf values averaged 2140, 2490, and 2200 h*ng/mL with low variability, as reflected by CV % ranging from 8.2 to 19.4%.

TABLE 35
Noncompartmental Pharmacokinetic Parameters for Compound
1(200 mg/m²) with Topotecan Day 4

| | Cmax | t½ | AUCτ |
|---|---|---|---|
| Subject | (ng/mL) | (h) | (h*ng/mL) |
| 1 | 897 | 6.96 | 1960 |
| 2 | 1100 | 9.15 | 2710 |
| N | 2 | 2 | 2 |
| Mean | 999 | 8.06 | 2330 |
| SD | 144 | 1.54 | 531 |
| Min | 897 | 6.96 | 1960 |
| Median | 999 | 8.06 | 2330 |
| Max | 1100 | 9.15 | 2710 |
| CV % | 14.4 | 19.2 | 22.8 |
| Geometric | 993 | 7.98 | 2300 |
| Mean | | | |
| 3 | 904 | 7.15 | 2770 |
| 4 | 781 | 9.10 | 2190 |
| 5 | 571 | 10.6 | 1970 |
| N | 3 | 3 | 3 |
| Mean | 752 | 8.95 | 2310 |
| SD | 168 | 1.73 | 412 |
| Min | 571 | 7.15 | 1970 |
| Median | 781 | 9.10 | 2190 |
| Max | 904 | 10.6 | 2770 |
| CV % | 22.4 | 19.3 | 17.8 |
| Geometric | 739 | 8.84 | 2290 |
| Mean | | | |
| 6 | 1570 | 10.7 | 1670 |
| 7 | 520 | 8.09 | 1700 |
| 8 | 1610 | 9.54 | 2310 |
| 9 | 1600 | 6.87 | 3030 |
| N | 4 | 4 | 4 |
| Mean | 1330 | 8.81 | 2180 |
| SD | 537 | 1.69 | 638 |
| Min | 520 | 6.87 | 1670 |
| Median | 1590 | 8.82 | 2000 |
| Max | 1610 | 10.7 | 3030 |
| CV % | 40.5 | 19.2 | 29.3 |
| Geometric | 1200 | 8.69 | 2110 |
| Mean | | | |

Noncompartmental PK parameters Day 4 of Compound 1 dosing are summarized in Table 35. The average Cmax values on Day 4 were 999, 752, and 1330 ng/mL, for Cohorts 1, 2, and 3, respectively. Half-lives averaged 8.06, 8.95, and 8.81 hours. AUCτ values averaged 2330, 2310, and 2180 h*ng/mL with low variability, as reflected by a range of CV % values from 17.8 to 29.3%.

On Day 1, the mean maximum concentration was 955 ng/mL for Cohort 1, for Cohort 2 it was 904 ng/mL, and for Cohort 3 1140 ng/mL. For Day 4, the mean maximum concentrations averaged 999, 752, and 1330 ng/mL, for Cohorts 1, 2, and 3, respectively. At 24.5 hours after the start of infusion on Day 1, all subjects had measurable G1T28-1 concentrations averaging 10.8, 19.5, and 13.3 ng/mL. On Day 4, there were measurable pre-dose concentrations averaging 17.2, 33.3, and 27.1 ng/mL. Concentrations at 24.5 hours after dosing on Day 4 averaged 19.2, 22.7, and 18.2 ng/mL.

### Example 20

### Administration of Compound 1Results in a Reduction in the Biologically Effective Dose of Topotecan

In Cohort 1 subjects received 1.5 mg/m² of topotecan administered over a 30-minute infusion duration once daily on Days 1 to 5 of each 21-day cycle following a 30-minute infusion of 200 mg/m² Compound 1. Blood samples were obtained prior to dosing, at the end of the 30-minute infusion (EOI), and at 0.5, 1, 1.5, 2, 2.5, 4, 6, 8, and 24 hours after the end of infusion. Analysis of plasma concentration versus time data for calculation of standard pharmacokinetic (PK) parameters following intravenous infusion administration was conducted using Phoenix WinNonlin version 6.3 using a nominal infusion duration and scheduled blood sampling times. In Cohort 2 there were 3 subjects who received 1.25 mg/m² of topotecan once daily on Days 1 to 5. In Cohort 3 there were 4 subjects who received 0.75 mg/m² of topotecan once daily on Days 1 to 5.

On Day 1, the Cmax averaged 69.5 ng/mL for the 1.5 mg/m² dose, 63.7 ng/mL for the 1.25 mg/m² dose and 27.3 ng/mL for the 0.75 mg/m² dose. Half-lives averaged 4.33 hours for Cohorts 1 and 3 and 4.93 hours for Cohort 2 on Day 1 indicating that accumulation during multiple daily dosing is not likely. This was confirmed by the Cmax values for Day 4 which averaged 89.3 ng/mL for the 1.5 mg/m² dose, 59.6 ng/mL for the 1.25 mg/m² dose and 24.5 ng/mL for the 0.75 mg/m² dose. AUCinf values on Day 1 averaged 152 h*ng/mL, 180 h*ng/mL, and 94.4 h*ng/mL for the 1.5, 1.25, and 0.75 mg/m² doses, respectively, and were very similar for Day 4.

TABLE 36
Noncompartmental Pharmacokinetic Parameters for Topotecan (1.5
mg/m², or 1.25 mg/m² 0.75 mg/m²) with Compound 1 (200 mg/m²) Day 1

| | | | Cmax | AUC_{24.5} | t½ | AUCinf | AUCinf | AUCx5 |
|---|---|---|---|---|---|---|---|---|
| Day | Cohort | Subject | (ng/mL) | (h*ng/mL) | (h) | (h*ng/mL) | (min*nM) | (min*nM) |
| 1 | Cohort 1 | 118-3001 | 40.2 | 130 | 4.56 | 132 | 17300 | 86600 |
| | | 118-3002 | 98.8 | 169 | 4.11 | 171 | 22500 | 112000 |
| | | N | 2 | 2 | 2 | 2 | 2 | 2 |
| | | Mean | 69.5 | 150 | 4.33 | 152 | 19900 | 99400 |
| | | SD | 41.4 | 27.9 | 0.322 | 27.7 | 3630 | 18200 |
| | | Min | 40.2 | 130 | 4.11 | 132 | 17300 | 86600 |
| | | Median | 69.5 | 150 | 4.33 | 152 | 19900 | 99400 |
| | | Max | 98.8 | 169 | 4.56 | 171 | 22500 | 112000 |
| | | CV % | 59.6 | 18.7 | 7.4 | 18.3 | 18.3 | 18.3 |
| 1 | Cohort 2 | 118-3004 | 57.7 | 159 | 4.78 | 162 | 21300 | 106000 |
| | | 118-3005 | 94.1 | 247 | 5.09 | 254 | 33300 | 166000 |
| | | 118-3006 | 39.3 | 120 | 4.92 | 123 | 16100 | 80400 |
| | | N | 3 | 3 | 3 | 3 | 3 | 3 |
| | | Mean | 63.7 | 175 | 4.93 | 180 | 23500 | 118000 |
| | | SD | 27.9 | 65.0 | 0.156 | 67.4 | 8830 | 44100 |
| | | Min | 39.3 | 120 | 4.78 | 123 | 16100 | 80400 |
| | | Median | 57.7 | 159 | 4.92 | 162 | 21300 | 106000 |
| | | Max | 94.1 | 247 | 5.09 | 254 | 33300 | 166000 |
| | | CV % | 43.8 | 37.1 | 3.2 | 37.5 | 37.5 | 37.5 |
| 1 | Cohort 3 | 102-3001 | 20.8 | 83.5 | 4.32 | 84.8 | 11100 | 55600 |
| | | 105-3001 | 17.9 | 75.0 | 4.04 | 75.9 | 9950 | 49700 |
| | | 118-3007 | 38.5 | 118 | 4.27 | 120 | 15700 | 78600 |
| | | 162-3001 | 32.0 | 94.7 | 4.70 | 96.8 | 12700 | 63400 |
| | | N | 4 | 4 | 4 | 4 | 4 | 4 |
| | | Mean | 27.3 | 92.8 | 4.33 | 94.4 | 12400 | 61800 |
| | | SD | 9.63 | 18.7 | 0.275 | 19.1 | 2500 | 12500 |
| | | Min | 17.9 | 75.0 | 4.04 | 75.9 | 9950 | 49700 |
| | | Median | 26.4 | 89.1 | 4.30 | 90.8 | 11900 | 59500 |
| | | Max | 38.5 | 118 | 4.70 | 120 | 15700 | 78600 |
| | | CV % | 35.3 | 20.1 | 6.3 | 20.2 | 20.2 | 20.2 |

TABLE 37
Noncompartmental Pharmacokinetic Parameters
for Topotecan (1.5 mg/m², or 1.25 mg/m² 0.75 mg/m²)
with Compound 1 (200 mg/m²) Day 4

| | | | Cmax | t½ | AUCτ | AUCτ |
|---|---|---|---|---|---|---|
| Day | Cohort | Subject | (ng/mL) | (h) | (h*ng/mL) | (min*nM) |
| 4 | Cohort 1 | 1 | 56.5 | 5.11 | 143 | 18800 |
| | | 2 | 122 | 4.64 | 181 | 23700 |
| | | N | 2 | 2 | 2 | 2 |
| | | Mean | 89.3 | 4.87 | 162 | 21200 |
| | | SD | 46.3 | 0.330 | 26.8 | 3510 |
| | | Min | 56.5 | 4.64 | 143 | 18800 |
| | | Median | 89.3 | 4.87 | 162 | 21200 |
| | | Max | 122 | 5.11 | 181 | 23700 |
| | | CV % | 51.9 | 6.8 | 16.5 | 16.5 |
| 4 | Cohort 2 | 3 | 41.8 | 5.39 | 158 | 20700 |
| | | 4 | 104 | 5.97 | 231 | 30300 |
| | | 5 | 33.1 | 4.98 | 124 | 16300 |
| | | N | 3 | 3 | 3 | 3 |
| | | Mean | 59.6 | 5.45 | 171 | 22500 |
| | | SD | 38.7 | 0.497 | 54.7 | 7170 |
| | | Min | 33.1 | 4.98 | 124 | 16300 |
| | | Median | 41.8 | 5.39 | 158 | 20700 |
| | | Max | 104 | 5.97 | 231 | 30300 |
| | | CV % | 64.8 | 9.1 | 31.9 | 31.9 |
| 4 | Cohort 3 | 6 | 26.4 | 4.63 | 83.7 | 11000 |
| | | 7 | 18.4 | 5.07 | 74.4 | 9740 |
| | | 8 | 30.7 | 4.68 | 99.7 | 13100 |
| | | 9 | 22.6 | 4.73 | 112 | 14700 |
| | | N | 4 | 4 | 4 | 4 |
| | | Mean | 24.5 | 4.78 | 92.4 | 12100 |
| | | SD | 5.26 | 0.199 | 16.7 | 2190 |
| | | Min | 18.4 | 4.63 | 74.4 | 9740 |
| | | Median | 24.5 | 4.70 | 91.7 | 12000 |
| | | Max | 30.7 | 5.07 | 112 | 14700 |
| | | CV % | 21.4 | 4.2 | 18.1 | 18.1 |

Preliminary noncompartmental PK parameters for topotecan for the subjects in Cohorts 1, 2, and 3 are displayed in Table 36 and Table 37 for Days 1 and 4, respectively. On Day 1, the Cmax averaged 69.5 ng/mL for the 1.5 mg/m² dose, 63.7 ng/mL for the 1.25 mg/m² dose and 27.3 ng/mL for the 0.75 mg/m² dose. Half-lives averaged 4.33 hours for Cohorts 1 and 3 and 4.93 hours for Cohort 2 on Day 1. The Cmax values for Day 4 averaged 89.3 ng/mL for the 1.5 mg/m² dose, 59.6 ng/mL for the 1.25 mg/m² dose and 24.5 ng/mL for the 0.75 mg/m² dose.

AUC_{inf} values on Day 1 averaged 152 h*ng/mL, 180 h*ng/mL, and 94.4 h*ng/mL for the 1.5, 1.25 and 0.75 mg/m² doses, respectively, and were very similar for Day 4.

### Example 21

### Compound 1 Dosed with Etoposide and Carboplatin Reduced Tumor Volume in 6 of 6 Patients Over the Course of 2-6 Cycles of Treatment

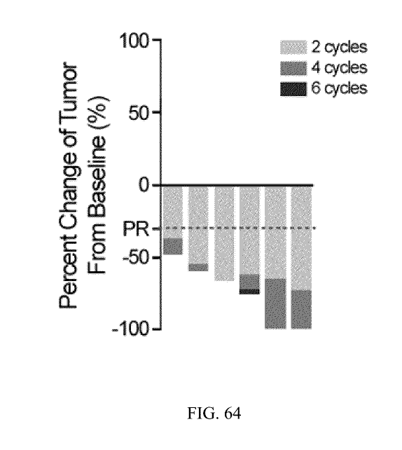

Subjects received 200 mg/m² of Compound 1 administered over a 30-minute infusion duration once daily on Days 1 to 3 of each 21-day cycle prior to chemotherapy. Carboplatin was dosed with a target AUC=5 min*mg/mL IV over 30 minutes on Day 1 and Etoposide was dosed at 100 mg/m² administered by IV over 60 minutes daily on days 1, 2, and 3 on each 21 day cycle. As shown in FIG. 64 all 6 patients had at least partial responses during the course of 2 to 6 cycles of chemotherapy. Target lesions were summed and response tabulated as follows:

TABLE 38
Response rate of subjects treated with Compound 1 (200 mg/m²)
followed by chemotherapy.

| | Baseline | Cycle 2 | Cycle4 | Cycle 6 | |
|---|---|---|---|---|---|
| | Target | Target | Target | Target | |
| | Lesion | Lesion | Lesion | Lesion | |
| Subject | Sum | Sum | Sum | Sum | Response |
| 1 | 120 | 35 | 0 | 0 | CR |
| 2 | 184 | 74 | 54 | 47 | PR |
| 3 | 59 | 22 | 0 | | PR |
| 4 | 117 | 76 | 62 | | PR |
| 5 | 344 | 161 | 146 | | PR |
| 6 | 130 | 50 | | | PR |

### Example 22

### Compound 1 Dosed with Topotecan Resulted in Partial Response or Stable Disease State in 6 of 6 Patients

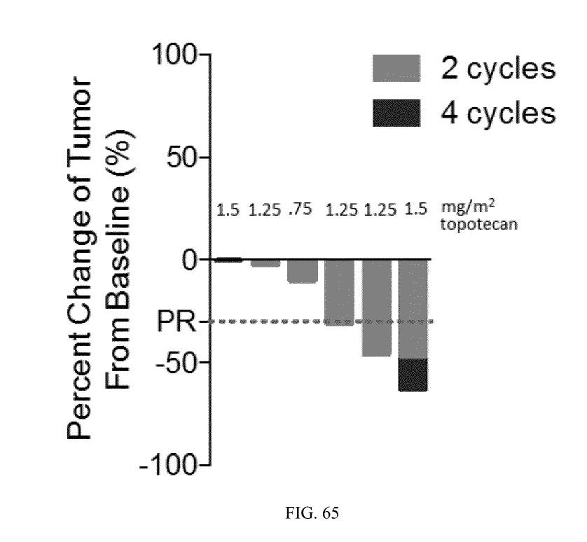

In Cohort 1 subjects received 1.5 mg/m² of topotecan administered over a 30-minute infusion duration once daily on Days 1 to 5 of each 21-day cycle following a 30-minute infusion of 200 mg/m² Compound 1. In Cohort 2 there were 3 subjects who received 1.25 mg/m² of topotecan once daily on Days 1 to 5. In Cohort 3 there were 4 subjects who received 0.75 mg/m² of topotecan once daily on Days 1 to 5. As shown in FIG. 65 stable disease resulted in 3 subjects whilst partial responses were achieved in 3 subjects over the course of 2 to 4 cycles of chemotherapy. Target lesions were summed and response tabulated as follows:

TABLE 39
Response rate of subjects treated with Compound 1 (200 mg/m²) followed
by Topotecan (1.5 mg/m², 1.25 mg/m², 0.75 mg/m²).

| | | | | | |
|---|---|---|---|---|---|
| | | Best | Baseline | Cycle 2 | Cycle 4 |
| | | Response | Target | Target | Target |
| | | to | Lesion | Lesion | Lesion |
| Subject | Prior Treatment | 1st Line | Sum | Sum | Sum |

1.5 mg/m² Topotecan

| | | | | | |
|---|---|---|---|---|---|
| 1 | E/Cb × 3 cycles | SD | 88 | 88 | 88 |
| 2 | E/Cb × 4 cycles | SD | 19 | 10 | 7 |

1.25 mg/m² Topotecan

| | | | | |
|---|---|---|---|---|
| 3 | E/Cisplatin × 4 | SD | 41 | 22 |
| | cycles | | | |
| 4 | E/Cb × 4 cycles | SD | 179 | 123 |
| 5 | E/Cb × 6 cycles | SD | 106 | 104 |

.75 mg/m² Topotecan

| | | | | |
|---|---|---|---|---|
| 6 | E/Cb × 6 cycles | CR | 135 | |
| 7 | E/Cb × 6 cycles | PR | 101 | |
| 8 | 1. E/P ± anti- | 1. PD | 175 | 157 |
| | notch antibody × | | | |
| | 2 cycles | | | |
| | 2. Camptosar × 4 | 2. SD | | |
| | cycles | | | |
| 9 | E/Cb + anti-notch | PD | 53 | |
| | antibody × 2 | | | |
| | cycles | | | |

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

We claim:

1. A method of treating a human subject having small cell lung cancer comprising:
intravenously administering to the subject a composition comprising Compound 1, or an acceptable salt thereof, in a single dose that provides a blood plasma profile mean Cmax (ng/ml) for Compound 1 of between about 1000 (ng/ml) and 3500 (ng/ml), and,
subsequently administering to the subject a therapeutically effective amount of at least one chemotherapeutic agent selected from etoposide, carboplatin, topotecan, or a combination thereof,
wherein the chemotherapeutic agent is administered to the subject within about four hours or less of the administration of Compound 1.

2. The method of claim 1, wherein the blood plasma profile mean Cmax is between about 1400 ng/ml and 3100 ng/ml.

3. The method of claim 1, wherein the blood plasma profile mean Cmax is about 2030 ng/ml+/− about 20%.

4. The method of claim 1, wherein the blood plasma profile dosage-corrected mean Cmax is about 6.0 ((mg/ml)/(mg/m²))+/−20%.

5. The method of claim 1, wherein Compound 1 is administered in a dose of between about 180 mg/m² and 280 mg/m².

6. The method of claim 5, wherein Compound 1 is administered in a dose of about 200 mg/m².

7. The method of claim 5, wherein Compound 1 is administered in a dose of about 240 mg/m².

8. The method of claim 1, wherein Compound 1 is administered to the subject about 30 minutes prior to the administration of the at least one chemotherapeutic agent.

9. The method of claim 1, wherein the at least one chemotherapeutic agent is etoposide.

10. The method of claim 1, wherein the at least one chemotherapeutic agent is carboplatin.

11. The method of claim 1, wherein the at least one chemotherapeutic agent is topotecan.

12. The method of claim 11, wherein the topotecan is administered at a dose of 1.5 mg/m² or less.

13. The method of claim 12, wherein the topotecan is administered at a dose of about 1.25 mg/m².

14. The method of claim 12, wherein the topotecan is administered at a dose of about 0.75 mg/m².

15. The method of claim 1, wherein administration of a single dose of Compound 1 further provides a blood plasma profile mean AUCₜ measured at 24.5 hours of at least about 2040 (ng*hr/ml).

16. A method of treating a human subject having small cell lung cancer comprising:
intravenously administering to the subject a composition comprising Compound 1, or an acceptable salt thereof, in a single dose that provides a blood plasma profile mean AUCₜ of Compound 1 measured at about 24.5 hours following administration of Compound 1 of about 2000 (h*ng/ml) to about 4500 (h*ng/ml), and
subsequently administering to the subject a therapeutically effective amount of at least one chemotherapeutic agent selected from etoposide, carboplatin, topotecan, or a combination thereof,
wherein the chemotherapeutic agent is administered to the subject within about four hours or less of the administration of Compound 1.

17. The method of claim 16, wherein the blood plasma profile mean AUCₜ measured at 24.5 hours is between about 2300 (ng*hr/ml) and 4000 (ng*hr/ml).

18. The method of claim 16, wherein the blood plasma profile mean AUCₜ measured at 24.5 hours is about 2830 (ng*hr/ml)+/−about 20%.

19. The method of claim 16, wherein the blood plasma profile mean AUCₜ measured at 24.5 hours is at least 2040 (ng*hr/ml).

20. The method of claim 16, wherein the blood plasma profile dosage-corrected mean AUCₜ measured at 24.5 hours is at least about 8.35 ((ng*hr/ml)/(mg/m²)).

21. The method of claim 16, wherein Compound 1 is administered in a dose of between about 180 mg/m² and 280 mg/m².

22. The method of claim 21, wherein Compound 1 is administered in a dose of about 200 mg/m².

23. The method of claim 21, wherein Compound 1 is administered in a dose of about 240 mg/m².

24. The method of claim 16, wherein Compound 1 is administered to the subject about 30 minutes prior to the administration of the at least one chemotherapeutic agent.

25. The method of claim 16, wherein the at least one chemotherapeutic agent is etoposide.

26. The method of claim 16, wherein the at least one chemotherapeutic agent is carboplatin.

27. The method of claim 16, wherein the at least one chemotherapeutic agent is topotecan.

28. The method of claim 27, wherein the topotecan is administered at a dose of 1.5 mg/m² or less.

29. The method of claim 28, wherein the topotecan is administered at a dose of about 1.25 mg/m².

30. The method of claim 28, wherein the topotecan is administered at a dose of about 0.75 mg/m².

31. The method of claim 16, wherein administration of a single dose of Compound 1 further provides a blood plasma profile mean Cmax of between about 1000 (ng/ml) and 3500 (ng/ml).

32. A method of treating a human subject having small cell lung cancer comprising:
intravenously administering to the subject a composition comprising Compound 1, or an acceptable salt thereof, in a single dose of between about 180 mg/m² and 280 mg/m², and
subsequently administering to the subject a therapeutically effective amount of at least one chemotherapeutic agent selected from etoposide, carboplatin, topotecan, or a combination thereof,
wherein the chemotherapeutic agent is administered to the subject within about four hour or less of the administration of Compound 1.

33. The method of claim 32, wherein Compound 1 is administered in a dose of about 200 mg/m².

34. The method of claim 32, wherein Compound 1 is administered in a dose of about 240 mg/m².

35. The method of claim 32, wherein Compound 1 is administered to the subject about 30 minutes prior to the administration of the at least one chemotherapeutic agent.

36. The method of claim 32, wherein the at least one chemotherapeutic agent is etoposide.

37. The method of claim 32, wherein the at least one chemotherapeutic agent is carboplatin.

38. The method of claim 32, wherein the at least one chemotherapeutic agent is topotecan.

39. The method of claim 38, wherein the topotecan is administered at a dose of 1.5 mg/m² or less.

40. The method of claim 39, wherein the topotecan is administered at a dose of about 1.25 mg/m².

41. The method of claim 39, wherein the topotecan is administered at a dose of about 0.75 mg/m².

42. The method of claim 32, wherein administration of a single dose of Compound 1 further provides a blood plasma profile mean Cmax of between about 1000 (ng/ml) and 3500 (ng/ml).

43. The method of claim 32, wherein administration of a single dose of Compound 1 further provides a blood plasma profile mean AUCₜ measured at 24.5 hours of at least about 2040 (ng*hr/ml).

44. A method of treating a human subject having small cell lung cancer comprising:
intravenously administering to the subject on each of day 1, day 2, and day 3 of a 21-day cycle a composition comprising Compound 1, or an acceptable salt thereof, that provides a blood plasma profile mean AUCₜ of Compound 1 measured at about 24.5 hours following administration of Compound 1 on day 3 of about 2000 (h*mg/ml) to about 4500 (ng*hr/ml),
subsequently administering to the subject a therapeutically effective amount of one or more chemotherapeutic agents on each of day 1, day 2, and day 3,
wherein the subject is administered carboplatin and etoposide on day 1,
wherein the subject is administered etoposide on day 2 and day 3, and
wherein at least one of the one or more chemotherapeutic agents is administered to the subject within about four hours or less of the administration of Compound 1.

45. The method of claim 44, wherein the blood plasma profile mean AUCₜ is between about 2300 (ng*hr/ml) and 4000 (ng*hr/ml).

46. The method of claim 44, wherein the blood plasma profile mean AUCₜ is about 2830 (ng*hr/ml)+/−about 20%.

47. The method of claim 44, wherein the blood plasma profile mean AUCₜ is at least 2040 (ng*hr/ml).

48. The method of claim 44, wherein the blood plasma profile mean AUCₜ is about 3020 (ng*hr/ml)+/−about 20%.

49. The method of claim 44, wherein the blood plasma profile mean dosage-corrected mean AUCₜ is at least about 8.35 ((ng*hr/ml)/(mg/m²)).

50. The method of claim 44, wherein Compound 1 is administered in a dose of between about 180 mg/m² and 280 mg/m².

51. The method of claim 44, wherein Compound 1 is administered each day in a dose of about 200 mg/m².

52. The method of claim 44, wherein Compound 1 is administered each day in a dose of about 240 mg/m².

53. The method of claim 44, wherein Compound 1 is administered to the subject about 30 minutes prior to the administration of at least one of the one or more chemotherapeutic agents.

54. The method of claim 44, wherein the 21 day cycle is repeated one or more times.

55. The method of claim 44, wherein the etoposide is administered to the subject at 100 mg/m².

56. The method of claim 44, wherein the carboplatin is administered to the subject at a dosage that achieves a target AUC of about 5 min*mg/m².

57. The method of claim 44, wherein administration of Compound 1 further provides a blood plasma profile mean Cmax of between about 1000 (ng/ml) and 3500 (ng/ml).

58. A method of treating a human subject having small cell lung cancer comprising:
intravenously administering to the subject on each of day 1, day 2, day 3, day 4, and day 5 of a 21-day cycle a composition comprising Compound 1, or an acceptable salt thereof, that provides a blood plasma profile mean AUCₜ of Compound 1 measured at about 24.5 hours following administration of Compound 1 on day 5 of about 2000 (h*mg/ml) to about 4500 (ng*hr/ml),
subsequently administering to the subject a therapeutically effective amount of topotecan on each of day 1, day 2, day 3, day 4, and day 5,
wherein topotecan is administered to the subject within about four hours or less of the administration of Compound 1.

59. The method of claim 58, wherein the blood plasma profile mean AUCₜ is between about 2300 (ng*hr/ml) and 4000 (ng*hr/ml).

60. The method of claim 58, wherein the blood plasma profile mean AUCₜ is about 2830 (ng*hr/ml)+/−about 20%.

61. The method of claim 58, wherein the blood plasma profile mean AUCₜ is at least 2040 (ng*hr/ml).

62. The method of claim 58, wherein the blood plasma profile mean AUCₜ is about 3020 (ng*hr/ml)+/−about 20%.

63. The method of claim 58, wherein the blood plasma profile dosage-corrected mean AUCₜ is at least about 8.35 ((ng*hr/ml)/(mg/m²)).

64. The method of claim 58, wherein Compound 1 is administered in a dose of between about 180 mg/m² and 280 mg/m².

65. The method of claim 65, wherein Compound 1 is administered each day in a dose of about 200 mg/m².

66. The method of claim 65, wherein Compound 1 is administered each day in a dose of about 240 mg/m².

67. The method of claim 58, wherein Compound 1 is administered to the subject about 30 minutes prior to the administration of topotecan.

68. The method of claim 58, wherein the 21 day cycle is repeated one or more times.

69. The method of claim 58, wherein the topotecan is administered to the subject each day at a dosage of 1.5 mg/m².

70. The method of claim 58, wherein the topotecan is administered to the subject each day at a dosage of 1.25 mg/m².

71. The method of claim 58, wherein the topotecan is administered to the subject each day at a dosage of 0.75 mg/m².

72. The method of claim 58, wherein administration of a single dose of Compound 1 further provides a blood plasma profile mean Cmax of between about 1000 (ng/ml) and 3500 (ng/ml).

73. A method of treating a human subject having small cell lung cancer comprising:
intravenously administering to the subject on each of day 1, day 2, and day 3 of a 21-day cycle a composition comprising Compound 1, or an acceptable salt thereof, that provides a blood plasma profile mean AUCₜ of Compound 1 measured at about 24.5 hours following administration of Compound 1 on day 3 of about 2000 (h*mg/ml) to about 4500 (ng*hr/ml),
subsequently administering to the subject a therapeutically effective amount of topotecan on each of day 1, day 2, and day 3,
wherein topotecan is administered to the subject within about four hours or less of the administration of Compound 1, and
wherein topotecan is administered in a dosage of 1.25 mg/m².

74. The method of claim 73, wherein the blood plasma profile mean AUCₜ is between about 2300 (ng*hr/ml) and 4000 (ng*hr/ml).

75. The method of claim 73, wherein the blood plasma profile mean AUCₜ is about 2830 (ng*hr/ml)+/−about 20%.

76. The method of claim 73, wherein the blood plasma profile mean AUCₜ is at least 2040 (ng*hr/ml).

77. The method of claim 73, wherein the blood plasma profile mean AUCₜ is about 3020 (ng*hr/ml)+/−about 20%.

78. The method of claim 73, wherein the blood plasma profile dosage-corrected mean AUCₜ is at least about 8.35 ((ng*hr/ml)/(mg/m²)).

79. The method of claim 73, wherein Compound 1 is administered in a dose of between about 180 mg/m² and 280 mg/m².

80. The method of claim 79, wherein Compound 1 is administered each day in a dose of about 200 mg/m².

81. The method of claim 79, wherein Compound 1 is administered each day in a dose of about 240 mg/m².

82. The method of claim 73, wherein Compound 1 is administered to the subject about 30 minutes prior to the administration of topotecan.

83. The method of claim 73, wherein the 21 day cycle is repeated one or more times.

84. The method of claim 73, wherein administration of a single dose of Compound 1 further provides a blood plasma profile mean Cmax of between about 1000 (ng/ml) and 3500 (ng/ml).
